(19) United States
(12) Patent Application Publication
(10) Pub. No.: US 2025/0381261 A1
(43) Pub. Date: Dec. 18, 2025

(54) ZIKA VACCINES AND IMMUNOGENIC COMPOSITIONS, AND METHODS OF USING THE SAME

(71) Applicant: Takeda Vaccines, Inc., Cambridge, MA (US)

(72) Inventors: Camilo Acosta, Cambridge, MA (US); Htay Htay Han, Cambridge, MA (US); John Boslego, Cambridge, MA (US); Gary Dubin, Zurich (CH)

(21) Appl. No.: 18/835,922

(22) PCT Filed: Feb. 9, 2022
(86) PCT No.: PCT/US2022/015821

§ 371 Date: Aug. 5, 2024

(51) Int. Cl. A61K 39/12; A61K 39/00; A61P 37/04

(52) U.S. Cl. CPC A61K 39/12; A61P 37/04; A61K 2039/5252; A61K 2039/545; A61K 2039/55505

(57) ABSTRACT

The present disclosure relates to vaccines and immunogenic compositions comprising an antigen from a Zika virus (e.g., wherein the antigen is an inactivated whole Zika virus), and their use in medical applications (such as methods of treatment).

### CROSS REFERENCE TO RELATED APPLICATIONS

This application is a National Phase application filed under 35 USC § 371 of International Application No. PCT/US2022/015821, filed on Feb. 9, 2022, which is incorporated by reference in its entirety.

### STATEMENT REGARDING FEDERALLY-SPONSORED RESEARCH

This invention was made with government support under Contract No. HHSO100201600015C awarded by the Department of Health and Human Services, Office of the Assistant Secretary for Preparedness and Response, Biomedical Advanced Research and Development Authority. The Government has certain rights in the invention.

### SEQUENCE LISTING

This application incorporates by reference in its entirety the Sequence Listing entitled “T08289WO6_Sequence Listing” created on Feb. 9, 2022 at 5:48 pm that is 115 KB and filed electronically herewith.

### FIELD OF THE INVENTION

The present disclosure relates to Zika virus vaccines and immunogenic compositions comprising a Zika virus and medical applications thereof.

### BACKGROUND

Zika virus is a flavivirus classified with other mosquito-borne viruses (e.g., yellow fever, dengue, West Nile, and Japanese encephalitis viruses) within the Flaviviridae family. Initially isolated in 1947 in Uganda, Zika virus was first linked to human disease in 1952, and has been recognized sporadically as a cause of mild, self-limited febrile illness in Africa and Southeast Asia (Weaver et al. (2016) Antiviral Res. 130:69-80; Faria et al. (2016) Science. 352 (6283): 345-349). However, in 2007, an outbreak appeared in the North Pacific island of Yap, and then disseminated from island to island across the Pacific, leading to an extensive outbreak in 2013-2014 in French Polynesia, spreading then to New Caledonia, the Cook Islands, and ultimately, to Easter Island. An Asian lineage virus was subsequently transferred to the Western Hemisphere by routes that remain undetermined (Faria et al. (2016) Science. 352 (6283): 345-349).

Zika virus may be transmitted zoonotically by Aedes aegypti, A. albopictus, and possibly by A. hensilli and A. polynieseinsis (Weaver et al. (2016) Antiviral Res. 130:69-80). Additionally, it is thought that other vectors for transmitting the virus may exist, and the virus may be transmitted by blood transfusion, transplacentally, and/or through sexual transmission.

Although in the general population, most Zika virus infections are asymptomatic, and symptomatic infections are generally mild, the infection can result in serious neurological complications such as meningoencephalitis, myelitis, and Guillain-Barre syndrome (GBS). GBS is a rapid-onset muscle weakness caused by the immune system damaging the peripheral nervous system occurring post-infection. GBS has a mortality rate of 5%, and 20% of the affected patients remain with significant disability. The mortality rate increases even to 10-35% if the affected individual is a pregnant woman. Moreover, a distinct pattern of birth defects and disabilities, called congenital Zika syndrome (CZS), has been found with Zika virus infection during pregnancy. Several features are observed in the context of CZS such as severe microcephaly in which the skull has partially collapsed, decreased brain tissue with a specific pattern of brain damage including subcortical calcifications, damage to the back of the eye including macular scarring and focal retinal pigmentary mottling, congenital contractures, such as clubfoot or arthrogryposis, and hypertonia restricting body movement soon after birth. Fetuses of women infected with Zika virus during pregnancy have a 5 to 14% risk of developing CZS. Both symptomatic and asymptomatic Zika virus infections in pregnancy have been reported to cause Zika virus-related birth abnormalities (Marban-Castro et al., 2021, European Journal of Obstetrics & Gynecology and Reproductive biology, 265, 162-168).

Due to the significant increase in fetal abnormalities (e.g., microcephaly) and higher incidences of Guillain-Barré syndrome (GBS) in areas of widespread Zika virus infection, the World Health Organization (WHO) declared Zika a Public Health Emergency of International Concern (PHEIC) (Heymann et al. (2016) Lancet 387 (10020): 719-21). Although the WHO has since declared an end to the PHEIC and the number of Zika cases have declined over the last few years, Zika continues to pose a significant threat, in particular, for pregnant women and their unborn babies. Further major outbreaks of Zika can occur without prior warning.

However, as there is neither an FDA-approved Zika vaccine yet nor an efficient treatment existing, the only preventative measures for controlling Zika virus involve managing mosquito populations. Thus, there is an urgent need to develop vaccines or immunogenic compositions well-suitable for inducing an immune response against Zika virus and/or for preventing Zika virus disease in the subject that is administered the vaccines or immunogenic composition.

### OBJECTS AND SUMMARY

To meet the above mentioned and other needs, the present disclosure is directed, at least in part, to the provision of a vaccine or immunogenic composition comprising an antigen of a Zika virus, preferably wherein the antigen is an inactivated whole Zika virus. The vaccine or immunogenic composition of the present disclosure is well-tolerated and highly immunogenic in human subjects even after one single administration (cf. also Example 6 below).

In particular, it is an object of the present disclosure to provide a method for inducing an immune response against Zika virus and/or for preventing Zika virus disease and/or for preventing Zika virus infection in a human subject or in individuals of a human subject population. It is a specific object of the present disclosure to provide a method for inducing an immune response against Zika virus and/or for preventing Zika virus disease and/or for preventing Zika virus infection in a human subject or individuals in a human subject population, wherein the human subjects or the individuals of the human subject population are flavivirus-primed subjects with exposure to flavivirus(es) prior to vaccination.

Moreover, it is one object of the present disclosure to provide an administration regimen for the vaccine or immunogenic composition of the present disclosure, the administration regimen resulting in the induction of high seroconversion and/or seropositivity rates in human subjects and in the long persistence of such high seroconversion and/or seropositivity rates. The administration regimen of the present disclosure is thus offering multiple advantages, for instance, reducing costs for vaccination and increasing patient comfort due to the lower number of administrations.

It is a further object of the present disclosure to provide a vaccine or immunogenic composition that is safe and well-tolerated in a huge number of human subjects, in particular flavivirus-naïve and flavivirus-primed human subjects. It is a further object of the present disclosure to provide a vaccine or immunogenic composition which does, when administered to a human subject, not lead to serious adverse events during a clinical study duration as long as 2 years after the last vaccination.

Moreover, it is an object of the present disclosure to provide a vaccine or immunogenic composition that can be administered to pregnant women and/or women of childbearing potential and/or women that intend to become pregnant (shortly) after vaccination. Female human subjects that were administered the vaccine or immunogenic composition according to the present disclosure gave birth to healthy newborns (cf. Example 6 below). “Of childbearing potential” is defined as status post onset of menarche and not meeting any of the following conditions: menopausal for at least 2 years without any other alternative medical cause (as confirmed by healthcare professional), status after bilateral tubal ligation for at least 1 year, status after bilateral oophorectomy, or status after hysterectomy.

Further, it is an object of the present disclosure to provide a vaccine or immunogenic composition that is highly immunogenic in both flavivirus-naïve and flavivirus-primed human subjects, already after only one administration even with doses as low as, for instance, 2 μg of Zika virus. This is particular advantageous in an outbreak situation or for a traveler visiting an endemic area within a short period of time from the administration of the vaccine or immunogenic composition.

It is a further object of the present disclosure to provide a vaccine or immunogenic composition that provides for long-lasting immunogenicity in both, flavivirus-naïve and flavivirus-primed human subjects. In particular, it is an object of the present disclosure to provide a vaccine or immunogenic composition that provides for high seroconversion and/or seropositivity up to 6 months or up to 12 months or up to 24 months after the last vaccination. Such high seroconversion and/or seropositivity avoids the need for a booster administered early after the last dose of the primary administration regimen. Long last immunity is a highly desired feature of a vaccine/immunogenic composition. However, inactivated vaccines do usually not provide for high immune responses (high seroconversion/seropositivity rates) and require the administration of a booster soon after the second dose.

It is a further object of the present disclosure to provide a Zika virus for application in the vaccine or immunogenic composition of the present disclosure, which is on the one hand adapted to efficient growth in non-human cells (such as Vero cells) and at the same time genetically stable during cell culture passaging, allowing for the production of high titers of genetically homogenous viruses. This is particularly beneficial for vaccine production, as repeatability of the production process can be guaranteed and a lot-to-lot consistency. The Zika virus according to the present disclosure is thus particularly useful as a master virus seed (MVS) for vaccine production and manufacturing, such as, for instance, production and manufacturing of an inactivated whole virus vaccine, where no mutations (in particular in the immunogenic epitopes, such as epitopes on the envelope protein of Zika virus) are desired. In general, by using the Zika virus according to the present disclosure as a MVS, the risk of the development of further undesirable mutations during further passaging and/or virus production is markedly reduced. In certain embodiments, the genetically stable Zika virus of the present disclosure harbors an adaptation mutation in the non-structural protein 1 (NS1). Without wishing to be bound by theory, the adaptation mutation is thought to suppress the occurrence of further mutations during passaging, in particular, in the structural envelope (E) protein, while at the same time resulting in increased/enhanced replication efficiency allowing for efficient vaccine production. The term “adaptation mutation” refers to a mutation that occurs and/or accumulates during cell culture passaging, such as passaging on Vero cells (“non-human cell culture adaptation mutation” refers to a mutation that occurs and/or accumulates during cell culture passaging in non-human cells, such as Vero cells). In some embodiments further outlined below, the adaptation mutation occurs at position 98 of SEQ ID NO: 9, or at a position corresponding to position 98 of SEQ ID NO: 9. In some embodiments, the adaptation mutation is a Trp98Gly mutation. The genetically stable Zika virus of the present disclosure can be used in the vaccine or immunogenic compositions of the present disclosure and the methods described herein below.

The Zika virus for application in the vaccines or immunogenic compositions of the present disclosure is described in more detail in the description below (including the Examples), in the sequence listing, and in the claims. Further, methods for production and inactivation of a Zika virus, as well as for determining the completeness of inactivation of said Zika virus are provided in the description below (including the Examples) and in the claims. Further components that might be included in the vaccines or immunogenic compositions of the present disclosure such as adjuvants are also described in more detail below. Other embodiments (such as methods and uses) are further described in more detail in the description (including the Examples) below and in the claims.

As will be readily understood by the person skilled in the art, the various embodiments disclosed herein may be combined amongst each other to form new embodiments that are also within the teaching of the present disclosure.

### BRIEF DESCRIPTION OF THE DRAWINGS

FIG. 1 Amino acid sequence alignment comparing regions of the NS1 protein sequence of Zika virus near residue 98 of NS1 from Zika virus strains PRVABC59 P6e (Pre-MVS; SEQ ID NO: 12) and PRVABC59 wild type (GenBank reference sequence KU501215; SEQ ID NO: 13) with corresponding regions of the NS1 protein sequence of several other flaviviruses, i.e. West Nile virus (WNV; SEQ ID NO: 14), Japanese Encephalitis virus (JEV; SEQ ID NO: 15), St. Louis Encephalitis virus (SLEV; SEQ ID NO: 16), Yellow Fever virus (YFV; SEQ ID NO: 17), dengue serotype 1 (DENV 1) strain 16007 (SEQ ID NO: 18), dengue serotype 2 (DENV 2) strain 16681 (SEQ ID NO: 19), dengue serotype 3 (DENV 3) strain 16562 (SEQ ID NO: 20), and dengue serotype 4 (DENV 4) strain 1036 (SEQ ID NO: 21).

FIG. 2 Bright field microscopy images of Vero cell monolayers mock infected (top) or infected with Zika virus strain PRVABC59 (bottom).

FIG. 3 Growth kinetics of Zika virus PRVABC59 P1 on Vero cell monolayers, as determined by TCID₅₀.

FIG. 4 Potency assay testing (TCID₅₀) of Zika virus PRVABC59 P5 clones a-f.

FIG. 5 Bright-field microscopy images depicting the cytopathic effect (CPE) of growth of Zika virus PRVABC59 P6 clones a-f on Vero cell monolayers.

FIG. 6 Potency assay testing (TCID₅₀) of Zika virus PRVABC59 P6 clones a-f

FIG. 7 Plaque phenotype of Zika virus PRVABC59 P6 virus clones a-f compared to Zika virus PRVABC59 P1 virus.

FIG. 8 Mean plaque size of Zika virus PRVABC59 P6 virus clones compared to Zika virus PRVABC59 P1 virus

FIG. 9 Growth kinetics of Zika virus PRVABC59 P6 clones a-f in Vero cells under serum-free growth conditions.

FIG. 10 Schematic of the steps taken to prepare PRVABC59 P6b and P6e formulated drug product for the immunization experiments.

FIG. 11 Schedule of dosing of CD-1 mice with vaccine formulations derived from the Zika virus PRVABC59 P6b and P6e clones. PBS was used as placebo (upper section). Serum Zika virus neutralizing antibody titers of CD-1 mice immunized as shown in the upper panel using vaccine formulations derived from Zika virus PRVABC59 P6b and P6e clones. Zika virus neutralizing antibody titers were determined by Reporter Virus Particle (RVP) neutralization assay. Solid lines represent the geometric mean of a group. The limit of detection (1.93 log 10) is represented by a dashed line (lower section). Alum=aluminum hydroxide adjuvant.

FIG. 12 Schedule of dosing of AG129 mice with vaccine formulations derived from the Zika virus PRVABC59 P6b and P6e clones. PBS was used as a placebo (upper section). Serum Zika virus neutralizing antibody titers of AG129 mice immunized as shown in the upper panel using vaccine formulations derived from Zika virus PRVABC59 P6b and P6e clones. Solid lines represent the geometric mean of a group. The limit of detection (1.30 log 10) is represented by a dashed line. Animals with no detectable titer (<1.30) were assigned a titer of 0.5 (lower section). Alum=aluminum hydroxide adjuvant.

FIG. 13 Mean weight of AG129 test groups post-challenge, represented as a percentage of starting weight. Error bars represent standard deviation. Alum=aluminum hydroxide adjuvant.

FIG. 14 Serum viremia of individual AG129 mice two days post-challenge, reported as PFU/mL. Solid lines represent the mean of a group. The limit of detection (2.0 log 10) is represented by a dashed line. Alum=aluminum hydroxide adjuvant.

FIG. 15 Survival analysis of AG129 test groups post-challenge. Alum=aluminum hydroxide adjuvant.

FIG. 16 Pre-challenge serum circulating Zika virus neutralizing antibody (Nab) titers following passive transfer of pooled sera from vaccinated and challenged AG129 mice.

FIG. 17 Mean body weight of passive transfer and control mice challenged with Zika virus.

FIG. 18 Serum viremia of individual AG129 mice three days post-challenge, reported as PFU/mL.

FIG. 19 Survival analysis of passive transfer and control mice challenged with Zika virus.

FIG. 20 Correlation between Zika virus neutralizing antibody titers (EC₅₀) and viremia (PFU/mL) observed in passive transfer mice.

FIG. 21 Survival analysis of AG129 mice after challenge with stocks of P6a and P6e using a Kaplan Meier survival curve. (PRV=Puerto Rican Virus)

FIG. 22 Mean body weight as expressed in percentage of starting weight at different time points after challenge with stocks of P6a and P6e. The dashed line represents 100% of starting weight for reference.

FIG. 23 Serum viremia of individual AG129 mice three days post-challenge with stocks of P6a and P6e, reported as PFU/mL. The dashed line represents the limit of detection of the assay.

FIG. 24 Compiled kinetics of inactivation data. Data compares infectious potency (TCID₅₀) to RNA copy, and completeness of inactivation (COI) for samples from the four toxicology lots. These data indicate that the sensitivity of the COI assay (double-infectivity assay) is greater than TCID₅₀.

FIG. 25 Comparison of C6/36 and Vero sensitivity in the assay as demonstrated with an input virus titer of 0.31 TCID₅₀.

FIG. 26 shows a logistic regression analysis of CPE vs. log TCID₅₀ using C6/36 cells site that include 99% confidence intervals around a target value of 0.01 TCID₅₀/well (−2 log TCID₅₀/well); the model predicts 0.85% of wells will be positive.

FIG. 27 Chromatograms of PBS (a) and PBS solutions containing 0.049 μg/mL (b), 0.098 μg/mL (c), 0.196 μg/mL (d), 0.491 μg/mL (e), 0.982 μg/mL (f), and 1.964 μg/mL (g) formaldehyde.

FIG. 28 Clinical study design for ZIK-101 (Phase I study as described in Example 6). CSR=Clinical study report. μ=μg.

FIG. 29 Clinical trial profile for flavivirus naïve individuals (Part 1 of ZIK-101).

FIG. 30 Clinical trial profile for flavivirus primed individuals (Part 2 of ZIK-101).

FIG. 31 Clinical trial profile and number of subjects throughout the study duration.

FIG. 32 GMTs (Geometric mean titers) of neutralizing antibodies until study day 57 measured by plaque reduction neutralization test (PRNT) shown as PRNT₅₀. Error bars show 95% Confidence Intervals.

FIG. 33 GMTs (Geometric mean titers) of neutralizing antibodies until study day 57 measured by reporter virus particle (RVP) assay shown as RVP₅₀ (i.e. EC₅₀). Error bars show 95% Confidence Intervals.

FIG. 34 Neutralizing antibodies measured by PRNT (presented as geometric mean titers, GMT) in flavivirus naïve and flavivirus primed participants receiving either placebo or 10 μg PIZV throughput the study duration (PRNT₅₀ referred to as EC₅₀).

FIG. 35 Neutralizing antibodies measured by RVP assay (presented as geometric mean titers, GMT) in flavivirus naïve and flavivirus primed participants receiving either placebo or 10 μg PIZV throughput the study duration. Presented are EC₅₀ (or RVP₅₀, respectively).

FIG. 36 Plot of the percentage of subjects achieving a particular GMT (determined using PRNT) on day 29 (day 28 after prime dose).

FIG. 37 Plot of the percentage of subjects achieving a particular GMT (determined using PRNT) on day 57 (day 56 after prime dose).

FIG. 38 Seropositivity rates in flavivirus-primed subjects measured with PRNT at study days 57, 211, and 393.

FIG. 39 Seropositivity rates in flavivirus-primed subjects measured with PRNT at study days 393 and 757.

FIG. 40 Seropositivity rates in flavivirus-primed subjects measured with RVP assay at study days 1, 29, 57, 211, 393, and 757 (visits 2, 4, 6, 8, 9, and 11, respectively). PB=placebo.

FIG. 41 Seroconversion rates in flavivirus naïve subjects as determined with the PRNT on study days 29 and 57.

FIG. 42 Seroconversion rates in flavivirus naïve subjects as determined with the PRNT on study days 57, 211, and 393.

FIG. 43 Seroconversion rates in flavivirus naïve subjects as determined with the PRNT on study days 393 and 757.

FIG. 44 Seroconversion rates in flavivirus primed subjects as determined with the PRNT on study days 29 and 57.

FIG. 45 Seroconversion rates in flavivirus primed subjects as determined with the PRNT on study days 57, 211, and 393.

FIG. 46 Seroconversion rates in flavivirus primed subjects as determined with the PRNT on study days 393 and 757.

FIG. 47 Seroconversion rates in flavivirus naïve subjects as determined with the RVP assay on study days 29, 57, 211, 393 and 757.

FIG. 48 Seroconversion rates in flavivirus primed subjects as determined with the RVP assay on study days 29, 57, 211, 393 and 757.

FIG. 49 Schematic representation of Phase II trial design (Example 7).

### DETAILED DESCRIPTION

Unless clearly indicated otherwise, use of the terms “a”, “an”, “the” and the like refer to one or more.

The term “A and/or B” is intended to encompass “A”, “B”, and “A and B”.

Within the meaning of this disclosure, when numbers (e.g. percentages) are given as whole numbers, the numbers (e.g. percentages) are to be understood to cover values that result, when rounded up, in this whole number. For instance, a percentage of 90% is to be understood to also cover percentages of 89.5% to 90.4%.

### General Techniques and Methods

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Antibodies, A Laboratory Manual (Harlow and Lane, eds. (1988), and Animal Cell Culture (R. I. Freshney, ed. (1987)); Oligonucleotide Synthesis (M. J. Gait, ed., 1984); Methods in Molecular Biology (J. M. Walker, ed. Humana Press (1983)); Cell Biology: A Laboratory Notebook (J. E. Celis, ed., Academic Press (1998)) Academic Press; Animal Cell Culture (R. I. Freshney), ed., 1987); Introduction to Cell and Tissue Culture (J. P. Mather and P. E. Roberts, eds. Plenum Press (1998)); Cell and Tissue Culture: Laboratory Procedures (A. Doyle, J. B. Griffiths, and D. G. Newell, eds., J. Wiley and Sons (1993-8)); Handbook of Experimental Immunology (D. M. Weir and C. C. Blackwell, eds.); Gene Transfer Vectors for Mammalian Cells (J. M. Miller and M. P. Calos, eds., Cold Spring Harbor Laboratory (1987)); PCR: The Polymerase Chain Reaction, (Mullis et al., eds., Springer (1994)); Current Protocols in Immunology (J. E. Coligan et al., eds., Wiley (1991)); Short Protocols in Molecular Biology (Wiley and Sons, 1999); Immunobiology (C. A. Janeway and P. Travers, (1997)); Antibodies (P. Finch, 1997); Antibodies: A Practical Approach (D. Catty., ed., IRL Press, (1988-1989)); Monoclonal Antibodies: A Practical Approach (P. Shepherd and C. Dean, eds., Oxford University Press, (2000)); Using Antibodies: A Laboratory Manual (E. Harlow and D. Lane (Cold Spring Harbor Laboratory Press, (1999)); and The Antibodies (M. Zanetti and J. D. Capra, eds., Harwood Academic Publishers, (1995)). The above-mentioned references are hereby incorporated by reference.

### Determination of Sequence Identities and Preparing Sequence Alignments

“Sequence Identity”, “% sequence identity”, “% identity”, or “% identical” refers to the degree of identity of a first amino acid sequence to a second amino acid sequence, or to the degree of identity of a first nucleic acid sequence to a second nucleic acid sequence and is calculated as a percentage based on a comparison between the two sequences.

According to the present disclosure, the sequence identity of two sequences is determined by counting mismatches at a single position and gaps at a single position as non-identical positions in the final sequence identity calculation. The sequence identity is determined by a program, which produces a pairwise alignment, and calculates the identity between the two aligned sequences counting both mismatches at a single position and gaps at a single position as non-identical positions.

Sequence identity can be calculated from a pairwise alignment of two sequences over the full length of both sequences (“global sequence identity”). A sequence identity can also be calculated from a pairwise alignment of the local regions of the first sequence and the second sequence that show identity or similarity (“local sequence identity”). For instance, if a first sequence has 1000 characters and a second sequence has 800 characters and the 800 characters of the second sequence are encompassed without gaps in the first sequence, the global sequence identity between the first and the second sequence is 80%, whereas the local sequence identity between the first and the second sequence is 100%.

If not indicated otherwise, a sequence identity within the meaning of this disclosure refers to a sequence identity that is calculated from a pairwise alignment taken into account both sequences over their full length (e.g. comparing Zika virus genomic sequences over their full lengths), i.e. refers to the “global sequence identity”.

An exemplary program for determining a “global sequence identity” is the “Needle” (The European Molecular Biology Open Software Suite, EMBOSS) program (https://www.ebi.ac.uk/Tools/psa/emboss_needle/), which has implemented the algorithm of Needleman and Wunsch (Needleman and Wunsch, 1970, J. Mol. Biol. 48:443-453) and which calculates sequence identity per default settings by first producing an alignment between a first sequence and a second sequence, then counting the number of identical positions over the length of the alignment, then dividing the number of identical residues by the length of an alignment, then multiplying this number by 100 to generate the % sequence identity [% sequence identity=(# of Identical residues/length of alignment)×100)]. As explained, the alignment produced by the Needle program is produced over the complete sequence lengths, resulting in the “global sequence identity”.

In preferred embodiments, a % sequence identity and/or a sequence alignment is generated by using the algorithm of Needleman and Wunsch (J. Mol. Biol. (1979) 48, p. 443-453). In preferred embodiments, the program “Needle” (EMBOSS) is used. In even more preferred embodiments, the program “Needle” (EMBOSS) is used with the programs default parameter (gap opening penalty=10.0, gap extension penalty=0.5 and matrix=EBLOSUM62 for proteins and matrix=EDNAFULL for nucleotides).

Alignments showing the “local sequence identity” can, for example, be produced by the Blast algorithm (NCBI).

### SEQUENCE LISTING

The following Table (Table 1) provides an overview of the sequences in the sequence listing of the present application:

| SEQ ID NO: | Description |
|---|---|
| 1 | Zika virus strain PRVABC59 genome sequence |
| | Presented as DNA |
| | Refers to GenBank ID No. KU501215.1 |
| 2 | Zika virus strain PRVABC59 polyprotein sequence |
| | Refers to GenBank ID No. AMC13911.1 |
| 3 | Zika Pre-Master Virus Seed (Pre-MVS) genome sequence |
| | Presented as DNA; carrying a guanine (G) at position 292 of the NS1 gene |
| | Refers to PRVABC59-P6e as described in the Examples |
| 4 | Zika Pre-Master Virus Seed (Pre-MVS) and Master Virus Seed (MVS) |
| | polyprotein sequence |
| | Carrying a glycine (Gly) at position 98 of the NS1 protein |
| | Refers to PRVABC59-P6e and -P7e as described in the Examples |
| 5 | Zika Master Virus Seed (MVS) genome sequence |
| | Presented as DNA; carrying a guanine (G) at position 292 of the NS1 gene |
| | Refers to PRVABC59-P7e as described in the Examples |
| 6 | Zika virus strain PRVABC59, Pre-MVS and MVS envelope (E) protein |
| | sequence |
| 7 | Zika virus strain PRVABC59, Pre-MVS and MVS premembrane/membrane |
| | (prM/M) protein sequence |
| 8 | Zika Pre-MVS and MVS NS1 protein sequence |
| | Carrying a glycine (Gly) at position 98 |
| 9 | Zika virus strain PRVABC59 NS1 protein sequence |
| 10 | Zika Pre-MVS and MVS NS1 gene sequence |
| | Carrying a guanine (G) at position 292 |
| 11 | Zika virus strain PRVABC59 NS1 gene sequence |

The above listed nucleotide sequences disclosed in the sequence listing are presented as DNA in the 5′ (free phosphate-group)→3′ (free hydroxyl-group) direction. A Zika virus is an RNA virus comprising a positive-sense (5′→3′), single-stranded RNA genome. Thus, certain aspects of the present disclosure refer to a Zika virus comprising an RNA genome sequence “characterized by” a certain DNA sequence, such as the DNA sequences of the sequence listing. As the skilled person will appreciate, a Zika virus RNA genome sequence referred to as being “characterized by” a certain DNA sequence, refers to a Zika virus RNA genome sequence being the corresponding RNA to the DNA sequence. A corresponding RNA to a DNA sequence can be generated/determined by replacing the nucleotide thymine (T) with the nucleotide uracil (U). As the DNA sequences of the sequence listing are presented in the 5′→3′ direction, the corresponding Zika virus RNA genomes (which are positive-sense) can be determined by replacing the nucleotide T with U. No further corrections for the nucleotide sense have to be made.

As will be appreciated by a skilled artisan, the length of the sequenced genome may vary, depending on the sequencing strategy and primers used. In the present disclosure, the length of the wild-type PRVABC59 genomic sequence (SEQ ID NO: 1), the Pre-MVS genomic sequence (SEQ ID NO: 3), and the MVS genomic sequence (SEQ ID NO: 5) slightly vary due to different sequencing set-ups/strategies (e.g. due to different primers used). The variations do, however, occur at the terminal parts of the genome, i.e. the non-coding 3′- and 5′-regions.

### Zika Virus and Corresponding Antigens

Zika virus is a mosquito-borne flavivirus first isolated from a sentinel rhesus monkey in the Zika Forest in Uganda in 1947. Since that time, isolations have been made from humans in both Africa and Asia, and more recently, the Americas. Zika viruses that have been isolated from a sample of a patient who is infected with Zika virus are also referred to as clinical isolates. Zika virus is currently grouped in two lineages: an African lineage (possibly separate East and West African lineages) and an Asian lineage.

For the preparation of the vaccine or immunogenic composition of the present disclosure, an antigen from any Zika virus may be used. In some embodiments, the Zika virus is an African lineage virus. In some embodiments, the Zika virus is an Asian lineage virus. In preferred embodiments, the Zika virus is an Asian lineage virus. Suitable Zika viruses for use in the production of the vaccines or immunogenic compositions of the present disclosure are exemplary outlined below in this section.

In certain preferred embodiments, the antigen from Zika virus is an inactivated whole Zika virus. Suitable methods of virus inactivated are outlined below in the section “Zika virus inactivation”. Within the meaning of the disclosure and as will be appreciated by one skilled in the art, the term “whole Zika virus” refers to the complete virus and not to only a single protein or a subunit of a single protein of the virus. Suitable (whole) Zika viruses are described below in this section. When reference is made to “Zika virus” within the present disclosure, the reference refers to a whole virus unless indicated otherwise.

Multiple strains within the African and Asian lineages of Zika virus have been previously identified. Any one or more suitable strains of Zika virus known in the art may be used in the present disclosure, including, for examples, strains Mr 766, ArD 41519, IbH 30656, P6-740, EC Yap, FSS13025, ArD 7117, ArD 9957, ArD 30101, ArD 30156, ArD 30332, HD 78788, ArD 127707, ArD 127710, ArD 127984, ArD 127988, ArD 127994, ArD 128000, ArD 132912, 132915, ArD 141170, ArD 142623, ArD 149917, ArD 149810, ArD 149938, ArD 157995, ArD 158084, ArD 165522, ArD 165531, ArA 1465, ArA 27101, ArA 27290, ArA 27106, ArA 27096, ArA 27407, ArA 27433, ArA 506/96, ArA 975-99, Ara 982-99, ArA 986-99, ArA 2718, ArB 1362, Nigeria68, Malaysia66, Kedougou84, Suriname, MR1429, PRVABC59, ECMN2007, DakAr41524, H/PF/2013, R103451, 103344, 8375, JMB-185, ZIKV/H, sapiens/Brazil/Natal/2015, SPH2015, ZIKV/Hu/Chiba/S36/2016, and/or Cuba2017. In some embodiments, Zika virus strain PRVABC59 (GenBank Ref. KU501215.1) is used for production of the vaccines or immunogenic compositions of the present disclosure.

Zika viruses are enveloped viruses possessing a positive sense, single-stranded RNA genome encoding both structural and nonstructural proteins. The genome also contains non-coding sequences at both the 5′- and 3′-terminal regions that play a role in virus replication. The RNA genome is composed of approximately 10.8 kilobases (kb) encoding 10 genes within one single open reading frame (ORF). The Zika virus RNA genome is expressed as a single polyprotein (derived from the single ORF) that is processed inside of the host cell. The polyprotein encoded by the Zika virus RNA genome comprises the Zika virus structural proteins and the Zika virus non-structural proteins. The structural proteins are capsid (C) protein, precursor membrane (“premembrane”)/membrane (prM/M) protein, and envelope (E) protein. The non-structural proteins (NS) are NS1, NS2A, NS2B, NS3, NS4A, NS4B, and NS5. A schematic representation of the Zika virus genome is reproduced below (black parts=non-coding regions):

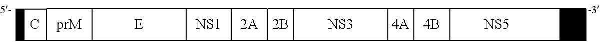

Determining which regions of a Zika virus genome encode which structural or non-structural proteins or which regions of a Zika virus polyprotein represent which structural or non-structural proteins is within the knowledge of the skilled person. Such sequence regions are referred to as “corresponding sequence parts” herein. For instance, the skilled person is able to determine the corresponding sequence part of a Zika virus genome encoding the envelope protein by comparison with known Zika virus envelope protein encoding sequences. The skilled person is, for instance, also able to determine the corresponding sequence part of a Zika virus polyprotein representing the envelope protein sequence by comparison with known Zika virus envelope protein sequences.

For example, corresponding sequence parts of SEQ ID NO: 1 (PRVABC59 genome) encoding the structural and non-structural proteins as well as corresponding sequence parts of SEQ ID NO: 2 (PRVABC59 polyprotein) representing the structural and non-structural proteins are described in Table 2 below.

TABLE 2
Genome and polyprotein structure of PRVABC59
(SEQ ID NO: 1 and SEQ ID NO: 2).

| | Gene | |
|---|---|---|
| | Corresponding sequence in | Corresponding part in |
| Protein | SEQ ID NO: 1 | SEQ ID NO: 2 |
| C | Nucleotides 107 to 472 | Amino acids 1 to 122 |
| prM/M | Nucleotides 473 to 976 | Amino acids 123 to 290 |
| E | Nucleotides 977 to 2488 | Amino acids 291 to 794 |
| NS1 | Nucleotides 2489 to 3544 | Amino acids 795 to 1146 |
| NS2A | Nucleotides 3545 to 4222 | Amino acids 1147 to 1372 |
| NS2B | Nucleotides 4223 to 4612 | Amino acids 1373 to 1502 |
| NS3 | Nucleotides 4613 to 6463 | Amino acids 1503 to 2119 |
| NS4A | Nucleotides 6464 to 6913 | Amino acids 2120 to 2269 |
| NS4B | Nucleotides 6914 to 7666 | Amino acids 2270 to 2520 |
| NS5 | Nucleotides 7667 to 10375 | Amino acids 2521 to 3423 |

For example, corresponding sequence parts of SEQ ID NO: 3 (Pre-MVS genome, P6e) encoding the structural and non-structural proteins as well as corresponding sequence parts of SEQ ID NO: 4 (Pre-MVS polyprotein, P6e) representing the structural and non-structural proteins are described in Table 3 below.

TABLE 3
Genome and polyprotein structure of Zika virus
Pre-MVS, P6e (SEQ ID NO: 3 and SEQ ID NO: 4).

| | Gene | |
|---|---|---|
| | Corresponding sequence in | Corresponding part in |
| Protein | SEQ ID NO: 3 | SEQ ID NO: 4 |
| C | Nucleotides 108 to 473 | Amino acids 1 to 122 |
| prM/M | Nucleotides 474 to 977 | Amino acids 123 to 290 |
| E | Nucleotides 978 to 2489 | Amino acids 291 to 794 |
| NS1 | Nucleotides 2490 to 3545 | Amino acids 795 to 1146 |
| NS2A | Nucleotides 3546 to 4223 | Amino acids 1147 to 1372 |
| NS2B | Nucleotides 4224 to 4613 | Amino acids 1373 to 1502 |
| NS3 | Nucleotides 4614 to 6464 | Amino acids 1503 to 2119 |
| NS4A | Nucleotides 6465 to 6914 | Amino acids 2120 to 2269 |
| NS4B | Nucleotides 6915 to 7667 | Amino acids 2270 to 2520 |
| NS5 | Nucleotides 7668 to 10376 | Amino acids 2521 to 3423 |

For example, corresponding sequence parts of SEQ ID NO: 5 (MVS genome, P7e) encoding the structural and non-structural proteins as well as corresponding sequence parts of SEQ ID NO: 4 (MVS polyprotein, P7e) representing the structural and non-structural proteins are described in Table 4 below.

TABLE 4
Genome and polyprotein structure of Zika virus
MVS, P7e (SEQ ID NO: 5 and SEQ ID NO: 4).

| | Gene | |
|---|---|---|
| | Corresponding sequence in | Corresponding part in |
| Protein | SEQ ID NO: 5 | SEQ ID NO: 4 |
| C | Nucleotides 88 to 453 | Amino acids 1 to 122 |
| prM/M | Nucleotides 454 to 957 | Amino acids 123 to 290 |
| E | Nucleotides 958 to 2469 | Amino acids 291 to 794 |
| NS1 | Nucleotides 2470 to 3525 | Amino acids 795 to 1146 |
| NS2A | Nucleotides 3526 to 4203 | Amino acids 1147 to 1372 |
| NS2B | Nucleotides 4204 to 4593 | Amino acids 1373 to 1502 |
| NS3 | Nucleotides 4594 to 6444 | Amino acids 1503 to 2119 |
| NS4A | Nucleotides 6445 to 6894 | Amino acids 2120 to 2269 |
| NS4B | Nucleotides 6895 to 7647 | Amino acids 2270 to 2520 |
| NS5 | Nucleotides 7648 to 10356 | Amino acids 2521 to 3423 |

An example of a Zika virus premembrane/membrane (prM/M) protein sequence is represented by SEQ ID NO: 7 as shown below. The prM/M protein sequence represented by SEQ ID NO: 7 is from Zika virus strain PRVABC59, Zika Pre-MVS (P6e) and Zika MVS (P7e) as described herein.

(SEQ ID NO: 7)
AEVTRRGSAYYMYLDRNDAGEAISFPTTLGMNKCYIQIMDLGHMCD
ATMSYECPMLDEGVEPDDVDCWCNTTSTWVVYGTCHHKKGEARRSR
RAVTLPSHSTRKLQTRSQTWLESREYTKHLIRVENWIFRNPGFALA
AAAIAWLLGSSTSQKVIYLVMILLIAPAYS

An example of a Zika virus envelope (E) protein sequence is represented by SEQ ID NO: 6 as shown below. The E protein sequence represented by SEQ ID NO: 6 is from Zika virus strain PRVABC59, Zika Pre-MVS (P6e) and Zika MVS (P7e) as described herein.

(SEQ ID NO: 6)
IRCIGVSNRDFVEGMSGGTWVDVVLEHGGCVTVMAQDKPTVDIELV
TTTVSNMAEVRSYCYEASISDMASDSRCPTQGEAYLDKQSDTQYVC
KRTLVDRGWGNGCGLFGKGSLVTCAKFACSKKMTGKSIQPENLEYR
IMLSVHGSQHSGMIVNDTGHETDENRAKVEITPNSPRAEATLGGFG
SLGLDCEPRTGLDFSDLYYLTMNNKHWLVHKEWFHDIPLPWHAGAD
TGTPHWNNKEALVEFKDAHAKRQTVVVLGSQEGAVHTALAGALEAE
MDGAKGRLSSGHLKCRLKMDKLRLKGVSYSLCTAAFTFTKIPAETL
HGTVTVEVQYAGTDGPCKVPAQMAVDMQTLTPVGRLITANPVITES
TENSKMMLELDPPFGDSYIVIGVGEKKITHHWHRSGSTIGKAFEAT
VRGAKRMAVLGDTAWDFGSVGGALNSLGKGIHQIFGAAFKSLFGGM
SWFSQILIGTLLMWLGLNTKNGSISLMCLALGGVLIFLSTAVSA

An example of a Zika virus non-structural protein 1 (NS1) sequence is represented by SEQ ID NO: 8 as shown below. The NS1 sequence represented by SEQ ID NO: 8 is from Zika Pre-MVS (P6e) and Zika MVS (P7e) as described herein. The NS1 sequence represented by SEQ ID NO: 8 shows a glycine at position 98 (indicated in bold in the sequence below).

(SEQ ID NO: 8)
DVGCSVDFSKKETRCGTGVFVYNDVEAWRDRYKYHPDSPRRLAAAV
KQAWEDGICGISSVSRMENIMWRSVEGELNAILEENGVQLTVVVGS
VKNPMGRGPQRLPVPVNELPHGWKAWGKSYFVRAAKTNNSFVVDGD
TLKECPLKHRAWNSFLVEDHGFGVFHTSVWLKVREDYSLECDPAVI
GTAVKGKEAVHSDLGYWIESEKNDTWRLKRAHLIEMKTCEWPKSHT
LWTDGIEESDLIIPKSLAGPLSHHNTREGYRTQMKGPWHSEELEIR
FEECPGTKVHVEETCGTRGPSLRSTTASGRVIEEWCCRECTMPPLS
FRAKDGCWYGMEIRPRKEPESNLVRSMVT

In some embodiments, the Zika virus is derived from strain PRVABC59.

In some embodiments, the Zika virus comprises an RNA genome sequence characterized by a DNA sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 3. In preferred embodiments, the Zika virus comprises an RNA genome sequence characterized by SEQ ID NO: 3.

In some embodiments, the Zika virus comprises an RNA genome sequence characterized by a DNA sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 5. In preferred embodiments, the Zika virus comprises an RNA genome sequence characterized by SEQ ID NO: 5.

In some embodiments, the Zika virus comprises an RNA genome sequence encoding a polyprotein having an amino acid sequence that has at least 90%, at least 91%, at least 92%, at least 93%, at least 94%, at least 95%, at least 96%, at least 97%, at least 98%, at least 99% or 100% sequence identity with SEQ ID NO: 4. In preferred embodiments, the Zika virus comprises an RNA genome sequence encoding a polyprotein represented by SEQ ID NO: 4.

In some embodiments, the Zika virus comprises at least one protein encoded by corresponding sequence parts of SEQ ID NO: 5 or by corresponding sequence parts of a sequence having at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity with SEQ ID NO: 5. In some embodiments, the at least one protein is selected from the group consisting of capsid (C) protein, precursor membrane/membrane (prM/M) protein, envelope (E) protein, NS1, NS2A, NS2B, NS3, NS4A, NS4B, and NS5. In some embodiments, the at least one protein is the envelope (E) protein.

In some embodiments, the Zika virus comprises at least one protein encoded by corresponding sequence parts of SEQ ID NO: 3 or by corresponding sequence parts of a sequence having at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity with SEQ ID NO: 3. In some embodiments, the at least one protein is selected from the group consisting of capsid (C) protein, precursor membrane/membrane (prM/M) protein, envelope (E) protein, NS1, NS2A, NS2B, NS3, NS4A, NS4B, and NS5. In some embodiments, the at least one protein is the envelope (E) protein.

In some embodiments, the Zika virus comprises at least one protein represented by corresponding sequence parts of SEQ ID NO: 4 or by corresponding sequence parts of a sequence having at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity with SEQ ID NO: 4. In some embodiments, the at least one protein is selected from the group consisting of capsid (C), precursor membrane/membrane (prM/M), envelope (E), NS1, NS2A, NS2B, NS3, NS4A, NS4B, and NS5. In specific embodiments, the at least one protein is the envelope (E).

In one embodiment, the Zika virus comprises a capsid protein, a pre-membrane/membrane (prM/M) protein, an envelope protein, as well as the non-structural proteins NS1, NS2A, NS2B, NS3, NS4A, NS4B, and NS5 represented by the corresponding sequence parts of SEQ ID NO: 4.

In one embodiment, the Zika virus comprises a capsid protein, a pre-membrane/membrane (prM/M) protein, an envelope protein, as well as the non-structural proteins NS1, NS2A, NS2B, NS3, NS4A, NS4B, and NS5 encoded by the corresponding sequence parts of SEQ ID NO: 5. In one embodiment, the Zika virus comprises a capsid protein, a pre-membrane/membrane (prM/M) protein, an envelope protein, as well as the non-structural proteins NS1, NS2A, NS2B, NS3, NS4A, NS4B, and NS5 encoded by the corresponding sequence parts of SEQ ID NO: 3.

In certain embodiments, the Zika virus comprises a pre-membrane/membrane (prM/M) protein having an amino acid sequence that has at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 7. In specific embodiments, the Zika virus comprises a pre-membrane/membrane (prM/M) protein represented by SEQ ID NO: 7.

In certain embodiments, the Zika virus comprises an envelope protein having an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 6. In specific embodiments, the Zika virus comprises an envelope protein represented by SEQ ID NO: 6.

In some embodiments, the Zika virus comprises a non-structural protein 1 (NS1) having an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 8. In specific embodiments, the Zika virus comprises a NS1 represented by SEQ ID NO: 8.

In one embodiment, the Zika virus comprises an envelope protein represented by SEQ ID NO: 6 and a pre-membrane/membrane (prM/M) protein represented by SEQ ID NO: 7.

In one embodiment, the Zika virus comprises an envelope protein represented by SEQ ID NO: 6, a pre-membrane/membrane (prM/M) protein represented by SEQ ID NO: 7, and a NS1 represented by SEQ ID NO: 8.

In some embodiments, the Zika virus comprises a mutation at nucleotide position 292 of the non-structural protein 1 (NS1) gene. In certain embodiments, the Zika virus comprises an uracil to guanine mutation at nucleotide position 292 (U292G) of the NS1 gene. In certain embodiments, the Zika virus comprises a guanine at nucleotide position 292 of the NS1 gene, encoding a glycine at amino acid position 98 of NS1. An example of a Zika virus genome having a guanine at nucleotide position 292 of the NS1 gene is represented by SEQ ID NO: 5, wherein position 292 of the NS1 gene refers to position 2761 of SEQ ID NO: 5. In certain embodiments, the Zika virus comprises a mutation at nucleotide position 292 of SEQ ID NO:11, or at a position equivalent to nucleotide position 292 of SEQ ID NO: 11. In certain embodiments, the Zika virus comprises an thymine to guanine mutation at nucleotide position 292 (T292G) of SEQ ID NO: 11, or at a position equivalent to nucleotide position 292 of SEQ ID NO: 11. In preferred embodiments, the Zika virus comprises a guanine at nucleotide position 292 of SEQ ID NO: 10 or at a position equivalent to nucleotide position 292 of SEQ ID NO: 10.

A position equivalent to nucleotide position 292 of SEQ ID NO: 10 (or SEQ ID NO: 11) in a given nucleotide sequence can be determined when aligning the given nucleotide sequence with SEQ ID NO: 10 (or SEQ ID NO: 11) using a pairwise alignment algorithm. A suitable program for generating such a pairwise alignment is the “Needle” program (EMBOSS) or an alignment program based on the Blast algorithm from NCBI. From the pairwise alignment, the skilled person can determine the equivalent position.

In certain embodiments, the Zika virus comprises a mutation at amino acid position 98 of NS1. In certain embodiments, the Zika virus comprises a tryptophan (W) to glycine (G) mutation at amino acid position 98 (Trp98Gly) of NS1. In certain embodiments, the Zika virus comprises a glycine at amino acid position 98 of NS1. An example of a Zika virus NS1 having a glycine at amino acid position 98 of NS1 is represented by SEQ ID NO: 8. In certain embodiments, the Zika virus comprises a mutation at amino acid position 98 of SEQ ID NO: 9, or at a position equivalent to amino acid position 98 of SEQ ID NO: 9. In certain embodiments, the Zika virus comprises a tryptophan to glycine mutation at amino acid position 98 (Trp98Gly) of SEQ ID NO: 9, or at a position equivalent to amino acid position 98 of SEQ ID NO: 9. In preferred embodiments, the Zika virus comprises a glycine at amino acid position 98 of SEQ ID NO: 8 or at a position equivalent to amino acid position 98 of SEQ ID NO: 8.

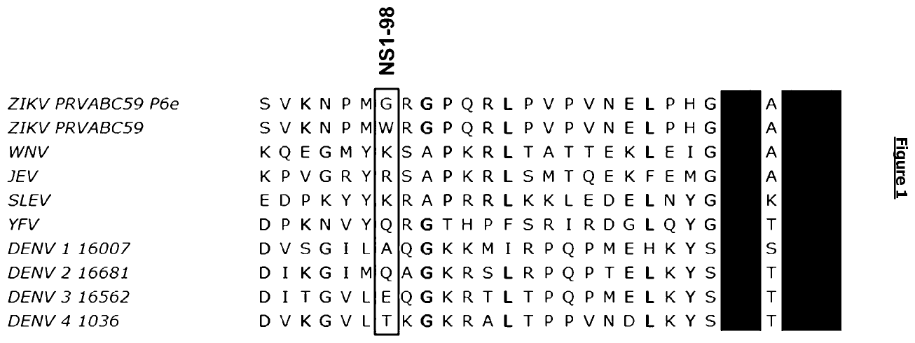

A position equivalent to amino acid position 98 of SEQ ID NO: 8 (or SEQ ID NO: 9) in a given amino acid sequence can be determined when aligning the given amino acid sequence with SEQ ID NO: 8 (or SEQ ID NO: 9) using a pairwise alignment algorithm. A suitable program for generating such a pairwise alignment is the “Needle” program (EMBOSS) an alignment program based on the Blast algorithm from NCBI. From the pairwise alignment, the skilled person can determine the equivalent position. For instance, positions equivalent to position 98 of the PRVABC59 NS1 protein (SEQ ID NO: 9) und the Pre-MVS/MVS NS1 protein (SEQ ID NO: 8) in flaviviruses other than Zika virus (West Nile virus (WNV); Japanese Encephalitis virus (JEV); St. Louis Encephalitis virus (SLEV); Yellow Fever Virus (YFV); and dengue virus (DENV) strains of the four serotypes) are shown in FIG. 1 of the present application, wherein the equivalent residues are boxed.

In certain embodiments, the mutation at position 98 of the NS1 or at position 292 of the NS1 gene, respectively, as described above is a non-human cell adaptation mutation. Non-human cell adaptation mutations may occur/accumulate by multiplying a Zika virus in a non-human cell line, such as a mammalian cell line. For generating and multiplying a Zika virus in a non-human cell line, the non-human cell line may be first transfected or electroporated with RNA representing the genome of the Zika virus and the Zika virus can be subsequently obtained from the surrounding cell culture media (as the virus is released into the media after cell lysis). The surrounding cell culture medium (in which the Zika virus is released) can be used for subsequent infection of an amount of fresh non-human cells leading to the generation of further Zika virus (i.e. to multiplication of the virus). This process can be repeated for several times, i.e. several passages can be performed. Passaging can also be carried out by plaque purification as described in Example 1 below. Non-human cell adaptation mutations may be point mutations (substitutions), insertion mutations, or deletion mutations. Such non-human cell adaptation mutations may also lead to amino acid changes within viral proteins. Non-human cell adaptations may lead to phenotypic changes in the virus, such as increased growth of the virus in the non-human cell line, thereby increasing virus yield, which can be useful in vaccine manufacture. Suitable non-human cells for multiplying the Zika virus are, for instance, VERO cells (from monkey kidneys), LLC-MK2 cells (from monkey kidneys), MDBK cells, MDCK cells, ATCC CCL34 MDCK (NBL2) cells, MDCK 33016 (deposit number DSM ACC 2219 as described in WO97/37001) cells, BHK21-F cells, HKCC cells, or Chinese hamster ovary cells (CHO cells). In certain embodiments, the non-human cells are monkey cells. In some embodiments, the monkey cells are Vero cells. Suitable Vero cells known in the art include, without limitation, WHO Vero 10-87, ATCC CCL-81, Vero 76 (ATCC Accession No. CRL-1587), or Vero C1008 (ATCC Accession No. CRL-1586). In some embodiments, the non-human cells are WHO Vero 10-87.

Without wishing to be bound by any theory, the mutation at position 98 of the NS1 or at position 292 of the NS1 gene, respectively, as described above, enhances genetic stability of the Zika virus, meaning that the Zika virus can be passaged multiple times (such as 7 times) in cell culture, such as on Vero cells, without acquiring any further mutations, in particular without acquiring any further amino acid mutations in the envelope protein. Moreover, the mutation at position 98 of the NS1 or at position 292 of the NS1 gene, respectively, as described above, at the same time also provides for efficient replication of the Zika virus in cell culture, such as in Vero cells. As in particular the envelope protein is the dominant immunogenic protein of the virus, a genetically stable Zika virus that does not accumulate mutations in the envelope protein, but at the same time can be produced at high amounts, is of particular advantage for the production of a vaccine and/or immunogenic composition, in particular, for the production of an inactivated whole virus vaccine and/or immunogenic composition. While in the production process of live, attenuated virus vaccines attenuation mutations are desired, mutations are not required and desired in the production process of an inactivated vaccine. There, it is preferred to work with a virus sample that resembles the wild-type virus as much as possible. However, as flaviviruses are generally regarded as genetically labile, acquiring multiple mutations also in the structural proteins upon replication/passaging in cell culture in the course of vaccine production is usual (even after one or two passages). Therefore, the generation of a genetically stable Zika virus that does not accumulate mutations in the envelope protein as the Zika virus comprising the mutation at position 98 of the NS1 or at position 292 of the NS1 gene according to certain aspects of the present disclosure was highly unlikely.

In certain embodiments, the Zika virus does not comprise a mutation in the envelope (E) protein, i.e. comprises a wild-type E protein.

In certain, preferred embodiments, the Zika virus comprises a glycine at amino acid position 98 of SEQ ID NO: 8 or at a position equivalent to amino acid position 98 of SEQ ID NO: 8. In certain, preferred embodiments, the Zika viruses comprises an E protein represented by SEQ ID NO: 6. In certain, preferred embodiments, the Zika virus comprises an E protein represented by SEQ ID NO: 6 and a glycine at amino acid position 98 of SEQ ID NO: 8 or at a position equivalent to amino acid position 98 of SEQ ID NO: 8.

As outlined above, the antigen of the Zika virus present in the vaccine and/or immunogenic composition according to the present disclosure is an inactivated whole Zika virus. The vaccines and/or immunogenic compositions comprising an antigen of a Zika virus described in this section are used for preventing Zika virus infection in a subject in need thereof and/or for inducing an immune response, such as a protective immune response, against Zika virus in a subject in need thereof, such as a human subject.

However, as will be appreciated by the skilled person, the Zika viruses described in this section may also be useful in the development of a live, attenuated Zika virus vaccine or in the development of Zika virus recombinant/subunit vaccines comprising one or more proteins or parts of one or more proteins of a Zika virus as described above. Such recombinant/subunit vaccines may comprise the prM and E proteins of a Zika virus as described above. The one or more proteins or parts of one or more proteins of a Zika virus as described above can be produced by well-established recombinant DNA techniques. For instance, an expression construct comprising a nucleotide sequence encoding the one or more proteins or parts of one or more proteins of a Zika virus as described above can be introduced in an appropriate host cell, which can be a eukaryotic cell (such as a yeast cell, an insect cell, or a mammalian cell) or a prokaryotic cell (such as E. coli), generating a genetically modified host cell. Under appropriate culture conditions, the one or more proteins or parts of one or more proteins are produced by the genetically modified cell. When administered to a subject (such as a human subject) such recombinant/subunit vaccines may stimulate an immune response (such as a protective immune response). Thus, the present disclosure is, at least in certain parts, also directed to live, attenuated Zika virus vaccines and recombinant/subunit Zika vaccines developed based on a Zika virus as described above.

### Production and Purification of Zika Virus

### Cell Lines and Culture Conditions for Zika Virus Production

To produce an amount of a Zika virus as described above, such as a Zika virus comprising an envelope protein having an amino acid sequence represented by SEQ ID NO: 6, sufficient for the application in the vaccines or immunogenic compositions of the present disclosure, the Zika virus may be multiplied in suitable cells known in the art for multiplying a Zika virus.

Cell lines suitable for Zika virus growth are in particular non-human cells. For instance, cell lines suitable for Zika virus growth may be insect cell lines or mammalian cell lines.

Suitable insect cells are cells from mosquito species such as Aedes aegypti, Aedes albopictus, Aedes pseudoscutellaris, Aedes triseriatus, Aedes vexans, Anopheles gambiae, Anopheles stephensi, Anopheles albimus, Culex quinquefasciatus, Culex theileri, Culex tritaeniorhynchus, Culex bitaeniorhynchus, and/or Toxorhynchites amboinensis. Particular suitable insect cells are CCL-125 cells, Aag-2 cells, RML-12 cells, C6/36 cells, C7-10 cells, AP-61 cells, A.t. GRIP-1 cells, A.t. GRIP-2 cells, A.t. GRIP-3 cells, UM-AVE1 cells, Mos.55 cells, Sua1B cells, 4a-3B cells, Mos.42 cells, MSQ43 cells, LSB-AA695BB cells, NIID-CTR cells, or TRA-171 cells.

Suitable mammalian cells include monkey cells, horse cells, cow cells, sheep cells, dog cells, rodent cells, and cat cells. Preferred are non-human mammalian cells. The mammalian cells may be obtained from a wide variety of developmental stages, including for example, adult, neonatal, fetal, and embryo. The mammalian cells may be selected from and/or derived from one or more of the following non-limiting cell types: fibroblast cells (e.g. dermal, lung), endothelial cells (e.g. aortic, coronary, pulmonary, vascular, dermal microvascular, umbilical), hepatocytes, keratinocytes, immune cells (e.g. T cell, B cell, macrophage, NK, dendritic), mammary cells (e.g. epithelial), smooth muscle cells (e.g. vascular, aortic, coronary, arterial, uterine, bronchial, cervical, retinal pericytes), melanocytes, neural cells (e.g. astrocytes), prostate cells (e.g. epithelial, smooth muscle), renal cells (e.g. epithelial, mesangial, proximal tubule), skeletal cells (e.g. chondrocyte, osteoclast, osteoblast), muscle cells (e.g. myoblast, skeletal, smooth, bronchial), liver cells, retinoblasts, and stromal cells. In certain embodiments, the cells are immortalized (e.g. PERC.6 cells, as described in WO 01/38362 and WO 02/40665, and as deposited under ECACC deposit number 96022940). For instance, VERO cells (from monkey kidneys), LLC-MK2 cells (from monkey kidneys), MDBK cells, MDCK cells, ATCC CCL34 MDCK (NBL2) cells, MDCK 33016 (deposit number DSM ACC 2219 as described in WO97/37001) cells, BHK21-F cells, HKCC cells, or Chinese hamster ovary cells (CHO cells) may be used. In preferred embodiments, the cells in which the Zika virus is multiplied are VERO cells.

Culture conditions for the above-mentioned cell types are known and described in a variety of publications. Culture medium and supplements may be purchased commercially, such as for example, described in the catalog and additional literature of Cambrex Bioproducts (East Rutherford, N.J.). Cells for viral growth may be cultured in suspension or under adherent conditions. For instance, WO97/37000 and WO97/37001 describe production of animal cells and cell lines that are capable of growth in suspension and serum free media, which are useful for the production of viruses.

In certain embodiments, the cells used for Zika virus production are cultured in serum free and/or protein free media. A serum free medium refers to a medium that lacks additives from serum of human or animal origin. Protein-free is understood to mean cultures in which multiplication of the cells occurs with exclusion of proteins, growth factors, other protein additives and non-serum proteins, but can optionally include proteins such as trypsin or other proteases that may be necessary for viral growth. The cells growing in such cultures naturally contain proteins themselves.

Known serum-free media include Iscove's medium, Ultra-CHO medium (BioWhittaker) or EX-CELL (JRH Bioscience). Ordinary serum-containing media include Eagle's Basal Medium (BME) or Minimum Essential Medium (MEM) (Eagle, Science, 130, 432 (1959)) or Dulbecco's Modified Eagle Medium (DMEM or EDM), which are ordinarily used with up to 10% fetal calf serum or similar additives. Optionally, Minimum Essential Medium (MEM) (Eagle, Science, 130, 432 (1959)) or Dulbecco's Modified Eagle Medium (DMEM or EDM) may be used without any serum containing supplement. Protein-free media like PF-CHO (JHR Bioscience), chemically-defined media like ProCHO 4CDM (BioWhittaker) or SMIF 7 (Gibco/BRL Life Technologies) and mitogenic peptides like Primactone, Pepticase or HyPep™ (all from Quest International) or lactalbumin hydrolysate (Gibco and other manufacturers) are also adequately known in the prior art. The media additives based on plant hydrolysates have the special advantage that contamination with viruses, mycoplasma or unknown infectious agents can be ruled out.

Cell culture conditions (temperature, cell density, pH value, etc.) are variable over a very wide range owing to the suitability of the cell line employed according to the present disclosure and can be adapted to the requirements of particular Zika virus strains.

To multiply the Zika virus in the cell line selected for virus production, the cultured cells are infected with a certain amount of the Zika virus. The cultured cells are usually infected at a high confluency. Cultured cells may be infected at a multiplicity of infection (“MOI”) of about 0.0001 to 10, preferably 0.002 to 5, more preferably to 0.001 to 2. Still more preferably, the cells are infected at an MOI of about 0.01. The virus is added to a suspension of the cells or is applied to a monolayer of the cells, dependent on the type of cell line used, and the virus is absorbed on the cells for at least 10 minutes, at least 20 minutes, at least 30 minutes, at least 40 minutes, at least 50 minutes, at least 60 minutes but usually less than 300 minutes at 25° C. to 40° C., preferably 28° C. to 38° C. During the adsorption period, the ratio of culture medium to the area of cell culture vessel is usually lower than during the culture of the cells, which maximizes the likelihood that the virus will infect the cells. After the adsorption period, additional cell culture medium may be added. During incubation, the virus produced will accumulate in the cell culture medium (also referred to as the cell culture supernatant in the case of adherent cells). The infected cells are cultivated for a desired time-period for virus propagation, which can be determined, for example, by measuring the virus titer and/or by monitoring the occurrence of cytopathic effects (CPE) on the cells. In certain embodiments, the infected cells are cultivated for 30 to 60 hours post infection, or 3 to 10 days post infection, or 3 to 7 days post infection, or 3 to 5 days post infection. Afterwards, the virus is harvested by collecting the medium surrounding the cells (also referred to as the cell culture supernatant in the case of adherent cells). Residual host cells in the collected medium are usually removed by centrifugation and/or filtration (resulting in the clarified virus harvest). The clarified harvest can be optionally stabilized by adding sugar, such as trehalose, and/or stored at a temperature of about −60 to −80° C.

Alternatively, if merely the genomic RNA of the Zika virus is available (which may be, for instance, synthesized by methods known in the art based on available sequence information), the cultured cells are first transfected with the RNA of the Zika virus to obtain the virus from the medium surrounding the cells, in which the virus is secreted. The virus obtained can then subsequently be used for infecting further cultured cells as described in the paragraph above.

In the manufacturing process of vaccines or immunogenic compositions it is usual that a virus is passaged for several times in order to purify and multiply said virus, as well as to adapt the virus to grow to high titers in certain cells (such as non-human cells, e.g. Vero cells). In certain embodiments, a harvest obtained as described above may be the first passage (P1), which is used for subsequent infection of further (“fresh”) cells thereby providing for a further harvest, i.e. the second passage (P2). Additionally, or alternatively, the harvested virus can also be further passaged by plaque purification. Methods of purifying a virus by performing plaque purification are known to one of ordinary skill in the art. Plaque purification allows for the substantial and/or complete separation of a (genetically homogenous) clonal isolate from a heterogeneous viral population. In some embodiments, a Zika virus can be passaged multiple times using plaque purification, such as two or more, three or more, four or more, or five or more times.

A suitable production process of a Zika virus including multiple passages is described in detail in Example 1 of the present application (see, for instance, Table 6).

During multiple passages in cell culture, viruses tend to accumulate adaptation mutations, often in the structural proteins. For instance, usually, upon multiple passages in cell culture, a Zika virus accumulates mutations in the envelope (E) protein.

Both, the virus used for infection of the cells, as well as the virus harvested from the cell culture medium are preferably free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses (see also WO2006/027698).

Where a virus has been grown on a cell line then it is standard practice to minimize the amount of residual cell line DNA in the final vaccine, in order to minimize any oncogenic activity of the host cell DNA. Therefore, in certain embodiments, contaminating DNA is removed from the (clarified) harvest using standard purification procedures e.g. chromatography, etc. Removal of residual host cell DNA can be enhanced by nuclease treatment e.g. by using a DNase. A convenient method for reducing host cell DNA contamination disclosed in references (Lundblad (2001) Biotechnology and Applied Biochemistry 34:195-197, Guidance for Industry: Bioanalytical Method Validation. U.S. Department of Health and Human Services Food and Drug Administration Center for Drug Evaluation and Research (CDER) Center for Veterinary Medicine (CVM). May 2001.) involves a two-step treatment, first using a DNase (e.g. Benzonase), which may be used during viral growth, and then a cationic detergent (e.g. CTAB), which may be used during virion disruption. In one embodiment, the contaminating DNA is removed from the virus harvest by benzonase treatment.

### Purification of the Zika Virus

The Zika virus may be purified before and/or after being inactivated as described in the chapter “Zika virus inactivation” below. In preferred embodiments, the Zika virus is purified before being inactivated.

The Zika virus may be purified from the (clarified) harvest obtained as described above using liquid chromatography, such as size exclusion chromatography (SEC), or affinity chromatography, or ion exchange chromatography. In certain embodiments, the Zika virus may be purified by high performance liquid chromatography (HPLC). The ion exchange chromatography may be anion or cation ion exchange chromatography. The Zika virus may be purified by multiple (different) liquid chromatography steps.

In one embodiment, the Zika virus is purified using anion exchange chromatography. In one embodiment, anion exchange chromatography uses an anion exchange membrane comprising quaternary ammonium ligands. In some embodiments, the virus is eluted from the anion exchange membrane by step elution, e.g. using 250 mM NaCl, 500 mM NaCl, and 750 mM NaCl. Exemplary suitable anion exchange columns are the Mustang® Q system (Pall Corporation), which uses an anion exchange membrane with a 0.8 μm pore size, and SartobindQ IEX Nano.

Prior to/after/or instead of purification by liquid chromatography as outlined above, the harvest may be filtered, for instance, by depth filtration and/or tangential flow filtration (TFF) and/or cross flow filtration (CFF). In addition, or alternatively, the harvest may be buffer exchanged and/or diluted. In some embodiments, the step of buffer exchange and/or dilution involves cross flow filtration (CFF).

Suitable depth filtration techniques and apparatus are known in the art and include Sartorius PP3 filters. In some embodiments, the depth filter has a pore size of between about 0.2 μm and about 3 μm.

A particular suitable purification process of a Zika virus clarified harvest is described in the Example section, in particular in Example 2. A suitable purification process is also described in WO 2019/090228 A2, in particular under the section “virus purification”, which is herewith incorporated by reference.

The purity of a Zika virus may be described by the percentage that the area under the curve of the main peak of the Zika virus makes up compared to the total area under the curve when analyzing the Zika virus by liquid chromatography (such as size exclusion chromatography). Determination of the area under the curve is within the common knowledge of the skilled person and can be assessed by integration of the curve. As will also be readily understood by the skilled person in the art, peaks in the chromatography that result from a component intended to be present in the Zika virus sample (such as a particular buffer in which the Zika virus is present, the buffer also showing a peak in the chromatography, for instance, due to absorbance at the measurement wavelength) are not to be taken into account when calculating the total area under the curve. The skilled person usually uses the buffer in which the Zika virus sample is present as the running buffer in which the column is equilibrated to avoid any signal interference due to buffer components. As will also be appreciated by the person skilled in the art, the “main peak of the Zika virus” as referred to herein corresponds to the peak of intact Zika virus, i.e. non-aggregated or degraded Zika virus. The elution time of the main peak can be readily determined by the skilled person for any chromatography column.

Within the meaning of this disclosure, a Zika virus is referred to as a “purified Zika virus” when the main peak of the Zika virus when analyzed by size exclusion chromatography is more than 85% of the total area under the curve in the size exclusion chromatography. In certain embodiments, the main peak of the Zika virus when analyzed by size exclusion chromatography can also be more than 90%, or more than 95%, or more than 98% or more than 99% of the total area under the curve in the size exclusion chromatography. In certain embodiments, the main peak of the Zika virus when analyzed by size exclusion chromatography can also be more than 65%, or more than 70%, or more than 75% or more than 80% of the total area under the curve in the size exclusion chromatography.

During the production process of the vaccines/immunogenic compositions of the present disclosure, samples may routinely be taken at certain points (such as before or after inactivation, or before formulating the final drug product) and analyzed by size exclusion chromatography in order to assess the stability/purity of the Zika virus. Additionally, or alternatively, the stability/purity of the Zika virus during the production process may also be assessed using gel electrophoresis (including one-dimensional gel electrophoresis, two-dimensional gel electrophoresis).

The purified Zika virus described in this section can be further inactivated as described in the section “Zika virus inactivation” to prepare an inactivated whole Zika virus.

### Zika Virus Inactivation

Certain aspects of the present disclosure relate to vaccines and/or immunogenic compositions comprising an inactivated whole Zika virus as an antigen.

Inactivating a Zika virus shall destroy the ability of the Zika virus to infect host cells (such as mammalian cells, that can be infected by a Zika virus that was not subjected to an inactivation step), but at the same time shall not destroy the structure of the virus, such as the secondary, tertiary, or quaternary structure of the virus, providing the immunogenic epitopes. An optimally inactivated Zika virus is no longer able to infect mammalian cells and thus is safe when administered to a subject (in particular a human subject), but at the same time still encompasses a sufficient amount of intact immunogenic epitopes capable of inducing neutralizing antibody titers when administered to a subject (in particular a human subject). Thereby, safety and immunogenicity are balanced.

A Zika virus can be inactivated by both chemical and physical means. Suitable physical means of inactivation are, for example, heat treatment, electromagnetic radiation, x-ray radiation, gamma radiation, and ultraviolet radiation (UV radiation), such as UV-A radiation, UV-B radiation, UV-C radiation. Suitable chemical means include, for example, treatment of the virus with an effective amount of one or more agents selected from a detergent, formaldehyde, hydrogen peroxide, beta-propiolactone (BPL), binary ethylamine (BEI), acetyl ethyleneimine, methylene blue, psoralen, carboxyfullerene (C60) and any combination of any thereof. In preferred embodiments, the Zika virus is chemically inactivated with one or more agents as described in this paragraph.

In embodiments wherein the Zika virus is chemically inactivated with BPL, formaldehyde, and/or BEI, the virus may contain one or more modifications, which may include modifications to the Zika virus genome and/or to the proteins of the Zika virus. Modifications may include alkylation of nucleic acid/amino acid residues and/or cross-linking of nucleic acid/amino acid residues.

In preferred embodiments, the Zika virus is inactivated with formaldehyde. When reference is made herein to a concentration of formaldehyde, it refers to the concentration of formaldehyde itself and not to the concentration of formalin, which is an aqueous composition of formaldehyde. A saturated aqueous solution comprising formaldehyde is commonly referred to as “100% formalin” or sometimes only as “formalin” and comprises 37% formaldehyde by mass (w/v). Accordingly, the concentrations of formaldehyde as given herein do not require any further correction for the formaldehyde concentration in a formalin solution. Therefore, for instance, a “formaldehyde concentration of 0.01% (w/v)” refers to 0.01% (w/v) formaldehyde, and no further correction of this concentration for the formaldehyde concentration in the formalin has to be made. For example, such a formaldehyde concentration in the virus preparation can be obtained by diluting 100% formalin to a working solution having a formaldehyde content of 1.85% (w/v) which is then further diluted to the required concentration by mixing the necessary amounts of the working solution with the virus preparation such as a Zika virus preparation.

In certain embodiments, the Zika virus is inactivated with 0.005 to 0.02% (w/v) formaldehyde. In other embodiments, the Zika virus is inactivated with 0.0075 to 0.015% (w/v) formaldehyde. In some embodiments, the Zika virus is inactivated with 0.02% (w/v) formaldehyde. In some embodiments, the Zika virus is inactivated with 0.01% (w/v) formaldehyde.

In certain embodiments, the Zika virus is obtainable/obtained by a method comprising a step of inactivating the Zika virus with 0.005 to 0.02% (w/v) formaldehyde, optionally at a temperature of 15° C. to 30° C. for six to fourteen days. In certain embodiments, the Zika virus is obtainable/obtained by a method comprising a step of inactivating the Zika virus with 0.01% (w/v) formaldehyde, optionally at a temperature of 22° C. for ten days. In other embodiments, the Zika virus is obtainable/obtained by a method comprising a step of inactivating the Zika virus with 0.02% (w/v) formaldehyde, optionally at a temperature of 22° C. for 14 days.

In certain embodiments, the Zika virus is inactivated with formaldehyde at a temperature that ranges from about 2° C. to about 42° C. For example, the Zika virus may be inactivated with formaldehyde at a temperature that ranges from about 2° C. to about 42° C., about 2° C. to about 8° C., about 15° C. to about 37° C., about 17° C. to about 27° C., about 20° C. to about 25° C., or at a temperature of about 2° C., about 4° C., about 8° C., about 10° C., about 15° C., about 17° C., about 18° C., about 19° C., about 20° C., about 21° C., about 22° C., about 23° C., about 24° C., about 25° C., about 26° C., about 27° C., about 28° C., about 29° C., about 30° C., about 37° C., or about 42° C. In certain embodiments, the Zika virus is inactivated with formaldehyde at a temperature of 15° C. to 30° C. In some embodiments, the Zika virus is inactivated with formaldehyde at a temperature of 18° C. to 25° C. In preferred embodiments, the Zika virus is inactivated with formaldehyde at a temperature of 20° C. to 24° C., such as 22° C.

In some embodiments, the Zika virus is inactivated with formaldehyde for at least about 1 day. For example, the Zika virus may be inactivated with formaldehyde for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or more. In some embodiments, the Zika virus is inactivated with formaldehyde for at least about 9 days. In some embodiments, the Zika is inactivated with formaldehyde for at least about 11 days. In some embodiments, the Zika virus is inactivated with formaldehyde for at least about 14 days. In some embodiments, the Zika virus is inactivated with formaldehyde for at least about 20 days. In some embodiments, the Zika virus is inactivated with formaldehyde for at least about 30 days. In preferred embodiments, the Zika virus is inactivated with formaldehyde for six to fourteen days. In preferred embodiments, the Zika virus is inactivated with formaldehyde for eight to twelve days. In other preferred embodiments, the Zika virus is inactivated with formaldehyde for nine to eleven days. In particular preferred embodiments, the Zika virus is inactivated with formaldehyde for ten days.

In some embodiments, the Zika virus is inactivated with 0.005 to 0.02% (w/v) formaldehyde for six to fourteen days at a temperature of 15° C. to 30° C. In some embodiments, the Zika virus is inactivated with 0.005 to 0.02% (w/v) formaldehyde for eight to twelve days at a temperature of 15° C. to 30° C. In some embodiments, the Zika virus is inactivated with 0.005 to 0.02% (w/v) formaldehyde for nine to eleven days at a temperature of 15° C. to 30° C. In some embodiments, the Zika virus is inactivated with 0.005 to 0.02% (w/V) formaldehyde for ten days at a temperature of 15° C. to 30° C.

In some embodiments, the Zika virus is inactivated with 0.008 to 0.015% (w/v) formaldehyde for six to fourteen days at a temperature of 15° C. to 30° C. In some embodiments, the Zika virus is inactivated with 0.008 to 0.015% (w/v) formaldehyde for eight to twelve days at a temperature of 15° C. to 30° C. In some embodiments, the Zika virus is inactivated with 0.008 to 0.015% (w/v) formaldehyde for nine to eleven days at a temperature of 15° C. to 30° C. In some embodiments, the Zika virus is inactivated with 0.008 to 0.015% (w/v) formaldehyde for ten days at a temperature of 15° C. to 30° C.

In some embodiments, the Zika virus is inactivated with 0.01% (w/v) formaldehyde for six to fourteen days at a temperature of 15° C. to 30° C. In some embodiments, the Zika virus is inactivated with 0.01% (w/v) formaldehyde for eight to twelve days at a temperature of 15° C. to 30° C. In some embodiments, the Zika is inactivated with 0.01% (w/v) formaldehyde for nine to eleven days at a temperature of 15° C. to 30° C. In some embodiments, the Zika virus is inactivated with 0.01% (w/v) formaldehyde for ten days at a temperature of 15° C. to 30° C.

In some embodiments, the Zika virus is inactivated with 0.005 to 0.02% (w/v) formaldehyde for six to fourteen days at a temperature of 18° C. to 25° C. In some embodiments, the Zika virus is inactivated with 0.005 to 0.02% (w/v) formaldehyde for eight to twelve days at a temperature of 18° C. to 25° C. In some embodiments, the Zika virus is inactivated with 0.005 to 0.02% (w/v) formaldehyde for nine to eleven days at a temperature of 18° C. to 25° C. In some embodiments, the Zika virus is inactivated with 0.005 to 0.02% (w/v) formaldehyde for ten days at a temperature of 18° C. to 25° C.

In some embodiments, the Zika virus is inactivated with 0.008 to 0.015% (w/v) formaldehyde for six to fourteen days at a temperature of 18° C. to 25° C. In some embodiments, the Zika virus is inactivated with 0.008 to 0.015% (w/v) formaldehyde for eight to twelve days at a temperature of 18° C. to 25° C. In some embodiments, the Zika is inactivated with 0.008 to 0.015% (w/v) formaldehyde for nine to eleven days at a temperature of 18° C. to 25° C. In some embodiments, the Zika virus is inactivated with 0.008 to 0.015% (w/v) formaldehyde for ten days at a temperature of 18° C. to 25° C.

In some embodiments, the Zika virus is inactivated with 0.01% (w/v) formaldehyde for six to fourteen days at a temperature of 18° C. to 25° C. In some embodiments, the Zika virus is inactivated with 0.01% (w/v) formaldehyde for eight to twelve days at a temperature of 18° C. to 25° C. In some embodiments, the Zika virus is inactivated with 0.01% (w/v) formaldehyde for nine to eleven days at a temperature of 18° C. to 25° C. In some embodiments, the Zika virus is inactivated with 0.01% (w/v) formaldehyde for ten days at a temperature of 18° C. to 25° C.

In some embodiments, the Zika virus is inactivated with 0.005 to 0.02% (w/v) formaldehyde for six to fourteen days at a temperature of 22° C. In some embodiments, the Zika virus is inactivated with 0.005 to 0.02% (w/v) formaldehyde for eight to twelve days at a temperature of 22° C. In some embodiments, the Zika virus is inactivated with 0.005 to 0.02% (w/v) formaldehyde for nine to eleven days at a temperature of 22° C. In some embodiments, the Zika virus is inactivated with 0.005 to 0.02% (w/v) formaldehyde for ten days at a temperature of 22° C.

In some embodiments, the Zika virus is inactivated with 0.008 to 0.015% (w/v) formaldehyde for six to fourteen days at a temperature of 22° C. In some embodiments, the Zika virus is inactivated with 0.008 to 0.015% (w/v) formaldehyde for eight to twelve days at a temperature of 22° C. In some embodiments, the Zika virus is inactivated with 0.008 to 0.015% (w/v) formaldehyde for nine to eleven days at a temperature of 22° C. In some embodiments, the Zika virus is inactivated with 0.008 to 0.015% (w/v) formaldehyde for ten days at a temperature of 22° C.

In some embodiments, the Zika virus is inactivated with 0.01% (w/v) formaldehyde for six to fourteen days at a temperature of 22° C. In some embodiments, the Zika virus is inactivated with 0.01% (w/v) formaldehyde for eight to twelve days at a temperature of 22° C. In some embodiments, the Zika virus is inactivated with 0.01% (w/v) formaldehyde for nine to eleven days at a temperature of 22° C. In some embodiments, the Zika virus is inactivated with 0.01% (w/v) formaldehyde for ten days at a temperature of 22° C.

In some embodiments, the Zika virus is inactivated with 0.02% (w/v) formaldehyde for 14 days at a temperature of 22° C.

In preferred embodiments, the Zika virus is inactivated with 0.01% (w/v) formaldehyde for 10 days at a temperature of 22° C.

In embodiments, wherein the Zika virus is inactivated with formaldehyde, the inactivation conditions can also be described with the numerical result of the multiplication of the formaldehyde concentration as measured in % (w/v) with the period of incubation with formaldehyde as measured in days. For instance, when the formaldehyde concentration is 0.01% (w/v) and the period of incubation with formaldehyde is 10 days, the numerical result of the multiplication of the formaldehyde concentration with the period of incubation with formaldehyde is 0.01×10=0.1.

In certain embodiments, the Zika virus is inactivated with formaldehyde, wherein the numerical result of the multiplication of the formaldehyde concentration as measured in % (w/v) with the period of incubation with formaldehyde as measured in days is 0.025 to 0.5. In some embodiments, the numerical result of the multiplication of the formaldehyde concentration as measured in % (w/v) with the period of incubation with formaldehyde as measured in days is 0.05 to 0.25. In some embodiments, the numerical result of the multiplication of the formaldehyde concentration as measured in % (w/v) with the period of incubation with formaldehyde as measured in days is 0.075 to 0.15. In some embodiments, the numerical result of the multiplication of the formaldehyde concentration as measured in % (w/v) with the period of incubation with formaldehyde as measured in days is 0.1.

In some embodiments, the numerical result of the multiplication of the formaldehyde concentration as measured in % (w/v) with the period of incubation with formaldehyde as measured in days is 0.025 to 0.5 and the formaldehyde concentration is 0.005% (w/v) to 0.02% (w/v). In some embodiments, the numerical result of the multiplication of the formaldehyde concentration as measured in % (w/v) with the period of incubation with formaldehyde as measured in days is 0.025 to 0.5 and the formaldehyde concentration is 0.0075% (w/v) to 0.015% (w/v). In some embodiments, the numerical result of the multiplication of the formaldehyde concentration as measured in % (w/v) with the period of incubation with formaldehyde as measured in days 0.025 to 0.5 and the formaldehyde concentration is 0.01% (w/v).

In some embodiments, the numerical result of the multiplication of the formaldehyde concentration as measured in % (w/v) with the period of incubation with formaldehyde as measured in days is 0.05 to 0.25 and the formaldehyde concentration is 0.005% (w/v) to 0.02% (w/v). In some embodiments, the numerical result of the multiplication of the formaldehyde concentration as measured in % (w/v) with the period of incubation with formaldehyde as measured in days is 0.05 to 0.25 and the formaldehyde concentration is 0.0075% (w/v) to 0.015% (w/v). In some embodiments, the numerical result of the multiplication of the formaldehyde concentration as measured in % (w/v) with the period of incubation with formaldehyde as measured in days 0.05 to 0.25 and the formaldehyde concentration is 0.01% (w/v).

In some embodiments, the numerical result of the multiplication of the formaldehyde concentration as measured in % (w/v) with the period of incubation with formaldehyde as measured in days is 0.075 to 0.15 and the formaldehyde concentration is 0.005% (w/v) to 0.02% (w/v). In some embodiments, the numerical result of the multiplication of the formaldehyde concentration as measured in % (w/v) with the period of incubation with formaldehyde as measured in days is 0.075 to 0.15 and the formaldehyde concentration is 0.0075% (w/v) to 0.015% (w/v). In some embodiments, the numerical result of the multiplication of the formaldehyde concentration as measured in % (w/v) with the period of incubation with formaldehyde as measured in days 0.075 to 0.15 and the formaldehyde concentration is 0.01% (w/v).

In some embodiments, the numerical result of the multiplication of the formaldehyde concentration as measured in % (w/v) with the period of incubation with formaldehyde as measured in days is 0.1 and the formaldehyde concentration is 0.005% (w/v) to 0.02% (w/v). In some embodiments, the numerical result of the multiplication of the formaldehyde concentration as measured in % (w/v) with the period of incubation with formaldehyde as measured in days is 0.1 and the formaldehyde concentration is 0.0075% (w/v) to 0.015% (w/v). In some embodiments, the numerical result of the multiplication of the formaldehyde concentration as measured in % (w/v) with the period of incubation with formaldehyde as measured in days 0.1 and the formaldehyde concentration is 0.01% (w/v).

In some embodiments, the numerical result of the multiplication of the formaldehyde concentration as measured in % (w/v) with the period of incubation with formaldehyde as measured in days is 0.025 to 0.5 and the period of incubation with formaldehyde is eight to twelve days. In some embodiments, the numerical result of the multiplication of the formaldehyde concentration as measured in % (w/v) with the period of incubation with formaldehyde as measured in days is 0.025 to 0.5 and the period of incubation with formaldehyde is nine to eleven days. In some embodiments, the numerical result of the multiplication of the formaldehyde concentration as measured in % (w/v) with the period of incubation with formaldehyde as measured in days is 0.025 to 0.5 and the period of incubation with formaldehyde is ten days.

In some embodiments, the numerical result of the multiplication of the formaldehyde concentration as measured in % (w/v) with the period of incubation with formaldehyde as measured in days is 0.05 to 0.25 and the period of incubation with formaldehyde is eight to twelve days. In some embodiments, the numerical result of the multiplication of the formaldehyde concentration as measured in % (w/v) with the period of incubation with formaldehyde as measured in days is 0.05 to 0.25 and the period of incubation with formaldehyde is nine to eleven days. In some embodiments, the numerical result of the multiplication of the formaldehyde concentration as measured in % (w/v) with the period of incubation with formaldehyde as measured in days is 0.05 to 0.25 and the period of incubation with formaldehyde is ten days.

In some embodiments, the numerical result of the multiplication of the formaldehyde concentration as measured in % (w/v) with the period of incubation with formaldehyde as measured in days is 0.075 to 0.15 and the period of incubation with formaldehyde is eight to twelve days. In some embodiments, the numerical result of the multiplication of the formaldehyde concentration as measured in % (w/v) with the period of incubation with formaldehyde as measured in days is 0.075 to 0.15 and the period of incubation with formaldehyde is nine to eleven days. In some embodiments, the numerical result of the multiplication of the formaldehyde concentration as measured in % (w/v) with the period of incubation with formaldehyde as measured in days is 0.075 to 0.15 and the period of incubation with formaldehyde is ten days.

In some embodiments, the numerical result of the multiplication of the formaldehyde concentration as measured in % (w/v) with the period of incubation with formaldehyde as measured in days is 0.1 and the period of incubation with formaldehyde is eight to twelve days. In some embodiments, the numerical result of the multiplication of the formaldehyde concentration as measured in % (w/v) with the period of incubation with formaldehyde as measured in days is 0.1 and the period of incubation with formaldehyde is nine to eleven days. In some embodiments, the numerical result of the multiplication of the formaldehyde concentration as measured in % (w/v) with the period of incubation with formaldehyde as measured in days is 0.1 and the period of incubation with formaldehyde is ten days.

In the middle of the inactivation treatment period, the mixture of Zika virus and formaldehyde may be filtered to remove aggregates. After filtration the mixture of Zika virus and formaldehyde is transferred to a new vessel and further treated with formaldehyde until the end of the inactivation treatment period. In some embodiments, the mixture of Zika virus and formaldehyde is filtered after four to six days of formaldehyde treatment, if the overall formaldehyde treatment period is eight to twelve days. In some embodiments, the mixture of Zika virus and formaldehyde is filtered after five to six days of formaldehyde treatment, if the overall formaldehyde treatment period is nine to eleven days. In some embodiments, the mixture of Zika virus and formaldehyde is filtered after five days of formaldehyde treatment, if the overall formaldehyde treatment period is ten days. A suitable filter for this step is a 0.2 μm filter.

In some embodiments wherein the Zika virus is chemically inactivated with formaldehyde, any residual unreacted formaldehyde may be removed by neutralization with sodium metabisulfite, dialysis, tangential flow filtration (TFF) and/or buffer exchange. By removing residual unreacted formaldehyde, the inactivated Zika virus may at the same time also be purified.

In some embodiments, residual unreacted formaldehyde is removed by neutralization with sodium metabisulfite. In some embodiments, residual unreacted formaldehyde is removed by neutralization with from about 0.01 mM to about 100 mM sodium metabisulfite. In some embodiments, the sodium metabisulfite concentration may be from about 0.1 mM to about 50 mM, from about 0.5 mM to about 20 mM, from about 1 mM to about 10 mM, or from about 2 mM to about 5 mM. In some embodiments, residual unreacted formaldehyde is removed by neutralization with about 0.01 mM, about 0.05 mM, about 0.1 mM, about 0.25 mM, about 0.5 mM, about 0.75 mM, about 1 mM, about 2 mM, about 3 mM, about 4 mM, about 5 mM, about 6 mM, about 7 mM, about 8 mM, about 9 mM, about 10 mM, about 20 mM, about 30 mM about 40 mM, about 50 mM, about 75 mM or about 100 mM sodium metabisulfite. In some embodiments, residual unreacted formaldehyde is removed by neutralization with about 2 mM sodium metabisulfite.

After removal of residual unreacted formaldehyde (or any other inactivating reagent), the inactivated virus may be further purified. Any method of purifying a virus known in the art (such as the methods described above under the section “Production and purification of Zika virus”) may be employed, including, without limitation, cross flow filtration (CFF), tangential flow filtration (TFF), multimodal chromatography, size exclusion chromatography, cation exchange chromatography, and/or anion exchange chromatography. In some embodiments, the inactivated virus is purified by cross flow filtration (CFF). In some embodiments, the inactivated virus is purified using size exclusion chromatography.

As outlined in detail in the section “Production and purification of Zika virus” above, the purity and/or integrity of an inactivated Zika virus can be routinely determined using size exclusion chromatography. In accordance with the above definition of a “purified Zika virus”, the term “purified inactivated Zika virus” means that the main peak of the inactivated Zika virus when analyzed using size exclusion chromatography is more than 85% of the total area under the curve in the size exclusion chromatography. In some embodiments, the main peak of the inactivated Zika virus when analyzed using size exclusion chromatography is more than 90%, or more than 95%, more than 98% or more than 99% of the total area under the curve in the size exclusion chromatography.

The (purified) inactivated Zika virus described in this section may be useful in vaccines and/or immunogenic compositions as described herein for preventing Zika virus infection in a subject in need thereof and/or for inducing an immune response, such as a protective immune response, against Zika virus in a subject in need thereof, such as a human subject.

### Residual Formaldehyde Content

As formaldehyde is known to be genotoxic and carcinogenic, it is important to keep residual levels of formaldehyde in vaccines or immunogenic compositions comprising viruses inactivated with formaldehyde as low as possible. According to the US pharmacopoeia, the upper limit for residual formaldehyde in vaccines comprising inactivated bacteria or viruses is 0.02% which is equivalent to 100 μg/ml formaldehyde.

Without wishing to be bound by any particular theory, it is postulated that a low content of residual formaldehyde in vaccines or immunogenic compositions, lowers the risk of a subject being administered the vaccine or immunogenic composition of developing adverse effects.

Therefore, in certain aspects of the present disclosure, the vaccines and/or immunogenic compositions comprising an inactivated whole Zika virus as described herein have a particularly low content of residual formaldehyde.

The term “residual formaldehyde content” refers to the amount of formaldehyde which is present in vaccines or immunogenic compositions comprising a Zika virus inactivated with formaldehyde as described above.

In certain embodiments, the vaccines or immunogenic compositions of the present disclosure comprising a Zika virus inactivated with formaldehyde have a residual formaldehyde content of less than 50 μg/mL. In one embodiment, the residual formaldehyde content in the vaccine or immunogenic composition is less than 45 μg/ml, less than 40 μg/ml, less than 35 μg/ml, less than 30 μg/ml, less than 25 μg/ml, less than 20 μg/ml, less than 15 μg/ml or less than 10 μg/ml. In one embodiment, the residual formaldehyde content in the vaccine or immunogenic composition is less than 9.5 μg/ml, less than 9 μg/ml, less than 8.5 μg/ml, less than 8 μg/ml, less than 7.5 μg/ml, less than 7 μg/ml, less than 6.5 μg/ml, less than 6 μg/ml, less than 5.5 μg/ml, less than 5 μg/ml, less than 4.5 μg/ml, less than 4 μg/ml, less than 3.5 μg/ml, less than 3 μg/ml, less than 2.5 μg/ml, less than 2 μg/ml, less than 1.5 μg/ml, less than 1 μg/ml or less than 0.5 μg/ml. In one embodiment, the residual formaldehyde content in the vaccine or immunogenic composition is less than 0.5 μg/ml.

Residual formaldehyde may be determined after residual unreacted formaldehyde has been removed, for instance, by neutralization with sodium metabisulfite as described above. The preparation may optionally also have been subjected to one or more further purification or filtration steps as described above.

### Method for Determining the Residual Formaldehyde Content

The residual formaldehyde content can be determined by any method known to the skilled person. One suitable method is described in EMEA, VICH Topic GL25, Biologicals: Testing of residual formaldehyde, 30 Apr. 2002 and involves the use of Methylbenzothiazolone hydrazone hydrochloride (MBTH). Other methods include acetyl acetone titration, ferric chloride titration and the basic fuchsin test.

A particularly suitable method for determining the residual formaldehyde content in the vaccines or immunogenic compositions comprising an inactivated Zika virus of the present disclosure (cf. also Example 5 below) comprises the steps of:
(a) providing a vaccine or immunogenic composition comprising a Zika virus which has been treated with formaldehyde;
(b) mixing the vaccine or immunogenic composition of (a) with phosphoric acid and 2,4-dinitrophenylhydrazine (DNPH), thereby providing a mixture;
(c) incubating the mixture of (b) under suitable conditions; and
(d) analyzing the mixture for the presence of residual formaldehyde.

The use of DNPH as detection reagent offers the following advantages: (1) high sensitivity, (2) UV detection of the derivatized formaldehyde and (3) one-step sample preparation without heating.

The method for determining the residual formaldehyde content using DNPH as described herein is particularly suitable for detecting residual formaldehyde in vaccines or immunogenic compositions containing an adjuvant such as aluminum hydroxide (cf. also Example 5 below). In certain embodiments, the method for determining the residual formaldehyde content using DNPH as described herein is particularly suitable for detecting residual formaldehyde in vaccines or immunogenic compositions containing from about 0.1 mg/ml to about 1.5 mg/mL aluminum hydroxide as an adjuvant, such as from about 0.4 mg/ml to about 1.2 mg/ml aluminum hydroxide adjuvant or from about 0.1 mg/mL to about 1.0 mg/mL aluminum hydroxide as an adjuvant.

Within the meaning of the present disclosure, the term “aluminum hydroxide” collectively refers to any aluminum hydroxide in a pharmaceutically acceptable form for as an adjuvant. For instance, “aluminum hydroxide” may refer to aluminum oxide hydroxide, precipitated aluminum hydroxide, or gel-like aluminum hydroxide as present in Alhydrogel.

Due to the varying amount of water and the complex stoichiometry of (hydrated) aluminum hydroxide, aluminum oxide hydroxide, and related aluminum compounds (often provided as wet gel suspensions) for use as adjuvants, the amount or concentration of aluminum-based adjuvant in a sample is commonly given as the amount or concentration of aluminum ions in the sample. Thus, as will be readily understood by a skilled artisan, when reference is made to an amount or a concentration of aluminum hydroxide or any other aluminum salt in the present disclosure, the amount or concentration refers to the aluminum content (i.e. the amount of aluminum ions) as such. For instance, when reference is made to 1.0 mg/ml aluminum hydroxide, reference is made to 1.0 mg/mL aluminum or when reference is made to 200 μg aluminum hydroxide, reference is made to 200 μg aluminum.

The method for determining the residual formaldehyde content using DNPH as described herein was validated in terms of specificity, linearity, accuracy, repeatability, robustness and stability according to the International Conference on Harmonization (ICH) Q2 guidelines (see Example 5).

In some embodiments 50 parts of the vaccine or immunogenic composition comprising a Zika virus which has been treated with formaldehyde are mixed with 1 part of 15 to 25% (v/v) phosphoric acid and 2.5 parts of 0.9 to 1.1 mg/ml DNPH. In some embodiments 50 parts of the vaccine or immunogenic composition comprising a Zika virus which has been treated with formaldehyde are mixed with 1 part of 20% (v/v) phosphoric acid and 2.5 parts of 1.0 mg/ml DNPH. In some embodiments, 1 mL of the vaccine or immunogenic composition comprising a Zika virus which has been treated with formaldehyde is mixed with 20 UL of 20% (v/v) phosphoric acid and 50 UL of 1.0 mg/mL DNPH.

In some embodiments the mixture of the vaccine or immunogenic composition comprising a Zika virus which has been treated with formaldehyde, phosphoric acid and DNPH is incubated at a temperature of 18° C. to 30° C. In some embodiments the mixture of the vaccine or immunogenic composition comprising a Zika virus which has been treated with formaldehyde, phosphoric acid and DNPH is incubated at a temperature of 20° C. to 25° C. In some embodiments the mixture of the vaccine or immunogenic composition comprising a Zika virus which has been treated with formaldehyde, phosphoric acid and DNPH is incubated at a temperature of 22° C.

In some embodiments the mixture of the vaccine or immunogenic composition comprising a Zika virus which has been treated with formaldehyde, phosphoric acid and DNPH is incubated for 10 to 30 minutes. In some embodiments the mixture of the vaccine or immunogenic composition comprising a Zika virus which has been treated with formaldehyde, phosphoric acid and DNPH is incubated for 15 to 25 minutes. In some embodiments the mixture of the vaccine or immunogenic composition comprising a Zika virus which has been treated with formaldehyde, phosphoric acid and DNPH is incubated for 20 minutes.

In some embodiments the mixture of the vaccine or immunogenic composition comprising a Zika virus which has been treated with formaldehyde, phosphoric acid and DNPH is incubated at a temperature of 18° C. to 30° C. for 10 to 30 minutes. In some embodiments the mixture of the vaccine or immunogenic composition comprising a Zika virus which has been treated with formaldehyde, phosphoric acid and DNPH is incubated at a temperature of 18° C. to 30° C. for 15 to 25 minutes. In some embodiments the mixture of the vaccine or immunogenic composition comprising a Zika virus which has been treated with formaldehyde, phosphoric acid and DNPH is incubated at a temperature of 18° C. to 30° C. for 20 minutes.

In some embodiments the mixture of the vaccine or immunogenic composition comprising a Zika virus which has been treated with formaldehyde, phosphoric acid and DNPH is incubated at a temperature of 20° C. to 25° C. for 10 to 30 minutes. In some embodiments the mixture of the vaccine or immunogenic composition comprising a Zika virus which has been treated with formaldehyde, phosphoric acid and DNPH is incubated at a temperature of 20° C. to 25° C. for 15 to 25 minutes. In some embodiments the mixture of the vaccine or immunogenic composition comprising a Zika virus which has been treated with formaldehyde, phosphoric acid and DNPH is incubated at a temperature of 20° C. to 25° C. for 20 minutes.

In some embodiments the mixture of the vaccine or immunogenic composition comprising a Zika virus which has been treated with formaldehyde, phosphoric acid and DNPH is incubated at a temperature of 22° C. for 10 to 30 minutes. In some embodiments the mixture of the vaccine or immunogenic composition comprising a Zika virus which has been treated with formaldehyde, phosphoric acid and DNPH is incubated at a temperature of 22° C. for 15 to 25 minutes. In some embodiments the mixture of the vaccine or immunogenic composition comprising a Zika virus which has been treated with formaldehyde, phosphoric acid and DNPH is incubated at a temperature of 22° C. for 20 minutes.

In some embodiments the mixture of the vaccine or immunogenic composition comprising a Zika virus which has been treated with formaldehyde, phosphoric acid and DNPH comprises 50 parts of the vaccine or immunogenic composition, 1 part of 20% (v/v) phosphoric acid and 2.5 parts of 1.0 mg/ml DNPH. In some embodiments, this mixture is incubated at a temperature of 18° C. to 30° C. for 10 to 30 minutes, 15 to 25 minutes, or 20 minutes. In other embodiments, this mixture is incubated at a temperature of 20° C. to 25° C. for 10 to 30 minutes, 15 to 25 minutes, or 20 minutes. In yet other embodiments, this mixture is incubated at a temperature of 22° C. for 10 to 30 minutes, 15 to 25 minutes, or 20 minutes.

After incubation, the mixture of the vaccine or immunogenic composition comprising a Zika virus which has been treated with formaldehyde, phosphoric acid and DNPH may be analyzed by any suitable method. In one embodiment, after incubation, the mixture of the vaccine or immunogenic composition comprising a Zika virus which has been treated with formaldehyde, phosphoric acid and DNPH is analyzed by HPLC. In one embodiment, the HPLC is a reversed-phase HPLC. In one embodiment, the ligand of the reversed-phase HPLC column is selected from C18, n-butal, n-octyl, phenyl and cyanopropyl. In one embodiment, the ligand of the reversed-phase HPLC column is C18. In one embodiment, a mixture of water and acetonitrile (1:1, v/v) is used as the mobile phase in the reversed-phase HPLC. In one embodiment, the detection wavelength is 360 nm.

In one embodiment, the method for determining the residual formaldehyde content in the vaccines or immunogenic compositions comprising an inactivated Zika virus of the present disclosure comprises the steps of:
(a) providing a vaccine or immunogenic composition comprising a Zika virus which has been treated with formaldehyde;
(b) mixing 50 parts of the vaccine or immunogenic composition of (a) with 1 part of 20% (v/v) phosphoric acid and 2.5 parts of 1 mg/ml 2,4-dinitrophenylhydrazine (DNPH), thereby providing a mixture;
(c) incubating the mixture of (b) for 20 minutes at 22° C.; and
(d) analyzing the mixture for the presence of residual formaldehyde by reversed-phase HPLC using a C18 column and a mixture of water and acetonitrile (1:1, v/v) as the mobile phase.

### Residual Replicating Virus

As outlined above, inactivating a Zika virus shall render the Zika virus unable to replicate in host cells, such as mammalian cells, in which a Zika virus that was not subjected to an inactivation step is capable to replicate. Without wishing to be bound by any particular theory, a particular low amount of a residual replicating virus in a vaccine or immunogenic composition lowers the risk of a subject being administered the vaccine or immunogenic composition of developing adverse effects, in particular when the vaccine or immunogenic composition comprises a pathogenic virus such as Zika that could cause inter alia fetal abnormalities. Thus, during the development of vaccines or immunogenic compositions that comprise an inactivated Zika virus, a key assurance is to ensure that only a low amount of infectious virus remains in the vaccines or immunogenic compositions.

Therefore, in certain aspects of the present disclosure, the vaccines and/or immunogenic compositions comprising an inactivated whole Zika virus as described herein have a particularly low content of residual replicating Zika virus.

The term “residual replicating virus” refers to the amount of virus which is still capable of replicating in host cells, such as mammalian cells, present in vaccines or immunogenic compositions comprising an inactivated whole Zika virus as described above.

In some embodiments, the vaccines or immunogenic compositions of the present disclosure comprising an inactivated whole Zika virus comprise less than 1.0 TCID₅₀ of residual replicating virus. In some embodiments, the vaccines or immunogenic compositions of the present disclosure comprising an inactivated whole Zika virus comprise less than 0.8 TCID₅₀ of residual replicating virus. In some embodiments, the vaccines or immunogenic compositions of the present disclosure comprising an inactivated whole Zika virus comprise less than 0.5 TCID₅₀ of residual replicating virus. In some embodiments, the vaccines or immunogenic compositions of the present disclosure comprising an inactivated whole Zika virus comprise less than 0.2 TCID₅₀ of residual replicating virus. In some embodiments, the vaccines or immunogenic compositions of the present disclosure comprising an inactivated whole Zika virus comprise less than 0.1 TCID₅₀ of residual replicating virus.

### Method for Determining the Completeness of Inactivation

The amounts of residual replicating virus as may be present in the vaccines or immunogenic compositions of the present disclosure may be determined by methods providing a suitable limit of detection.

A particular suitable method (termed herein as “method for determining the completeness of inactivation”; cf. also Example 4 below) is described herein. The method for determining the completeness of inactivation uses a sequential infection of two different cell types, thereby providing a particularly low limit of detection (LOD) compared to an assay, which only uses one cell type, such as the conventional TCID₅₀-method or plaque assays. A further advantage of the method for determining the completeness of inactivation is that it avoids the use of animals to determine infectivity of the inactivated virus.

With the method for determining the completeness of inactivation as described herein, a virus content of less than 1.0 TCID₅₀ can be detected. In some embodiments, a virus content of less than 0.8 TCID₅₀ can be detected. In some embodiments, a virus content of less than 0.5 TCID₅₀ can be detected. In some embodiments, a virus content of less than 0.2 TCID₅₀ can be detected. In some embodiments, a virus content of less than 0.1 TCID₅₀ can be detected.

The method for determining the completeness of inactivation of a Zika virus preparation comprises the steps of:
(i) inoculating cultured insect cells with a Zika virus preparation which was subjected to an inactivation step and incubating the insect cells for a first period of time, thereby producing an insect cell supernatant;
(ii) inoculating cultured mammalian cells with the insect cell supernatant produced in (i) and incubating the mammalian cells for a second period of time; and
(iii) determining whether the Zika virus preparation contains a residual replicating virus that produces a cytopathic effect on the mammalian cells.

The cultured insect cells are inoculated with the Zika virus preparation by adding the Zika virus preparation to the insect cell culture which contains insect cells and growth medium. The inoculated insect cells are then incubated for a first period of time with the Zika virus preparation under suitable conditions. In some embodiments, the first period of time is three to seven days. In some embodiments, the first period of time is five to seven days. In some embodiments, the first period of time is six days. Hence, in some embodiments the inoculated insect cells are incubated with the Zika virus preparation for three to seven days. In some embodiments, the inoculated insect cells are incubated with the Zika virus preparation for five to seven days. In some embodiments, the inoculated insect cells are incubated with the Zika virus preparation for six days. During the incubation, any live virus will be secreted into the insect cell supernatant.

The insect cells used may be any insect cells which can be infected by the Zika virus to be investigated and whose viability is not altered by Zika virus infection. The insect cells are selected such that the Zika virus does not have a cytopathic effect on the cells. Suitable insect cells include, but are not limited to, CCL-125 cells, Aag-2 cells, RML-12 cells, C6/36 cells, C7-10 cells, AP-61 cells, A.t. GRIP-1 cells, A.t. GRIP-2 cells, A.t. GRIP-3 cells, UM-AVE1 cells, Mos.55 cells, Sua1B cells, 4a-3B cells, Mos.42 cells, MSQ43 cells, LSB-AA695BB cells, NIID-CTR cells and TRA-171 cells. In some embodiments, the insect cells are C6/36 cells.

The insect cell supernatant produced by incubating the insect cells with the Zika virus preparation is then used to inoculate cultured mammalian cells. For inoculation the insect cell supernatant is transferred to the mammalian cells and incubated with the mammalian cells for 60 to 120 minutes or for 80 to 100 minutes or for 90 minutes. After the inoculation, cell culture medium is added and the mammalian cells are incubated with the insect cell supernatant for a second period of time under suitable conditions. In some embodiments, the second period of time is three to 14 days. In some embodiments, the second period of time is five to twelve days. In some embodiments, the second period of time is six to ten days. In some embodiments, the second period of time is seven to nine days. In some embodiments, the second period of time is eight days. Hence, in some embodiments the inoculated mammalian cells are incubated with the insect cell supernatant for three to 14 days. In some embodiments, the inoculated mammalian cells are incubated with the insect cell supernatant for five to twelve days. In some embodiments, the inoculated mammalian cells are incubated with the insect cell supernatant for seven to nine days. In some embodiments, the inoculated mammalian cells are incubated with the insect cell supernatant for eight days. During the incubation, any residual replicating virus will exert a cytopathic effect on the mammalian cells such as Vero cells.

The mammalian cells used may be any mammalian cells which can be infected by the Zika virus to be investigated and on which the Zika virus exerts a cytopathic effect. Suitable mammalian cells include, but are not limited to, VERO cells, LLC-MK2 cells, MDBK cells, MDCK cells, ATCC CCL34 MDCK (NBL2) cells, MDCK 33016 (deposit number DSM ACC 2219 as described in WO97/37001) cells, BHK21-F cells, HKCC cells, and Chinese hamster ovary cells (CHO cells). In some embodiments, the mammalian cells are Vero cells.

In one embodiment, the method for determining the completeness of inactivation of a Zika virus preparation comprises the steps of:
(i) inoculating cultured insect cells with a Zika virus preparation which was subjected to an inactivation step and incubating the insect cells for 3 to 7 days, thereby producing an insect cell supernatant;
(ii) inoculating cultured mammalian cells with the insect cell supernatant produced in (i) and incubating the mammalian cells 3 to 14 days; and
(iii) determining whether the virus preparation contains a residual replicating virus that produces a cytopathic effect on the mammalian cells.

In one embodiment, the method for determining the completeness of inactivation of a Zika virus preparation comprises the steps of:
(i) inoculating C6/36 cells with a Zika virus preparation which was subjected to an inactivation step and incubating the C6/36 cells for a first period of time, thereby producing an C6/36 cell supernatant;
(ii) inoculating Vero cells with the C6/36 cell supernatant produced in (i) and incubating the Vero cells for a second period of time; and
(iii) determining whether the virus preparation contains a residual replicating virus that produces a cytopathic effect on the Vero cells.

In one embodiment, the method for determining the completeness of inactivation of a Zika virus preparation comprises the steps of:
(i) inoculating C6/36 cells with a Zika virus preparation which was subjected to an inactivation step and incubating the C6/36 cells for 3 to 7 days, thereby producing an C6/36 cell supernatant;
(ii) inoculating Vero cells with the C6/36 cell supernatant produced in (i) and incubating the Vero cells for 3 to 14 days; and
(iii) determining whether the virus preparation contains a residual replicating virus that produces a cytopathic effect on the Vero cells.

In one embodiment, the method for determining the completeness of inactivation of a Zika virus preparation comprises the steps of:
(i) inoculating C6/36 cells with a Zika virus preparation which was subjected to an inactivation step and incubating the C6/36 cells for 6 days, thereby producing an C6/36 cell supernatant;
(ii) inoculating Vero cells with the C6/36 cell supernatant produced in (i) and incubating the Vero cells for 8 days; and
(iii) determining whether the virus preparation contains a residual replicating virus that produces a cytopathic effect on the Vero cells.

At the end of the second period of time it is determined whether the virus preparation has a cytopathic effect on the mammalian cells. A cytopathic effect is any change in the cell structure caused by viral invasion, infection, and budding from the cells during viral replication. In the method of the present disclosure, the cytopathic effect is determined by a change in the media color from pink to orange or yellow, if the cells are cultured in a medium containing phenol red, or by a microscopic examination of the mammalian cells. If the microscopic examination of the mammalian cells shows that the cells round, begin to pull away from the tissue culture vessel (plate, well or flask), or clear from the tissue culture plate/flask, it is considered that a cytopathic effect is present. Other indicia of a cytopathic effect include the fusion of adjacent cells to form syncytia and the appearance of nuclear or cytoplasmic inclusion bodies.

A Zika virus may be considered as completely inactivated if no cytopathic effect is observed in step (iii) of the method for determining the completeness of inactivation of a Zika virus preparation as described above. In some embodiments of the present disclosure, the term “inactivated whole Zika virus” may also refer to a whole Zika virus that does not produce a cytopathic effect in step (iii) of the method for determining the completeness of inactivation as described above.

### Adjuvants

Other aspects of the present disclosure relate to vaccines or immunogenic compositions comprising a Zika virus (which may be an inactivated Zika virus as described above) and further comprising one or more adjuvants. Such adjuvanted vaccines or immunogenic compositions of the present disclosure may be useful for preventing Zika virus infection in a human subject in need thereof and/or inducing an immune response, such as a protective immune response, against Zika virus in a human subject in need thereof.

Various methods of achieving an adjuvant effect for vaccines are known and may be used in conjunction with the Zika virus vaccines or immunogenic compositions disclosed herein. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9.

In some embodiments, the (inactivated whole) Zika virus may be mixed with at least one adjuvant, at a weight-based ratio of from about 10:1 to about 10¹⁰:1 antigen:adjuvant, e.g., from about 10:1 to about 100:1, from about 100:1 to about 10³:1, from about 10³:1 to about 10⁴:1, from about 10⁴:1 to about 10⁵:1, from about 10⁵:1 to about 10⁶:1, from about 10⁶:1 to about 10⁷:1, from about 10⁷:1 to about 10⁸:1, from about 10⁸:1 to about 10⁹:1, or from about 10⁹:1 to about 10¹⁰:1 antigen:adjuvant, wherein the antigen is the (inactivated whole) Zika virus.

Exemplary adjuvants may include, but are not limited to, aluminum salts, calcium phosphate, toll-like receptor (TLR) agonists (such as bacterial flagellin), monophosphoryl lipid A (MLA), MLA derivatives, synthetic lipid A, lipid A mimetics or analogs, cytokines, saponins, muramyl dipeptide (MDP) derivatives, CpG oligonucleotides, lipopolysaccharides (LPS) of gram-negative bacteria, polyphosphazenes, emulsions (such as oil emulsions), chitosan, vitamin D, stearyl or octadecyl tyrosine, virosomes, cochleates, poly(lactide-co-glycolides) (PLG) microparticles, poloxamer particles, microparticles, liposomes, Complete Freund's Adjuvant (CFA), and Incomplete Freund's Adjuvant (IFA). Further details on suitable adjuvants can be derived from WO 2019/090228 A2, the disclosure of which is herewith incorporated by reference.

In some embodiments, the adjuvant is an aluminum salt. In some embodiments, the aluminum salt is aluminum phosphate, aluminum hydroxide, or potassium aluminum sulfate. In some embodiments, the adjuvant is Alhydrogel® or Alhydrogel® 85. Both, Alhydrogel® and Alhydrogel® 85 are available, for instance, at 2% concentration, referring to about 10 mg/mL Aluminum content.

In some embodiments, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the (inactivated whole) Zika virus are adsorbed to the aluminum salt adjuvant. In some embodiments, at least 90% of the (inactivated whole) Zika virus are adsorbed to the aluminum salt adjuvant. In some embodiments, at least 95% of the (inactivated whole) Zika virus are adsorbed to the aluminum salt adjuvant. In some embodiments, at least 99% of the (inactivated whole) Zika virus are adsorbed to the aluminum salt adjuvant.

In some embodiments, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the (inactivated whole) Zika virus are adsorbed to the adjuvant, wherein the adjuvant is aluminum hydroxide. In some embodiments, at least 90% of the (inactivated whole) Zika virus are adsorbed to the adjuvant, wherein the adjuvant is aluminum hydroxide. In some embodiments, at least 95% of the (inactivated whole) Zika virus are adsorbed to the adjuvant, wherein the adjuvant is aluminum hydroxide. In some embodiments, at least 99% of the (inactivated whole) Zika virus are adsorbed to the adjuvant, wherein the adjuvant is aluminum hydroxide.

In some embodiments, the vaccine or immunogenic composition comprises from about 100 μg to about 700 μg, from about 100 μg to about 600 μg, from about 100 μg to about 500 μg, from about 125 μg to about 500 μg, from about 150 μg to about 500 μg, from about 175 μg to about 500 μg, from about 100 μg to about 450 μg, from about 125 μg to about 450 μg, from about 150 μg to about 450 μg, from about 175 μg to about 450 μg, from about 100 μg to about 400 μg, from about 125 μg to about 400 μg, from about 150 μg to about 400 μg, from about 175 μg to about 400 μg, from about 100 μg to about 350 μg, from about 125 μg to about 350 μg, from about 150 μg to about 350 μg, from about 175 μg to about 350 μg, from about 100 μg to about 300 μg, from about 125 μg to about 300 μg, from about 150 μg to about 300 μg, from about 175 μg to about 300 μg, from about 100 μg to about 250 μg, from about 125 μg to about 250 μg, from about 150 μg to about 250 μg, from about 175 μg to about 250 μg, from about 100 μg to about 225 μg, from about 125 μg to about 225 μg, from about 150 μg to about 225 μg, from about 175 μg to about 225 μg, or about 200 μg of an aluminum salt adjuvant. In some embodiments, the vaccine or immunogenic composition comprises from about 100 μg to about 600 μg, from about 100 μg to about 300 μg, from about 150 μg to about 250 μg, from about 175 μg to about 225 μg, or about 200 μg of an aluminum salt adjuvant. In some embodiments, the vaccine or immunogenic composition comprises from about 100 μg to about 300 μg of an aluminum salt adjuvant. In some embodiments, the vaccine or immunogenic composition comprises from about 150 μg to about 250 μg of an aluminum salt adjuvant. In some embodiments, the vaccine or immunogenic composition comprises from about 175 μg to about 225 μg of an aluminum salt adjuvant. In some embodiments, the vaccine or immunogenic composition comprises about 200 μg of an aluminum salt adjuvant.

In some embodiments, the vaccine or immunogenic composition comprises from about 100 μg to about 700 μg, from about 100 μg to about 600 μg, from about 100 μg to about 500 μg, from about 125 μg to about 500 μg, from about 150 μg to about 500 μg, from about 175 μg to about 500 μg, from about 100 μg to about 450 μg, from about 125 μg to about 450 μg, from about 150 μg to about 450 μg, from about 175 μg to about 450 μg, from about 100 μg to about 400 μg, from about 125 μg to about 400 μg, from about 150 μg to about 400 μg, from about 175 μg to about 400 μg, from about 100 μg to about 350 μg, from about 125 μg to about 350 μg, from about 150 μg to about 350 μg, from about 175 μg to about 350 μg, from about 100 μg to about 300 μg, from about 125 μg to about 300 μg, from about 150 μg to about 300 μg, from about 175 μg to about 300 μg, from about 100 μg to about 250 μg, from about 125 μg to about 250 μg, from about 150 μg to about 250 μg, from about 175 μg to about 250 μg, from about 100 μg to about 225 μg, from about 125 μg to about 225 μg, from about 150 μg to about 225 μg, from about 175 μg to about 225 μg, or about 200 μg of an adjuvant, wherein the adjuvant is aluminum hydroxide. In some embodiments, the vaccine or immunogenic composition comprises from about 100 μg to about 600 μg, from about 100 μg to about 300 μg, from about 150 μg to about 250 μg, or about 200 μg of an adjuvant, wherein the adjuvant is aluminum hydroxide. In some embodiments, the vaccine or immunogenic composition comprises from about 100 μg to about 300 μg of an adjuvant, wherein the adjuvant is aluminum hydroxide. In some embodiments, the vaccine or immunogenic composition comprises from about 150 μg to about 250 μg of an adjuvant, wherein the adjuvant is aluminum hydroxide. In some embodiments, the vaccine or immunogenic composition comprises from about 175 μg to about 225 μg of an aluminum salt adjuvant, wherein the adjuvant is aluminum hydroxide. In some embodiments, the vaccine or immunogenic composition comprises about 200 μg of an adjuvant, wherein the adjuvant is aluminum hydroxide.

As outlined above and will be appreciated by a person skilled in the art, the specification of a certain μg-amount of aluminum salt or aluminum hydroxide refers to the aluminum amount as such. For instance, “600 μg aluminum hydroxide” or “600 μg aluminum salt” are to be understood as “600 μg aluminum”, i.e. “600 μg Al³⁺”.

In some embodiments, the Zika virus is mixed with the adjuvant (such as an aluminum salt adjuvant) and the mixture is incubated under suitable conditions for a period that ranges from about 1 hour to about 24 hours (e.g., about 16 hours to about 24 hours). In some embodiments, the mixture is incubated at a temperature that ranges from about 2° C. to about 8° C. In some embodiments, the mixture is incubated under constant mixing using any suitable mixer known in the art. In some embodiments, the mixture is incubated at pH that ranges in value from about 6.5 to about 8.5, from about 6.5 to about 8, from about 6.8 to about 7.8, from about 6.9 to about 7.6, from about 7 to about 7.5, from about 6.8 to about 8.5, from about 6.9 to about 8.5, or from about 7 to about 8.5.

### Formulations and Dose

### Formulation

Further aspects of the present disclosure relate to formulations of the vaccines or immunogenic compositions of the present disclosure containing a (inactivated whole) Zika virus as the antigen as described herein.

Typically, the vaccines or immunogenic compositions of the present disclosure are prepared as injectables either as liquid solutions or suspensions. Solid forms (dry substances) suitable for solving in, or suspending in, a liquid prior to injection may also be prepared.

The vaccines or immunogenic compositions of the present disclosure may further comprise excipients which are pharmaceutically acceptable and compatible with the active ingredient (the (inactivated whole) Zika virus). Suitable excipients are, for example, water, saline, dextrose, sucrose, glycerol, ethanol, or the like, and combinations thereof. A particular suitable excipient is sucrose. In addition, if desired, the vaccine or immunogenic composition may contain auxiliary substances such as wetting or emulsifying agents or pH buffering agents. A thorough discussion of excipients and other components suitable for the application in the vaccines or immunogenic compositions of the present disclosure is available in Gennaro (2000) Remington: The Science and Practice of Pharmacy. 20th edition, ISBN: 0683306472.

In some embodiments, the vaccine or immunogenic composition has a unit dose volume of from about 0.1 mL to about 0.8 mL. In some embodiments, the vaccine or immunogenic composition has a unit dose volume of about 0.5 mL. In other embodiments, the vaccine or immunogenic composition has a unit dose volume of about 0.25 mL. A unit dose volume of about 0.5 mL is particularly suitable for intramuscular or subcutaneous administration.

The vaccines or immunogenic compositions of the present disclosure may be pre-filled into conventional syringes and needles for systemic, parenteral administration.

To control the tonicity of the vaccines or immunogenic compositions of the present disclosure, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, etc.

The vaccines or immunogenic compositions of the present disclosure may include one or more (pharmaceutically acceptable) buffers. Typical buffers are phosphate buffers; Tris buffers; borate buffers; succinate buffers; histidine buffers; and citrate buffers. Buffers will typically be included in the 5-20 mM range.

The pH of a vaccine or immunogenic composition will generally be between 5.0 and 8.5 or 5.0 and 8.1, and more typically between 6.0 and 8.5 e.g. between 6.0 and 8.0, between 6.5 and 8.0, between 6.5 and 7.5, between 7.0 and 8.5, between 7.0 and 8.0, or between 7.0 and 7.8. A manufacturing process of the present disclosure may therefore include a step of adjusting the pH of the bulk vaccine prior to packaging.

The vaccines or immunogenic compositions of the present disclosure are preferably sterile. They are preferably non pyrogenic, e.g. containing <1 EU (endotoxin unit, a standard measure) per dose, and preferably <0.1 EU per dose. They are preferably gluten free.

The vaccines or immunogenic compositions are preferably stored at between 2° C. and 8° C. They should ideally be kept out of direct light.

Suitable methods of preparing vaccines or immunogenic compositions can also be found in Remington's Pharmaceutical Sciences (Mack Publishing Co., Easton Pa., 1990 or later versions).

### Dose

In some embodiments, the vaccine or immunogenic composition comprises a dose (may also be referred to as dosage herein) of from about 1 μg to about 100 μg of Zika virus antigen. In some embodiments, the vaccine or immunogenic composition comprises a dose of from about 1 μg to about 40 μg of Zika virus antigen. In some embodiments, the vaccine or immunogenic composition comprises a dose of from about 1 μg to about 30 μg of Zika virus antigen. In some embodiments, the vaccine or immunogenic composition comprises a dose of from about 1 μg to about 20 μg of Zika virus antigen. In some embodiments, the vaccine or immunogenic composition comprises a dose of from about 1 μg to about 15 μg of Zika virus antigen. In some embodiments, the vaccine or immunogenic composition comprises a dose of from about 2 μg to about 15 μg, or from about 2 μg to about 10 μg, or from about 5 μg to about 15 μg, or from about 6 μg to about 15 μg, or from about 10 μg to about 15 μg of Zika virus antigen. In one embodiment, the vaccine or immunogenic composition comprises a dose of about 15 μg of Zika virus antigen. In one embodiment, the vaccine or immunogenic composition comprises a dose of about 20 μg of Zika virus antigen. In some embodiments, the vaccine or immunogenic composition comprises a dose of from about 5 μg to about 15 μg of Zika virus antigen. In some embodiments, the vaccine or immunogenic composition comprises a dose of from about 6 μg to about 15 μg of Zika virus antigen. In some embodiments, the vaccine or immunogenic composition comprises a dose of from about 7 μg to about 15 μg of Zika virus antigen. In some embodiments, the vaccine or immunogenic composition comprises a dose of from about 7 μg to about 13 μg of Zika virus antigen. In some embodiments, the vaccine or immunogenic composition comprises a dose of from about 7.5 μg to about 12.5 μg of Zika virus antigen. In some embodiments, the vaccine or immunogenic composition comprises a dose of from about 8 μg to about 12 μg of Zika virus antigen. In some embodiments, the vaccine or immunogenic composition comprises a dose of from about 8.5 μg to about 11.5 μg of Zika virus antigen. In some embodiments, the vaccine or immunogenic composition comprises a dose of from about 9 μg to about 11 μg of Zika virus antigen. In some embodiments, the vaccine or immunogenic composition comprises a dose of from about 9.5 μg to about 10.5 μg of Zika virus antigen.

In preferred embodiments, the vaccine or immunogenic composition comprises a dose of about 2 μg of Zika virus antigen. In other preferred embodiments, the vaccine or immunogenic composition comprises a dose of about 5 μg of Zika virus antigen. In other preferred embodiments, the vaccine or immunogenic composition comprises a dose of about 10 μg of Zika virus antigen.

In one preferred embodiment, the vaccine or immunogenic composition comprises a dose of from about 5 μg to about 15 μg of Zika virus antigen.

In one preferred embodiment, the vaccine or immunogenic composition comprises a dose of from about 6 μg to about 15 μg of Zika virus antigen.

In one preferred embodiment, the vaccine or immunogenic composition comprises a dose of from about 8 μg to about 12 μg of Zika virus antigen.

In one preferred embodiment, the vaccine or immunogenic composition comprises a dose of from about 9 μg to about 11 μg of Zika virus antigen, such as about 10 μg of Zika virus antigen.

In preferred embodiments, the Zika virus antigen is an inactivated whole Zika virus (wherein the term “whole Zika virus” may simply also be referred to as “inactivated Zika virus” herein). In certain embodiments, the Zika virus antigen is a purified, inactivated whole Zika virus (wherein the term “whole Zika virus” may simply also be referred to as “inactivated Zika virus” herein).

In one embodiment, the vaccine or immunogenic composition comprises a dose of from about 1 μg to about 20 μg of an antigen from a Zika virus, wherein the antigen is an inactivated whole Zika virus.

In one embodiment, the vaccine or immunogenic composition comprises a dose of from about 1 μg to about 20 μg of an antigen from a Zika virus, wherein the antigen is an inactivated whole Zika virus and wherein the mean peak of the inactivated whole Zika virus when analyzed by size exclusion chromatography is more than 85% of the total area under the curve in the size exclusion chromatography.

In one embodiment, the vaccine or immunogenic composition comprises a dose of from about 2 μg to about 10 μg of an antigen from a Zika virus, wherein the antigen is an inactivated whole Zika virus.

In one embodiment, the vaccine or immunogenic composition comprises a dose of from about 2 μg to about 10 μg of an antigen from a Zika virus, wherein the antigen is an inactivated whole Zika virus and wherein the mean peak of the inactivated whole Zika virus when analyzed by size exclusion chromatography is more than 85% of the total area under the curve in the size exclusion chromatography.

In one embodiment, the vaccine or immunogenic composition comprises a dose of about 2 μg of an antigen from a Zika virus, wherein the antigen is an inactivated whole Zika virus.

In one embodiment, the vaccine or immunogenic composition comprises a dose of about 2 μg of an antigen from a Zika virus, wherein the antigen is an inactivated whole Zika virus and wherein the mean peak of the inactivated whole Zika virus when analyzed by size exclusion chromatography is more than 85% of the total area under the curve in the size exclusion chromatography.

In one embodiment, the vaccine or immunogenic composition comprises a dose of about 5 μg of an antigen from a Zika virus, wherein the antigen is an inactivated whole Zika virus.

In one embodiment, the vaccine or immunogenic composition comprises a dose of about 5 μg of an antigen from a Zika virus, wherein the antigen is an inactivated whole Zika virus and wherein the mean peak of the inactivated whole Zika virus when analyzed by size exclusion chromatography is more than 85% of the total area under the curve in the size exclusion chromatography.

In one embodiment, the vaccine or immunogenic composition comprises a dose of about 10 μg of an antigen from a Zika virus, wherein the antigen is an inactivated whole Zika virus.

In one embodiment, the vaccine or immunogenic composition comprises a dose of about 10 μg of an antigen from a Zika virus, wherein the antigen is an inactivated whole Zika virus and wherein the mean peak of the inactivated whole Zika virus when analyzed by size exclusion chromatography is more than 85% of the total area under the curve in the size exclusion chromatography.

In one preferred embodiment, the vaccine or immunogenic composition comprises a dose of from about 9 μg to about 11 μg of Zika virus antigen wherein the antigen is an inactivated whole Zika virus.

In one preferred embodiment, the vaccine or immunogenic composition comprises a dose of from about 9 μg to about 11 μg of Zika virus antigen, such as about 10 μg of Zika virus antigen, wherein the antigen is an inactivated whole Zika virus and wherein the mean peak of the inactivated whole Zika virus when analyzed by size exclusion chromatography is more than 85% of the total area under the curve in the size exclusion chromatography.

In one preferred embodiment, the vaccine or immunogenic composition comprises a dose of from about 8 μg to about 12 μg of Zika virus antigen wherein the antigen is an inactivated whole Zika virus.

In one preferred embodiment, the vaccine or immunogenic composition comprises a dose of from about 8 μg to about 12 μg of Zika virus antigen wherein the antigen is an inactivated whole Zika virus and wherein the mean peak of the inactivated whole Zika virus when analyzed by size exclusion chromatography is more than 85% of the total area under the curve in the size exclusion chromatography.

In one preferred embodiment, the vaccine or immunogenic composition comprises a dose of from about 6 μg to about 15 μg of Zika virus antigen, wherein the antigen is an inactivated whole Zika virus.

In one preferred embodiment, the vaccine or immunogenic composition comprises a dose of from about 6 μg to about 15 μg of Zika virus antigen, wherein the antigen is an inactivated whole Zika virus and wherein the mean peak of the inactivated whole Zika virus when analyzed by size exclusion chromatography is more than 85% of the total area under the curve in the size exclusion chromatography.

In one embodiment, the vaccine or immunogenic composition comprises a dose of more than 5 μg, such as from about 7 μg to about 13 μg, of an antigen from a Zika virus, wherein the antigen is an inactivated whole Zika virus.

In one embodiment, the vaccine or immunogenic composition comprises a dose of more than 5 μg, such as from about 7 μg to about 13 μg, of an antigen from a Zika virus, wherein the antigen is an inactivated whole Zika virus and wherein the mean peak of the inactivated whole Zika virus when analyzed by size exclusion chromatography is more than 85% of the total area under the curve in the size exclusion chromatography.

In some embodiments, the dose (may also be referred to as dosage herein) is presented in a unit dose volume of about 0.5 mL.

The amount of the (purified or purified inactivated) Zika virus antigen, can be determined by a Bradford assay (Bradford et al. (1976) Anal. Biochem. 72:248-254) using defined amounts of recombinant Zika envelope protein to establish the standard curve. Thus, the dosage of the antigen as described in this section may also be referred to as micrograms (μg) of Zika virus envelope protein (μg E protein). μg antigen and μg E protein thus carry the same meaning within the present disclosure.

### Safety

The vaccines or immunogenic compositions of the present disclosure are particularly safe and thus well suited for administration to human subjects, in particular also human subjects being women of childbearing potential or women that intend to become pregnant or pregnant women. As outlined in Example 6 below, during the Phase I clinical trial with the vaccine/immunogenic composition according to the present disclosure, two women gave birth to healthy babies.

Certain embodiments of the present disclosure are thus directed to a vaccine or immunogenic composition comprising a Zika virus antigen, wherein the antigen is an inactivated whole virus and the vaccine or immunogenic composition induces fever in 7% or less, and/or fatigue in 28% or less, and/or arthralgia in 13% or less, and/or myalgia in 20% or less, and/or malaise in 25% or less, and/or headache in 25% or less of a human subject population of at least 20 flavivirus primed human subjects, wherein the occurrence of fever, and/or fatigue, and/or arthralgia, and/or myalgia, and/or malaise, and/or headache is determined up to 7 days after the administration of the vaccine or immunogenic composition. In certain embodiments, the administration encompasses two doses administered from about 1 to about 16 weeks apart, or from about 1 to about 6 weeks apart, or from about 25 to about 30 days apart. In certain embodiments, the vaccine or immunogenic composition comprises a dose of from about 2 μg to about 10 μg of a Zika virus antigen, wherein the antigen is an inactivated whole virus, optionally wherein the vaccine or immunogenic composition further comprises from about 150 μg to 250 μg of an aluminum salt adjuvant, such as aluminum hydroxide. In certain embodiments, the vaccine or immunogenic composition comprises a dose of from about 6 μg to about 15 μg of a Zika virus antigen, wherein the antigen is an inactivated whole virus, optionally wherein the vaccine or immunogenic composition further comprises from about 175 μg to 225 μg of an aluminum salt adjuvant, such as aluminum hydroxide.

Certain embodiments of the present disclosure are directed to a vaccine or immunogenic composition comprising a dose of about 2 μg of a Zika virus antigen, wherein the antigen is an inactivated whole virus and the vaccine or immunogenic compositions induces no fever, and/or fatigue in 10% or less, and/or arthralgia in 13% or less, and/or myalgia in 18% or less, and/or malaise in 13% or less, and/or headache in 16% or less of a human subject population of at least 20 flavivirus primed human subjects, wherein the occurrence of fever, and/or fatigue, and/or arthralgia, and/or myalgia, and/or malaise, and/or headache is determined up to 7 days after the administration of the vaccine or immunogenic composition. In certain embodiments, the administration encompasses two doses administered from about 1 to about 16 weeks apart, or from about 1 to about 6 weeks apart, or from about 25 to about 30 days apart. In certain embodiments, the vaccine or immunogenic composition further comprises from about 150 μg to about 250 μg, such as from about 175 μg to about 225 μg, of an aluminum salt adjuvant, such as aluminum hydroxide.

Certain embodiments of the present disclosure are directed to a vaccine or immunogenic composition comprising a dose of about 5 μg of a Zika virus antigen, wherein the antigen is an inactivated whole virus and the vaccine or immunogenic compositions induces fever in 7% or less, and/or fatigue in 25% or less, and/or arthralgia in 12% or less, and/or myalgia in 14% or less, and/or malaise in 17% or less, and/or headache in 23% or less of a human subject population of at least 20 flavivirus primed human subjects, wherein the occurrence of fever, and/or fatigue, and/or arthralgia, and/or myalgia, and/or malaise, and/or headache is determined up to 7 days after the administration of the vaccine or immunogenic composition. In certain embodiments, the administration encompasses two doses administered from about 1 to about 16 weeks apart, or from about 1 to about 6 weeks apart, or from about 25 to about 30 days apart. In certain embodiments, the vaccine or immunogenic composition further comprises from about 150 μg to 250 μg, such as from about 175 μg to about 225 μg, of an aluminum salt adjuvant, such as aluminum hydroxide.

Certain embodiments of the present disclosure are directed to a vaccine or immunogenic composition comprising a dose of from about 6 μg to about 15 μg, such as about 10 μg, of a Zika virus antigen, wherein the antigen is an inactivated whole virus and the vaccine or immunogenic compositions induces no fever, and/or headache in 20% or less, and/or fatigue in 20% or less, and/or arthralgia in 6% or less, and/or myalgia in 12% or less, and/or malaise in 25% or less of a human subject population of at least 20 flavivirus primed human subjects, wherein the occurrence of fever, and/or headache, and/or fatigue, and/or arthralgia, and/or myalgia, and/or malaise is determined up to 7 days after the administration of the vaccine or immunogenic composition. In certain embodiments, the administration encompasses two doses administered from about 1 to about 16 weeks apart, or from about 1 to about 6 weeks apart, or from about 25 to about 30 days apart. In certain embodiments, the vaccine or immunogenic composition further comprises from about 150 μg to 250 μg, such as from about 175 μg to about 225 μg, of an aluminum salt adjuvant, such as aluminum hydroxide.

Certain embodiments of the present disclosure are directed to a method for inducing an immune response against Zika virus and/or preventing Zika virus infection and/or preventing Zika virus disease in a human subject or a human subject population, the method comprising administering to the human subject or the individuals of the human subject population the vaccines or immunogenic compositions as described herein.

Certain embodiments of the present disclosure are directed to the use of the vaccines or immunogenic compositions as described herein in the manufacture of a medicament for inducing an immune response against Zika virus and/or preventing Zika virus infection and/or preventing Zika virus disease in a human subject or a human subject population.

Certain embodiments of the present disclosure are directed to the vaccines or immunogenic compositions as described herein for use in inducing an immune response against Zika virus and/or preventing Zika virus infection and/or preventing Zika virus disease in a human subject or a human subject population.

### Immunogenicity

The vaccines or immunogenic compositions of the present disclosure provide for high neutralizing antibody titers, seroconversion rates, and seropositivity rates in both flavivirus-naïve and flavivirus-primed human subjects. Further, high neutralizing antibody titers are not only induced shortly after administration of the vaccines or immunogenic compositions of the present disclosure, but also persist for a long time after completion of the primary administration such as for 6 months, 12 months, and/or 24 months after completion of the primary administration at a certain level.

Thus, the vaccines or immunogenic compositions according to the present disclosure are on the one hand beneficial in an outbreak situation or in a situation where a traveler from a Zika non-endemic region visits a Zika endemic region within a short period of time from the administration of the vaccine or immunogenic composition, where it is necessary to induce high amounts of neutralizing antibody titers shortly after administration. On the other hand, the vaccines or immunogenic compositions according to the present disclosure are beneficial as the immune response induced upon administration is long-lasting and does not require a booster administration up to 6 months, or up to 12 months, or up to 24 months after the primary administration scheme (including a first and a second dose), which lowers the costs for vaccination and also provides a higher comfort for the vaccinated subjects, as frequent booster administrations are avoided.

Within the meaning of this disclosure, a Zika endemic region is defined as an area with risk of infection as defined by the Centers for Disease Control and Prevention. For example, as of March 2018 these areas have been: Asia: Bangladesh, Burma (Myanmar), Cambodia, India, Indonesia, Laos, Malaysia, Maldives, Pakistan, Philippines, Singapore, Thailand, Timor-Leste (East Timor), Vietnam. The Pacific Islands: Fiji, Marshall Islands, Papua New Guinea, Samoa, Solomon Islands, Tonga. The Caribbean: Anguilla; Antigua and Barbuda; Aruba; Barbados; Bonaire; British Virgin Islands; Cuba; Curaçao; Dominica; Dominican Republic; Grenada; Haiti; Jamaica; Montserrat; the Commonwealth of Puerto Rico, a US territory; Saba; Saint Kitts and Nevis; Saint Lucia; Saint Martin; Saint Vincent and the Grenadines; Sint Eustatius; Sint Maarten; Trinidad and Tobago; Turks and Caicos Islands; US Virgin Islands. Mexico/Central America: Belize, Costa Rica, El Salvador, Guatemala, Honduras, Nicaragua, Panama. South America: Argentina, Bolivia, Brazil, Colombia, Ecuador, French Guiana, Guyana, Paraguay, Peru, Suriname, Venezuela. Africa: Angola, Benin, Burkina-Faso, Burundi, Cameroon, Cape Verde, Central African Republic, Chad, Congo (Congo-Brazzaville), Côthe d′Ivoire, Democratic Republic of the Congo (Congo-Kinshasa), Equatorial Guinea, Gabon, Gambia, Ghana, Guinea, Guinea-Bissau, Kenya, Liberia, Mali, Niger, Nigeria, Rwanda, Senegal, Sierra Leone, South Sudan, Sudan, Tanzania, Togo, Uganda. These areas may change.

Administration of the vaccines or immunogenic compositions of the present disclosure will generally result in the development of a secretory, cellular and/or antibody-mediated immune response to the vaccine in the subject. Usually, such a response includes, but is not limited to, one or more of the following effects: the production of antibodies from any of the immunological classes, such as immunoglobulins A, D, E, G, or M; the proliferation of B and T lymphocytes; the provision of activation, growth, and differentiation signals to immunological cells; expansion of helper T cells, suppressor T cells, and/or cytotoxic T cells.

Within the meaning of the present disclosure, flavivirus naïve human subjects are defined to be human subjects without detectable serum antibodies against a panel of flaviviruses, as measured by a reactive antibody-based assay. The assay is based on the Luminex platform to simultaneously detect multiple target antigens in the same sample. This bead-based assay is highly sensitive, specific and reproducible. For the present disclosure, the antigens targeted are from Zika virus, Dengue virus, Yellow fever virus (YFV), Japanese Encephalitis virus (JEV), Usutu-Virus (USUV), St. Louis Encephalitis virus (SLEV) and West Nile virus (WNV). Due to the antibodies being induced by infection with a certain flavivirus being usually cross reactive with other flaviviruses, the current antigen set is thought to detect any prior flavivirus exposure. For a protocol on how the Luminex assay for determination of the serostatus of subjects is conducted, reference is also made to Example 6 and Bohning et al., 2021 (A high throughput reporter virus like particle microneutralization assay for quantitation of Zika virus neutralizing antibodies in multiple species), PLOS One 16:e0250516. Further, references for the Luminex concept are: Dias D, Van Doren J, Schlottmann S, Kelly S, Puchalski D, Ruiz W, Boerckel P, Kessler J, Antonello J M, Green T, Brown M, Smith J, Chirmule N, Barr E, Jansen K U, Esser M T. 2005. Optimization and validation of a multiplexed Luminex assay to quantify antibodies to neutralizing epitopes on human papillomaviruses 6, 11, 16, and 18. Clin. Diagn. Lab. Immunol. 12:959-969 [PMC free article] [PubMed]. Ayouba A et al Development of a Sensitive and Specific Serological Assay Based on Luminex Technology for Detection of Antibodies to Zaire Ebola Virus. J Clin Microbiol. 2017 Dec. 28; 55 (1): 165-176. doi: 10.1128/JCM.01979-16. A further set-up of a Luminex assay using Zika virus antigens can be found in WO 2021/236845. These references are herewith incorporated by reference.

Within the meaning of the disclosure, flavivirus primed human subjects are human subjects that provide serum antibodies directed against at least one of the flaviviruses selected from the group consisting of Zika virus, Dengue virus, Yellow fever virus (YFV), Japanese Encephalitis virus (JEV), Usutu-Virus (USUV), St. Louis Encephalitis virus (SLEV) and West Nile virus (WNV) above the threshold of the Luminex assay described above and in Example 6.

Within the meaning of the present disclosure, the plaque reduction neutralization test (PRNT) refers to an assay for determining anti-Zika virus neutralizing antibody titers in human subjects. The serum sample or solution of antibody to be tested is diluted and mixed with a viral suspension. This mixture is incubated to allow the antibody to react with the virus. The mixture is then poured over a confluent monolayer of host cells. The surface of the cell layer is covered in a layer of agar or carboxymethyl cellulose to prevent the virus from spreading indiscriminately. The concentration of plaque forming units can be estimated by the number of plaques (regions of infected cells) formed after a few days. Depending on the virus, the plaque forming units are measured by microscopic observation, fluorescent antibodies or specific dyes that react with infected cells. The conduction of a PRNT is within the common skill of the skilled artisan. For detailed protocols, reference is made to Sun, W. et al. Protection of Rhesus monkeys against dengue virus challenge after tetravalent live attenuated dengue virus vaccination. J. Infect. Dis. 193, 1658-1665 (2006) and Muthumani K. et al. In vivo protection against ZIKV infection and pathogenesis through passive antibody transfer and active immunisation with a prMEnv DNA vaccine. NPJ Vaccines 1:16021 (2016). The protocol for the PRNT used for determining anti-Zika virus neutralizing antibody titers according to the present disclosure is also described in detail in Example 6 below.

Within the meaning of the present disclosure, the reporter virus particle (RVP) assay refers to an assay for determining anti-Zika virus neutralizing antibody titers in human subjects. Reporter virus particles (RVPs) are replication-incompetent virus particles engineered to express one or more reporter genes upon infecting susceptible cells. Since the RVP genome lacks genes essential for viral replication, RVPs are capable of only a single round of infection. Expression of a reporter such as luciferase can provide a quantitative readout of infection. The conduction of an RVP assay is within the common skill of the skilled artisan. For a detailed protocol, reference is made to also Bohning et al., 2021 (PLOS One 16:e0250516, “A high throughput reporter virus particle microneutralization assay for quantitation of Zika virus neutralizing antibodies in multiple species”). The protocol for the RVP assay used for determining anti-Zika virus neutralizing antibody titers according to the present disclosure is also described in detail in Example 6 below.

In certain, preferred embodiments in this section (“Immunogenicity”), the vaccine or immunogenic composition is administered as a first and a second administration and the first and the second administration take place from about 1 to about 6 weeks, from about 1 to about 4 weeks, or from about 25 to 30 days (such as 28 days) apart.

### Geometric Mean Neutralizing Antibody Titers (GMTs)

The determination of geometric mean (neutralizing antibody) titers (GMTs) from the single measured antibody titers is within the common skill of the skilled artisan. The geometric mean is defined as the n^{th} root of the product of n numbers (in this case, antibody titers).

### Flavivirus Naïve Subjects

In certain embodiments, the vaccine or immunogenic composition comprising an antigen from a Zika virus induces geometric mean neutralizing antibody titers in a population of at least 15 flavivirus naïve human subjects of greater than 40, or greater than 90, or greater than 150, or greater than 200, or greater than 300, or greater than 400, or greater than 500, or greater than 600, or greater than 1000, or greater than 1500, or greater than 2000, or greater than 3000 as determined by a plaque reduction neutralization test, wherein the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 15 flavivirus naïve human subjects of greater than greater than 150, or greater than 350, or greater than 600 as determined by a plaque reduction neutralization test, wherein the geometric mean neutralizing antibody titers are measured 182 days after a second administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 15 flavivirus naïve human subjects of greater than greater than 400 as determined by a plaque reduction neutralization test, wherein the geometric mean neutralizing antibody titers are measured 364 days after a second administration and the antigen is an inactivated whole virus. In certain embodiments, the vaccine or immunogenic composition comprising a dose of from about 6 to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces geometric mean neutralizing antibody titers in a population of at least 15 flavivirus naïve human subjects of greater than greater than 400 as determined by a plaque reduction neutralization test, wherein the geometric mean neutralizing antibody titers are measured 728 days after a second administration and the antigen is an inactivated whole virus. In certain embodiments, the vaccine or immunogenic composition comprising a dose of from about 6 to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces geometric mean neutralizing antibody titers in a population of at least 15 flavivirus naïve human subjects of greater than 300, or greater than 400, or greater than 600, or greater than 700, or greater than 1000, or greater than 3000, or greater than 6000, or greater than 10000 as determined by a reporter virus particle assay, wherein the antigen is an inactivated whole virus. In certain embodiments, the vaccine or immunogenic composition comprising a dose of from about 6 μg to about 15 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 15 flavivirus naïve human subjects of greater than greater than 300, or greater than 600, or greater than 1000 as determined by a reporter virus particle assay, wherein the geometric mean neutralizing antibody titers are measured 182 days after a second administration and the antigen is an inactivated whole virus. In certain embodiments, the vaccine or immunogenic composition comprising a dose of from about 6 μg to about 15 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 15 flavivirus naïve human subjects of greater than greater than 700 as determined by a reporter virus particle assay, wherein the geometric mean neutralizing antibody titers are measured 364 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces geometric mean neutralizing antibody titers in a population of at least 15 flavivirus naïve human subjects of greater than greater than 600 as determined by a reporter virus particle assay, wherein the geometric mean neutralizing antibody titers are measured 728 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the geometric mean neutralizing antibody titers are induced in a population of at least 20 flavivirus naïve human subjects.

### Flavivirus Primed Subjects

In certain embodiments, the vaccine or immunogenic composition comprising an antigen from a Zika virus induces geometric mean neutralizing antibody titers in a population of at least 20 flavivirus primed human subjects of greater than 100, or greater than 200, or greater than 150, or greater than 500, or greater than 800, or greater than 1000, or greater than 1500, or greater than 2000, or greater than 2500 as determined by a plaque reduction neutralization test, wherein the antigen is an inactivated whole virus. In certain embodiments, the vaccine or immunogenic composition comprising a dose of from about 2 to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 500, or greater than 1000, or greater than 1500 as determined by a plaque reduction neutralization test, wherein the geometric mean neutralizing antibody titers are measured 28 days after a single dose or first administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 500, or greater than 1000, or greater than 2500 as determined by a plaque reduction neutralization test, wherein the geometric mean neutralizing antibody titers are measured 28 days after a second administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 250, or greater than 500, or greater than 800 as determined by a plaque reduction neutralization test, wherein the geometric mean neutralizing antibody titers are measured 182 days after a second administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 500 as determined by a plaque reduction neutralization test, wherein the geometric mean neutralizing antibody titers are measured 364 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 100 as determined by a plaque reduction neutralization test, wherein the geometric mean neutralizing antibody titers are measured 728 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 800, or greater than 1000, or greater than 1500, or greater than 2000, or greater than 2500, or greater than 3000, or greater than 3500, or greater than 5000 as determined by a reporter virus particle assay, wherein the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 800, or greater than 1500, or greater than 2500 as determined by a reporter virus particle assay, wherein the geometric mean neutralizing antibody titers are measured 28 days after a single dose or first administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 1500, or greater than 3000, or greater than 5000 as determined by a reporter virus particle assay, wherein the geometric mean neutralizing antibody titers are measured 28 days after a second administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 1500, or greater than 2500, or greater than 3500 as determined by a reporter virus particle assay, wherein the geometric mean neutralizing antibody titers are measured 182 days after a second administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 1500 as determined by a reporter virus particle assay, wherein the geometric mean neutralizing antibody titers are measured 364 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces geometric mean neutralizing antibody titers in a population of at least 20 flavivirus primed human subjects of greater than 1000 as determined by a reporter virus particle assay, wherein the geometric mean neutralizing antibody titers are measured 728 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the geometric mean neutralizing antibody titers are induced in a population of at least 25 or at least 30 flavivirus primed human subjects.

### Seropositivity Rates

Within the meaning of the present disclosure, seropositivity is defined as titer≥10 as determined by the plaque reduction neutralization test (PRNT) or as a titer≥105 as determined by the reporter virus particle (RVP) assay.

Seropositivity rates are determined by comparing the number of subjects that are seropositive after vaccination with a certain dosage to the total number of subjects that have been vaccinated with a certain dosage. The determination of seropositivity rates is within the common skill of the skilled artisan.

### Flavivirus Naive Subjects

In certain embodiments, the vaccine or immunogenic composition comprising an antigen from a Zika virus induces a seropositivity rate in a population of at least 15 flavivirus naïve human subjects of greater than 60%, or greater than 70%, or greater than 80%, or greater than 90%, or 100% as determined by a plaque reduction neutralization test, wherein the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 15 flavivirus naïve human subjects of greater than 90%, or greater than 95%, or 100% as determined by a plaque reduction neutralization test, wherein the seropositivity rate is measured 182 days after a second administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 15 flavivirus naïve human subjects of greater than 90%, or greater than 95%, or 100% as determined by a plaque reduction neutralization test, wherein the seropositivity rate is measured 364 days after a second administration and the antigen is an inactivated whole virus. In certain embodiments, the vaccine or immunogenic composition comprising a dose of from about 6 to about 15 μg, such as about 10 μg, of the Zika virus antigen and optionally from about 150 to about 250 μg of aluminum hydroxide as adjuvant. In certain embodiments, the vaccine or immunogenic composition comprising a dose of from about 6 to about 15 μg, such as about 10 μg, of the Zika virus antigen and optionally from about 175 to about 225 μg of aluminum hydroxide as adjuvant.

In certain embodiments, the vaccine or immunogenic composition comprising an antigen from a Zika virus induces a seropositivity rate in a population of at least 15 flavivirus naïve human subjects of greater than 80%, or greater than 85%, or greater than 90% as determined by a plaque reduction neutralization test, wherein the seropositivity rate is measured 728 days after a second administration and the antigen is an inactivated whole virus. In certain embodiments, the vaccine or immunogenic composition comprising a dose of from about 6 to about 15 μg, such as about 10 μg, of the Zika virus antigen and optionally from about 150 to about 250 μg of aluminum hydroxide as adjuvant. In certain embodiments, the vaccine or immunogenic composition comprising a dose of from about 6 to about 15 μg, such as about 10 μg, of the Zika virus antigen and optionally from about 175 to about 225 μg of aluminum hydroxide as adjuvant.

In certain embodiments, the vaccine or immunogenic composition comprising an antigen from a Zika virus induces a seropositivity rate in a population of at least 15 flavivirus naïve human subjects of greater than 60%, or greater than 70%, or greater than 80%, or greater than 90%, or 100% as determined by a reporter virus particle assay, wherein the antigen is an inactivated whole virus. In certain embodiments, the vaccine or immunogenic composition comprising a dose of from about 6 to about 15 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 15 flavivirus naïve human subjects of greater 80%, or greater than 85%, or greater than 90%, or greater than 95%, or 100% as determined by a reporter virus particle assay, wherein the seropositivity rate is measured 182 days after a second administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 15 flavivirus naïve human subjects of greater than 90%, or greater than 95%, or 100% as determined by a reporter virus particle assay, wherein the seropositivity rate is measured 364 days after a second administration and the antigen is an inactivated whole virus. In certain embodiments, the vaccine or immunogenic composition comprising a dose of from about 6 to about 15 μg, such as about 10 μg, of the Zika virus antigen and optionally from about 150 to about 250 μg of aluminum hydroxide as adjuvant. In certain embodiments, the vaccine or immunogenic composition comprising a dose of from about 6 to about 15 μg, such as about 10 μg, of the Zika virus antigen and optionally from about 175 to about 225 μg of aluminum hydroxide as adjuvant.

In certain embodiments, the vaccine or immunogenic composition comprising an antigen from a Zika virus induces a seropositivity rate in a population of at least 15 flavivirus naïve human subjects of greater than 80%, or greater than 85%, or greater than 90% as determined by a reporter virus particle assay, wherein the seropositivity rate is measured 728 days after a second administration and the antigen is an inactivated whole virus. In certain embodiments, the vaccine or immunogenic composition comprising a dose of from about 6 to about 15 μg, such as about 10 μg, of the Zika virus antigen and optionally from about 150 to about 250 μg of aluminum hydroxide as adjuvant. In certain embodiments, the vaccine or immunogenic composition comprising a dose of from about 6 to about 15 μg, such as about 10 μg, of the Zika virus antigen and optionally from about 175 to about 225 μg of aluminum hydroxide as adjuvant.

In certain embodiments, the seropositivity rate is induced in a population of at least 20 flavivirus naïve human subjects.

### Flavivirus Primed Subjects

In certain embodiments, the vaccine or immunogenic composition comprising an antigen from a Zika virus induces a seropositivity rate in a population of at least 20 flavivirus primed human subjects of greater than 75%, or greater than 80%, or greater than 90%, or greater than 95%, or 100% as determined by a plaque reduction neutralization test, wherein the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 20 flavivirus primed human subjects of greater than 90%, or greater than 95%, or 100% as determined by a plaque reduction neutralization test, wherein the seropositivity rate is measured 28 days after a single dose or first administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 20 flavivirus primed human subjects of greater than 90%, or greater than 95%, or 100% as determined by a plaque reduction neutralization test, wherein the seropositivity rate is measured 28 days after a second administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 20 flavivirus primed human subjects of greater than 80%, or greater than 95%, or 100% as determined by a plaque reduction neutralization test, wherein the seropositivity rate is measured 182 days after a second administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 20 flavivirus primed human subjects of greater than 90%, or greater than 95%, or 100% as determined by a plaque reduction neutralization test, wherein the seropositivity rate is measured 364 days after a second administration and the antigen is an inactivated whole virus. In certain embodiments, the vaccine or immunogenic composition comprising a dose of from about 6 to about 15 μg, such as about 10 μg, of the Zika virus antigen and optionally from about 150 to about 250 μg of aluminum hydroxide as adjuvant. In certain embodiments, the vaccine or immunogenic composition comprising a dose of from about 6 to about 15 μg, such as about 10 μg, of the Zika virus antigen and optionally from about 175 to about 225 μg of aluminum hydroxide as adjuvant.

In certain embodiments, the vaccine or immunogenic composition comprising an antigen from a Zika virus induces a seropositivity rate in a population of at least 20 flavivirus primed human subjects of greater than 70%, or greater than 75% as determined by a plaque reduction neutralization test, wherein the seropositivity rate is measured 728 days after a second administration and the antigen is an inactivated whole virus. In certain embodiments, the vaccine or immunogenic composition comprising a dose of from about 6 to about 15 μg, such as about 10 μg, of the Zika virus antigen and optionally from about 150 to about 250 μg of aluminum hydroxide as adjuvant. In certain embodiments, the vaccine or immunogenic composition comprising a dose of from about 6 to about 15 μg, such as about 10 μg, of the Zika virus antigen and optionally from about 175 to about 225 μg of aluminum hydroxide as adjuvant.

In certain embodiments, the vaccine or immunogenic composition comprising an antigen from a Zika virus induces a seropositivity rate in a population of at least 20 flavivirus primed human subjects of greater than 75%, or greater than 80%, or greater than 90%, or greater than 95%, or 100% as determined by a reporter virus particle assay, wherein the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 20 flavivirus primed human subjects of greater than 90%, or greater than 95%, or 100% as determined by a reporter virus particle assay, wherein the seropositivity rate is measured 28 days after a single dose or first administration and the antigen is an inactivated whole virus. In certain embodiments, the vaccine or immunogenic composition comprising a dose of from about 2 μg to about 10 μg of the Zika virus antigen and optionally from about 150 to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 20 flavivirus primed human subjects of greater than 90%, or greater than 95%, or 100% as determined by a reporter virus particle assay, wherein the seropositivity rate is measured 28 days after a second administration and the antigen is an inactivated whole virus. In certain embodiments, the vaccine or immunogenic composition comprising a dose of from about 2 μg to about 10 μg of the Zika virus antigen and optionally from about 150 to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 20 flavivirus primed human subjects of greater than 90%, or greater than 95%, or 100% as determined by a reporter virus particle assay, wherein the seropositivity rate is measured 182 days after a second administration and the antigen is an inactivated whole virus. In certain embodiments, the vaccine or immunogenic composition comprising a dose of from about 2 μg to about 10 μg of the Zika virus antigen and optionally from about 150 to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seropositivity rate in a population of at least 20 flavivirus primed human subjects of greater than 90%, or greater than 95%, or 100% as determined by a reporter virus particle assay, wherein the seropositivity rate is measured 364 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 to about 225 μg of aluminum hydroxide as adjuvant.

In certain embodiments, the vaccine or immunogenic composition comprising an antigen from a Zika virus induces a seropositivity rate in a population of at least 20 flavivirus primed human subjects of greater than 90%, or greater than 95% as determined by a reporter virus particle assay, wherein the seropositivity rate is measured 728 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 to about 225 μg of aluminum hydroxide as adjuvant.

In certain embodiments, the seropositivity rate is induced in a population of at least 25 or at least 30 flavivirus primed human subjects.

### Seroconversion Rates

Within the meaning of the present disclosure, seroconversion is defined in the case of flavivirus naïve human subjects being Zika virus seronegative prior to vaccination (PRNT titer<10 or RVP titer<105) as the subjects having a PRNT titer≥10 or a RVP titer≥105 post-vaccination (i.e. being seropositive) as determined by the plaque reduction neutralization test (PRNT) or the reporter virus particle (RVP) assay, respectively.

Within the meaning of the present disclosure, seroconversion is defined in the case of flavivirus primed human subjects being Zika virus seropositive prior to vaccination (PRNT titer≥10 or RVP titer≥105) as the subjects having a post vaccination titer increase of ≥4-fold as determined by the plaque reduction neutralization test (PRNT) or the reporter virus particle (RVP) assay, respectively (“4-fold seroconversion”).

Seroconversion rates are determined by comparing the number of subjects that are seroconverted after vaccination with a certain dosage to the total number of subjects that have been vaccinated with a certain dosage. The determination of seroconversion rates is within the common skill of the skilled artisan.

### Flavivirus Naïve Subjects

In certain embodiments, the vaccine or immunogenic composition comprising an antigen from a Zika virus induces a seroconversion rate in a population of at least 15 flavivirus naïve human subjects of greater than 60%, or greater than 70%, or greater than 80%, or greater than 90%, or 100% as determined by a plaque reduction neutralization test, wherein the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seroconversion rate in a population of at least 15 flavivirus naïve human subjects of greater than 90%, or greater than 95%, or 100% as determined by a plaque reduction neutralization test, wherein the seroconversion rate is measured 182 days after a second administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seroconversion rate in a population of at least 15 flavivirus naïve human subjects of greater than 90%, or greater than 95%, or 100% as determined by a plaque reduction neutralization test, wherein the seroconversion rate is measured 364 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 to about 225 μg of aluminum hydroxide as adjuvant.

In certain embodiments, the vaccine or immunogenic composition comprising an antigen from a Zika virus induces a seroconversion rate in a population of at least 15 flavivirus naïve human subjects of greater than 80%, or greater than 85%, or greater than 90% as determined by a plaque reduction neutralization test, wherein the seroconversion rate is measured 728 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces a seroconversion rate in a population of at least 15 flavivirus naïve human subjects of greater than 60%, or greater than 70%, or greater than 80%, or greater than 90%, or 100% as determined by a reporter virus particle assay, wherein the antigen is an inactivated whole virus. In certain embodiments, the vaccine or immunogenic composition comprising a dose of from about 6 μg to about 15 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces a seroconversion rate in a population of at least 15 flavivirus naïve human subjects of greater 80%, or greater than 85%, or greater than 90%, or greater than 95%, or 100% as determined by a reporter virus particle assay, wherein the seropositivity rate is measured 182 days after a second administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a seroconversion rate in a population of at least 15 flavivirus naïve human subjects of greater than 90%, or greater than 95%, or 100% as determined by a reporter virus particle assay, wherein the seropositivity rate is measured 364 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 to about 225 μg of aluminum hydroxide as adjuvant.

In certain embodiments, the vaccine or immunogenic composition comprising an antigen from a Zika virus induces a seroconversion rate in a population of at least 15 flavivirus naïve human subjects of greater than 80%, or greater than 85%, or greater than 90% as determined by a reporter virus particle assay, wherein the seropositivity rate is measured 728 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the seroconversion rate is induced in a population of at least 20 flavivirus naïve human subjects.

### Flavivirus Primed Subjects

In certain embodiments, the vaccine or immunogenic composition comprising an antigen from a Zika virus induces a 4-fold seroconversion rate in a population of at least 20 flavivirus primed human subjects of greater than 20%, or greater than 30%, or greater than 35%, or greater than 40%, or greater than 45%, or greater than 50%, or greater than 55%, or greater than 60%, or greater than 65%, or greater than 70%, or greater than 75% as determined by a plaque reduction neutralization test, wherein the antigen is an inactivated whole virus. In certain embodiments, the vaccine or immunogenic composition comprising a dose of from about 2 to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 35%, or greater than 40%, or greater than 70% as determined by a plaque reduction neutralization test, wherein the seropositivity rate is measured 28 days after a single dose or first administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 40%, or greater than 50%, or greater than 60%, or greater than 70% as determined by a plaque reduction neutralization test, wherein the seropositivity rate is measured 28 days after a second administration and the antigen is an inactivated whole virus. In certain embodiments, the vaccine or immunogenic composition comprising a dose of from about 2 to about 10 μg of the Zika virus antigen and optionally from about 150 to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 20%, or greater than 30%, or greater than 60% as determined by a plaque reduction neutralization test, wherein the seropositivity rate is measured 182 days after a second administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 to about 250 μg, such as from about 175 μg to about 225 μg, of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 50%, or greater than 55%, or greater than 60% as determined by a plaque reduction neutralization test, wherein the seropositivity rate is measured 364 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 40% as determined by a plaque reduction neutralization test, wherein the seropositivity rate is measured 728 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 25%, or greater than 30%, or greater than 40%, or greater than 50%, or greater than 70% as determined by a reporter virus particle assay, wherein the antigen is an inactivated whole virus. In certain embodiments, the vaccine or immunogenic composition comprising a dose of from about 2 to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 25%, or greater than 30%, or greater than 55% as determined by a reporter virus particle assay, wherein the seropositivity rate is measured 28 days after a single dose or first administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 40%, or greater than 45%, or greater than 50%, or greater than 60%, or greater than 65%, or greater than 70% as determined by a reporter virus particle assay, wherein the seropositivity rate is measured 28 days after a second administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 40%, or greater than 45%, or greater than 60% or greater than 65% as determined by a reporter virus particle assay, wherein the seropositivity rate is measured 182 days after a second administration and the antigen is an inactivated whole virus. In certain embodiments, the vaccine or immunogenic composition comprising a dose of from about 2 μg to about 10 μg of the Zika virus antigen and optionally from about 150 μg to about 250 μg of aluminum hydroxide as adjuvant. In certain embodiments, the vaccine or immunogenic composition comprising a dose of from about 6 μg to about 15 μg of the Zika virus antigen and optionally from about 150 μg to about 250 μg, such as from about 175 μg to about 225 μg, of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 45%, or greater than 50%, or greater than 55% as determined by a reporter virus particle assay, wherein the seropositivity rate is measured 364 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the vaccine or immunogenic composition comprising an antigen from a Zika virus induces a 4-fold seroconversion rate in a population of at least 20 flavivirus primed human subjects of greater than 50%, or greater than 55%, or greater than 60% as determined by a reporter virus particle assay, wherein the seropositivity rate is measured 728 days after a second administration and the antigen is an inactivated whole virus. In certain embodiments, the vaccine or immunogenic composition comprising a dose of from about 6 μg to about 15 μg, such as about 10 μg, of the Zika virus antigen and optionally from about 150 μg to about 250 μg of aluminum hydroxide as adjuvant. In certain embodiments, the vaccine or immunogenic composition comprising a dose of from about 6 μg to about 15 μg, such as about 10 μg, of the Zika virus antigen and optionally from about 175 μg to about 225 μg of aluminum hydroxide as adjuvant.

In certain embodiments, the seroconversion rate is induced in a population of at least 25 or at least 30 flavivirus primed human subjects.

### Methods/Uses

Certain embodiments of the present disclosure are directed to a method for inducing an immune response against Zika virus and/or preventing Zika virus infection and/or preventing Zika virus disease in a human subject or a human subject population, the method comprising administering to the human subject or the individuals of the human subject population the vaccines or immunogenic compositions as described herein. Administration of the vaccine or immunogenic composition induces the geometric mean neutralizing antibody titers and/or seropositivity rates and/or seroconversion rates in said human subject population as outlined above.

Certain embodiments of the present disclosure are directed to the use of the vaccines or immunogenic compositions as described herein in the manufacture of a medicament for inducing an immune response against Zika virus and/or preventing Zika virus infection and/or preventing Zika virus disease in a human subject or a human subject population. In certain embodiments, the use comprises that the vaccines or immunogenic compositions are to be administered to the human subject or the individuals of the human subject population, wherein administering of the vaccine or immunogenic composition induces the geometric mean neutralizing antibody titers and/or seropositivity rates and/or seroconversion rates in said human subject population as outlined above.

Certain embodiments of the present disclosure are directed to the vaccines or immunogenic compositions as described herein for use in inducing an immune response against Zika virus and/or preventing Zika virus infection and/or preventing Zika virus disease in a human subject or a human subject population. In certain embodiments, the vaccines or immunogenic compositions are administered to the human subject or the individuals of the human subject population, wherein administering of the vaccine or immunogenic composition induces the geometric mean neutralizing antibody titers and/or seropositivity rates and/or seroconversion rates in said human subject population as outlined above.

As regards the administration regimen, reference is also made to the chapter “Administration regimen” below. As regards the dose and the adjuvant, reference is also made to the chapters “Adjuvants” and “Formulations and Dose” above.

### Administration Regimen

The vaccines and/or immunogenic compositions of the present disclosure (in particular as described above under the sections “Safety” and “Immunogenicity”) may be administered in a manner compatible with the dosage formulation, and in such amount as will be therapeutically effective and immunogenic.

The vaccines or immunogenic compositions of the present disclosure are usually administered parenterally, by injection, for example, either subcutaneously, transcutaneously, intradermally, subdermally or intramuscularly. In certain embodiments, the vaccine or immunogenic composition is administered intramuscularly or subcutaneously. In a preferred embodiment, the vaccine or immunogenic composition is administered intramuscularly.

The vaccines or immunogenic compositions of the present disclosure can also be formulated in a way suitable for other modes of administration, including oral, peroral, intranasal, buccal, sublingual, intraperitoneal, intravaginal, anal and intracranial formulations. Such formulations and administration routes are described in WO 2019/108970 A1, the disclosure of which is hereby incorporated by reference.

In some embodiments, the administering step includes one or more administrations. Administration can be by a single dose schedule or a multiple dose schedule. In a multiple dose schedule the various doses may be given by the same or different routes e.g. a parenteral prime and mucosal boost, a mucosal prime and parenteral boost, etc. Typically, they will be given by the same route, such as by intramuscular or subcutaneous administration. In a preferred embodiment, the administrations are given intramuscularly.

Multiple doses will typically be administered at least 1 week apart. In certain embodiments, a first and a second administration take place about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 16 weeks apart. In some embodiments, a first and a second administration take place from about 1 to about 16 weeks apart, from about 1 to about 6 weeks apart, or from about 1 to about 4 weeks apart. In some embodiments, a first and a second administration take place from about 25 to about 30 days apart. In some embodiments, a first and a second administration take place 28 days (4 weeks) apart. In certain such embodiments the mode of administration is intramuscular or subcutaneous administration, preferably the mode of administration is intramuscular administration.

In a preferred embodiment, the vaccine or immunogenic composition as administered as first and second administration from about 25 to about 30 days, such as 28 days, apart.

Within the meaning of the disclosure, a first and a second administration refer to a primary administration. In certain embodiments, the primary administration is followed by a third (booster) administration.

In some embodiments of the disclosure, a third (booster) administration is not required. In certain such embodiments, the vaccine or immunogenic composition is administered as a first and a second administration that take place from about 1 to about 16 weeks apart, from about 1 to about 6 weeks apart, or from about 1 to about 4 weeks apart. In certain such embodiments, the vaccine or immunogenic composition is administered as a first and a second administration that take place from about 25 to about 30 days, such as 28 days, apart. In this embodiments, the administration consists of a first and a second administration.

In some embodiments, the administration comprises a first, a second, and a third (booster) administration. In certain such embodiments, the third (booster) administration is administered not earlier than about 6 months after the second administration. In one embodiment, the third (booster) administration is administered not earlier than about 170 days after the second administration. In one embodiment, the third (booster) administration is administered not earlier than about 175 days after the second administration. In one embodiment, the third (booster) administration is administered not earlier than about 180 days after the second administration. In one embodiment, the third (booster) administration is administered not earlier than about 182 days after the second administration. In certain embodiments, the first and the second administration take place from about 25 to about 30 days apart, such as 28 days, apart. In certain such embodiments the mode of administration is intramuscular or subcutaneous administration, preferably the mode of administration is intramuscular administration. In certain embodiments, the vaccine or immunogenic composition that is administered comprises a Zika virus antigen at a dose from about 2 to about 15 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 8 to about 12 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 9 to about 11 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose of about 10 μg, optionally the vaccine or immunogenic composition that is administered further comprises from about 150 μg to about 250 μg or from about 175 μg to about 225 μg, such as about 200 μg, of aluminum hydroxide as the adjuvant.

In some embodiments, the administration comprises a first, a second, and a third (booster) administration. In certain such embodiments, the third (booster) administration is administered from about 6 to about 24 months after the second administration. In one embodiment, the third (booster) administration is administered from about 6 to about 12 months after the second administration. In one embodiment, the third (booster) administration is administered from about 12 to about 24 months after the second administration. In one embodiment, the third (booster) administration is administered about 6 months after the second administration. In one embodiment, the third (booster) administration is administered about 12 months after the second administration. In one embodiment, the third (booster) administration is administered about 24 months after the second administration. In one embodiment, the third (booster) administration is administered from about 170 to about 200 days after the second administration. In one embodiment, the third (booster) administration is administered from about 175 to about 190 days after the second administration. In one embodiment, the third (booster) administration is administered from about 180 to about 185 days after the second administration. In one embodiment, the third (booster) administration is administered about 182 days after the second administration. In certain such embodiments the mode of administration is intramuscular or subcutaneous administration, preferably the mode of administration is intramuscular administration. In certain embodiments, the vaccine or immunogenic composition that is administered comprises a Zika virus antigen at a dose from about 2 to about 15 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 8 to about 12 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 9 to about 11 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose of about 10 μg, optionally the vaccine or immunogenic composition that is administered further comprises from about 150 μg to about 250 μg or from about 175 μg to about 225 μg, such as about 200 μg, of aluminum hydroxide as the adjuvant.

In some embodiments, the administration comprises a first, a second, and a third (booster) administration. In certain such embodiments, the first and the second administration take place from about 25 to about 30 days apart and the third (booster) administration is administered not earlier than 6 months after the second administration. In one embodiment, the first and the second administration take place from about 25 to about 30 days apart and the third (booster) administration is administered from about 6 to about 24 months after the second administration. In one embodiment, the first and the second administration take place from about 25 to about 30 days apart and the third (booster) administration is administered from about 6 to about 12 months after the second administration. In one embodiment, the first and the second administration take place from about 25 to about 30 days apart and the third (booster) administration is administered from about 12 to about 24 months after the second administration. In one embodiment, the first and the second administration take place from about 25 to about 30 days apart and the third (booster) administration is administered about 6 months after the second administration. In one embodiment, the first and the second administration take place from about 25 to about 30 days apart and the third (booster) administration is administered about 12 months after the second administration. In one embodiment, the first and the second administration take place from about 25 to about 30 days apart and the third (booster) administration is administered about 24 months after the second administration. In one embodiment, the first and the second administration take place from about 25 to about 30 days apart and the third (booster) administration is administered from about 170 to about 200 days after the second administration. In one embodiment, the first and the second administration take place from about 25 to about 30 days apart and the third (booster) administration is administered from about 175 to about 190 days after the second administration. In one embodiment, the first and the second administration take place from about 25 to about 30 days apart and the third (booster) administration is administered from about 180 to about 185 days after the second administration. In one embodiment, the first and the second administration take place from about 25 to about 30 days apart and the third (booster) administration is administered about 182 days after the second administration. In certain such embodiments the mode of administration is intramuscular or subcutaneous administration, preferably the mode of administration is intramuscular administration. In certain embodiments, the vaccine or immunogenic composition that is administered comprises a Zika virus antigen at a dose from about 2 to about 15 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 8 to about 12 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 9 to about 11 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose of about 10 μg, optionally the vaccine or immunogenic composition that is administered further comprises from about 150 μg to about 250 μg or from about 175 μg to about 225 μg, such as about 200 μg, of aluminum hydroxide as the adjuvant.

In some embodiments, the administration comprises a first, a second, and a third (booster) administration. In certain such embodiments, the first and the second administration take place about 28 days apart and the third (booster) administration is administered not earlier than 6 months after the second administration. In one embodiment, the first and the second administration take place about 28 days apart and the third (booster) administration is administered from about 6 to about 24 months after the second administration. In one embodiment, the first and the second administration take place about 28 days apart and the third (booster) administration is administered from about 6 to about 12 months after the second administration. In one embodiment, the first and the second administration take place about 28 days apart and the third (booster) administration is administered from about 12 to about 24 months after the second administration. In one embodiment, the first and the second administration take place about 28 days and the third (booster) administration is administered about 6 months after the second administration. In one embodiment, the first and the second administration take place about 28 days apart and the third (booster) administration is administered about 12 months after the second administration. In one embodiment, the first and the second administration take place about 28 days apart and the third (booster) administration is administered about 24 months after the second administration. In one embodiment, the first and the second administration take place about 28 days apart and the third (booster) administration is administered from about 170 to about 200 days after the second administration. In one embodiment, the first and the second administration take place about 28 days apart and the third (booster) administration is administered from about 175 to about 190 days after the second administration. In one embodiment, the first and the second administration take place about 28 days apart and the third (booster) administration is administered from about 180 to about 185 days after the second administration. In one embodiment, the first and the second administration take place about 28 days apart and the third (booster) administration is administered about 182 days after the second administration. In certain such embodiments the mode of administration is intramuscular or subcutaneous administration, preferably the mode of administration is intramuscular administration. In certain embodiments, the vaccine or immunogenic composition that is administered comprises a Zika virus antigen at a dose from about 2 to about 15 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 8 to about 12 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 9 to about 11 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose of about 10 μg, optionally the vaccine or immunogenic composition that is administered further comprises from about 150 μg to about 250 μg or from about 175 μg to about 225 μg, such as about 200 μg, of aluminum hydroxide as the adjuvant.

In some embodiments, the administration includes a first, a second, and a third (booster) administration. In certain such embodiments, the third (booster) administration is administered at least 6 months after the second administration. In one embodiment, the third (booster) administration is administered at least 8 months, at least 10 months, at least 12 months, at least 18 months, or at least 24 months after the second administration. In one embodiment, the third (booster) administration is administered at least 6 months after the second administration. In one embodiment, the third (booster) administration is administered at least 12 months after the second administration. In one embodiment, the third (booster) administration is administered at least 24 months after the second administration. In one embodiment, the third (booster) administration is administered at least about 170 days after the second administration. In one embodiment, the third (booster) administration is administered at least about 175 days after the second administration. In one embodiment, the third (booster) administration is administered at least about 180 days after the second administration. In one embodiment, the third (booster) administration is administered at least about 182 days after the second administration. In certain such embodiments the mode of administration is intramuscular or subcutaneous administration, preferably the mode of administration is intramuscular administration. In certain embodiments, the vaccine or immunogenic composition that is administered comprises a Zika virus antigen at a dose from about 2 to about 15 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 8 to about 12 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 9 to about 11 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose of about 10 μg, optionally the vaccine or immunogenic composition that is administered further comprises from about 150 μg to about 250 μg or from about 175 μg to about 225 μg, such as about 200 μg, of aluminum hydroxide as the adjuvant.

In some embodiments, the administration includes a first, a second, and a third (booster) administration. In certain such embodiments, the first and the second administration take place from about 25 to about 30 days apart and the third (booster) administration is administered at least 6 months, at least 8 months, at least 10 months, at least 12 months, at least 18 months, or at least 24 months after the second administration. In more specific embodiments, the first and the second administration take place from about 25 to about 30 days apart and the third (booster) administration is administered at least 6 months after the second administration. In more specific embodiments, the first and the second administration take place from about 25 to about 30 days apart and the third (booster) administration is administered at least 12 months after the second administration. In more specific embodiments, the first and the second administration take place from about 25 to about 30 days apart and the third (booster) administration is administered at least 24 months after the second administration. In one embodiment, the first and the second administration take place from about 25 to about 30 days apart and the third (booster) administration is administered at least about 170 days after the second administration. In one embodiment, the first and the second administration take place from about 25 to about 30 days apart and the third (booster) administration is administered at least about 175 days after the second administration. In one embodiment, the first and the second administration take place from about 25 to about 30 days apart and the third (booster) administration is administered at least about 180 days after the second administration. In one embodiment, the first and the second administration take place from about 25 to about 30 days apart and the third (booster) administration is administered at least about 182 days after the second administration. In certain such embodiments the mode of administration is intramuscular or subcutaneous administration, preferably the mode of administration is intramuscular administration. In certain embodiments, the vaccine or immunogenic composition that is administered comprises a Zika virus antigen at a dose from about 2 to about 15 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 8 to about 12 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 9 to about 11 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose of about 10 μg, optionally the vaccine or immunogenic composition that is administered further comprises from about 150 μg to about 250 μg or from about 175 μg to about 225 μg, such as about 200 μg, of aluminum hydroxide as the adjuvant.

In some embodiments, the administration includes a first, a second, and a third (booster) administration. In certain such embodiments, the first and the second administration take place about 28 days apart and the third (booster) administration is administered at least 6 months after the second administration. In more specific embodiments, the first and the second administration take place about 28 days apart and the third (booster) administration is administered at least 12 months after the second administration. In more specific embodiments, the first and the second administration take place about 28 days apart and the third (booster) administration is administered at least 24 months after the second administration. In one embodiment, the first and the second administration take place about 28 days apart and the third (booster) administration is administered at least about 170 days after the second administration. In one embodiment, the first and the second administration take place about 28 days apart and the third (booster) administration is administered at least about 175 days after the second administration. In one embodiment, the first and the second administration take place about 28 days apart and the third (booster) administration is administered at least about 180 days after the second administration. In one embodiment, the first and the second administration take place about 28 days apart and the third (booster) administration is administered at least about 182 days after the second administration. In certain such embodiments the mode of administration is intramuscular or subcutaneous administration, preferably the mode of administration is intramuscular administration. In certain embodiments, the vaccine or immunogenic composition that is administered comprises a Zika virus antigen at a dose from about 2 to about 15 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 8 to about 12 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose from about 9 to about 11 μg, such as about 10 μg, optionally the vaccine or immunogenic composition that is administered further comprises from about 150 μg to about 250 μg or from about 175 μg to about 225 μg, such as about 200 μg, of aluminum hydroxide as the adjuvant. In certain embodiments, the vaccine or immunogenic composition that is administered comprises a Zika virus antigen at a dose of about 10 μg, optionally the vaccine or immunogenic composition that is administered further comprises from about 150 μg to about 250 μg or from about 175 μg to about 225 μg, such as about 200 μg, of aluminum hydroxide as the adjuvant.

In one embodiment, the vaccine or immunogenic composition is administered as a first, second, and third (booster) administration, wherein the first and the second administration take place from about 25 to about 30 days, such as about 28 days apart, and the third administration takes place from about 180 to about 185 days, such as 182 days, after the second administration.

In one embodiment, the vaccine or immunogenic composition is administered as a first, second, and third (booster) administration, wherein the first administration takes place on day 1, the second administration takes place on day 29, and the third administration takes place on day 211.

As regards the dose, the adjuvant, and the immunogenicity, reference is also made to the chapters “Adjuvants” and “Formulations and Dose” and “Immunogenicity” above.

### Methods of the Present Invention

The present disclosure is further directed to the use of the vaccine or immunogenic composition as described in this application and in particular in the sections “Safety” and “Immunogenicity” above in the manufacture of a medicament for inducing an immune response against Zika virus in a human subject or in a human subject population. The present disclosure is also directed to the use of the vaccine or immunogenic composition as described in this application and in particular in the sections “Safety” and “Immunogenicity” above in the manufacture of a medicament for preventing Zika virus infection and/or for preventing Zika virus disease in a human subject or in a human subject population. In certain embodiments, the human subject or the individuals of the human subject population are flavivirus naïve. In certain embodiments, the human subject or the individuals of the human subject population are flavivirus primed. In certain embodiments, the use comprises that the vaccine or immunogenic composition is to be administered to the human subject or the individuals of the human subject population as outlined in the section “Administration regimen” above.

The present disclosure is further directed to a method for inducing an immune response against Zika virus in a human subject or in a human subject population in need thereof, the method comprising administering (a therapeutically effective amount) of the vaccine or immunogenic composition as described in this application and in particular in the sections “Safety” and “Immunogenicity” above to the human subjects or the individuals of the human subject population. The present disclosure is also directed to a method for preventing Zika virus infection and/or for preventing Zika virus disease in a human subject or in a human subject population, the method comprising administering (a therapeutically effective amount) of the vaccine or immunogenic composition as described in this application and in particular in the sections “Safety” and “Immunogenicity” above to the human subjects or the individuals of the human subject population. In certain embodiments, the human subject or the individuals of the human subject population are flavivirus naïve. In certain embodiments, the human subject or the individuals of the human subject population are flavivirus primed. In certain embodiments, the method comprises administering the vaccine or immunogenic composition to the human subject or the individuals of the human subject population as outlined in the section “Administration regimen” above.

The present disclosure is further directed to the vaccine or immunogenic composition as described in this application and in particular in the sections “Safety” and “Immunogenicity” above for use in inducing an immune response against Zika virus in a human subject or in a human subject population. The present disclosure is also directed to the vaccine or immunogenic composition as described in this application and in particular in the sections “Safety” and “Immunogenicity” above for use in preventing Zika virus infection and/or for preventing Zika virus disease in a human subject or in a human subject population. In certain embodiments, the human subject or the individuals of the human subject population are flavivirus naïve. In certain embodiments, the human subject or the individuals of the human subject population are flavivirus primed. In certain embodiments, the vaccine or immunogenic composition is administered to the human subject, or the individuals of the human subject population as outlined in the section “Administration regimen” above.

In certain embodiments, the immune response is a protective immune response. Protective immune response refers to an immune response sufficient to prevent Zika virus infection and/or Zika virus disease.

One embodiment of the present disclosure relates to the use the vaccine or immunogenic composition as described in this application and in particular in the sections “Safety” and “Immunogenicity” above in the manufacture of a medicament for preventing Zika virus disease and/or preventing Zika virus infection in a fetus or newborn, the use comprises that the medicament is to be administered to a pregnant human subject or a human subject that intends to become pregnant or woman of childbearing potential. In certain embodiments, the vaccine or immunogenic composition is to be administered to the pregnant human subject or the human subject that intends to become pregnant or woman of childbearing potential as outlined in the section “Administration regimen” above.

One embodiment of the present disclosure relates to a method of preventing Zika virus disease and/or preventing Zika virus infection in a fetus or newborn, the method comprising administering to a pregnant human subject or a human subject that intends to become pregnant or woman of childbearing potential the vaccine or immunogenic composition as described in this application and in particular in the sections “Safety” and “Immunogenicity” above. In certain embodiments, the vaccine or immunogenic composition is administered to the pregnant human subject or human subject that intends to become pregnant or woman of childbearing potential as outlined in the section “Administration regimen” above.

One embodiment of the present disclosure relates to the vaccine or immunogenic composition as described in this application and in particular in the sections “Safety” and “Immunogenicity” above for use in preventing Zika virus disease and/or preventing Zika virus infection in a fetus or newborn, comprising administering the vaccine or immunogenic composition to a pregnant human subject or a human subject that intends to become pregnant or woman of childbearing potential. In certain embodiments, the vaccine or immunogenic composition is administered to the pregnant human subject or the human subject that intends to become pregnant or woman of childbearing potential as outlined in the section “Administration regimen” above.

Within the meaning of the present disclosure, “preventing Zika virus infection” means that the immune response induced in a human subject by a vaccine or immunogenic composition is sufficient to prevent a Zika virus from replicating in the human subject thereby preventing infection.

Within the meaning of the present disclosure, “preventing Zika virus disease” means that a Zika virus is able to infect a human subject and replicate within the human subject even after being vaccinated with a vaccine or immunogenic composition, but that the immune response induced in the human subject by the vaccine or immunogenic composition is sufficient to prevent the Zika virus from replicating at a level insufficient to cause Zika virus disease. Within the meaning of the present disclosure, the expression “preventing Zika virus disease” can also be referred to as “vaccinating against Zika virus disease” or “effective vaccination against Zika virus disease”.

Within the meaning of the present disclosure, “Zika virus disease” refers to usually a mild disease state of short duration. Some clinical manifestations include, but are not limited to, mild fever, maculopapular rash, conjunctivitis and arthralgia. Despite the rather mild clinical symptoms Zika virus usually causes, the term “Zika virus disease” can also refer to a more severe manifestation, which may in particular result from a Zika virus infection during pregnancy. Zika virus infection during pregnancy has been associated with serious outcomes for the fetus and newborn child. The spectrum of congenital anomalies in the fetus and newborn child associated with Zika virus infection of the pregnant woman, known as Congenital Zika Syndrome (CZS), consists of severe microcephaly with partially collapsed skull, cerebral cortices with subcortical calcifications, macular scarring and focal pigmentary retinal mottling, congenital contractures, and marked early hypertonia with symptoms of extrapyramidal involvement. Microcephaly is a condition in which a newborn's head circumference is significantly less than expected based on the average for their age, sex, and ethnicity. This is a result of the brain failing to undergo proper embryonic development and in most cases this condition is associated with mental retardation. Furthermore, another more severe manifestation which is encompassed by the meaning of “Zika virus disease” is Guillain-Barre Syndrome (GBS), a neurological disorder in which the individual's immune system attacks and destroys the myelin sheath surrounding axons of the peripheral nervous system.

A “human subject population” as referred to in the present disclosure is considered to encompass more than one individual, e.g. 2 or more individuals. In certain embodiments, a human subject population comprises at least 15 individuals. In certain embodiments, a human subject population comprises at least 20 individuals. In certain embodiments, a human subject population comprises at least 25 individuals. In certain embodiments, a human subject population comprises at least 30 individuals.

In some embodiments, the human subject or one or more individuals of the human subject population are female. In some embodiments, the human subject or one or more individuals of the human subject population are pregnant or intend to become pregnant or women of childbearing potential.

In certain embodiments, the human subject or one or more individuals of the human subject population are from a Zika endemic region, optionally subject to an outbreak. In other embodiments, the human subject or one or more individuals of the human subject population are from a Zika non-endemic region, optionally travelling to an endemic region.

In certain embodiments, the human subject or one or more individuals of the human subject population are Hispanic, Latino, African American, Black, White, Multiracial, Native Hawaiian, Pacific Islander, American Indian, or Alaska Native.

In certain embodiments, the human subject or one or more individuals of the human subject population are from about 18 to about 49 years of age. In certain embodiments, the human subject or one or more individuals of the human subject population are from about 18 to about 29 years of age. In certain embodiments, the human subject or one or more individuals of the human subject population are from about 30 to about 49 years of age. In certain embodiments, the human subject or one or more individuals of the human subject population are from about 13 to about 17 years of age. In certain embodiments, the human subject or one or more individuals of the human subject population are from about 50 to about 64 years of age. In certain embodiments, the human subject or one or more individuals of the human subject population are from about 9 to about 12 years of age. In certain embodiments, the human subject or one or more individuals of the human subject population are from about 9 to about 64 years of age.

In certain embodiments, the human subject or the individual human subjects of the human subject population are flavivirus naïve. In certain embodiments, the human subject or the individual human subjects of the human subject population are flavivirus primed.

Further embodiments of the disclosure, that can be combined with any of the embodiments mentioned above, are given in the “further item lists” and the claims below.

The disclosure of international patent publications WO 2019/090233, WO 2019/108970, WO 2019/090238, WO 2019/090228, WO 2019/108976, and WO 2020/226831 is herewith incorporated by reference in its full. In case of conflict, the disclosure of the present application prevails.

The present disclosure will be more fully understood by reference to the following Examples. They should not, however, be construed as limiting any aspect or scope of the present disclosure in any way.

### EXAMPLES

### Example 1: Clonal Zika Virus Strain Generation

This example describes the production of Zika virus strains with a known research history.

### Materials and Methods

### Vero Cell Maintenance

One vial of WHO Vero 10-87 cells was rapidly thawed in a water bath and directly inoculated into 19 mL pre-warmed DMEM (Dulbecco's modified minimal essential medium) containing penicillin-streptomycin, L-glutamine 40 mM, and 10% FBS in a T-75 cm² flask at 36° C.+/2° C., at 5% CO₂. Cells were allowed to grow to confluency and sub-cultured using TryplE. This flask was expanded to two T-185 cm² flasks, grown to confluency and sub-cultured to 31×T-185 cm² flasks and grown until the cells reached 100% confluency. Cells were harvested by trypsinization, centrifuged at 800×g for 10 minutes, and resuspended in DMEM containing 10% FBS and 10% DMSO at a concentration of 1.9×10⁷ cells/mL. One vial of the Vero cells was rapidly thawed and resuscitated as described above into a T-75 cm² flask. These were sub-cultured twice to produce a cell bank in 13×T-185 cm² flasks. After trypsinization, the cells were centrifuged at 800×g and resuspended in freezing media (DMEM containing 10% FBS, and 10% DMSO) at a concentration of 4.68×10⁵ cells/mL. This cell bank was aliquoted into cryovials.

The Vero cells were grown and maintained in DMEM containing penicillin-streptomycin, L-glutamine and 10% FBS (cDMEM-10%-FBS). TryplExpress was used to maintain and trypsinize cells. Two days before viral adsorption, 6-well plates were seeded with 4-5×10⁵ cells/well in 3 mL of cDMEM-10%-FBS or 7×10⁵ cells in T-25 cm² flasks in 5 mL cDMEM-10%-FBS, or 1×10⁴ cells/well in 96-well plates in 0.1 mL cDMEM-10%-FBS. Incubators were monitored daily to maintain indicated temperatures. The Vero cell lines were stored in liquid nitrogen.

### Plaque Assay

Viral titers were determined by plaque titration in freshly confluent monolayers of Vero cells grown in 6-well plates. Frozen aliquots were thawed and ten-fold dilution series of the aliquots were made in CDMEM-0%-FBS in 96-well plates. The diluted viruses were maintained on ice prior to inoculation of the Vero cell monolayers. At the time of assay, the growth medium was aspirated from the 6-well plate, and 100 μL of each virus dilution was added to the wells. Virus was adsorbed for 60 min at 36° C.±2° C., at 5% CO₂, with frequent (every 10 min) rocking of the plates to prevent drying of the cell sheets. Following viral adsorption, 4 mL of a first agarose overlay (1× cDMEM-2%-FBS+0.8% agarose) maintained at 40-41° C. was added to each well. The agarose was allowed to solidify for 30 min at room temperature, and the plates were then incubated upside down for 4-6 days at 36° C.±2° C., at 5% CO₂. Two mL of a second agarose overlay containing 160 μg/mL of neutral red vital dye was added on day 4. Plaques were visualized on days 5 and 6.

### Virus Quantification by TCID₅₀ Assay

Viral titers were also determined by titration in freshly confluent monolayers of Vero cells grown in 96-well plates. Frozen aliquots were thawed and ten-fold dilution series of the aliquots were made in CDMEM-2%-FBS diluent in 96-well plates. The diluted viruses were maintained on ice prior to inoculation of the Vero cell monolayers. At the time of assay, the growth medium was aspirated from the 96-well plate, and 100 μL of each virus dilution was added to the wells. The plates were incubated for 5 days at 36° C.±2° C., at 5% CO₂. The 50% Tissue Culture Infective Dose (TCID₅₀) titer was calculated using the Reed/Muench calculator.

### Test Articles

Zika virus strain PRVABC59 was isolated from serum from a person who had traveled to Puerto Rico in 2015. The genome of the PRVABC59 strain was derived directly from the patient serum and was identified to belong to the Asian genotype (Lanciotti et al. Emerg. Infect. Dis. 2016 May; 22 (5): 933-5 and GenBank Accession Number KU501215.1).

Zika virus strain PRVABC59 (one 0.5 mL vial on dry ice) was received from the Centers for Disease Control and Prevention (CDC). Zika virus identification was confirmed through RT-PCR. The strain tested negative for Alphavirus and mycoplasma contamination by PCR. This information is summarized in Table 5.

TABLE 5
PRVABC59 strain information

| | Isolation | Patient | | | |
|---|---|---|---|---|---|
| Strain | Information | information | Prep info | Analyses | PFU |
| PRVABC59 | Human | None | Passage: | Sequencing by ion torrent: | 6.7 log |
| (Asian) | serum; travel | provided | Vero(2)C6/36(1) | gene accession #KU501215 | pfu/mL |
| | to Puerto Rico | | Prep: 29 Jan. 2016 | PFU by plaque assay | |
| | in 2015 | | Host: C6/36 | Identity by RT-PCR | |
| | | | | (−) For alphaviruses by PCR | |
| | | | | (−) for mycoplasma by ATCC | |
| | | | | and ABM PCR | |

### Sequencing

A QIAampViral RNA Mini Spin kit was used to extract RNA from stabilized virus harvests of each isolate according to manufacturer protocols. Extracted RNA from each isolate was used to create and amplify 6 cDNA fragments encompassing the entire Zika viral genome. Amplified cDNA fragments were analyzed for size and purity on a 1% Agarose/TBE gel and subsequently gel purified using a Qiagen Quick Gel Extraction Kit. An ABI 3130XL Genetic Analyzer sequencer was used to conduct automatic sequencing reactions. Lasergene SeqMan software was used to analyze sequencing data.

### Results

A Zika virus strain with a known research history that was relevant to the current Zika virus outbreak in the America's was sought. For this reason, Zika virus strain PRVABC59 was chosen. To generate a well-characterized virus adapted for growth in Vero cells, the Zika virus PRVABC59 was first amplified in Vero cells (P1).

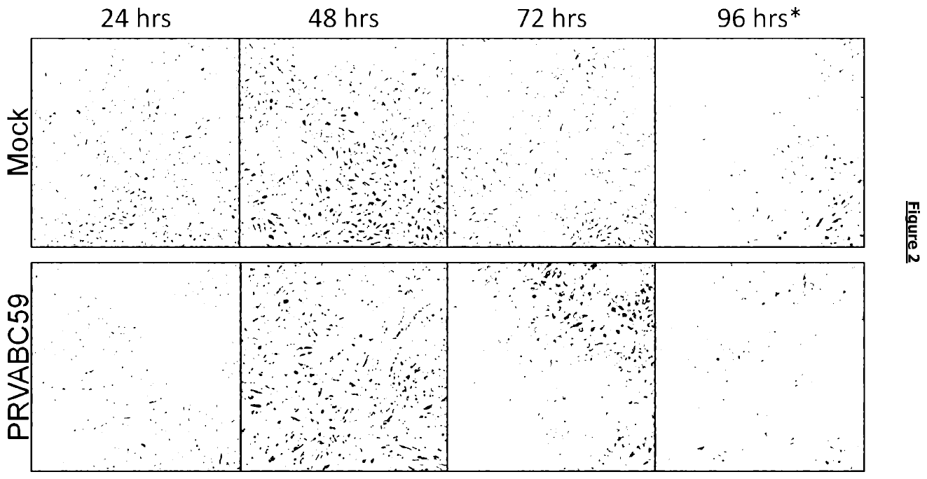

Flasks of Vero cells (T-175 cm²), 100% confluent, were infected at an MOI of 0.01 in 4 mL of CDMEM-0%-FBS. Virus was adsorbed to the monolayer for 60 minutes at 36° C.±2° C., at 5% CO₂, then 20 mL of cDMEM-0%-FBS was applied for viral amplification at 36° C.±2° C., at 5% CO₂. The cell layer was monitored daily for cytopathic effect (CPE) following inoculation (FIG. 2). The supernatant was harvested after 96 hours by collecting the media and clarifying by centrifugation (600×g, 4° C., 10 min). The harvest was stabilized by adding trehalose to a final concentration of 18% w/v. The bulk was aliquoted into 0.5 mL cryovials and stored at −80° C.

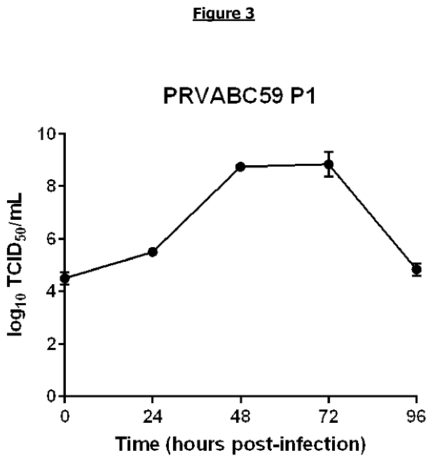

The stabilized P1 harvest was analyzed for the presence of infectious virus on Vero cell monolayers by a TCID₅₀ assay. Growth kinetics were monitored by taking daily aliquots beginning on hour 0. Peak titer was reached by hour 72 (FIG. 3).

P1 material was plaque-purified by titrating the harvest from day 3 on 6-well monolayers of Vero cells. Plaques were visualized on day 6, and 10 plaques to be isolated were identified by drawing a circle around a distinct and separate plaque on the bottom of the plastic plate. Plaques were picked by extracting the plug of agarose using a sterile wide bore pipette while scraping the bottom of the well and rinsing with cDMEM-10%-FBS. The agarose plug was added to 0.5 mL of cDMEM-10%-FBS, vortexed, labeled as PRVABC59 P2a-j and placed in an incubator overnight at 36° C.±2° C., at 5% CO₂.

Three plaques (PRVABC59 P2a-c) were carried forward for additional purification. Each isolate was plated neat in duplicate onto a fresh 6-well monolayer of Vero cells. This P2/P3 transition was plaque purified, and labeled PRVABC59 P3a-j.

Six plaques (PRVABC59 P3a-f) were carried forward for a final round of purification. Each isolate was plated neat in duplicate onto a fresh 6-well monolayer of Vero cells. This P3/P4 transition was plaque purified, and labeled PRVABC59 P4a-j.

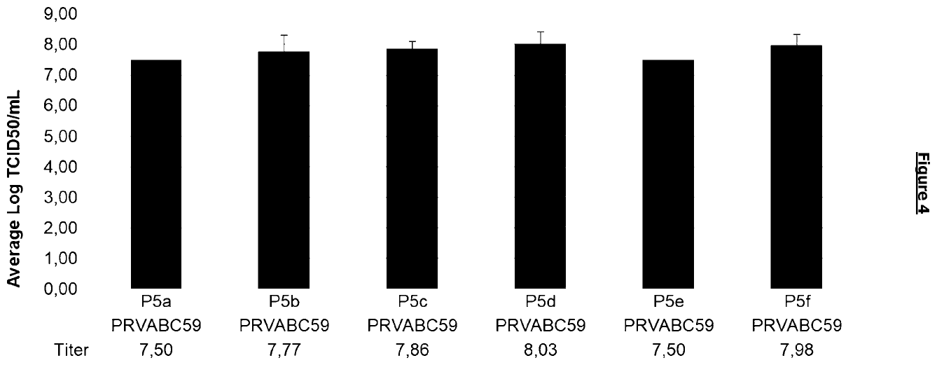

Six plaques (PRVABC59 P4a-f) from the P4 plaque purification were blind passaged on monolayers of Vero cells in T-25 cm² flasks. Each plaque pick was diluted in 2 mL cDMEM-0%-FBS-1 mL was adsorbed for 1 hour at 36° C.±2° C., at 5% CO₂; the other 1 mL was stabilized with trehalose (18% v/v final) and stored at <−60° C. Following virus adsorption, cDMEM-0%-FBS was added to each flask and allowed to grow at 36° C.±2° C., at 5% CO₂ for 4 days. Virus supernatants were harvested, clarified by centrifugation (600×g, 4° C., 10 min), stabilized in 18% trehalose and aliquoted and stored at <−60° C. This P5 seed was tested by TCID₅₀ for Zika virus potency (FIG. 4).

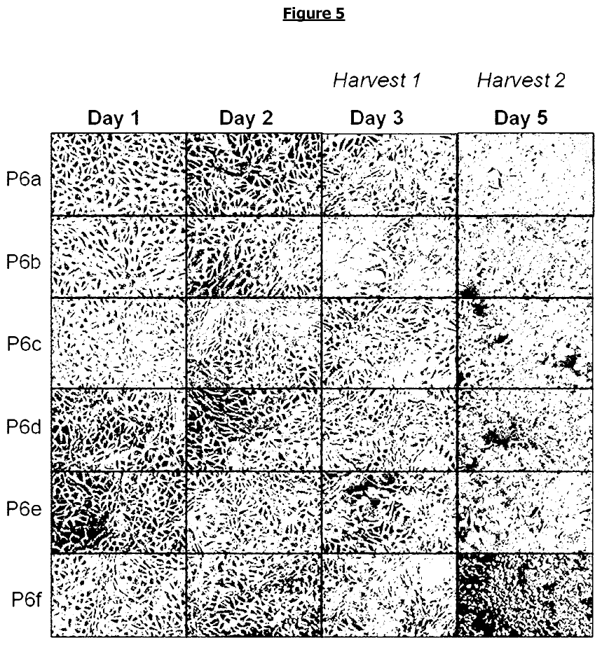

Confluent monolayers of T-175 cm² flasks of Vero cells were infected with each of the six clones of PRVABC59 (P5a-f) at an MOI of 0.01 in 4 mL cDMEM-0%-FBS. The virus was allowed to adsorb for 60 minutes at 36° C.±2° C., at 5% CO₂, after which 20 mL of cDMEM-0%-FBS was added to each flask and allowed to grow at 36° C.±2° C., at 5% CO₂. Vero cell monolayer health and CPE was monitored daily. Virus was harvested on days 3 and 5 as indicated (FIG. 5). The P6 strain harvests from days 3 and 5 were pooled, stabilized with 18% trehalose, aliquoted and stored <−60° C.

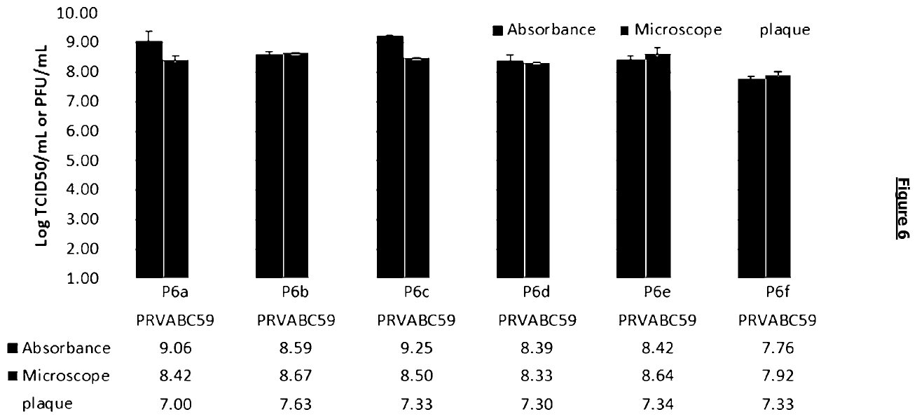

Each of the six clones of PRVABC59 (P6a-f) were tested for Zika virus in vitro potency (FIG. 6). The potency was determined by two different methods, TCID₅₀ and plaque titration. The TCID₅₀ was calculated by visual inspection of CPE (microscope) and by measuring the difference in absorbance (A560-A420) of the wells displaying CPE (yellow in color) compared with red (no CPE). The plates were read on a plate reader, and applied to the same calculator as the microscopically read-plates (absorbance). The values in TCID₅₀ between the two scoring techniques are quite similar, while the values obtained by plaque titration are lower.

A summary of the generation of the P6 virus and characterization is shown in Table 6 below.

TABLE 6
Summary of virus passage and characterization
for the generation of clonal Zika virus strains

| Passage | Seed production/purification | Characterization |
|---|---|---|
| P1 | Virus amplification in Vero | TCID₅₀ titer |
| P2 | Amplify P1 by plaque titration | plaque purification |
| P3 | Pick and passage plaques from | plaque purification |
| | P2 plaque assay | |
| P4 | Pick and passage plaques from | plaque purification |
| | P3 plaque assay | |
| P5 | Amplify P4 plaques (a-f) | TCID₅₀ titer |
| P6 | Amplify P5 (a-f) virus | TCID₅₀ titer, |
| | | plaque phenotype, |
| | | genotype, growth kinetics |

An isolated Zika virus clone that closely resembled the envelope glycoprotein sequence of the original isolate was sought, since the envelope protein of flaviviruses is the dominant immunogenic portion of the virus. PRVABC59 clones P6a, P6c, P6d and P6f contained a G→T mutation at nucleotide 990 in the envelope region (G990T), resulting in an amino acid mutation of Val→Leu at envelope residue 330, whereas the envelope gene of PRVABC59 clones P6b and P6e were identical relative to the reference strain (GenBank ref KU501215.1) (Table 7).

TABLE 7
Sequencing of PRVABC59 P6 clones

| | | | | |
|---|---|---|---|---|
| Strain | Nucleotide | Amino Acid | Mutation | Comments |

Envelope sequencing (reference gene from PRVABC59; accession #KU501215)

| | | | | |
|---|---|---|---|---|
| PRVABC59 P6a | Env-990: | Env-330: | Val/Leu | Mutation in 3 of 4 reads. |
| | G→T | Val330→Leu | | |
| PRVABC59 P6b | Env-1404: | Wild type | Wild type | Wild type relative to |
| | T→G | | | reference. |
| | silent | | | |
| PRVABC59 P6c | Env-990: | Env-330: | Val/Leu | Mutation in 3 of 4 reads. |
| | G→T | Val330→Leu | | |
| PRVABC59 P6d | Env-990: | Env-330: | Val/Leu | Mutation in 2 of 2 reads. |
| | G→T | Val330→Leu | | |
| PRVABC59 P6e | Wild type | Wild type | Wild type | Wild type relative to |
| | | | | reference. |
| PRVABC59 P6f | Env-990: | Env-330: | Val/Leu | Mutation in 2 of 2 reads. |
| | G→T | Val330→Leu | | 190 bp not sequenced |
| | | | | (aa 421-484). |

Full genome sequencing (reference gene from PRVABC59; accession #KU501215)

| | | | | |
|---|---|---|---|---|
| PRVABC59 P6b | Env-1404 | Wild-type | Silent | Mutation in 2 of 2 reads |
| | T→G | | | |
| | NS1-292 | NS1-98 | Trp/Gly | Mutation in 2 of 2 reads |
| | T→G | Trp98→Gly | | |
| PRVABC59 P6e | NS1-292 | NS1-98 | Trp/Gly | Mutation in 2 of 2 reads |
| | T→G | Trp98→Gly | | |

The two clones lacking mutations in the envelope sequence were then subjected to full genome sequencing. Sequencing results are summarized in Table 7 above. Sequence analysis revealed a T→G substitution at nucleotide 292 in the NS1 region for both clones, resulting in a Trp→Gly mutation at NS1 residue 98. This mutation was also later confirmed through deep sequencing. The NS1 W98G mutation is located in the intertwined loop of the wing domain of Zika virus NS1, which has been implicated in membrane association, interaction with envelope protein and potentially hexameric NS1 formation. While other tryptophan residues (W115, W118), are highly conserved across flaviviruses, W98 is not (FIG. 1). Interestingly, however, 100% conservation of the W98 residue is observed across 11 different Zika virus strains, including those from the African and Asian lineages, indicating that the residue might be important for certain phenotypic properties. The identified mutations in each strain are summarized in Table 8. It was surprising that no mutations in the envelope protein were observed, given the fact that flaviviruses are genetically highly labile, in particular, when cultured in vitro.

TABLE 8
Summary of mutations identified in PRVABC59 P6 clones

| | | |
|---|---|---|
| Clone | Nucleotide | Amino Acid |

Mutations identified in envelope

| | | |
|---|---|---|
| P6a | G990T | V330L |
| P6b | T1404G | (silent) |
| P6c | G990T | V330L |
| P6d | G990T | V330L |
| P6e | none | none |
| P6f | G990T | V330L |

Additional mutations identified in genome

| | | |
|---|---|---|
| P6b | NS1-T292G | NS1-W98G |
| P6e | NS1-T292G | NS1-W98G |

Ref sequence: KU501215.1 (PRVABC59)

The genomic sequence of Clone P6e is characterized by SEQ ID NO: 3, the polyprotein sequence of Clone P6e by SEQ ID NO: 4. The envelope and NS1 protein sequences of Clone P6e are represented by SEQ ID NO: 6 und SEQ ID NO: 8, respectively.

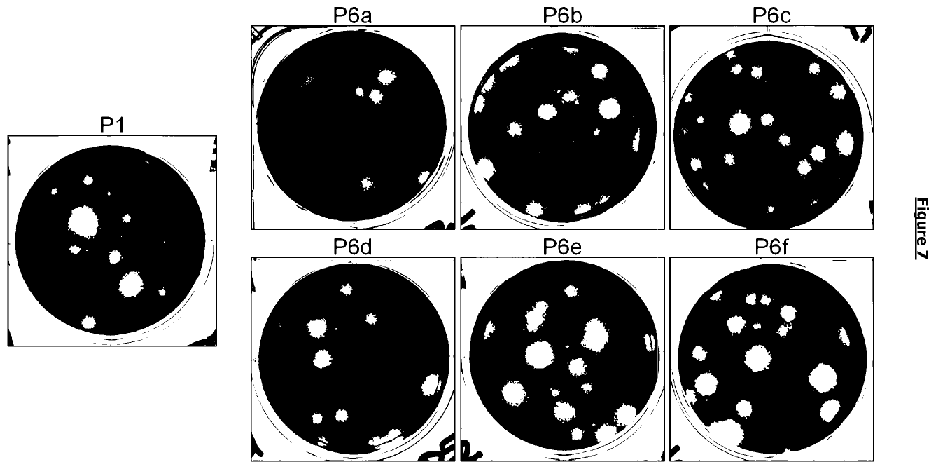

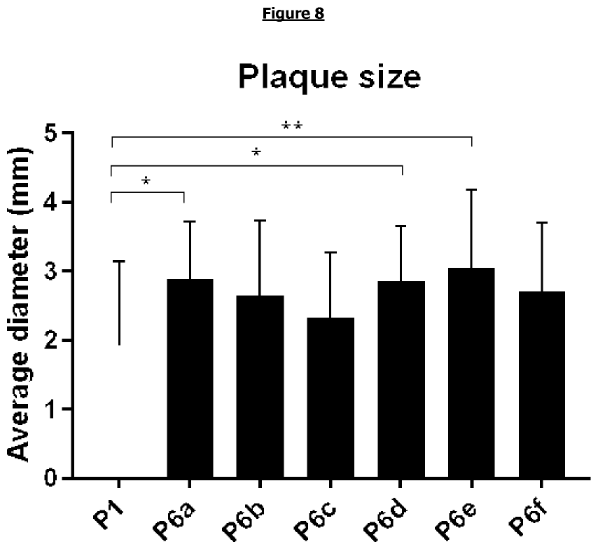

Phenotypic analysis of the Zika virus PRVABC59 P6 stocks was conducted to characterize the Zika virus clones. As illustrated in FIG. 7 and quantified in FIG. 8, each clonal isolate consisted of a relatively homogeneous population of large-sized plaques as compared to the P1 virus, which had a mixed population of large and small plaques. These data suggest the successful isolation of single Zika virus clones.

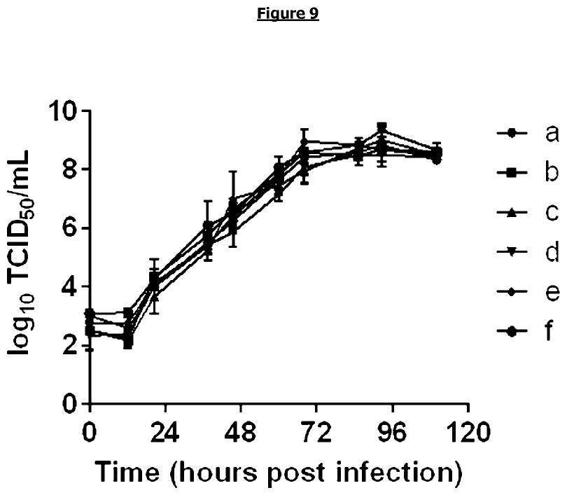

Next, growth kinetics analyses in Vero cells of the Zika virus PRVABC59 P6 clones were analyzed. Vero cells were infected with 0.01 TCID₅₀/cell of each Zika virus P6 clones in serum free growth medium. Viral supernatant samples were taken daily and simultaneously assayed for infectious titer by TCID₅₀ assay. For all P6 clones, peak titer occurred between day 3 and 4 (˜9.0 log₁₀ TCID₅₀/mL). There was no significant difference in growth kinetics of the various P6 clones (FIG. 9).

Taken together, the results indicate that a Zika virus seed was successfully generated. This seed selection required understanding of growth history, kinetics, yield, genotype, and phenotype of the virus. Importantly, clonal isolation of the Zika virus strains allowed for the successful purification of the virus away from contaminating agents (e.g., adventitious agents that may be in the parental human isolate). Interestingly, three sequential plaque purifications succeeded in quickly selecting Vero-cell adapted virus (strains P6a-f), where these strains were able to replicate well in serum-free Vero cell cultures, with strain P6a, c, d, and f harboring a mutation in the viral envelope protein, while strains P6b and P6e obtained a mutation in the viral NS1 protein (with no amino acid modification to the viral envelope protein). Additionally, the Vero-adapted strains enabled efficient and reproducible growth and manufacture of subsequent viral passages propagated from these strains.

Without wishing to be bound by theory, the Env-V330L mutation observed in strains P6a, c, d, and f may potentially be a result of in vitro adaptation, as a mutation at Env 330 was also observed upon passaging in Vero cells (Weger-Lucarelli et al. 2017. Journal of Virology). Because the envelope protein is the dominant immunogenic epitope of Zika virus, strains containing a Vero adaptive mutation in Env may negatively impact vaccine immunogenicity and ultimately degree of protection. Without wishing to be bound by theory, the adaptation mutation in protein NS1 appears not only to enhance viral replication, but may also reduce or otherwise inhibit the occurrence of undesirable mutations, such as in the envelope protein E (Env) of the Zika virus. In addition, NS1 may be known to bind to the Envelope protein during the life cycle of the virus. This mutation (NS1 W98G) may be implicated in changing the ability of the NS1 to associate, and possibly co-purify, with the virus during downstream processing. NS1 is also known to be immunogenic, and could be implicated in the immune response to the vaccine.

### Example 2: Preclinical Immunogenicity and Efficacy of a Purified Inactivated Zika Virus Vaccine (PIZV) Derived from the P6b and P6e Strains

The following example describes the preclinical immunogenicity and efficacy in CD1 and AG129 mice of an inactivated Zika virus vaccine (PIZV) derived from the P6b and P6e strains. As described in Example 1, six clones were generated from the epidemically relevant PRVABC59 strain, and two (P6b and P6e) were chosen for further preclinical immunogenicity and efficacy studies.

### Materials and Methods

### Purification, Inactivation and Formulation of a Zika Virus Vaccine

A lot of inactivated Zika virus vaccine, suitable for use in preclinical immunogenicity and efficacy studies, was generated and characterized. Virus was amplified from the P6b and P6e strains by infecting flasks of confluent Vero cells at a MOI of 0.01. Virus was adsorbed for 1 hour at 36° C.±2° C./5% CO₂. Following adsorption, 20 mL of cDMEM-0%-FBS was added to each flask, and incubated at 36° C.±2° C./5% CO₂ for five days. Cell supernatants were harvested on day 3 and 5 post-infection, and cell debris was clarified by centrifugation.

For each isolate, clarified supernatants were pooled, stabilized in DMEM containing 18% trehalose and stored at <−60° C. Pooled, clarified virus supernatants were thawed in a 37° C. water bath and treated with benzonase overnight at 4° C. Following benzonase treatment, each sample was applied to a Sartorius PP3 depth filter. Following depth filtration, each sample was applied to a Centricon Plus-70 tangential flow filtration (TFF) device. Retentate was buffer exchanged, diluted, and applied to a Sartorius SartobindQ IEXNano. Each sample was applied to a second Sartorius SartobindQ IEXNano and eluted using a 3 step-elution process with 250 mM, 500 mM, and 750 mM NaCl. Following MonoQ chromatography and dilution, each 250 mM eluate was applied to a Centricon Plus-70 cross flow filtration (CFF) device for buffer exchange, diluted to 35 mL with PBS, and stored at 2-8° C.

For formalin inactivation, freshly prepared 1% formaldehyde was added dropwise to each purified sample with gentle swirling to obtain a final formaldehyde concentration of 0.02% (w/v). Samples were incubated at room temperature (˜22° C.) for 14 days with daily inversion. Formaldehyde was neutralized with sodium metabisulfite for 15′ at room temperature before being applied to a Centricon Plus-70 tangential flow filtration (TFF) device. Buffer exchange was performed four times by the addition of 50 mL Drug Substance Buffer (10 mM NaH₂PO₄, 50 mM NaCl, 6% sucrose, pH 7.4). Each sample was then diluted to 15 mL with Drug Substance Buffer, sterilized using a 0.2 m syringe filter, aliquoted into sterile stoppered glass vials (0.5 mL per vial) and frozen at <−60° C.

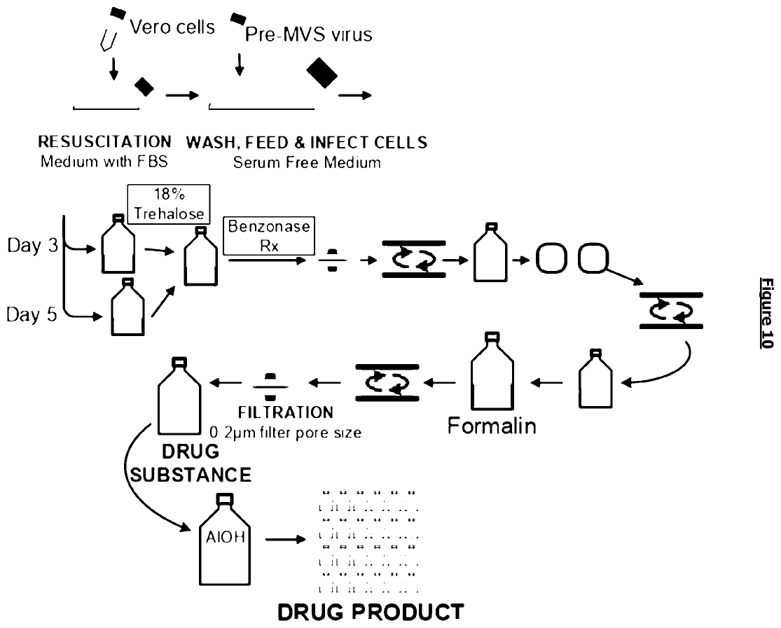

Virus inactivation was confirmed by TCID₅₀ assay and double infectivity assay (cf. also Example 4). Briefly drug substance sample was applied to C6/36 cells and allowed to amplify for 6 days. Supernatant from C6/36 cells was applied to Vero cells and CPE was monitored for 8 days. For drug product formulation, vials of PIZV drug substance were thawed, pooled according to sample type, and diluted to 1 μg/mL or 10 μg/mL in PBS with or without aluminum hydroxide adjuvant (Alhydrogel, Brenntag; 0.5 mg/ml final, referring to 0.050 mg aluminum/dose) and incubated overnight at 2-8° C. with gentle agitation. The resulting drug product lots were then aliquoted into sterile stoppered glass vials and stored at 2-8° C. until use. FIG. 10 provides a summary of the steps used to prepare drug product.

### Mouse Immunization and Challenge

For the immunogenicity study, six-week old male and female Swiss-ICR (CD-1) mice were divided into 6 groups (n=10/group). On Day 0, mice in groups 1-5 were inoculated with 0.1 mL of vaccine by the intramuscular (i.m.) route (2×0.05 mL injections). Mice in group 6 were inoculated with PBS as a placebo control. Mice were boosted on day 28 and 56 using the same dosage and vaccine type as day 0. Blood samples were collected on day-1 (pre-immune), day 27 (prime), day 42 (boost 1) and day 70 (boost 2).

For the immunogenicity and efficacy study, four-week old male and female AG129 mice were divided into 7 groups (n=5/group). On Day 0, mice in groups 1-6 were inoculated with 0.1 ml of vaccine by the intramuscular (i.m.) route (2×0.05 mL injections). Mice in group 7 were inoculated with PBS as a placebo control. Mice were boosted on day 28 using the same dosage and vaccine type as on day 0. Blood samples were collected from the tail vein on day-1 (pre-immune), day 27 (prime) and day 55 (boost). At the time of euthanization, mice were bled via cardiac puncture under deep anesthesia with isofluorane (terminal). On day 56, mice were intraperitoneally challenged with 10⁴ plaque forming units (PFU) of Zika virus PRVABC59.

### Serum Transfer

Serum was collected from PIZV-vaccinated and challenged AG129 mice, and were frozen after pooling (groups 1, 2, 4, and 5 of Table 6). The serum pool was thawed, and the test articles were generated by three-fold dilutions of the serum pool in PBS. A placebo was generated using 3-fold dilutions of AG129 normal mouse serum in PBS.

The test articles were administered as 0.1 mL intraperitoneal injections into AG129 mice (an equivalent volume of the placebo article was administered to control mice). Animals were then challenged intraperitoneally with 10⁴ plaque forming units of Zika virus strain PRVABC59 in 100 μL.

Allowable blood volume by weight was collected as whole blood by tail bleeding from ten mice on day-11 (pre-immunization). Whole blood was collected from each mouse on day 1 (primary, circulating Nab) and day 4 (viremia) by tail bleeding. Terminal bleeding after lethal challenge was performed by heart puncture under deep anesthesia for larger volume before euthanization by cervical dislocation. Blood samples were collected in microtainer SST serum separation gel tubes and allowed to clot for at least 30 min before separation of serum by centrifugation (10,000×g for 2 min) and frozen at −80° C.

### Plaque Reduction Neutralization Test

Neutralizing antibody titers were determined by a plaque reduction neutralization test (PRNT) as described previously (See e.g., Osorio et al. Lancet Infect Dis. 2014 September; 14 (9): 830-8).

### Reporter Virus Particle (RVP) Neutralization Assay

Neutralizing antibody titers were analyzed by titration of serum samples with a constant amount of Zika RVPs in Vero cells grown in 96-well plates. RVPs contained the prME proteins of Zika (strain SPH2012) and a Dengue-based Renilla luciferase reporter. Briefly, sera were heat inactivated at 56° C. for 30 min, diluted, and then incubated at 37° C. with RVPs. The serum/RVP mixture was then mixed with Vero cells and incubated for 72 hours at 37° C.±2° C./5% CO₂ before detection with luciferase substrate. Data was analyzed using JMP11 non-linear 4 parameter analysis, normalized to a positive tracking control and effective dose 50% (EC₅₀, also termed RVP₅₀) was reported.

Unless indicated to the contrary, all additional experimental methods were carried out as described in Example 1 above.

### Results

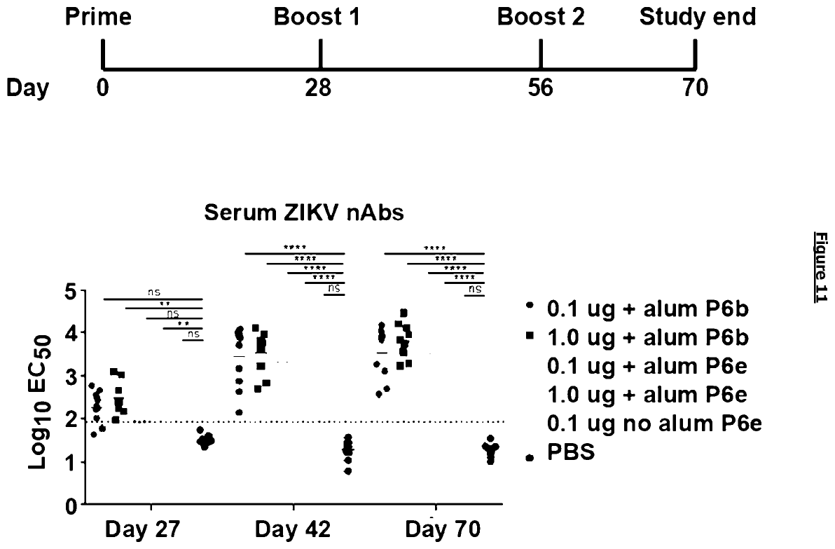

To assess the immunogenicity of the PIZV candidates in 6 week old male and female CD-1 mice, groups of CD-1 mice (N=10/group) were immunized by the i.m. route with either a 0.1 μg (+ aluminum adjuvant) or a 1.0 μg (+ aluminum adjuvant) dose of a vaccine derived from either Zika virus PRVABC69 P6b or P6e virus strains. To assess the need for adjuvant, a group of animals was vaccinated with 0.1 μg of vaccine derived from P6e and lacking aluminum adjuvant. Vaccinations occurred on days 0, 28, and 56, with group 6 receiving PBS as a placebo control (upper section of FIG. 11 and Table 9).

TABLE 9
PIZV formulations and challenges in CD-1 mice

| Group | Strain | Dose (μg) | Aluminum/dose (μg) | N |
|---|---|---|---|---|
| 1 | P6b | 0.1 | 50 | 10 |
| 2 | P6b | 1.0 | 50 | 10 |
| 3 | P6e | 0.1 | 50 | 10 |
| 4 | P6e | 1.0 | 50 | 10 |
| 5 | P6e | 0.1 | — | 10 |
| 6 | Placebo (PBS) | — | — | 10 |

Following vaccination, serum samples collected after primary (day 27), secondary (day 40) and tertiary (day 70) immunizations were tested for Zika virus-specific neutralizing antibodies by RVP neutralization assay (lower section of FIG. 11). Twenty-seven days after receiving the first dose, a slight neutralizing antibody response was observed in mice vaccinated with PIZV derived from either clone containing aluminum adjuvant, as compared to the PBS placebo control group. Importantly, this response increased significantly upon a second immunization (day 40), but was not additionally enhanced upon immunization with a third dose (day 70). No neutralizing antibody response was observed in mice vaccinated with non-adjuvanted vaccine (lower section of FIG. 11).

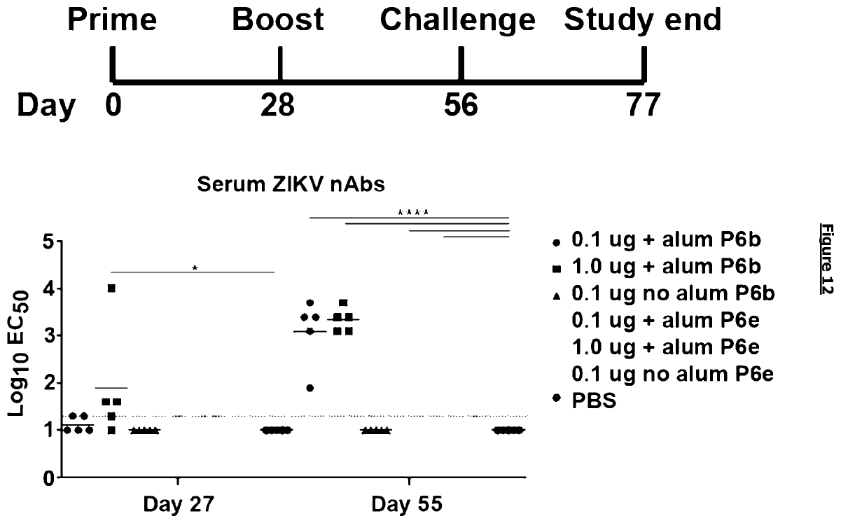

To assess the immunogenicity and protective efficacy of the PIZV candidates, groups of 4 week old AG129 mice (n=5/group) were immunized by the i.m. route with either a 0.1 μg dose (+ aluminum adjuvant), 1.0 μg dose (+ aluminum adjuvant) or 0.1 μg dose (− aluminum adjuvant) of a vaccine derived from either the Zika virus PRVABC59 P6b or P6e stocks on days 1 and 28 (upper section of FIG. 12 and Table 10).

TABLE 10
PIZV formulations and challenges in AG129 mice

| Group | Sex | Strain | Dose (μg) | Aluminum/dose (μg) | N |
|---|---|---|---|---|---|
| 1 | F | P6b | 0.1 | 50 | 5 |
| 2 | F | P6b | 1.0 | 50 | 5 |
| 3 | F | P6b | 0.1 | — | 5 |
| 4 | M | P6e | 0.1 | 50 | 5 |
| 5 | M | P6e | 1.0 | 50 | 5 |
| 6 | M | P6e | 0.1 | — | 5 |
| 7 | M | Placebo (PBS) | — | — | 5 |

Following vaccination, vaccinated and control mice were intraperitoneally challenged at day 56 with 10⁴ PFU of Zika virus PRVABC59 (low passage). Serum samples collected after primary (D27) and secondary (D55) immunizations were tested for Zika virus-specific neutralizing antibody response (lower section of FIG. 12 and Table 11). Only groups receiving the high dose of alum-adjuvanted vaccine (groups 2 and 5) elicited a neutralizing antibody response after a single immunization, which increased dramatically after boosting. In contrast, groups receiving either the low or high dose of alum-adjuvanted vaccine produced a high neutralizing antibody response after a second dose. Upon receiving two doses of vaccine, there was no statistical difference between groups of mice receiving alum-adjuvanted vaccine, regardless of the dosage or the derivation from the P6 clone.

TABLE 11
Zika virus-specific neutralizing antibody response

| | | Serum neutralizing antibody titers | | | |
|---|---|---|---|---|---|
| | | D27 (prime) | | D55 (boost) | |
| Group | Formulation | GMT | % sc | GMT | % sc |
| 1 | P6b 0.1 μg + | <20 | 40 | 1280 | 100 |
| | aluminum | | | | |
| | adjuvant | | | | |
| 2 | P6b 1.0 μg + | 135 | 80 | 2229 | 100 |
| | aluminum | | | | |
| | adjuvant | | | | |
| 3 | P6b 0.1 μg − | <20 | 0 | <20 | 0 |
| | aluminum | | | | |
| | adjuvant | | | | |
| 4 | P6e 0.1 μg + | <20 | 20 | 640 | 100 |
| | aluminum | | | | |
| | adjuvant | | | | |
| 5 | P6e 1.0 μg + | 30 | 100 | 905 | 100 |
| | aluminum | | | | |
| | adjuvant | | | | |
| 6 | P6e 0.1 μg − | <20 | 0 | <20 | 20 |
| | aluminum | | | | |
| | adjuvant | | | | |
| 7 | PBS | <20 | 0 | <20 | 0 |

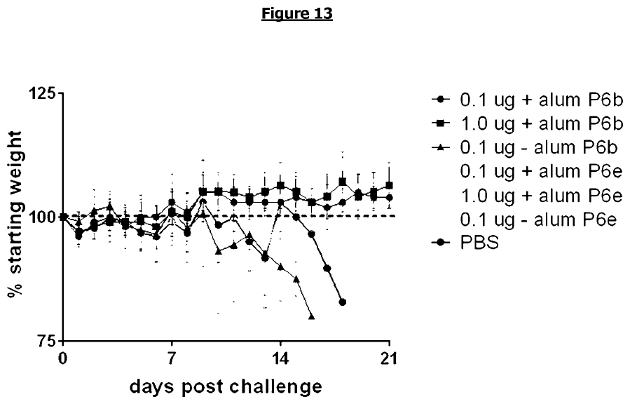

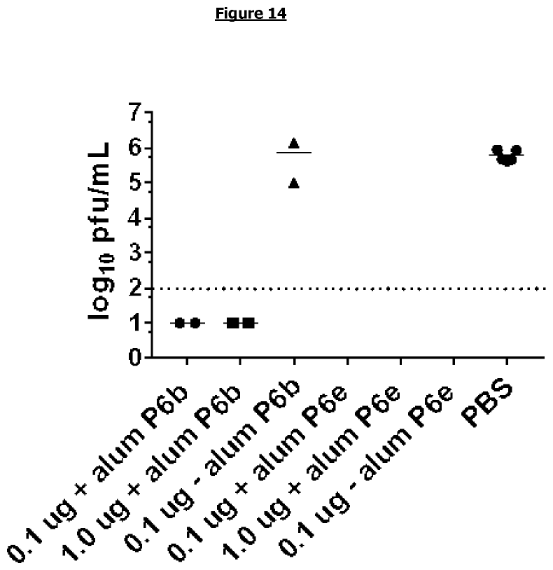

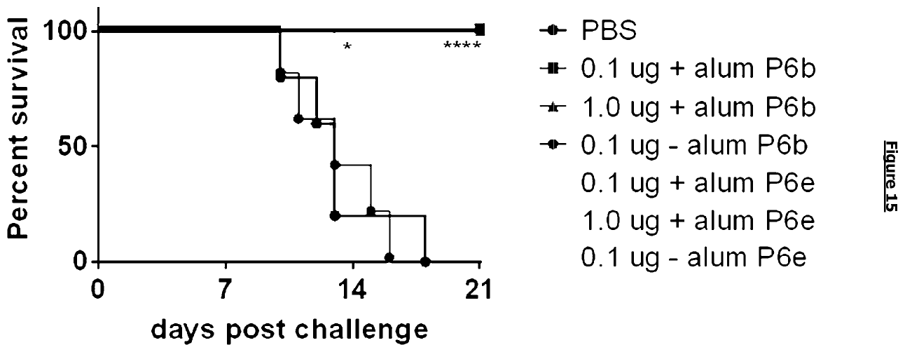

All groups were also monitored for mortality, morbidity and weight loss for 21 days post challenge. Viremia following challenge was detected and quantitated by plaque titration. Mice vaccinated with a low or high dose of PIZV candidates formulated with aluminum hydroxide adjuvant (groups 1, 2, 4 and 5) were fully protected from lethal Zika virus challenge, as assessed by the plaque reduction neutralization test (PRNT) assay, as well as a comparable secondary neutralization assay (Table 12). No weight loss or clinical signs of illness were observed in vaccinated mice, none had detectable infectious viremia three days post challenge, and all mice vaccinated with either low or high dose antigen+ aluminum adjuvant survived to 21 days post-challenge (FIGS. 13 to 15). In contrast, challenge of all naïve mice resulted in high viremia on day 2 post challenge and morbidity/mortality between day 10 and 18 post challenge (median survival=D13). Additionally, challenge of mice vaccinated with a non-alum-adjuvanted low dose vaccine derived from strain P6b resulted in high viremia on day 2 post challenge and a median survival day similar to the placebo control group, while mice vaccinated with a non-aluminum-adjuvanted low dose derived from clone e remained partially protected with a median survival of 19 days. These results indicate immunization is more effective with aluminum adjuvant, secondary immunization may be a requirement, and that low dose was as effective as high dose.

TABLE 12
Serum neutralizing antibody titers

Serum neutralizing antibody
titers
Terminal (post challenge)

| Pool | PRNT₅₀ | Secondary assay |
|---|---|---|
| Aluminum adjuvant (1, 2, 4, 5) | 10240 | 20480 |
| No aluminum adjuvant (3, 6) | 2560 | 2560 |
| PBS (7) | 1280 | 1280 |

Additionally, the presence of NS1 in the vaccine drug substance (DS) produced from whole inactivated P7b and P7e virus (one additional passage from the P6b and P6e strains, respectively) was tested. A sandwich ELISA was performed using plates pre-coated with a monoclonal antibody reactive to both Asian and African lineages of Zika virus NS1, but non-cross-reactive to Dengue NS1. Duplicate 2-, 4-, 8-, 16-, and 32-fold dilutions of DS were prepared, and were compared to a standard curve using recombinant purified NS1 in duplicate at a concentration of 0-8 ng/ml. Duplicate dilutions of DS buffer alone were prepared as negative controls. Bound NS1 was detected with anti-NS1 HRP-conjugate, and absorbance (A450-A630) of the wells with DS buffer alone was subtracted from the absorbance measured in the wells containing the matching DS samples. Results of the sandwich ELISA are shown in Table 13 below. Interestingly, NS1 was observed to co-purify with the vaccine drug substance preparations, suggesting that viral NS1 may be an immunogenic component of the whole inactivated virus vaccine.

TABLE 13
NS1 ELISA

| Strain in | | | | | | | Predicted |
|---|---|---|---|---|---|---|---|
| vaccine | Sample | Predicted | Std | Lower | Upper | Dilution | concentration |
| preparation | OD | log ng/mL | Error | 95% | 95% | Factor | (ng/mL) |
| P7b | 3.61 | 0.951 | 0.018 | 0.915 | 0.986 | 32 | ~285 |
| P7e | 3.79 | 0.980 | 0.023 | 0.935 | 1.024 | 32 | ~306 |

The threshold of neutralizing antibody (Nab) needed to confer protection from wild-type Zika virus challenge after passive transfer of antibodies was next tested. (Tables 14A and B).

TABLE 14A
Design of passive transfer study in AG129 mice

| | | | Predicted Nab |
|---|---|---|---|
| Group | Test Article | Serum dilution | titer before IP |
| 1 | 100 μL | 1/3 | 6827/3.83 |
| 2 | 100 μL | 1/9 | 2276/3.36 |
| 3 | 100 μL | 1/27 | 759/2.88 |
| 4 | 100 μL | 1/81 | 253/2.40 |
| 5 | 100 μL | 1/243 | 84/1.93 |
| 6 | 100 μL | 1/729 | 28/1.45 |
| 7 | 100 μL | 1/2187 | 9/0.97 |
| 8 | 100 μL | PBS | — |

TABLE 14B
Timing of passive transfer study in AG129 mice

| Description | Study Day |
|---|---|
| Passive transfer | Day 0 |
| Primary Bleed (AM) | Day 1 |
| Challenge (PM) | Day 1 |
| Viremia Bleed | Day 4 |
| Terminal Bleed | Day 29 for survivors |

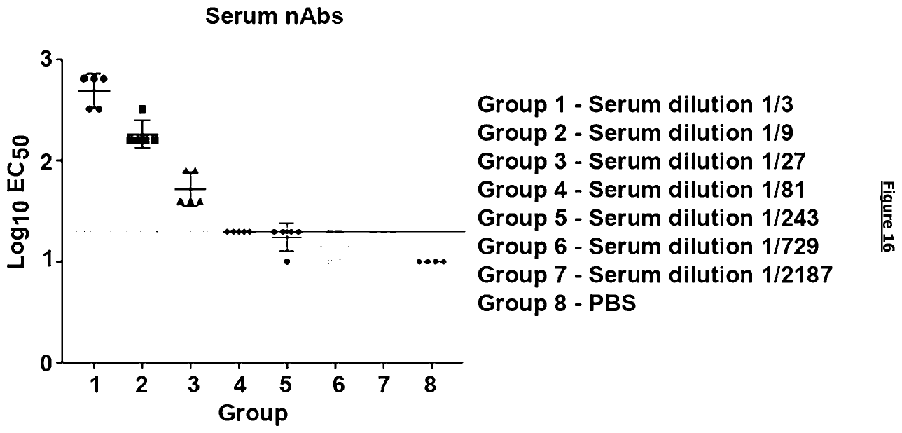

Pooled serum from vaccinated and challenged AG129 mice was serially diluted 3-fold in PBS and intraperitoneally injected into 7 groups (N=5/group) of 5-6 week old AG129 mice. Pre-immune AG129 mouse serum was used as placebo control (group 8). Following passive transfer (˜16-19 hours later), whole blood was collected and serum was separated by centrifugation from each mouse prior to virus challenge for determination of circulating neutralizing antibody titer (FIG. 16). Just prior to virus challenge, groups of mice (designated groups 1, 2, 3, 4, 5, 6, 7, 8) had mean log₁₀ neutralizing antibody titers of 2.69, 2.26, 1.72, 1.30, <1.30, <1.30, <1.30, <1.30, respectively.

Twenty-four hours following passive transfer of ZIKV neutralizing antibodies (nAbs), mice were intraperitoneally challenged with 10⁴ pfu of ZIKV PRVABC59. Following challenge, animals were weighed daily and monitored 1-3 times a day for 28 days for signs of illness. A clinical score was given to each animal based on the symptoms (Table 15). Animals that were moribund and/or showed clear neurological signs (clinical score≥2) were humanely euthanized and counted as non-survivors.

TABLE 15
Description of clinical scores given while
monitoring for morbidity and mortality

| Score | Description |
|---|---|
| 0 | Normal appearance and behavior |
| 1 | Slightly ruffled fur and/or general loss of condition |
| 2 | Increases in above behavior/appearance, breathing |
| | changes, twitching, anti-social behavior |
| 3 | First signs of neuropathy - Severely hunched posture, |
| | partial paralysis (immobility, unsteady gait, flaccid |
| | hind legs, severe twitching), or full paralysis |
| 4 | Found dead without showing signs of score of 2 or 3 first |

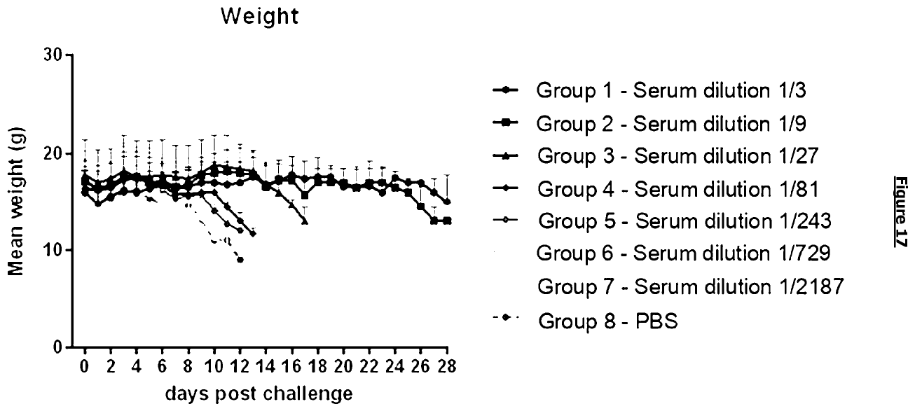

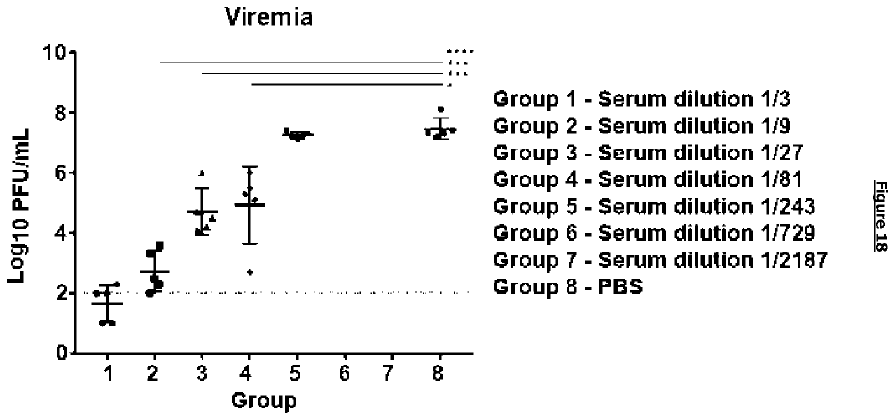

Signs of disease began appearing nine days after challenge in the control group (group 8) and groups 5-7, with a corresponding loss in weight (FIG. 17). Whole blood was collected and serum was separated by centrifugation from each animal three days post challenge. Serum samples were analyzed for the presence of infectious ZIKV using a plaque titration assay (FIG. 18). The mean infectious titer (log 10 pfu/mL) for mice in groups 1-8 were: 1.66, 2.74, 4.70, 4.92, 7.24, 7.54, 7.54 and 7.46, respectively. Importantly, mice in groups 1˜4 with detectable levels of ZIKV neutralizing antibodies (≥1.30 log 10) had statistically significant lower levels (102.5- to 106.0-fold lower titers) of viremia (p=0.0001, 0.0003, 0.0007 and 0.0374) than control mice. These results suggested that detectable levels of ZIKV neutralizing antibodies (≥1.30 log 10) reduced viremia in a dose-dependent manner.

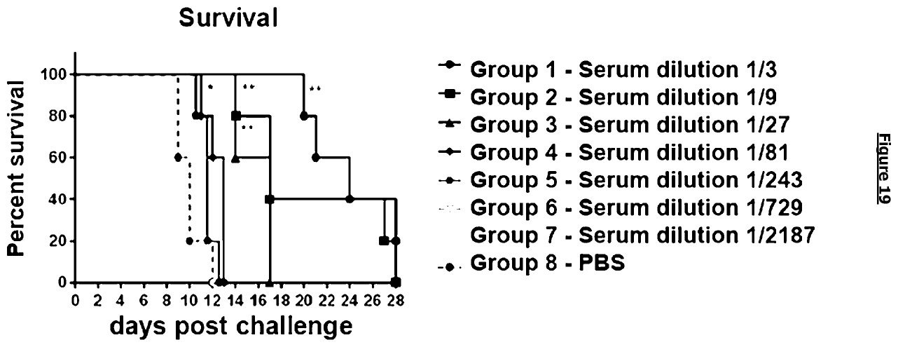

The median survival day of mice in groups 1-8 were: not determined, day 17, day 17, day 13, day 11, day 11, day 11, and day 10, respectively (FIG. 19). Importantly, the survival curves for groups of mice with detectable ZIKV neutralizing antibody titers (groups 1-4) were statistically different compared to the control group (group 8) (p=0.0019, 0.0019, 0.0019, 0.0153, respectively). These results suggested that detectable levels (≥1.30 log 10) of ZIKV neutralizing antibodies delayed onset of disease in a dose-dependent manner.

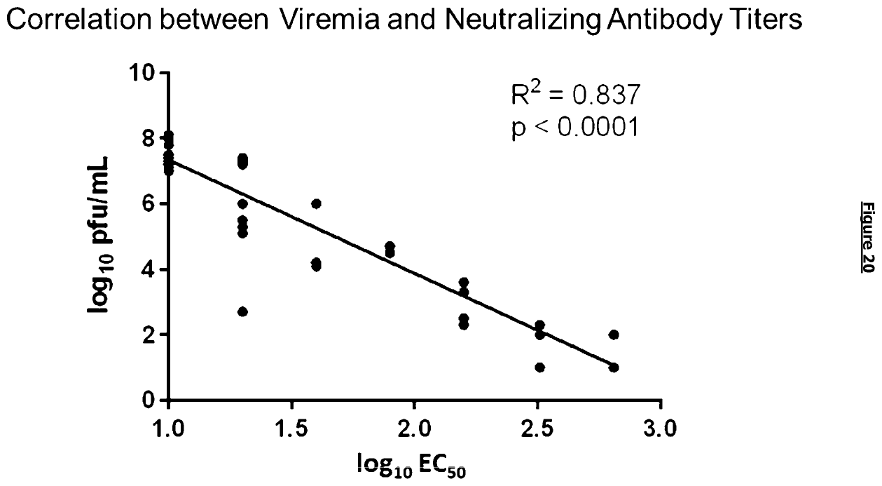

Finally, the ZIKV neutralizing antibody titer of each animal was graphed against its corresponding viremia titer and linear regression analysis was performed. A highly inversely correlated relationship between ZIKV neutralizing antibody titers and viremia levels at day 3 post-challenge was observed (FIG. 20). A summary of the results from the passive transfer studies is shown in Table 16 below.

TABLE 16
Summary of passive transfer results (D 28 = day 28 post challenge)

| | | Circulating | | % | Median |
|---|---|---|---|---|---|
| | Serum | ZIKV nAb | Viremia (D 3) | survival | survival |
| Group | dilution | GMT | log10 pfu/mL | (D 28) | day |
| 1 | 1/3 | 2.69 ± 0.17 | 1.66 ± 0.62 | 20 | 24 |
| 2 | 1/9 | 2.26 ± 0.13 | 2.73 ± 0.68 | 0 | 17 |
| 3 | 127 | 1.72 ± 0.16 | 4.69 ± 0.77 | 0 | 17 |
| 4 | 1/81 | 1.30 ± 0.16 | 4.94 ± 1.29 | 0 | 13 |
| 5 | 1/243 | <1.30 | 7.25 ± 0.10 | 0 | 11 |
| 6 | 1/729 | <1.30 | 7.54 ± 0.31 | 0 | 11 |
| 7 | 1/2187 | <1.30 | 7.52 ± 0.39 | 0 | 11 |
| 8 | PBS | <1.30 | 7.47 ± 0.37 | 0 | 10 |

While no groups of mice receiving Zika virus neutralizing antibodies were fully protected from lethal Zika virus challenge in this experiment, reduced viremia levels and delayed onset of disease in a dose-dependent manner among the groups of mice with detectable levels of circulating Zika virus neutralizing antibody titers was demonstrated.

Taken together, preclinical data from both CD-1 and AG129 mouse studies indicate that a PIZV derived from separate and well-characterized viral clones are immunogenic and able to provide protection against challenge with wild-type Zika virus. Importantly, a low and high vaccine dose elicited a similar neutralizing antibody response after two doses, and provided similar levels of protection against lethal Zika virus challenge. Interestingly, mice vaccinated with an unadjuvanted PIZV candidate also showed partial protection from Zika virus challenge. Vaccine antisera significantly diminished viremia in passively immunized AG129 mice, and prolonged survival against lethal Zika virus challenge. These results also demonstrate that the well-characterized PIZV candidates were highly efficacious against Zika virus infection in the highly Zika virus-susceptible AG129 mouse model.

Additionally, it was found that the sequence of a PRVABC59 (from PRVABC59 P6e) at passage 7 (P7e) was genetically identical to that of passage 6. As outlined above, given that flaviviruses are generally regarded as genetically labile, this was surprising. PRVABC59 P6e was selected as the master virus seed due in part to its genetic stability over 7 passages. Without wishing to be bound by theory, it is believed that this enhanced genetic stability may be due to the single amino acid substitution (W98G) in the wing domain of NS1, as this was the only mutation observed in the Vero cell-adapted PRVABC59 P6 genome. Additionally, genetic stability and homogenicity is advantageous in that it reduces variability and increases reproducible production of subsequent strains that may be used for vaccine formulation.

Strain P6e is referred to herein as pre-master virus seed (Pre-MVS). Strain P7e is referred to herein as master virus seed (MVS). These strains are particularly useful for the development of vaccines as, as outlined above, they are genetically stable and therefore allow for the production of high titers of homogenous virus. The genomic sequence of Clone P7e is characterized by SEQ ID NO: 5, the polyprotein sequence of Clone P7e by SEQ ID NO: 4. The envelope and NS1 protein sequences of Clone P7e are represented by SEQ ID NO: 6 und SEQ ID NO: 8, respectively.

### Example 3: Preclinical Assessment of the Phenotype of the P6a and P6e Strains

### Materials and Methods

AG129 mice (lacking interferon α/β and γ receptors) are susceptible to zika virus (ZIKV) infection and disease, including severe pathologies in the brain. 14-week-old AG129 mice were intraperitoneally infected with with 10⁴ and 10³ pfu of the ZIKV passage 6 clones a (P6a) and e (P6e).

Mice were weighed and monitored daily (up to 28 days) for clinical signs of illness (weight loss, ruffled fur, hunched posture, lethargy, limb weakness, partial/full paralysis). Additionally, analysis of viremia was performed by plaque titration of serum samples collected three days post-challenge as described in Example 1.

### Results

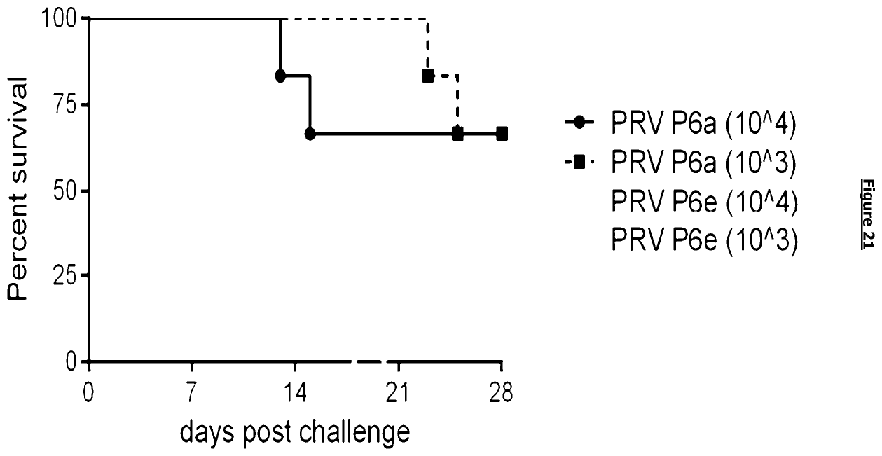

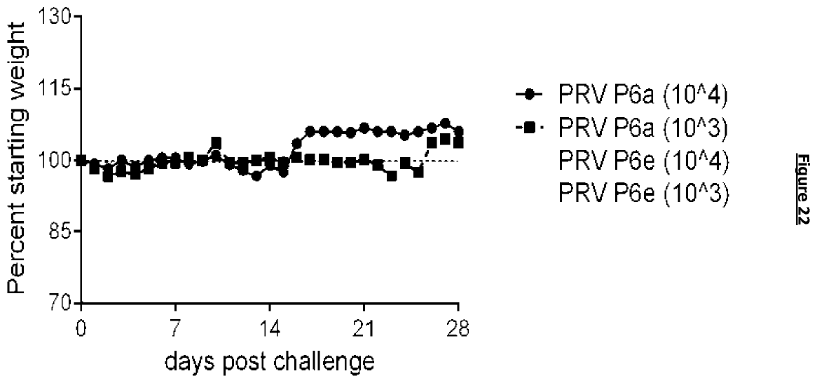

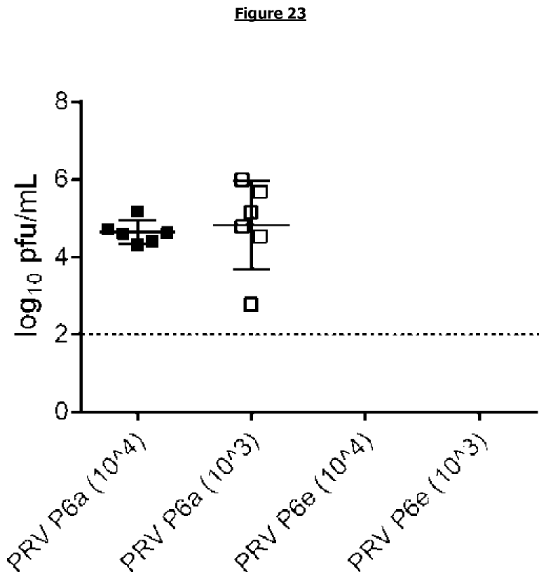

Infection with P6e resulted in 100% mortality (median survival time=12.5 days), while infection with P6a resulted in only 33% mortality (median survival time=undetermined) (FIG. 21). In agreement with this, pre-MVS P6e infected mice showed greater weight loss as compared to PRVABC59 P6a infected mice (FIG. 22). No statistical difference was found in mean group viremia levels between groups of mice infected with PRVABC59 P6a or P6e (FIG. 23). These data suggest that growth kinetics alone may not be a key determinant (since both strains produced similar viremia, and similar peak titers in vitro) and that a characteristic of the Envelope protein could be important for virulence (of a wildtype strain) and immunogenicity (of an inactivated candidate).

### Example 4: Completeness of Inactivation Assay to Determine Effectiveness of Inactivation

A double-infectivity assay, also called completeness of inactivation (COI) assay herein, was developed to determine the effectiveness of formalin-inactivation (0.01% (w/v) formaldehyde) and potential residual infectious viral activity of purified inactivated zika virus (PIZV) bulk drug substance (BDS).

Sample preparation: Four Purified Inactivated Zika Vaccine (PIZV) lots (Tox lots 1-4) of clone P6e as described above were manufactured by growth in Vero cells. Supernatants from 4 daily harvests (totaling about 4000 mL) were purified by chromatography followed by addition of formaldehyde to a final concentration of 0.01% (w/v). Inactivation was allowed to proceed for 10 days at 22° C. In Process Control (IPC) samples were removed on a daily basis from the bulk drug substance (BDS) during inactivation for characterization and analytics. The daily IPC samples were neutralized with sodium metabisulfite and dialyzed into DMEM (viral growth media). The samples contain the purified inactivated Zika virus. On the final day of inactivation, the remaining volume of BDS samples was not neutralized, but was processed with tangential flow filtration (TFF) to remove formaldehyde and buffer exchanged into PBS.

Completeness of inactivation assay (COI): The COI assay was used for analysis of the effectiveness of inactivation in the daily IPC samples to understand the kinetics of inactivation, and the final BDS. For maximum sensitivity, two cell lines, Vero and C6/36, were initially utilized in this assay to detect potential live virus in the IPC and DS samples. When Zika virus infects Vero cells in the presence of growth medium containing phenol red, the by-products of cell death cause a drop in pH. Consequently, the media color changes from red/pink to yellow, indicative of this acidic shift in the media pH. This phenomenon is caused by the apoptosis and cytopathic effects (CPE), which refers to the observed changes in the cell structure of host cells that are caused by viral invasion, infection, and budding from the cells during viral replication. Ultimately, while both C6/36 mosquito and Vero cells are a permissive cell line for infection, Zika virus infection kills only Vero cells in vitro. Therefore, Vero cells were used as the indicator cell line for the assay. In contrast, C6/36 cells which are derived from a natural host vector for Zika virus do not exhibit a CPE upon Zika infection and do not lyse. The media does not change color and the viability of the C6/36 cells is not altered.

The assay is thus split in two parts: The first part of the assay allows for parallel amplification of potentially live viral particles on 96-well plates of the two susceptible cell lines for six days. The second step of the assay involves the transfer of the supernatant of the 96-well plates (including potentially amplified particles) onto 6-well plates containing monolayers of Vero cells, and incubation for another 8 days to allow for viral infection and a cytopathic effect to develop on the Vero cells. Any CPE observed was confirmed using a light microscope.

Although described in detail with respect to the use of 96 well plates in the first part of the assay, i.e. the culture in C6/36 cells, and six well plates in the second part of the assay, i.e. the culture of Vero cells to observe a cytopathic effect, the assay can be easily scaled up according to Table 17.

TABLE 17
Scaling of the COI assay.

| | | | | | | | | | Assay part 2: | |
|---|---|---|---|---|---|---|---|---|---|---|
| | | | | | | | | | transfer to Vero | |
| | | | | Assay part 1: BDS application (must | | | | | (must accommodate | |
| | | | | fall within recommended vol range) | | | | | pooled volume | |
| | | | | | | # vessels | # vessels | | for transfer) | |
| | | | | | vol | required | required | pooled | | vol |
| | | Recommended | | | inoculum | for 15X | for 15X | volume | | transferred |
| | Surface | volume | | mL | per well | scale-up; | scale-up; | for | mL | inoculum |
| plate or | area | range | | sample | (or per | 2-fold | 5-fold | transfer | sample | per well |
| flask | (cm²) | (for growth) | | per cm² | flask) | dilution | dilution | (mL) | per cm² | (or flask) |
| 96-well | 0.32 | 100-200 | μL | 0.3125 | 0.1 | | | | | |
| format | | | | | | | | | | |
| 12-well | 3.8 | 0.076-1.14 | ml | 0.3125 | 1.188 | 6.48 | 16.21 | 11.88 | | |
| format | | | | | | | | | | |
| 6-well | 9.5 | 1.9-2.9 | mL | 0.3125 | 2.969 | 4.32 | 10.81 | 17.81 | 0.0526 | 0.1 |
| format | | | | | | | | | | |
| T25 flask | 25 | 5-7.5 | mL | 0.3125 | 7.813 | 9.86 | 24.64 | 7.813 | 0.0526 | 1.32 |
| format | | | | | | | | | | |
| T75 flask | 75 | 15-22.5 | mL | 0.3125 | 23.438 | 3.29 | 8.21 | 23.438 | 0.0526 | 3.95 |
| format | | | | | | | | | | |
| T150 | 150 | 30-45 | mL | 0.3125 | 46.875 | 1.64 | 4.11 | 46.88 | 0.0526 | 7.89 |
| flask | | | | | | | | | | |
| format | | | | | | | | | | |
| T175 | 175 | 35-52.5 | | 0.3125 | 54.688 | 1.41 | 3.52 | 54.69 | 0.0526 | 9.21 |
| flask | | | | | | | | | | |
| format | | | | | | | | | | |
| T235 | 235 | 47-70.5 | | 0.3125 | 73.438 | 1.05 | 2.62 | 73.44 | 0.0526 | 12.36 |
| flask | | | | | | | | | | |
| format | | | | | | | | | | |
| T300 | 300 | 30-40 | mL? | 0.3125 | 93.750 | 0.82 | 2.05 | 93.75 | 0.0526 | 15.78 |
| flask | | | | | | | | | | |
| format | | | | | | | | | | |

| | | | | | | | |
|---|---|---|---|---|---|---|---|
| CF1 | 6/36 | 150-200 | 0.3125 | 198.750 | 0.39 | 0.0526 | 33.45 |
| CF2 | 1272 | 300-400 | 0.3125 | 397.500 | 0.19 | 0.0526 | 66.91 |
| CF10 | 63360 | 1500-2000 | 0.3125 | 19800.000 | 0.00 | 0.0526 | 3332.74 |

It is apparent that during the scale up the volume of sample per cm² of vessel remains constant for part 1 and the same viral infection conditions are kept in part 2.

COI assay control: The titer and back titration controls for this assay were performed using Vero indicator cells and scored in a TCID₅₀ 96-well format with wells scored positive based on the media color change from pink to yellow, as a surrogate for cell death, or the presence of CPE.

Virus titer control test: Two independent replicates of the control virus (PRVABC59) of known titer were subjected to a 10-fold dilution series in media containing 2% FBS, and 100 UL of each dilution was added to four wells of a 96-well plate containing Vero cells. Plates were incubated for 5 days, then wells containing CPE were recorded and virus titer was calculated using the Reed-Meunch calculator.

Virus back titration control test: The control virus of known titer was serially diluted to 200 TCID₅₀. Two independent replicates of the 200 TCID₅₀ control virus were subjected to a 2-fold dilution series in media containing 2% FBS, and 100 μL of each dilution was added to four wells of a 96-well plate containing Vero cells. Cells were incubated for 5 days, then wells containing CPE were recorded and virus titer was calculated using the Reed-Meunch calculator.

### Detailed COI Protocol:

1. First part of the assay: Vero (1.4E⁺⁰⁵ cells/mL) and Aedes aegypti mosquito C6/36 (4E⁺⁰⁵ cells/mL) cells were seeded in 96-well plates two days prior to addition of the samples. The Vero cells were cultured in DMEM+10% final FBS+2% L-glutamine+1% penicillin/streptomycin at 37° C. C6/36 cells were cultured in DMEM+10% FBS+2% L-glutamine+1% Penicillin/streptomycin+1% nonessential amino acids at 28° C.
2. Three independent replicates of the 200 TCID₅₀ control virus (prepared in the virus back titration control test) or the DS samples were diluted (5-fold and 10-fold dilutions) into media containing 2% FBS.
3. The cells in 96-well plates were inoculated with the samples. Prior to the infection of the cell monolayers in the 96-well plates, the sample was vortexed to disrupt any possible aggregation. 100 μL of each dilution was applied to each of 5 wells into two separate 96-well plates containing Vero and C6/36 cells, respectively.
4. Media alone was included in another well for each cell type as a negative CPE control.
5. Plates were incubated for 6 days at the appropriate temperature for the cell line.
6. Second part of the assay: To allow live virus to be further amplified and visualized by CPE on a permissive cell line, the entire volume of each 96-well supernatant from both Vero and C6/36 cells was transferred to individual wells of 6-well plates of Vero cells. Inoculation proceeded for 90 minutes with rocking at 15 minutes intervals.
7. Medium containing 2% FBS was added to the wells and plates were incubated for an additional 8 days for subsequent detection of the amplified samples as a function of CPE. The inactivation was considered to be incomplete if any of the replicates of the DS showed CPE at the end of day 8.
8. The presence of live/replicating virions was visualized by the formation of plaques or CPE on susceptible cell monolayers after transfer to the 6-well plate, and incubation for 8 days to allow for viral replication. The % CPE scoring in the 6-well plates at the end of the assay was calculated as follows:
   Each 6-well plate of Vero cells was examined for CPE by visualization of colorimetric change, followed by confirmation of CPE by inspection under an inverted light microscope.
   Each 6-well plate represented one of the replicates of the DS dilutions prepared in the 5 and 10-fold dilutions described above (5 wells, plus one well containing media alone).
      Therefore, % CPE for each replicate reflected the number of wells with CPE out of 5 total wells per sample (120 total wells are used per assay). Mean % CPE and standard deviation were calculated based on three replicates of each dilution.

### Results

The daily samples were analyzed in each of the Tox lots #1-4 as shown in Tables 18 to 21.

TABLE 18
Kinetics of Inactivation, Tox lot #1

| Sample | Transfer | Mean % CPE | STDV |
|---|---|---|---|
| 1:10 Day −1 | Vero-to-Vero | 100 | 0 |
| 1:10 Day 0 | Vero-to-Vero | 100 | 0 |
| 1:10 Day 1 | Vero-to-Vero | 0 | 0 |
| 1:10 Day 2 | Vero-to-Vero | 0 | 0 |
| 1:10 Day 3 | Vero-to-Vero | 0 | 0 |
| 1:10 Day 4 | Vero-to-Vero | 0 | 0 |
| 1:10 Day 7 | Vero-to-Vero | 0 | 0 |
| 1:10 Day 8 | Vero-to-Vero | 0 | 0 |
| 1:10 Day 9 | Vero-to-Vero | 0 | 0 |
| 1:10 Day 10 | Vero-to-Vero | 0 | 0 |
| 100TCID50/mL | Vero-to-Vero | 100 | 0 |
| 1:10 Day −1 | C6/36-to-Vero | 100 | 0 |
| 1:10 Day 0 | C6/36-to-Vero | 100 | 0 |
| 1:10 Day 1 | C6/36-to-Vero | 6.7 | 12 |
| 1:10 Day 2 | C6/36-to-Vero | 13.3 | 12 |
| 1:10 Day 3 | C6/36-to-Vero | 0 | 0 |
| 1:10 Day 4 | C6/36-to-Vero | 0 | 0 |
| 1:10 Day 7 | C6/36-to-Vero | 0 | 0 |
| 1:10 Day 8 | C6/36-to-Vero | 0 | 0 |
| 1:10 Day 9 | C6/36-to-Vero | 0 | 0 |
| 1:10 Day 10 | C6/36-to-Vero | 0 | 0 |
| 100TCID50/mL | C6/36-to-Vero | 100 | 0 |

TABLE 19
Kinetics of Inactivation, Tox lot #2

| Sample | Transfer | Mean % CPE | STDV |
|---|---|---|---|
| 1:10 Day −1 | Vero-to-Vero | 100 | 0 |
| 1:10 Day 0 | Vero-to-Vero | 100 | 0 |
| 1:10 Day 1 | Vero-to-Vero | 100 | 0 |
| 1:10 Day 2 | Vero-to-Vero | 0 | 0 |
| 1:10 Day 3 | Vero-to-Vero | 0 | 0 |
| 1:10 Day 4 | Vero-to-Vero | 0 | 0 |
| 1:10 Day 7 | Vero-to-Vero | 0 | 0 |
| 1:10 Day 8 | Vero-to-Vero | 0 | 0 |
| 1:10 Day 9 | Vero-to-Vero | 0 | 0 |
| 1:10 Day 10 | Vero-to-Vero | 0 | 0 |
| 100TCID50/mL | Vero-to-Vero | 100 | 0 |
| 1:10 Day −1 | C6/36-to-Vero | 100 | 0 |
| 1:10 Day 0 | C6/36-to-Vero | 100 | 0 |
| 1:10 Day 1 | C6/36-to-Vero | 100 | 12 |
| 1:10 Day 2 | C6/36-to-Vero | 13.3 | 12 |
| 1:10 Day 3 | C6/36-to-Vero | 0 | 0 |
| 1:10 Day 4 | C6/36-to-Vero | 0 | 0 |
| 1:10 Day 7 | C6/36-to-Vero | 0 | 0 |
| 1:10 Day 8 | C6/36-to-Vero | 0 | 0 |
| 1:10 Day 9 | C6/36-to-Vero | 0 | 0 |
| 1:10 Day 10 | C6/36-to-Vero | 0 | 0 |
| 100TCID50/mL | C6/36-to-Vero | 100 | 0 |

TABLE 20
Kinetics of Inactivation, Tox lot #3

| Sample | Transfer | Mean % CPE | STDV |
|---|---|---|---|
| 1:10 Day −1 | Vero-to-Vero | 100 | 0 |
| 1:10 Day 0 | Vero-to-Vero | 100 | 0 |
| 1:10 Day 1 | Vero-to-Vero | 27 | 12 |
| 1:10 Day 2 | Vero-to-Vero | 0 | 0 |
| 1:10 Day 3 | Vero-to-Vero | 0 | 0 |
| 1:10 Day 4 | Vero-to-Vero | 0 | 0 |
| 1:10 Day 7 | Vero-to-Vero | 0 | 0 |
| 1:10 Day 8 | Vero-to-Vero | 0 | 0 |
| 1:10 Day 9 | Vero-to-Vero | 0 | 0 |
| 1:10 Day 10 | Vero-to-Vero | 0 | 0 |
| 100TCID50/mL | Vero-to-Vero | 100 | 0 |
| 1:10 Day −1 | C6/36-to-Vero | 100 | 0 |
| 1:10 Day 0 | C6/36-to-Vero | 100 | 0 |
| 1:10 Day 1 | C6/36-to-Vero | 87 | 12 |
| 1:10 Day 2 | C6/36-to-Vero | 27 | 12 |
| 1:10 Day 3 | C6/36-to-Vero | 0 | 0 |
| 1:10 Day 4 | C6/36-to-Vero | 0 | 0 |
| 1:10 Day 7 | C6/36-to-Vero | 0 | 0 |
| 1:10 Day 8 | C6/36-to-Vero | 0 | 0 |
| 1:10 Day 9 | C6/36-to-Vero | 0 | 0 |
| 1:10 Day 10 | C6/36-to-Vero | 0 | 0 |
| 100TCID50/mL | C6/36-to-Vero | 100 | 0 |

TABLE 21
Kinetics of Inactivation, Tox lot #4

| Sample | Transfer | Mean % CPE | STDV |
|---|---|---|---|
| 1:10 Day −1 | Vero-to-Vero | 100 | 0 |
| 1:10 Day 0 | Vero-to-Vero | 93 | 12 |
| 1:10 Day 1 | Vero-to-Vero | | 0 |
| 1:10 Day 2 | Vero-to-Vero | 0 | 0 |
| 1:10 Day 3 | Vero-to-Vero | 0 | 0 |
| 1:10 Day 4 | Vero-to-Vero | 0 | 0 |
| 1:10 Day 7 | Vero-to-Vero | 0 | 0 |
| 1:10 Day 8 | Vero-to-Vero | 0 | 0 |
| 1:10 Day 9 | Vero-to-Vero | 0 | 0 |
| 1:10 Day 10 | Vero-to-Vero | 0 | 0 |
| 100TCID50/mL | Vero-to-Vero | 100 | 0 |
| 1:10 Day −1 | C6/36-to-Vero | 100 | 0 |
| 1:10 Day 0 | C6/36-to-Vero | 100 | 0 |
| 1:10 Day 1 | C6/36-to-Vero | 33 | 23 |
| 1:10 Day 2 | C6/36-to-Vero | 7 | 12 |
| 1:10 Day 3 | C6/36-to-Vero | 0 | 0 |
| 1:10 Day 4 | C6/36-to-Vero | 0 | 0 |
| 1:10 Day 7 | C6/36-to-Vero | 0 | 0 |
| 1:10 Day 8 | C6/36-to-Vero | 0 | 0 |
| 1:10 Day 9 | C6/36-to-Vero | 0 | 0 |
| 1:10 Day 10 | C6/36-to-Vero | 0 | 0 |
| 100TCID50/mL | C6/36-to-Vero | 100 | 0 |

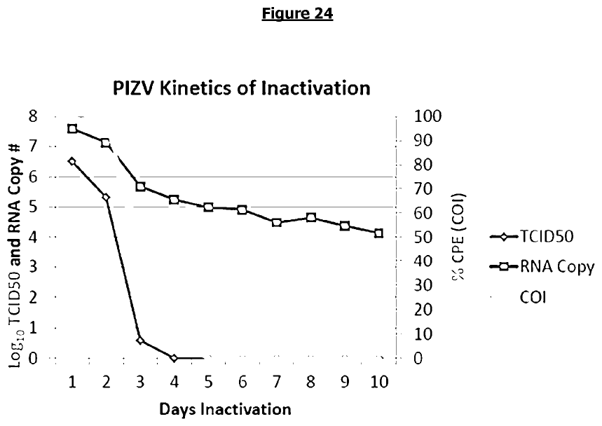

Compiled kinetics of inactivation data: COI data for samples from the four toxicology lots were compared to infectious potency (TCID₅₀) determined as described above and to RNA copy. The RNA copy was determined by purifying nucleic acids from the sample and amplifying Zika RNA with serotype-specific primers using an RT-PCR kit. The result shown in FIG. 24 demonstrate that the sensitivity of the COI assay is significantly greater than that of TCID₅₀.

Performance characteristics of the COI assay-Accuracy: The target dilutions (TCID₅₀/well) and their respective proportions of CPE were used to determine relative accuracy. For the Vero cells, there was a statistically significant linear relationship between the observed and expected proportions of positive CPE. The slope of the line relating observed and expected results is 0.92 with a 95% confidence interval (CI) of 0.83 to 1.01 that overlaps 1 indicate 100% accuracy. For the C6/36 cells, there is a statistically significant linear relationship between the observed and expected proportions of positive CPE. The slope of the line relating observed and expected results is 0.88 with a 95% confidence interval (CI) of 0.80 to 0.95 indicate that a slight bias (5-20%) was seen with this cell line. Both cell lines demonstrate satisfactory accuracy (relative).

Performance characteristics of the COI assay-Limit of Detection (LoD): The sensitivity of the assay was assessed for both the C6/36-to-Vero and Vero-to-Vero plates. As described above, the data was fitted using least squares regression of the proportion of +ve CPE observed per total wells plated with titer dilutions plated starting at 10.00 TCID₅₀/well down to a lower titer of 0.08 TCID₅₀. Furthermore, negative controls (0.00 TCID₅₀/well) were included for each dilution within the plates. CPE scoring was performed for each dilution across both the C6/36-to-Vero and Vero-to-Vero plates. A clear relationship between the CPE and log input virus titer was seen. This displays the logistic (sigmoidal) relationship between the proportion of CPE positive wells relative to the log₁₀ concentration of TCID₅₀/well together with a lower and upper 99% confidence limit. At a-2 log 10 concentration (=0.01 TCID₅₀/well), a model based on and accounting for all fixed and random sources variation in the qualification data predicted 0.85%, or 0.01 when rounded up at 0.01 TCID₅₀/well, with a lower 99% confidence limit of 0.42%. Since the lower 99% confidence limit does not include zero, there is a very small quantifiable (<1%) chance the 0.85% CPE wells could have arisen from 0 TCID₅₀/well (i.e., due to noise). This establishes a detection limit for the assay of at least 0.01 TCID₅₀/well (i.e., the lowest amount of live Zika particles in the sample which can be detected). That is, when rounded up, 1 in 60 wells will be CPE positive or given these parameters, the lowest theoretical proportion of the CPE +ve that could be detected in 60 wells would be 1.67%, or 0.0167.

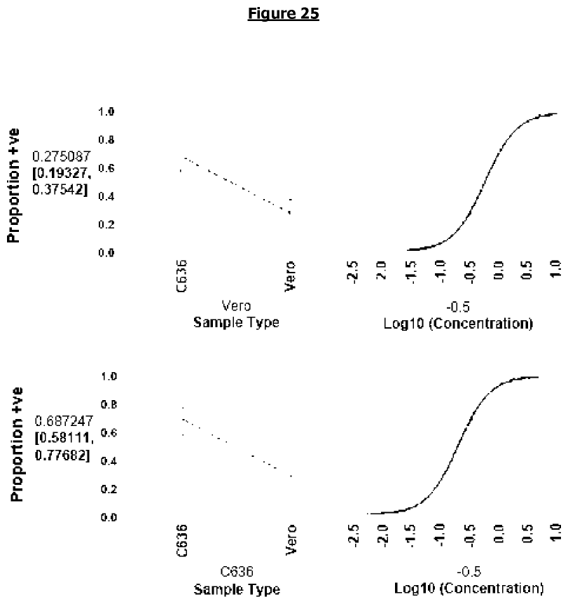

The cell types (C6/36 and Vero) were compared for relative sensitivity, with the C6/36 demonstrating that a lower dilution of virus titer could be detected compared to Vero cells as shown in FIG. 25; at the same virus input level (0.31 TCID₅₀), the proportion of CPE positive wells is higher for C6/36 relative to Vero cells.

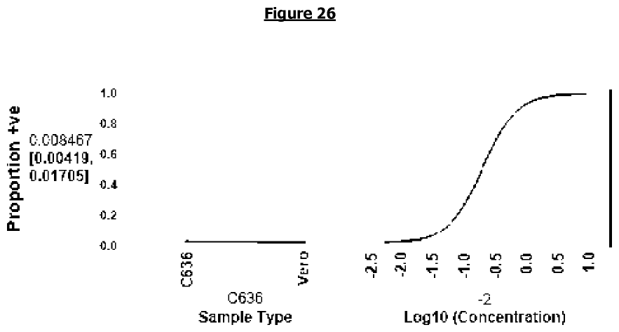

The lowest virus input value used during the qualification of this assay was 0.02 TCID₅₀ (−1.61 log TCID₅₀). Using the fitted curve for C6/36 cells, this results in 0.035 or 3.5% of the wells scoring CPE positive (1 in 28 wells). If the curve is extrapolated towards the lowest practical level of 0.167 or 1.6%, then this equates to a virus input level of 0.015 TCID₅₀ (−1.82 log TCID₅₀). However, the impact of transmitted assay variance needs to be considered when determining the lowest levels of infectious virus that can be detected as reflected in the +ve CPE results. This noise arises from generation of the working stock of input virus. Comparison of the target TCID₅₀ and the back-titration calculation shows the TCID₅₀ of the working stock virus exhibited a standard deviation (SD) of 85 TCID₅₀/mL, derived from a mean of 213 when targeting a stock TCID₅₀/mL concentration of 200. The % CV calculates to ˜40% with a bias of about +7%. This noise was factored into the logistic regression model to generate confidence intervals around the targeted values for the virus dilutions. At a target value of 0.01 TCID₅₀/well, a model based on and accounting for all fixed and random sources of variation in the qualification data across the two sites predicts 0.86% of wells will be CPE positive (1 in 60 wells). Since the lower 99% confidence limit does not include zero, there is a very small quantifiable (<1%) chance the 0.85% CPE-positive wells could have arisen from 0 TCID₅₀/well due to noise (FIG. 26). This establishes a detection limit for the assay: 0.01 TCID₅₀/well is the lowest amount of live Zika particles in the sample which can be detected.

Performance characteristics of the COI assay-Range: The range of the assay was 0.01 TCID₅₀/well to 4.5 TCID₅₀/well and is defined as the range of input virus that resulted in a CPE +ve proportion scoring of more than 0% but less than 100%.

Conclusion: Analysis of the four Tox revealed that inactivation was complete after incubation in 0.01% (w/v) formaldehyde for 10 days at room temperature. Inactivation was achieved by days 3-4 in all lots produced, as measured by the COI assay. The COI assay is more sensitive than TCID₅₀ potency or RNA measurements; the increased sensitivity has also been observed by LoD.

### Example 5: Determining Residual Formalin Content in a Pharmaceutical Composition

### Materials and Methods

### Materials

Formaldehyde standard solution (in methanol) (982 μg/mL), DNPH, HPLC-grade acetonitrile, and phosphoric acid were purchased from Wako Pure Chemicals Co. (Tokyo, Japan). Distilled water used for diluting phosphoric acid was obtained from Otsuka Pharmaceutical (Tokushima, Japan). Alhydrogel® 2% (corresponding to 10 mg/ml aluminum) used as aluminum hydroxide gel was obtained from Brenntag (Frederikssund, Denmark). PBS was prepared in-house, and the Zika vaccine drug product containing aluminum hydroxide gel was manufactured as described below. The Zika virus was purified with various techniques after harvest. After inactivation with formaldehyde, the virus was concentrated, and the buffer was exchanged with PBS by filtration. The bulk drug substance was diluted with PBS and formulated with aluminum hydroxide gel (0.4 mg/mL aluminum) to form the final drug product.

### HPLC Conditions

A Waters HPLC alliance system equipped with a UV detector (Milford, USA) and a reverse-phase column (YMC-Pack ODS-A, 4.6 mm×250 mm, 5 μm (Kyoto, Japan)) was used. A mixture of water and acetonitrile (1:1, v/v) was used as the mobile phase, the detection wavelength was set at 360 nm, and the flow rate was 1.0 mL/min. The column temperature and injection volume were 25° C. and 50 UL, respectively.

### Sample Preparation

The vaccine drug product (1.2 mL) was centrifuged at 15000 rpm for 10 min, and the supernatant (1 mL) was transferred into a 2-mL HPLC glass vial purchased from Waters (Milford, USA). Next, 20 μL of 20% (v/v) phosphoric acid and 50 μL of 1.0 mg/mL DNPH solution in acetonitrile were added, and the mixture was stirred and left at room temperature for 20 min before injection.

### Method Validation

According to the ICH Q2 guidelines, the method was validated in terms of specificity, linearity, accuracy, repeatability, intermediate precision, robustness, and stability of the sample. In the accuracy study, the Zika vaccine drug product and aluminum hydroxide gel solution were spiked with a specific amount of formaldehyde, and the sample was mixed well by vortex before analysis.

### Results and Discussion

### Linearity and Specificity

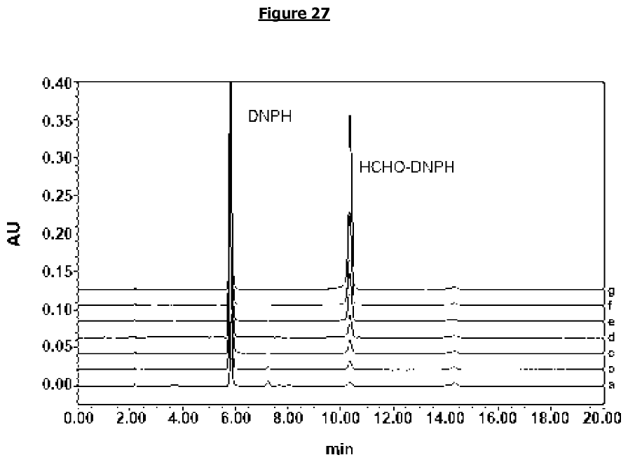

Six standard solutions of formaldehyde (0.049, 0.098, 0.196, 0.491, 0.982, and 1.964 μg/mL) were prepared by dilution with PBS. Next, 20% (v/v) phosphoric acid and 1 mg/mL DNPH solution in acetonitrile were added to each solution, and the corresponding chromatograms are shown in FIG. 27. Clearly, the 10.4-min peak area showed linearity with the regression equation: y=1075730x+11731 (where y is the area of the 10.4-min peak and x is the concentration of formaldehyde in μg/mL) (correlation coefficient: 0.9998), indicating that it was due to HCHO-DNPH (i.e., formaldehyde derivatized with DNPH). Moreover, the peak at 5.8 min was attributed to DNPH as it was detected in all samples added with DNPH. Hence, the HCHO-DNPH peak area was used for evaluation of linearity and accuracy after subtracting the background peak area in PBS.

### Accuracy and Precision (Repeatability)

The effect of aluminum hydroxide adjuvant was evaluated by recovery studies, which were carried out by spiking three samples of aluminum hydroxide (0.1, 0.4, and 1.0 mg/mL aluminum) in PBS with 0.05 μg/mL of formaldehyde in the absence of the vaccine drug substance. The average recoveries were 102% (n=3), 100% (n=3), and 100% (n=3), respectively, with low relative standard deviation (RSD) values (Table 22). The RSD of the accuracy data was calculated to evaluate the repeatability, and was found to be 1.0%, indicating that aluminum amounts up to 1.0 mg/mL did not interfere with the recovery of formaldehyde.

TABLE 22
Accuracy and repeatability evaluated using aluminum hydroxide
samples spiked with 0.05 μg/mL of formaldehyde

| Aluminum hydroxide concentration | |
|---|---|
| [mg/mL aluminum] | Average (n = 3) [%] (RSD [%]) |
| 0.1 | 102 (0.2) |
| 0.4 | 100 (0.8) |
| 1.0 | 100 (0.3) |
| Repeatability [%] (n = 9) | 1.0 |

The accuracy of the method was evaluated by recovery studies, which were carried out by spiking the Zika vaccine drug product containing aluminum hydroxide adjuvant with three concentrations of formaldehyde (0.05, 0.10, and 1.00 μg/mL), and the average recovery results are shown in Table 23. The RSD of the accuracy data was calculated to evaluate the repeatability, and was found to be 3.7%, indicating that Zika vaccine drug products formulated with aluminum hydroxide do not interfere with the recovery of formaldehyde between 0.05 and 1.00 μg/mL.

TABLE 23
Accuracy and repeatability evaluated using Zika vaccine drug products
containing aluminum hydroxide spiked with formaldehyde

| Spiked formaldehyde | | |
|---|---|---|
| concentration [μg/mL] | Average (n = 3) [%] (RSD [%]) | |
| 0.05 | 102 | (5.6) |
| 0.10 | 97 | (0.3) |
| 1.00 | 98 | (0.7) |

| | |
|---|---|
| Repeatability [%] (n = 9) | 3.7 |

### Robustness

The robustness of the method was evaluated to determine how concentration of formaldehyde in samples would be affected by variations in experimental parameters during sample preparation. Considering impact on the derivatization efficacy, concentration of DNPH and phosphoric acid were selected as the monitored parameters in this study. The effect was examined by varying the concentrations of DNPH and phosphoric acid by ±0.1 mg/ml and ±5%, respectively. Formaldehyde was determined in two development drug product lots under each condition, and the results, shown in Table 24, suggest that variations in DNPH and phosphoric acid concentrations had no significant impact on the determination of formaldehyde.

TABLE 24
Robustness of the method

| | Concen- | Concen- | Concentration of | |
|---|---|---|---|---|
| Condi- | tration of | tration of | formaldehyde [μg/mL] | |
| tion | DNPH [mg/mL] | phosphoric acid [%] | Lot B | Lot C |
| 1* | 1.0 | 20 | 0.51 | 0.45 |
| 2 | 1.1 | 20 | 0.53 | 0.48 |
| 3 | 0.9 | 20 | 0.49 | 0.47 |
| 4 | 1.0 | 15 | 0.52 | 0.49 |
| 5 | 1.0 | 25 | 0.52 | 0.48 |

*Defined conditions of the method

### Example 6: Immunogenicity and Safety of a Purified Inactivated Zika Virus Vaccine (PIZV) Based on P6e Strain Evaluated in a Phase I Clinical Trial

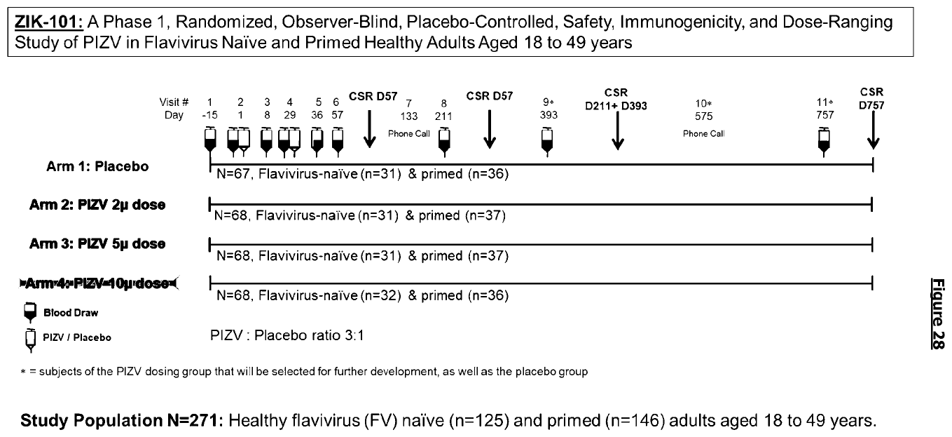

The following Example describes a Randomized, Observer-Blind, Placebo-Controlled, Safety, Immunogenicity, and Dose-Ranging Phase 1 Study of Purified Inactivated Zika Virus Vaccine (PIZV) in Flavivirus Naïve and Flavivirus Primed Healthy Adults Aged 18 to 49 Years (ZIK-101, see study outline in FIG. 28).

### Methods

### PIZV and Placebo

The purified inactivated Zika virus vaccine (PIZV) applied for the clinical study as described below was manufactured by growth on Vero cells as described above using P6e as a Pre-Master Virus Seed. Supernatants from daily harvests (each daily harvest 1000 mL) were purified by filtration and chromatography, concentrated, and inactivated by addition of formaldehyde to a final concentration of 0.01% (w/v). Inactivation was allowed to proceed for 10 days at about 22° C., before the sample was buffer exchanged into phosphate buffer containing NaCl. The PIZV applied in the clinical study below has been tested for completeness of inactivation as described above under Example 4 (see results for Phase I clinical trial lots in Example 4). No Cytopathic Effect (CPE) was visible as outlined above.

The purified formaldehyde-inactivated Zika virus in the investigational vaccine (PIZV) was formulated with 200 μg aluminum hydroxide (Al(OH)₃) per dose as adjuvant in phosphate buffered saline solution (PBS). After incubation of the antigen with aluminum hydroxide adjuvant, vaccine samples were centrifuged, the supernatant was isolated and analyzed by Western Blot and size exclusion chromatography. Complete adsorption of the antigen to the adjuvant was observed (>95%). The final liquid formulated product is filled into single-use vials and sealed with tamper-evident seals. The investigational vaccine is administered intramuscularly as a 2-dose regimen of 0.5 mL at 2, 5, or 10 μg antigen per dose, 28 days apart as outlined below.

The amount of the purified inactivated Zika virus can be determined by a Bradford assay (Bradford et al. (1976) Anal. Biochem. 72:248-254) using defined amounts of recombinant Zika envelope protein to establish the standard curve. In the current example, the main peak of the purified Zika virus when analyzed by size exclusion chromatography was more than 85% of the total area under the curve in the size exclusion chromatography.

Sodium chloride (NaCl) 0.9% solution for injection is being used as placebo. It is supplied in single-use vials. It is a sterile, clear, colorless liquid solution of sodium chloride without preservative designed for parenteral use only. As the investigational vaccine, the placebo is administered as a 2-dose regimen intramuscularly at 0.5 mL per dose, 28 days apart.

### Clinical Study Description

The Phase 1 clinical study described herein is a two-part, multicenter, randomized, observed-blind, placebo-controlled trial conducted at seven medical clinics in the USA and two in Puerto Rico from November 2017 to November 2020. The study design is shown in FIG. 1. The study protocol was approved by the local ethics committees or institutional review board of each study center. The study is being done in accordance with the guidelines of the International Council for Harmonization, Good Clinical Practice, the Declaration of Helsinki, and applicable local regulatory requirements. Written informed consent was obtained from all participants.

For the study, 894 volunteers were screened between November 2017 and October 2018. All flavivirus naïve participants (Part 1 of the study, as described below) were recruited from sites on mainland USA. Of the flavivirus primed participants (Part 2 of the study, as described below), 58% (84 of 146) were recruited from mainland USA and 42% (62 of 146) from Puerto Rico.

At the first visit (day-15 of the study), subjects were screened for eligibility. In general, eligible participants were adults aged 18-49 years of either gender. Major eligibility criteria were being healthy in the opinion of the investigator on the basis of a medical examination with no chronic viral infections (Hepatitis B or C or HIV) or relevant medical history, safety laboratory tests within normal limits (hematology, biochemistry, and urinalysis), and availability for the entire duration of the study. Sexually active female participants of childbearing potential were required to have a negative pregnancy test at screening and before the second vaccination, and to practice an approved form of contraception from 2 months before screening to 2 months after the second dose of vaccine or placebo. Major exclusion criteria included any self-reported previous exposure of participants or their partners to Zika virus, any planned travel to Zika-endemic regions, known hypersensitivity to any vaccine component, or any known condition likely to interfere with an immune response (immunodeficiency, splenic, or thymic dysfunction, or recent treatment with immunosuppressive or immunostimulatory drugs, immunoglobulins, or steroids), or participation in another clinical trial. In addition, safety laboratory parameters and vital signs were checked at study entry as part of inclusion criteria. These specified that vital signs had to be within normal limits (i.e., below Grade 1 as indicated in the FDA Toxicity Grading Scale) and that safety laboratory tests had to be within normal limits or not above Grade 1 as defined in the FDA Toxicity Grading Scale. Further, individuals who received any other vaccines within 14 days (for inactivated vaccines) or 28 days (for live vaccines) prior to enrollment in this trial or who are planning to receive any vaccine within 28 days of investigational vaccine/placebo administration were excluded.

Further at the first visit, blood samples were taken to determine flavivirus serostatus. Serological screening for previous flavivirus exposure of each volunteer was done for antibodies against 13 flavivirus antigens (including antigens of Yellow Fever, Dengue, Japanese encephalitis, West Nile, Usutu Virus, Saint Louis Encephalitis, and Zika) using a Multiplex Luminex IgG ELISA assay (Luminex, Austin, TX, USA). Serum samples were diluted 1:2,000-fold in sample dilution buffer (PBS, 0.05% ProClin 300). 30 μL of the diluted sample was transferred to a 96-well plate including negative, low and high positive controls. 10 μL diluted multiplexed serological panel bead mix (1500 beads per antigen) was added to each sample followed by a 60-minute incubation (room temperature, in the dark, orbital shaker 850 rpm). The beads were washed three times with 100 μL of assay buffer (PBS, 0.05% Tween 20, 0.05% ProClin 300). 40 UL Anti-IgG PE (phycoerythrin) was added to each well and incubated 30 min at room temperature with shaking. The beads were washed three times as above and resuspended in 100 UL assay buffer and analyzed using a Luminex 200™ reader. Greater than 35 beads per antigen were read and plates accepted based on control ranges and IgG bead having >20,000 median fluorescent intensity (MFI; cf. also Bhoring et al., 2021, PLOS One 16: e0250516). Participants with Medium Fluorescence Intensity (MFI)>500 to one of the 13 flavivirus antigens were assigned “flavivirus primed”.

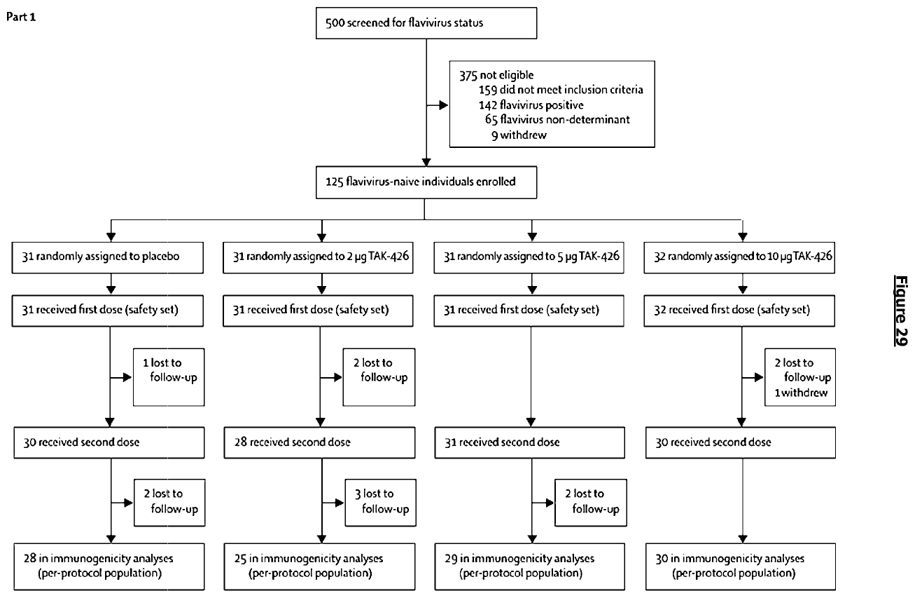

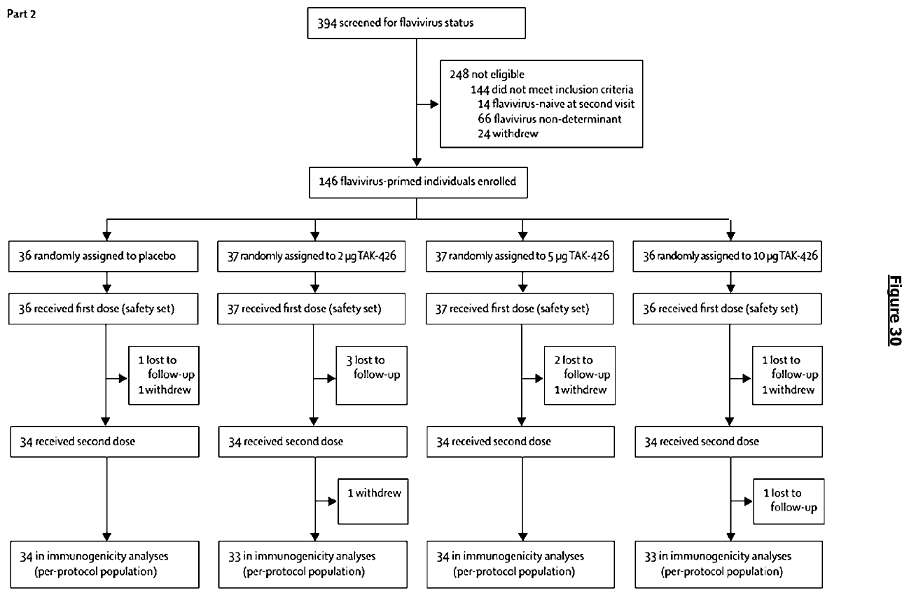

In summary, 271 subjects were enrolled. In the first part of the study, 125 eligible flavivirus naïve subjects, who were seronegative to all tested flavivirus antigens and who had no previous known vaccination against relevant flaviviruses, were enrolled. In the second part of the study, which started after an external data monitoring committee composed of independent vaccine clinical trial experts issued a positive recommendation following the review of the safety and tolerability data accrued in the flavivirus-naïve cohort up to 28 days after dose 1, 146 flavivirus primed subjects were enrolled. The subject sample size was not determined on the basis of formal statistical power calculations, but stochastic simulations based on 1 million simulation runs suggested that 60 participants per group is adequate based on the relative frequency of the decision to select one of the three tested doses on the basis of the ratios of GMTs between the dosing groups. The study flowchart is shown in FIGS. 29, 30, and 31.

The enrolled subjects in each cohort (flavivirus naïve and primed) were randomly assigned (1:1:1:1) to one of 4 groups, to receive either one of three dosages of the PIZV vaccine (2, 5, or 10 μg adsorbed to 200 μg aluminum hydroxide as adjuvant) or saline placebo. The randomization scheme used interactive response technology, which also included a stratification for two age groups, i.e. age 18-29 years and 30-49 years. Participants, investigators, and vaccine administrating personnel were masked to group assignment. An unmasked pharmacist prepared each vaccine or placebo dose in a single use syringe, which were similar in appearance. Analysis of the data was performed by a separate set of unblinded statisticians and programmers, who had access to the individual treatment assignments. The vaccination regimen consists of 2 doses (each presented in 0.5 mL) administered by intramuscular injection in the deltoid 28 days apart (for PIZV and placebo), i.e. at study days 1 and 29. In addition, also blood samples were taken on days 1 (baseline) and 29 before administration for immunogenicity assessment. Further immunogenicity assessment was carried out by taken blood samples on days 57, 211, 393, and 757 of the study, wherein samples on days 393 (12 months post-dose 2) and 757 (24 months post-dose 2) were only taken from the subjects that received the placebo and the vaccine dose selected for further clinical development, i.e. 10 μg PIZV. Safety lab testing was carried out on days 8 and 36 (cf. also FIG. 28).

Safety assessments were done in all randomly assigned participants who received at least one dose of investigational vaccine or placebo (Safety Set, SS). Immunogenicity assessments were based on the per-protocol set (PPS), comprising all participants who received at least one dose of the investigational vaccine or placebo and provided valid serology results at baseline and at least at one timepoint post-vaccination, with no major protocol violations relevant for the immunogenicity analysis. Statistical Analysis System, SAS, (version 9.2) was used for statistical analysis.

All participants were monitored for 30 min after each vaccination and provided with diary cards on which to record solicited local reactions (pain, erythema, induration, and swelling at injection site), systemic adverse events (headache, fatigue, malaise, arthralgia, and myalgia) and body temperature for 7 days after each vaccination. Any solicited adverse event which persisted or occurred beyond 7 days after vaccination was considered to be an unsolicited adverse event and these were recorded for 28 days after each vaccination.

Solicited and unsolicited adverse events were graded according the FDA Toxicity Grading Scale for Healthy Adult and Adolescent Volunteers as mild, moderate or severe (US Department of Health and Human Services. Toxicity grading scale for healthy adult and adolescent volunteers enrolled in preventive vaccine clinical trials. September 2007. https://www.fda.gov/media/73679/download (accessed Mar. 2, 2020)). Further, the investigator assessed whether they were related to study vaccines or procedures.

Solicited systemic AEs (headache, fatigue, malaise, arthralgia, and myalgia) were graded from 0 to 3 by severity; where 0=None, 1=Mild: No interference with daily activity, 2=Moderate: Interference with daily activity, 3=Severe: Prevents daily activity; A systemic AE of fever (defined as ≥38° C. or ≥100.4° F.) was graded from 1 to 4 by severity; where 1=Mild: 38.0-38.4° C., 2=Moderate: 38.5-38.9° C., 3=Severe: 39.0-40° C., and 4=Potentially life threatening: >40° C.

Solicited local AEs (at injection site) were graded: pain (none, mild: no interference with daily activity, moderate: interference with daily activity with or without treatment and severe: prevents daily activity with or without treatment), erythema (<25 mm, mild: >=25 to <=50 mm, moderate: >50 to <=100 mm, severe: >100 mm), and swelling and induration (<25 mm, mild: >=25 to <=50 mm, moderate: >50 to <=100 mm, severe: >100 mm).

Laboratory safety assessments were done before vaccination and 7 days after each vaccination.

Serious adverse events were to be reported immediately to the investigator. Serious adverse events were monitored throughout the complete duration of the study. A serious adverse event was defined as any untoward medical occurrence that: 1) results in death, 2) is life-threatening, 3) requires inpatient hospitalization or prolongation of existing hospitalization, 4) results in persistent or significant disability/incapacity, 5) leads to a congenital anomaly/birth defect in the offspring of the participant or 6) is an medically important event that satisfies any of the following: a) May require intervention to prevent items 1 through 5 above. b) May expose the participant to danger, even though the event is not immediately life threatening or fatal or does not result in hospitalization.

### Primary Objectives:

The primary objectives of the study were to describe the safety, tolerability, and immunogenicity of three increasing dosages of purified inactivated Zika vaccine given as two doses of PIZV given 28 days apart to adults with and without prior exposure to flaviviruses and to select a dose level from the three different antigen concentrations (2, 5 or 10 μg) for use in subsequent clinical studies. Dose selection was to be based on safety and an ANOVA analysis of immunogenicity as measured by the pairwise ratios of GMT of Zika virus neutralizing antibodies and differences in seroconversion rates between the dosing groups.

The primary endpoints were: the percentages of human subjects experiencing solicited local and systemic adverse events (AEs) during the 7-day period after administration of each dose of PIZV or placebo, and the percentages of human subjects experiencing unsolicited non-serious AEs and serious adverse events (SAEs) during the 28-day period after vaccination. Immunogenicity was assessed as geometric mean titers (GMTs) of neutralizing anti-Zika virus antibody levels at 28 days after the second dose (also including determination of seroconversion rates (SCR) and seropositivity rates (SPR)).

### Secondary Objectives:

Secondary outcomes were the long-term safety and immune responses up to the end of the study, as follows: serious adverse events throughout the trial; Geometric mean titers (GMTs) of neutralizing antibody levels, SCR, and SPR at 28 days after the first dose, and 6, 12, and 24 months after the second dose (wherein immunogenicity was assessed at 12 and 24 months after the second dose only for the subjects in the PIZV dosing group that will be selected for further development).

### Determination of Neutralizing Antibody Titers, Seropositivity, and Seroconversion

Immunogenicity of the subjects was assessed by measuring neutralizing antibody titers in the blood samples taken using the plaque reduction neutralization test (PRNT) and the reporter virus particle test (RVP).

### PRNT:

Neutralizing antibody titers were determined by a plaque reduction neutralization test (PRNT) as described previously (See Sun et al., Protection of Rhesus monkeys against dengue virus challenge after tetravalent live attenuated dengue virus vaccination. J. Infect. Dis. 193, 1658-1665 (2006); Muthumani K, Griffin B D, Agarwal S, et al., In vivo protection against ZIKV infection and pathogenesis through passive antibody transfer and active immunisation with a prMEnv DNA vaccine, NPJ Vaccines 2016; 1:16021). Briefly, the Zika PRNT assay was carried out according to the protocol developed by Q2 Lab Solutions Vaccines as described below.

In the Zika PRNT, human serum was 2-fold serially diluted from 1:5 to 1:10,240 and mixed with an equal volume of diluted Zika virus (i.e. the PIZV strain PRVABC59) to obtain a final dilution of 1:10 to 1:20,480. Neutralization was allowed to proceed 20±2 hours at 2-8° C. after which the serum/virus mixture was used to inoculate Vero E6 cells. Virus adsorption was done at 37±2° C. with humidity and CO₂ for 60±10 minutes then a methylcellulose overlay was added. The infected cells were incubated at 37±2° C. with humidity and CO₂ for 72±2 hours. Plaques were visualized by using crystal violet staining and were counted using a CTL (Cellular Technology Limited) reader. Determination of the fifty percent neutralizing titer (PRNT₅₀) was based upon the percent reduction in viral plaques in the presence of serum compared to that of the virus control without serum and was calculated by linear regression. The titers represent the reciprocal of the serum dilution resulting in a 50% reduction in the number of plaques. Acceptance was assessed by evaluating the virus control (targeting ˜60 pfu/well), cell control, positive control (PRNT₅₀ of 173-658) and negative control (PRNT₅₀<10) tested in parallel with clinical samples. Individual samples and positive control results were accepted if the correlation coefficient of the titration curve generated by linear regression is ≥0.85. Additional acceptance criteria were based on the quality of the crystal violet stain and plaques generated for the plate or run. PRNT₅₀ results are reported down to the starting dilution of the assay (1:10). PRNT₅₀ results that are above the Upper-limit-of-quantitation (ULOQ) will be repeated at a pre-dilution to generate a result within the quantifiable range of the assay. The result from the pre-diluted sample will be multiplied by the dilution factor to generate a final result.

The limit-of-detection (LOD) of the PRNT is a dilution of 1:10, i.e. a titer of 10 (titer=reciprocal dilution), the lower-limit-of-quantitation (LLOQ) is a dilution of 1:26, i.e. a titer of 26. Titers<10 were assigned a titer of 5, titers between ≥10 (LOD) and <26 (LLOQ) were assigned a value of 13.

Seropositivity was defined as a titer≥10 (LOD), seronegativity was defined as a titer<10 (LOD). In the flavivirus naïve cohort (i.e. seronegative subjects at baseline with a PRNT titer<10), seroconversion was defined as post-vaccination titer≥10 (i.e. becoming seropositive after vaccination). In the flavivirus primed cohort (i.e. seropositive subjects at baseline with a PRNT titer≥10), seroconversion refers to 4-fold seroconversion, defined as a post vaccination titer increase of ≥4-fold compared to baseline.

### RVP:

Neutralizing antibody titers were analyzed by titration of serum samples with a constant amount of Zika RVPs in Vero cells grown in 96-well plates. RVPs contained the prME proteins of Zika (strain SPH2012) and a Dengue-based Renilla luciferase reporter. Briefly, sera were heat inactivated at 56° C. for 30 min, diluted, and then incubated at 37° C. with RVPs. The serum/RVP mixture was then mixed with Vero cells and incubated for 72 hours at 37° C.±2° C. and 5% CO₂ before detection with luciferase substrate. Data was analyzed using JMP11 non-linear 4 parameter analysis, normalized to a positive tracking control and the effective dose 50% (RVP₅₀) was reported. See also Bohning et al., 2021 (PLOS One 16: e0250516, “A high throughput reporter virus particle microneutralization assay for quantitation of Zika virus neutralizing antibodies in multiple species”).

For the RVP, seropositivity was defined as a titer≥105 (the LLOQ). In the flavivirus naïve cohort (i.e. seronegative subjects at baseline with a RVP titer<105), seroconversion was defined as post-vaccination titer≥150. In the flavivirus primed cohort (i.e. seropositive subjects at baseline with a RVP titer≥105), seroconversion refers to 4-fold seroconversion, defined as a post vaccination titer increase of ≥4-fold compared to baseline.

Titers for both, the RVP and PRNT, are reported as Geometric Mean Titers (GMT) together with 95% Confidence Intervals (95% CI), calculated using the exact Clopper-Pearson method for each group and timepoint. If titers are indicated as EC₅₀, this refers to PRNT₅₀ or RVP₅₀, respectively.

### Results

### Study Population

A total of 125 human subjects were enrolled in the flavivirus-naïve cohort and a total of 146 human subjects were enrolled in the flavivirus-primed cohort (cf. FIG. 28). These subjects were included in the Safety Set (SS), comprised of all randomized human subjects who have received at least one dose of PIZV or placebo. Most of those were also included in the Full Analysis Set (FAS) of randomized human subjects, who had received at least one dose of the investigational vaccine (PIZV)/placebo, provided valid serology results at baseline and at least one post-vaccination serology result. A high rate of the FAS subjects was also included in the Per Protocol Set (PPS). Those subjects had no major protocol violations relevant for the immunogenicity analysis. The number of human subjects in the SS, FAS, and PPS can be taken from FIGS. 29 to 31 as well as Table 25 below.

TABLE 25
Analysis sets in the different cohorts. Presented are the number of subjects
in the different analysis sets and the percentage (%) made up by this
number compared to the subjects that were randomly assigned (N).
Flavivirus-naïve cohort

| | Placebo | | 2 μg PIZV | | 5 μg PIZV | | 10 μg PIZV | | Total | |
|---|---|---|---|---|---|---|---|---|---|---|
| | (N = 31) | | (N = 31) | | (N = 31) | | (N = 32) | | (N = 125) | |
| Safety Set (SS) | 31 | (100%) | 31 | (100%) | 31 | (100%) | 32 | (100%) | 125 | (100%) |
| Full Analysis Set | 30 | (96.8%) | 28 | (90.3%) | 31 | (100%) | 30 | (96.8%) | 119 | (95.2%) |
| (FAS) | | | | | | | | | | |
| Per-Protocol Set | 28 | (90.3%) | 25 | (80.7%) | 29 | (93.5%) | 30 | (93.8%) | 112 | (89.6%) |
| (PPS) | | | | | | | | | | |

Flavivirus-primed cohort

| | Placebo | | 2 μg PIZV | | 5 μg PIZV | | 10 μg PIZV | | Total | |
|---|---|---|---|---|---|---|---|---|---|---|
| | (N = 36) | | (N = 37) | | (N = 37) | | (N = 36) | | (N = 146) | |
| Safety Set (SS) | 36 | (100%) | 37 | (100%) | 37 | (100%) | 36 | (100%) | 146 | (100%) |
| Full Analysis Set | 34 | (94.4%) | 34 | (91.9%) | 34 | (91.9%) | 34 | (94.4%) | 136 | (93.2%) |
| (FAS) | | | | | | | | | | |
| Per-Protocol Set | 34 | (94.4%) | 33 | (89.2%) | 34 | (94.4%) | 33 | (91.7%) | 134 | (91.8%) |
| (PPS) | | | | | | | | | | |

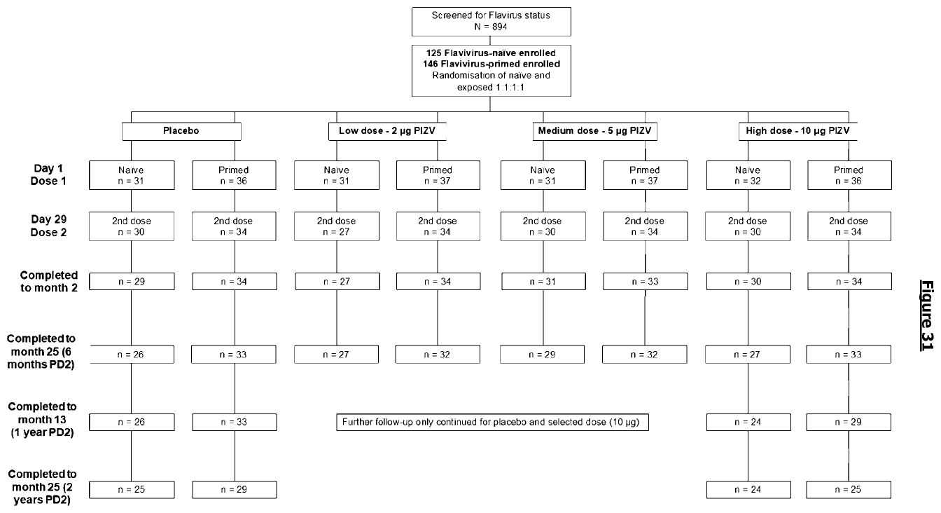

The PIZV Phase 1 clinical trial was completed with high retention rates of 93% 28 days post dose 2 (visit 6 on day 57), 88% 6 months post dose 2 (Visit 8 on day 211), 83% 12 months post dose 2 (visit 9 on day 393), and 76% 24 months post dose 2 (visit 11 on day 757; Table 26). The number of subjects per group can be taken from FIG. 31.

TABLE 26
Retention Rates during PIZV Phase 1 clinical trial.

| | Subjects completing | | | | | |
|---|---|---|---|---|---|---|
| | the study visit | | | Retention Rate | | |
| Visit | Naive | Primed | Total | Naive | Primed | Total |
| Visit 1: | 346 | 554 | 894 | N/A | N/A | N/A |
| Screening | | | | | | |
| Screen Failed | 221 | 408 | 623 | N/A | N/A | N/A |
| Visit 2 (Day 1): | 125 | 146 | 271 | N/A | N/A | N/A |
| Randomized, | | | | | | |
| Dose 1 | | | | | | |
| Visit 3 (Day 8): | 122 | 141 | 263 | 97.6% | 96.6% | 97.0% |
| Safety, 7 days PD1 | | | | | | |
| Visit 4 (Day 29): | 119 | 136 | 255 | 95.2% | 93.2% | 94.1% |
| 28 days PD1, | | | | | | |
| Dose 2 | | | | | | |
| Visit 5 (Day 36): | 119 | 135 | 254 | 95.2% | 92.5% | 93.7% |
| Safety, 7 days PD2 | | | | | | |
| Visit 6 (Day 57): | 117 | 135 | 252 | 93.6% | 92.5% | 93.0% |
| 28 days PD2 | | | | | | |
| Visit 8 (Day 211): | 109 | 130 | 239 | 87.2% | 89.0% | 88.2% |
| 6 months PD2 | | | | | | |
| Visit 9 (Day 393): | 50 | 62 | 112 | 79.4% | 86.1% | 83.0% |
| 12 months PD2 | | | | | | |
| Visit 11 (Day 757): | 49 | 54 | 103 | 77.8% | 75.0% | 76.3% |
| 24 months PD2 | | | | | | |

PD = Post-Dose.

The demographics of the study groups in the safety set were consistent across groups and between the flavivirus-naïve and flavivirus-primed cohorts, with similar age, gender, race, and BMI characteristics, with the exception of ethnicity. Most of the Hispanic and Latino participants were flavivirus primed, whereas most of those who were not Hispanic or Latino were flavivirus naïve. Mean age of participants was 35.5 years (SD 8.69), with 76 (28%) of 271 aged 18-29 years and 195 (72%) aged 30-49 years; 158 (58%) were women and 113 (42%) were men; 151 (56%) were Hispanic or Latino and 119 (44%) were not; and 215 (79%) were White, 45 (17%) Black or African American, six (2%) multiracial, and two (1%) Native Hawaiian or Pacific Islander (Table 27). The mean BMI of the participants was 27.39 (SD: 4.143). Demographics in the Per Protocol set used for immunogenicity analyses were similar.

TABLE 27
Demographics and baseline characteristics of the subjects in the Safety Set, SS. Data
are mean with standard deviation (SD) or number of subjects (n) and corresponding
percent (%). One flavivirus-primed participant in the 5 μg PIZV group did not
report ethnicity. Multiracial means that multiple race categories were selected.

| | Placebo group | | 2 μg PIZV | | 5 μg PIZV | | 10 μg PIZV | |
|---|---|---|---|---|---|---|---|---|
| | Naïve | Primed | Naïve | Primed | Naïve | Primed | Naïve | Primed |
| | (n = 31) | (n = 36) | (n = 31) | (n = 37) | (n = 31) | (n = 37) | (n = 32) | (n = 36) |
| Age (Years) | 36.7 | 34.7 | 34.9 | 35.5 | 35.8 | 37.0 | 34.1 | 35.4 |
| | (8.93) | (8.20) | (9.52) | (8.71) | (8.86) | (8.89) | (8.50) | (8.45) |
| Sex: Female | 21 | 21 | 16 | 18 | 18 | 26 | 14 | 24 |
| | (68%) | (58%) | (52%) | (49%) | (58%) | (70%) | (44%) | (67%) |
| Sex: Male | 10 | 15 | 15 | 19 | 13 | 11 | 18 | 12 |
| | (32%) | (42%) | (48%) | (51%) | (42%) | (30%) | (56%) | (33%) |
| Ethnicity: | 1 | 36 | 1 | 36 | 2 | 36 | 4 | 35 |
| Hispanic or | (3%) | (100%) | (3%) | (97%) | (7%) | (97%) | (13%) | (97%) |
| Latino | | | | | | | | |
| Ethnicity: Not | 30 | 0 | 30 | 1 | 29 | 0 | 28 | 1 |
| Hispanic or | (97%) | | (97%) | (3%) | (94%) | | (88%) | (3%) |
| Latino | | | | | | | | |
| Race: American | 2 | 1 | 0 | 0 | 0 | 0 | 0 | 0 |
| Indian or Alaska | (6%) | (3%) | | | | | | |
| Native | | | | | | | | |
| Race: Black or | 6 | 6 | 5 | 4 | 3 | 7 | 4 | 10 |
| African American | (19%) | (17%) | (16%) | (11%) | (10%) | (19%) | (13%) | (28%) |
| Race: Native | 0 | 1 | 0 | 1 | 0 | 0 | 0 | 0 |
| Hawaiian or | | (3%) | | (3%) | | | | |
| Pacific Islander | | | | | | | | |
| Race: White | 23 | 27 | 26 | 31 | 26 | 30 | 27 | 25 |
| | (74%) | (75%) | (84%) | (84%) | (84%) | (81%) | (84%) | (69%) |
| Race: Multiracial | 0 | 1 | 0 | 1 | 2 | 0 | 1 | 1 |
| | | (3%) | | (3%) | (6%) | | (3%) | (3%) |
| Race: Unknown | 0 | 0 | 0 | 0 | 0 | 0 | 0 | 0 |
| or not reported | | | | | | | | |
| Body-mass index, | 27.92 | 27.39 | 27.53 | 27.05 | 27.54 | 26.98 | 26.75 | 28.07 |
| BMI (kg/m²) | (4.205) | (3.524) | (4.763) | (4.711) | (3.717) | (4.124) | (3.651) | (4.473) |

BMI = Weight (kg)/height²(m²). Age is calculated using the date of informed consent.

Demographics in the Per Protocol Set, PSS, used for immunogenicity analyses were similar.

### Safety/Reactogenicity

No deaths were reported in any group throughout the complete study, nor were there any withdrawals due to adverse events.

### Serious Adverse Events (SAE)

Eleven serious adverse events were reported, four in flavivirus-naïve and seven in flavivirus-primed participants, none of which were considered to be related to vaccination (Table 28). All serious adverse events were reported within 12 months post-dose 2. Specifically, one flavivirus-naïve participant who had a history of pancreatitis had a pancreatitis episode 2 months and 23 days after dose 2. The event resolved 3 days after the episode. Another subject developed post-partum hemorrhage >8 months after dose 2. This subject reported pregnancy 22 days after the second vaccination with 2 μg PIZV (unmasked by request). The pregnancy resulted in normal delivery of a healthy girl at week 39 of gestation after induction because of high blood pressure. In the flavivirus-primed cohort, there was one incident of a transient ischemic attack 3 days after dose 1 in a participant with a history of hypertension, another participant was admitted to hospital for 1 day having developed major depression due to personal problems 4 months 3 days after dose 2, and a third participant who had a history of migraines developed a migraine 2 months and 10 days after dose 2 and was admitted to hospital for 2 days. After treatment, the migraine event resolved after 7 days.

TABLE 28
Number of participants who experienced at least one serious adverse event during
the study (from day 1 to day 757 in Safety Set). Data refer to number of participants,
n (%). There were no serious adverse events related to vaccination.

| | Placebo | | 2 μg PIZV | | 5 μg PIZV | | 10 μg PIZV | |
|---|---|---|---|---|---|---|---|---|
| | Naïve | Primed | Naïve | Primed | Naïve | Primed | Naïve | Primed |
| | (n = 31) | (n = 36) | (n = 31) | (n = 37) | (n = 31) | (n = 37) | (n = 32) | (n = 36) |
| Any serious | 1 | 1 | 1 | 2 | 0 | 0 | 2 | 4 |
| adverse event | (3.2%) | (2.8%) | (3.2%) | (5.4%) | | | (6.3%) | (11.1%) |

### Unsolicited Adverse Events

Unsolicited adverse events (cf. Tables 29 to 31) were reported by 19-39% of the flavivirus naïve groups who received PIZV, compared with 42% of the placebo group. Three (2%) of 125 flavivirus-naïve participants, one in each PIZV group, reported adverse events considered possibly related to PIZV. In addition to the participant with the prolonged headache after the first dose, the events consisted of dizziness for one participant in the 5 μg group and flushing for another participant in the 2 μg group after dose 1, and eye pruritus and increased lacrimation for another participant in the 10 μg dose after dose 2. All were mild to moderate in intensity. Rates in the flavivirus-primed groups were similar to those in the flavivirus-naïve groups, with 22-30% of the PIZV groups and 31% of the placebo group reporting unsolicited adverse events. Four flavivirus-primed participants reported unsolicited adverse events considered possibly related to vaccination, three in the PIZV groups and one in the placebo group. All these adverse events, consisting of individual cases of headache and dizziness after the first dose, were graded as mild to moderate. Unsolicited adverse events other than the prolonged headache had resolved by the subsequent study visit.

TABLE 29
Unsolicited adverse events (any, vaccine related, or serious) up to 28 days
after the second vaccination (in safety set). Data refer to number of participants,
n (%). Participants could have more than one adverse event and could have
adverse events related and unrelated to vaccination.

| | Placebo | | 2 μg PIZV | | 5 μg PIZV | | 10 μg PIZV | |
|---|---|---|---|---|---|---|---|---|
| | Naïve | Primed | Naïve | Primed | Naïve | Primed | Naïve | Primed |
| | (n = 31) | (n = 36) | (n = 31) | (n = 37) | (n = 31) | (n = 37) | (n = 32) | (n = 36) |
| Any | 13 | 11 | 8 | 10 | 12 | 11 | 6 | 8 |
| | (42%) | (31%) | (26%) | (27%) | (39%) | (30%) | (19%) | (22%) |
| Vaccine related | 0 | 1 | 1 | 1 | 1 | 0 | 1 | 2 |
| | | (3%) | (3%) | (3%) | (3%) | | (3%) | (6%) |
| Serious | 0 | 0 | 0 | 1 | 0 | 0 | 0 | 0 |
| | | | | (3%) | | | | |

TABLE 30
Percentage of participants (in safety set) who experienced at least one non-serious
or serious unsolicited adverse event (AE) within 28 days after dose 1 (until day 29).

| | Placebo | | 2 μg PIZV | | 5 μg PIZV | | 10 μg PIZV | |
|---|---|---|---|---|---|---|---|---|
| | Naïve | Primed | Naïve | Primed | Naïve | Primed | Naïve | Primed |
| | (n = 31) | (n = 36) | (n = 31) | (n = 37) | (n = 31) | (n = 37) | (n = 32) | (n = 36) |
| Non-serious AE | 29.0% | 22.2% | 19.4% | 21.6% | 29.0% | 21.6% | 9.4% | 16.7% |
| Serious AE | 0 | 0 | 0 | 2.7% | 0 | 0 | 0 | 0 |

TABLE 31
Percentage of participants (in safety set) who experienced at least one non-serious
or serious unsolicited adverse event (AE) within 28 days after dose 2 (until day 57).

| | Placebo | | 2 μg PIZV | | 5 μg PIZV | | 10 μg PIZV | |
|---|---|---|---|---|---|---|---|---|
| | Naïve | Primed | Naïve | Primed | Naïve | Primed | Naïve | Primed |
| | (n = 31) | (n = 36) | (n = 31) | (n = 37) | (n = 31) | (n = 37) | (n = 32) | (n = 36) |
| Non-serious AE | 16.1% | 8.3% | 16.1% | 13.5% | 9.7% | 13.5% | 12.5% | 13.9% |
| Serious AE | 0 | 0 | 0 | 0 | 0 | 0 | 0 | 0 |

### Solicited Adverse Events

In flavivirus naïve participants, reported rates of solicited local adverse events were higher in the PIZV groups than in the placebo group after the first injection (four (13%) of 30 in the placebo group, nine (30%) of 30 in the 2 μg group, 12 (39%) of 31 in the 5 μg group, and 13 (42%) of 31 in the 10 μg group; cf. Table 32). There was no increase in these rates after the second dose, and no severe local reactions were reported after either dose. Pain at the injection site was the most frequent solicited adverse event. The majority of these events were reports of mild to moderate pain at the injection site, most with onset on day 1 (57 (92%) of 62) with a mean duration of 1.5 days (SD 0.74).

TABLE 32
Incidence of solicited local and systemic adverse events 7 days after vaccination in flavivirus naïve subjects (Safety set).

| Dose 1 | | | | Dose 2 | | | |
|---|---|---|---|---|---|---|---|
| | 2 μg | 5 μg | 10 μg | | 2 μg | 5 μg | 10 μg |
| Placebo | PIZV | PIZV | PIZV | Placebo | PIZV | PIZV | PIZV |

Local AEs n (%)

| N | 30 | | 30 | | 31 | | 31 | | 28 | | 27 | | 30 | | 30 | |
|---|---|---|---|---|---|---|---|---|---|---|---|---|---|---|---|---|
| Pain | 4 | (13.3) | 9 | (30.0) | 10 | (32.3) | 12 | (38.7) | 4 | (14.3) | 8 | (29.6) | 11 | (36.7) | 12 | (40) |

| | | | | | | | | | | | | |
|---|---|---|---|---|---|---|---|---|---|---|---|---|
| Erythema | 0 | 0 | 0 | 1 | (3.2) | 1 | (3.6) | 0 | 1 | (3.3) | 1 | (3.3) |

| Swelling | 0 | 0 | 0 | | 0 | | 0 | 0 | 2 | (6.7) | 0 |
|---|---|---|---|---|---|---|---|---|---|---|---|
| Induration | 0 | 0 | 3 | (9.7) | 2 | (6.5) | 0 | 0 | 1 | (3.3) | 0 |

Systemic AEs n (%)

| N | 30 | | 30 | | 31 | | 31 | | 28 | | 27 | | 30 | | 30 | |
|---|---|---|---|---|---|---|---|---|---|---|---|---|---|---|---|---|
| Fever | 1 | (3.3) | 0 | | 0 | | 1 | (3.2) | 2 | (7.1) | 0 | | 0 | | 0 | |
| Headache | 10 | (33.3) | 5 | (16.6) | 8 | (25.9) | 4 | (12.9) | 3 | (10.3) | 4 | (14.8) | 1 | (3.3) | 6 | (20.0) |
| Fatigue | 7 | (23.4) | 7 | (23.4) | 6 | (19.3) | 10 | (32.3) | 6 | (20.7) | 3 | (11.1) | 2 | (6.6) | 5 | (16.6) |
| Arthralgia | 1 | (3.3) | 1 | (3.3) | 1 | (3.2) | 3 | (9.7) | 1 | (3.4) | 2 | (7.4) | 0 | | 1 | (3.3) |
| Myalgia | 3 | (10.0) | 3 | (10.0) | 5 | (16.1) | 4 | (12.9) | 2 | (6.9) | 2 | (7.4) | 1 | (3.3) | 2 | (6.6) |

| | | | | | | | | | | | | | | |
|---|---|---|---|---|---|---|---|---|---|---|---|---|---|---|
| Malaise | 4 | (13.4) | 2 | (6.6) | 2 | (6.4) | 4 | (12.9) | 2 | (6.4) | 0 | 0 | 3 | (10.0) |

N = number of human subjects with information available; n (%) = number (percentage) of human subjects reporting a specific AE.

The local reactogenicity profile in flavivirus-primed participants was generally similar to that of the flavivirus-naïve groups (Table 33). As in the flavivirus-naïve groups, these reactions consisted mainly of reports of injection site pain, with onset on day 1 after the first dose in 36 (86%) of 42 cases and a mean duration of 1.8 days (SD 1.04). Most reports were of mild or moderate pain, but one case was described as severe. There were more cases of erythema, induration, and swelling in the flavivirus-primed groups than in the flavivirus-naïve PIZV groups, but these consisted of a small fraction of local reactions and there were never more than two reports in any single study group.

TABLE 33
Incidence of solicited local and systemic adverse events 7 days after vaccination in flavivirus primed subjects (Safety set).

| Dose 1 | | | | Dose 2 | | | |
|---|---|---|---|---|---|---|---|
| | 2 μg | 5 μg | 10 μg | | 2 μg | 5 μg | 10 μg |
| Placebo | PIZV | PIZV | PIZV | Placebo | PIZV | PIZV | PIZV |
| (N = 36) | (N = 33) | (N = 36) | (N = 35) | (N = 33) | (N = 32) | (N = 32) | (N = 34) |

Local AEs n (%)

| N | 36 | | 33 | | 36 | | 35 | | 33 | | 32 | | 32 | | 34 | |
|---|---|---|---|---|---|---|---|---|---|---|---|---|---|---|---|---|
| Pain | 3 | (8.3) | 15 | (45.4) | 14 | (38.9) | 13 | (37.2) | 6 | (18.2) | 7 | (21.9) | 9 | (28.2) | 10 | (29.4) |

| | | | | | | | | | | | | | | |
|---|---|---|---|---|---|---|---|---|---|---|---|---|---|---|
| Erythema | 2 | (5.6) | 1 | (3.0) | 1 | (2.8) | 0 | 0 | 2 | (6.3) | 1 | (3.1) | 1 | (2.9) |

| | | | | | | | | | | | | |
|---|---|---|---|---|---|---|---|---|---|---|---|---|
| Swelling | 0 | 2 | (6.1) | 2 | (5.6) | 1 (2.9) | 0 | 1 | (3.1) | 0 | 2 | (5.9) |

| | | | | | | | | | | | | |
|---|---|---|---|---|---|---|---|---|---|---|---|---|
| Induration | 0 | 1 | (3.0) | 2 | (5.6) | 0 | 0 | 0 | 1 | (3.1) | 1 | (2.9) |

Systemic AEs n (%)

| N | 36 | | 33 | | 36 | | 35 | | 33 | | 32 | | 32 | | 34 | |
|---|---|---|---|---|---|---|---|---|---|---|---|---|---|---|---|---|
| Fever | 0 | | 0 | | 1 | (2.8) | 0 | | 0 | | 0 | | 2 | (6.4) | 0 | |
| Headache | 5 | (13.9) | 5 | (15.1) | 8 | (22.3) | 6 | (17.2) | 3 | (9.4) | 2 | (6.3) | 5 | (15.6) | 4 | (11.7) |
| Fatigue | 9 | (25.0) | 3 | (9.1) | 9 | (25.0) | 6 | (17.1) | 3 | (9.1) | 1 | (3.1) | 4 | (12.5) | 5 | (14.7) |
| Arthralgia | 3 | (8.3) | 4 | (12.1) | 4 | (11.1) | 2 | (5.7) | 0 | | 3 | (9.4) | 3 | (9.3) | 2 | (5.8) |
| Myalgia | 7 | (19.4) | 6 | (17.8) | 5 | (13.9) | 4 | (11.5) | 3 | (9.1) | 4 | (12.5) | 3 | (9.4) | 4 | (11.7) |
| Malaise | 8 | (22.2) | 4 | (12.1) | 6 | (16.7) | 8 | (22.9) | 5 | (15.1) | 1 | (3.1) | 4 | (12.5) | 3 | (8.8) |

N = number of human subjects with information available; n (%) = number (percentage) of human subjects reporting a specific AE.

In the flavivirus-naïve participants and in flavivirus-primed participants, rates of solicited systemic adverse events were similar between PIZV and placebo groups after the first dose (Tables 32 and 33). These rates were generally lower after the second doses in both flavivirus-naïve and flavivirus-primed cohorts. The most frequent events after either dose in all flavivirus-naïve groups were headache and fatigue, whereas the most frequent events in flavivirus-primed participants were headache, fatigue, and malaise. All systemic adverse events were described as mild to moderate and were transient, with the exception of one case of mild headache in a flavivirus-naïve participant that persisted for 36 days after the first dose, so the participant did not receive the second dose of PIZV. The median duration of reported fatigue or headache after both doses in the PIZV groups was 3 days or less.

### Laboratory Safety Assessment

There was no trend in changes in the safety laboratory parameters assessed at 7 days after vaccination in any PIZV group in either cohort, with mild transitory changes in blood chemistry and hematology values from baseline observed in similar proportions of participants across the groups in the flavivirus-naïve and the flavivirus-primed cohorts. In grade, the highest changes were in two flavivirus-naïve participants with grade 4 prothrombin time (>1.25×upper limit of normal) on day 8 and 36, which had returned to normal values at their next visits. There were grade 3 laboratory fibrinogen results observed in one (1%) of 125 flavivirus-naïve participant and two (1%) of 146 flavivirus-primed participants, and grade 3 laboratory urine results in five (4%) of the flavivirus-naïve cohort and four (3%) flavivirus-primed participants, all of which occurred in women and could be related to menstruation at the time of urinalysis.

### Pregnancy

As outlined above, one woman in the 2 μg PIZV group reported pregnancy 22 days after the second vaccination. The pregnancy resulted in normal delivery of a healthy girl at week 39 of gestation after induction because of high blood pressure.

Another subject in the flavivirus-primed cohort delivered at 1 year, 4 months, and 14 days after dose 2 a newborn weighing 4.023 kg at birth and length of 52 cm after an emergency cesarean section due to gestational hypertension at 36 weeks of gestation (blinding was not broken as this study subject did not express this as per the protocol). This subject did not encounter serious adverse events.

### Summary Safety/Reactogenicity

In summary, PIZV was shown to be safe and well-tolerated in both, flavivirus-naïve and primed subjects. Safety and reactogenicity profiles across all dose levels were comparable to the placebo groups on both flavivirus-naïve and -primed subjects with no significant difference between dose levels. No serious adverse events (SAEs) were assesses as causally related to PIZV. Further, no SAEs were reported during the last year follow-up of the study. Also, no deaths were reported up to 24 months post-dose 2.

### Immunogenicity

### Geometric Mean Neutralizing Antibody Titers as Measured by PRNT and RVP

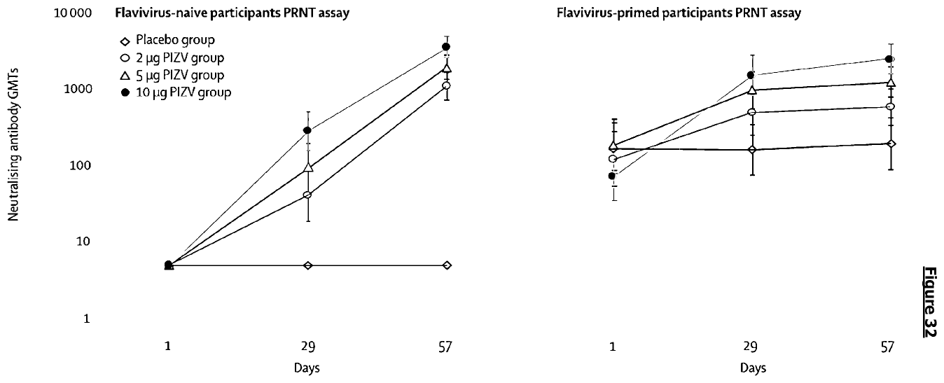

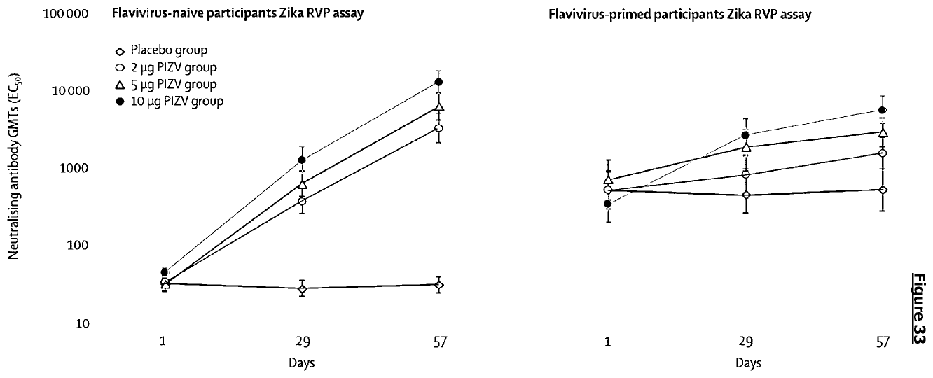

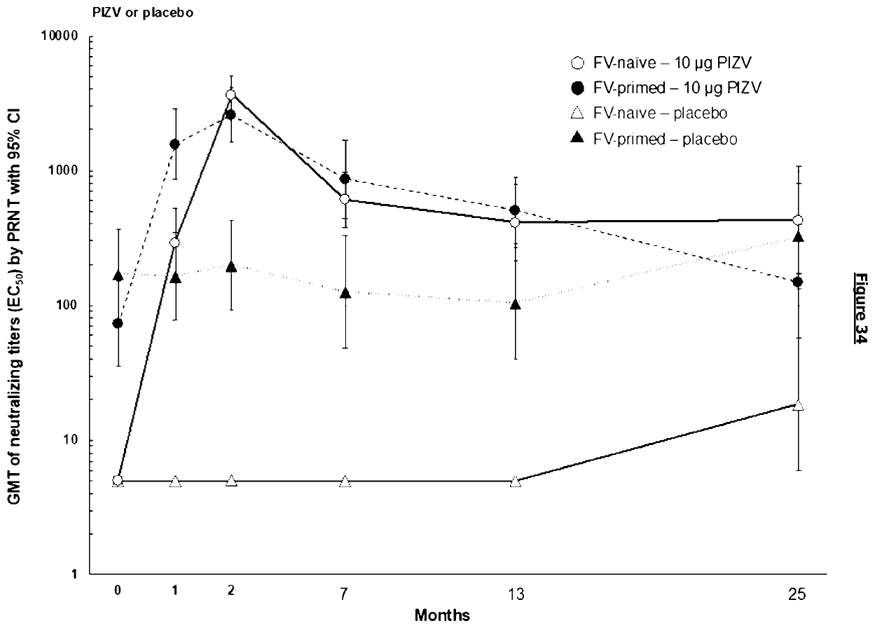

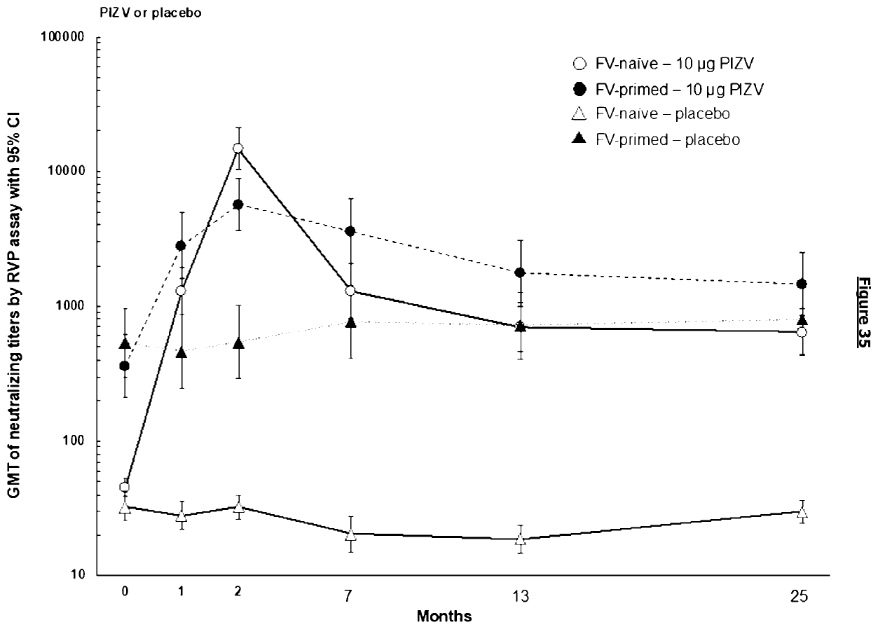

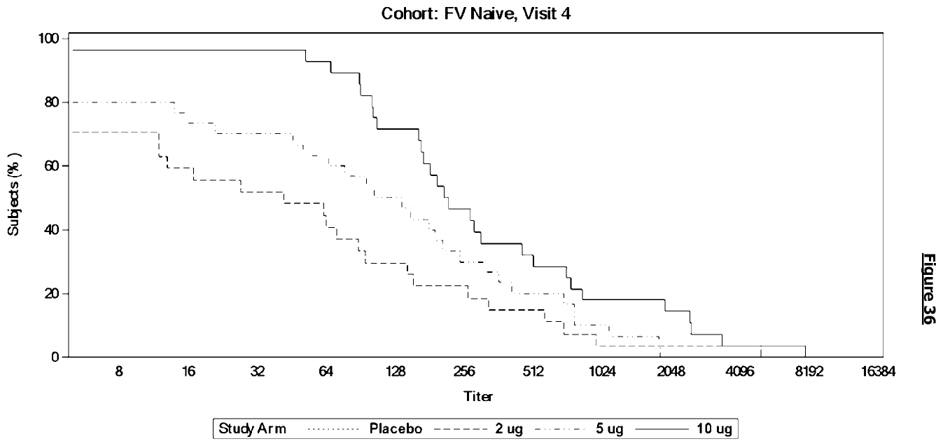

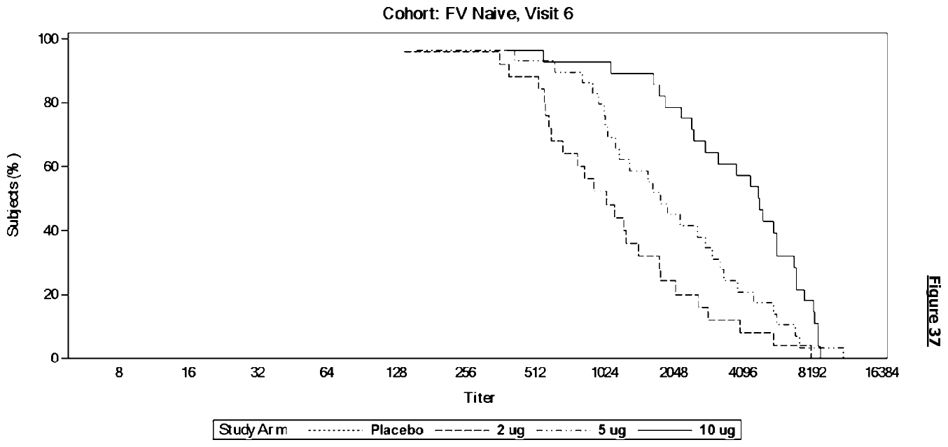

Tables 34 to 37 show the neutralizing antibody titers measured in subject samples and reported as geometric mean antibody titers (GMT) and as the half maximal effective concentration (PRNT₅₀ or RVP₅₀). In addition, FIGS. 32 to 35 show the GMTs expressed as EC₅₀ values (either PRNT₅₀ or RVP₅₀) as measured by PRNT and RVP. The distribution of neutralization titers after dose 1 and after dose 2 are shown in reverse cumulative distribution curves in FIGS. 36 and 37, respectively.

TABLE 34
Geometric mean titers (GMTs) of Zika virus neutralizing antibodies as
measured by PRNT (PRNT₅₀) in the flavivirus naïve subject set (Per Protocol
Set, PPS) with 95% confidence interval (GMTs (95% CI)). The number of
subjects (n) refers to the number of subjects in the per-protocol analysis
set with available PRNT data at the indicated time-point.

| Study | | | | |
|---|---|---|---|---|
| Day | Placebo | 2 μg PIZV | 5 μg PIZV | 10 μg PIZV |
| 1 | n = 28 | n = 25 | n = 29 | n = 30 |
| | 5 | 5 | 5 | 5 |
| 29 | n = 28 | n = 25 | n = 28 | n = 28 |
| | 5 | 41.28 | 93.76 | 291.41 |
| | | (18.74, 90.95) | (44.34, 198.30) | (161.74, 525.06) |
| 57 | n = 27 | n = 23 | n = 28 | n = 28 |
| | 5 | 1129.69 | 1992.33 | 3689.89 |
| | | (749.38 to 1703.1) | (1401.28, 2832.70) | (2676.75, 5086.49) |
| 211 | n = 21 | n = 20 | n = 25 | n = 23 |
| | 5 | 151.24 | 364.10 | 613.28 |
| | | (87.27, 262.10) | (237.97, 557.08) | (382.29, 983.83) |
| 393 | n = 23 | N/A | N/A | n = 21 |
| | 5 | N/A | N/A | 413.71 |
| | | | | (215.67, 793.58) |
| 757 | n = 19 | N/A | N/A | n = 16 |
| | 18.48 | N/A | N/A | 427.41 |
| | (6.00, 56.91) | | | (171.06, 1067.93) |

TABLE 35
Geometric mean titers (GMTs) of Zika virus neutralizing antibodies
(EC₅₀) as measured by PRNT in the flavivirus primed subject set (Per
Protocol Set, PPS) with 95% confidence interval (GMTs (95% CI)). The
number of subjects (n) refers to the number of subjects in the per-protocol
analysis set with available PRNT data at the indicated time-point.

| Study | | | | |
|---|---|---|---|---|
| Day | Placebo | 2 μg PIZV | 5 μg PIZV | 10 μg PIZV |
| 1 | n = 34 | n = 33 | n = 34 | n = 33 |
| | 170 | 123 | 190 | 73 |
| | (79, 367) | (53, 286) | (87, 413) | (36, 152) |
| 29 | n = 34 | n = 31 | n = 34 | n = 33 |
| | 164.06 | 513.62 | 1002.69 | 1572.58 |
| | (77.37, 347.88) | (259.90, 1015.03) | (566.72, 1774.06) | (864.63, 2860.20) |
| 57 | n = 31 | n = 30 | n = 31 | n = 30 |
| | 198.82 | 597.63 | 1276.92 | 2590.54 |
| | (91.73, 430.97) | (340.48, 1049.00) | (806.00, 2022.97) | (1649.18, 4069.22) |
| 211 | n = 30 | n = 25 | n = 26 | n = 26 |
| | 125.80 | 258.44 | 506.97 | 866.26 |
| | (48.02, 329.55) | (93.32, 715.73) | (228.56, 1124.50) | (445.59, 1684.08) |
| 393 | n = 32 | N/A | N/A | n = 25 |
| | 103.88 | N/A | N/A | 505.10 |
| | (39.85, 270.78) | | | (287.58, 887.15) |
| 757 | n = 27 | N/A | N/A | n = 21 |
| | 324.26 | N/A | N/A | 149.68 |
| | (131.54, 799.36) | | | (56.68, 395.24) |

TABLE 36
Geometric mean titers (GMTs) of Zika virus neutralizing antibodies
as measured by RVP (RVP₅₀) in the flavivirus naïve subject
set (Per Protocol Set, PPS) with 95% confidence interval
(GMTs (95% CI)). The number of subjects (n) refers to the
number of subjects in the per-protocol analysis set with
available RVP data at the indicated time-point.

| Study | | | | |
|---|---|---|---|---|
| Day | Placebo | 2 μg PIZV | 5 μg PIZV | 10 μg PIZV |
| 1 | n = 28 | n = 25 | n = 29 | n = 30 |
| | 34 | 37 | 34 | 48 |
| | (27, 44) | (29, 47) | (28, 40) | (41, 56) |
| 29 | n = 28 | n = 25 | n = 28 | n = 28 |
| | 29 | 408 | 688 | 1419 |
| | (23, 37) | (274, 607) | (468, 1010) | (942, 2137) |
| 57 | n = 27 | n = 23 | n = 28 | n = 28 |
| | 32 | 3701 | 6977 | 14790 |
| | (26, 40) | (2330, 5878) | (4663, 10438) | (10289, 21262) |
| 211 | n = 21 | n = 20 | n = 25 | n = 23 |
| | 20 | 332 | 690 | 1298 |
| | (15, 28) | (218, 506) | (470, 1012) | (809, 2082) |
| 393 | n = 23 | N/A | N/A | n = 21 |
| | 19 | N/A | N/A | 705 |
| | (15, 24) | | | (465, 1067) |
| 757 | n = 19 | N/A | N/A | n = 16 |
| | 30 | N/A | N/A | 642 |
| | (25, 36) | | | (432, 955) |

TABLE 37
Geometric mean titers (GMTs) of Zika virus neutralizing antibodies
as measured by RVP (RVP₅₀) in the flavivirus primed subject
set (Per Protocol Set, PPS) with 95% confidence interval
(GMTs (95% CI)). The number of subjects (n) refers to the
number of subjects in the per-protocol analysis set with
available RVP data at the indicated time-point.

| Study | | | | |
|---|---|---|---|---|
| Day | Placebo | 2 μg PIZV | 5 μg PIZV | 10 μg PIZV |
| 1 | n = 34 | n = 33 | n = 34 | n = 33 |
| | 538 | 533 | 733 | 386 |
| | (300, 962) | (305, 933) | (402, 1335) | (220, 678) |
| 29 | n = 34 | n = 31 | n = 34 | n = 33 |
| | 464 | 845 | 1965 | 2720 |
| | (247, 874) | (448, 1592) | (1075, 3590) | (1521, 4864) |
| 57 | n = 31 | n = 30 | n = 31 | n = 30 |
| | 544 | 1624 | 3048 | 5703 |
| | (291, 1017) | (1007, 2617) | (1966, 4728) | (3666, 8872) |
| | n = 30 | n = 25 | n = 26 | n = 26 |
| 211 | 773 | 1615 | 2663 | 3574 |
| | (416, 1436) | (896, 2912) | (1576, 4500) | (2048, 6234) |
| 393 | n = 32 | N/A | N/A | n = 25 |
| | 720 | N/A | N/A | 1767 |
| | (404, 1282) | | | (1002, 3113) |
| 757 | n = 27 | N/A | N/A | n = 21 |
| | 803 | N/A | N/A | 1461 |
| | (435, 1481) | | | (848, 2516) |

### Seropositivity Rates as Measured by PRNT and RVP

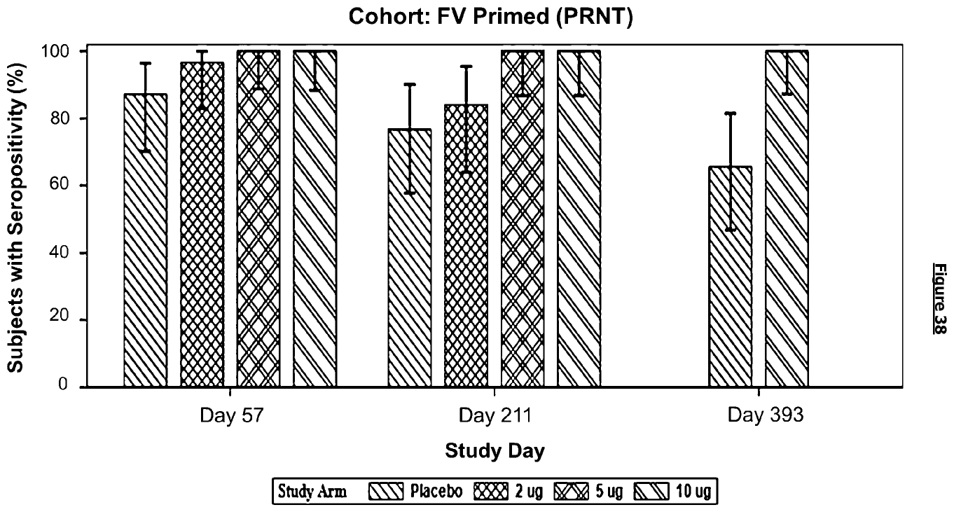

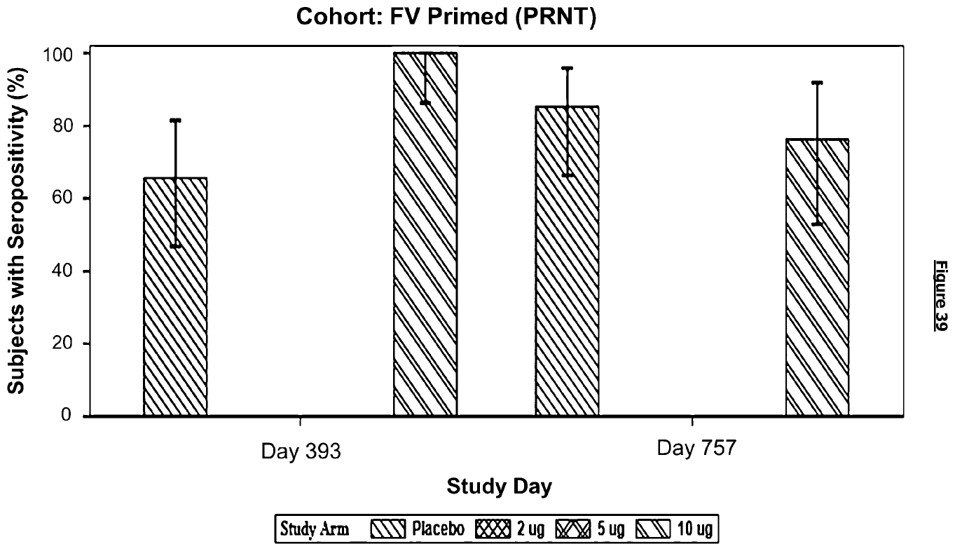

Tables 38 and 39 show the seropositivity rates as measured by PRNT. In addition, seropositivity rates as measured by PRNT for the flavivirus-primed cohort are also shown graphically in FIGS. 38 and 39.

TABLE 38
Seropositivity rates as measured by PRNT in the flavivirus
naïve subject set (Per Protocol Set, PPS) with 95% confidence
interval (GMTs (95% CI)). The number of subjects (n) at the
specific time points can be taken from Table 34 above.

| Study | | | | |
|---|---|---|---|---|
| Day | Placebo | 2 μg PIZV | 5 μg PIZV | 10 μg PIZV |
| 1 | 0 | 0 | 0 | 0 |
| 29 | 0 | 72.00 | 82.14 | 96.43 |
| | | (50.61, 87.93) | (63.11, 93.94) | (81.65, 99.91) |
| 57 | 0 | 100.00 | 100.00 | 100.00 |
| | | (85.18, 100.00) | (87.66, 100.00) | (87.66, 100.00) |
| 211 | 0 | 100.00 | 100.00 | 100.00 |
| | | (83.16, 100.00) | (86.28, 100) | (85.18, 100) |
| 393 | 0 | N/A | N/A | 100.0 |
| | | | | (83.89, 100.0) |
| 757 | 26.32 | N/A | N/A | 93.75 |
| | (9.15, 51.20) | | | (69.77, 99.84) |

TABLE 39
Seropositivity rates as measured by PRNT in the flavivirus
primed subject set (Per Protocol Set, PPS) with 95% confidence
interval (GMTs (95% CI)). The number of subjects (n) at the
specific time points can be taken from Table 35 above.

| Study | | | | |
|---|---|---|---|---|
| Day | Placebo | 2 μg PIZV | 5 μg PIZV | 10 μg PIZV |
| 1 | 88.2 | 75.8 | 85.3 | 75.8 |
| | (72.6, 96.7) | (57.7, 88.9) | (68.9, 95.1) | (57.7, 88.9) |
| 29 | 85.29 | 96.77 | 100.00 | 100.00 |
| | (68.94, 95.05) | (83.30, 99.92) | (89.72, 100.00) | (89.42, 100.00) |
| 57 | 87.10 | 96.67 | 100.00 | 100.00 |
| | (70.17, 96.37) | (82.78, 99.92) | (88.78, 100.00) | (88.43, 100.00) |
| 211 | 76.67 | 84.00 | 100 | 100.00 |
| | (52.72, 90.07) | (63.92, 95.46) | (86.77, 100.00) | (86.77, 100.00) |
| 393 | 65.63 | N/A | N/A | 100.0 |
| | (46.81, 81.43) | | | (86.28, 100.0) |
| 757 | 85.19 | N/A | N/A | 76.19 |
| | (66.27, 95.81) | | | (52.83, 91.78) |

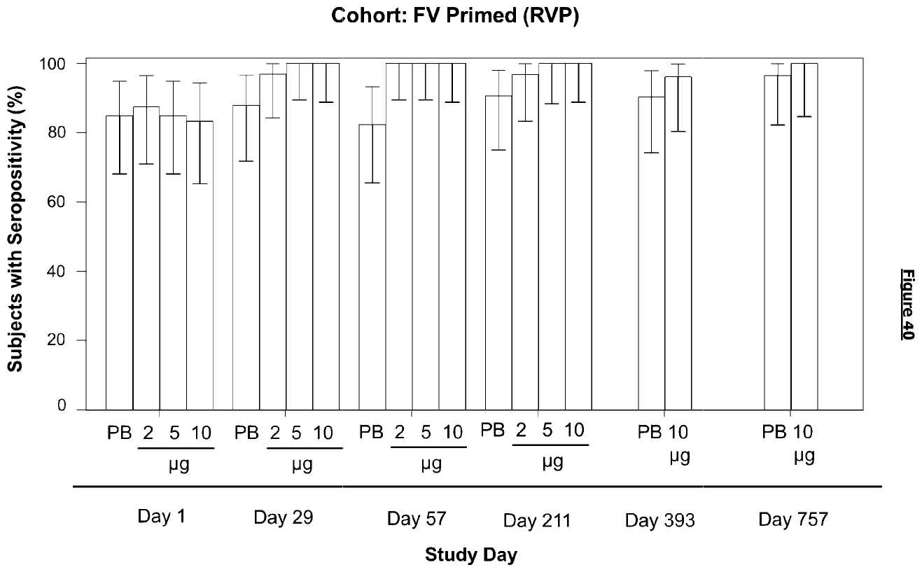

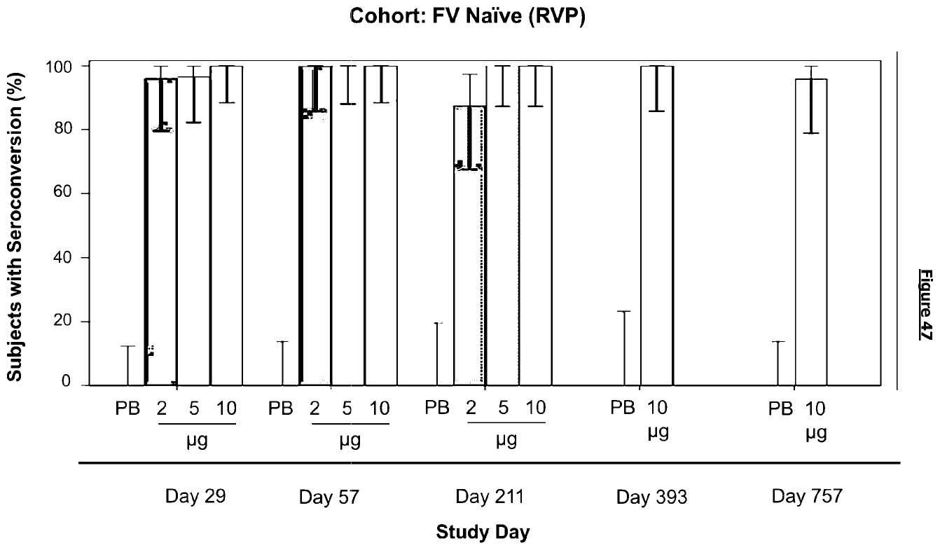

The seropositivity rates as measured by RVP in the flavivirus naïve subject set correspond to the seroconversion rates as measured by RVP in the flavivirus naïve subject set (cf. FIG. 47). The seropositivity rates as measured by RVP in the flavivirus primed subject set can be taken from FIG. 40. For both, flavivirus primed and naïve subjects, the corresponding subject numbers per timepoint can be taken from Tables 36 and 37, respectively.

### Seroconversion Rates as Measured by PRNT and RVP

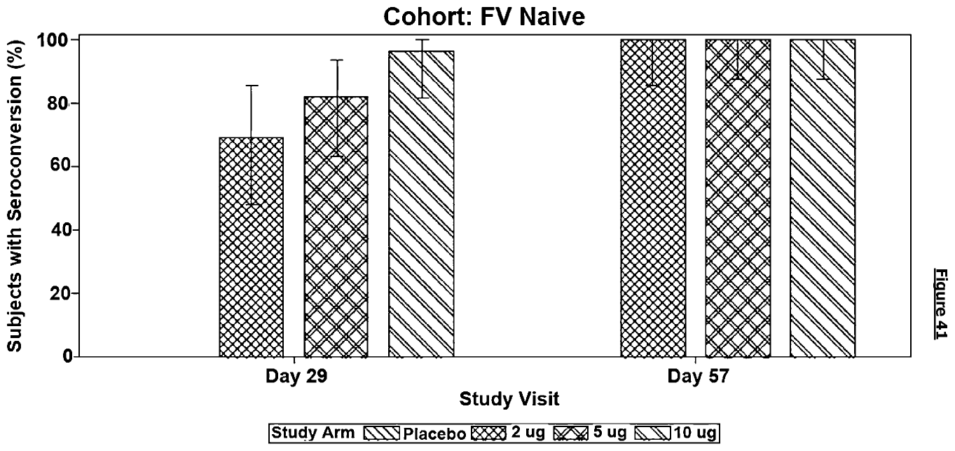

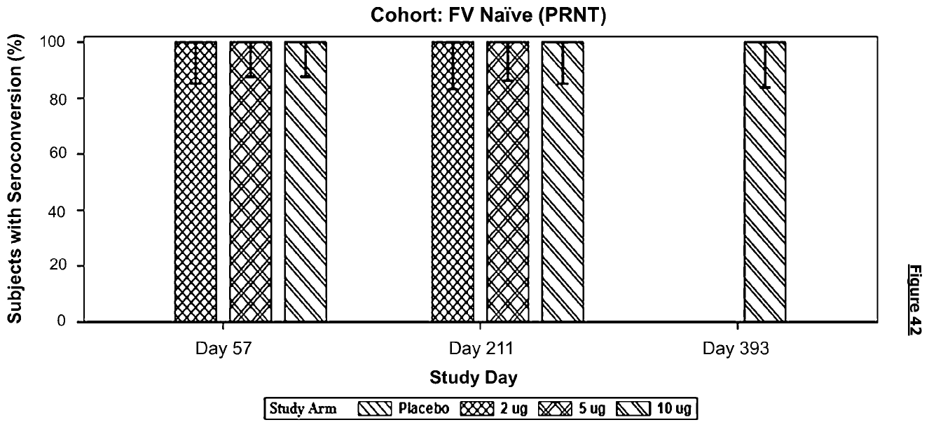

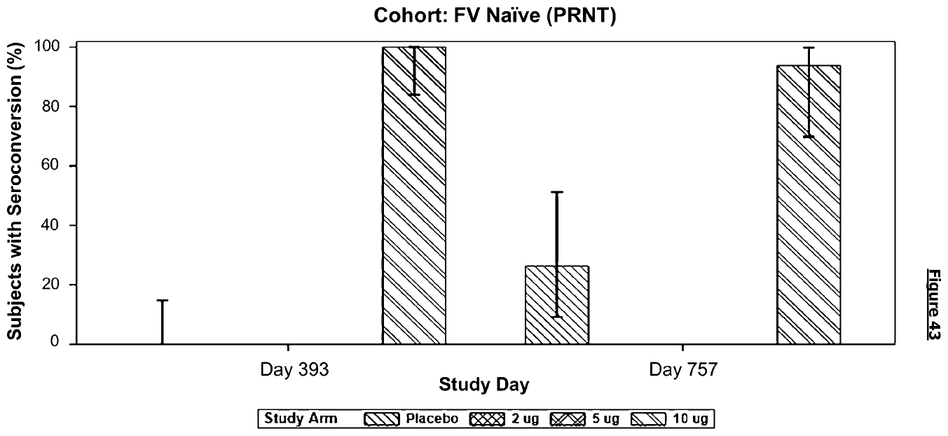

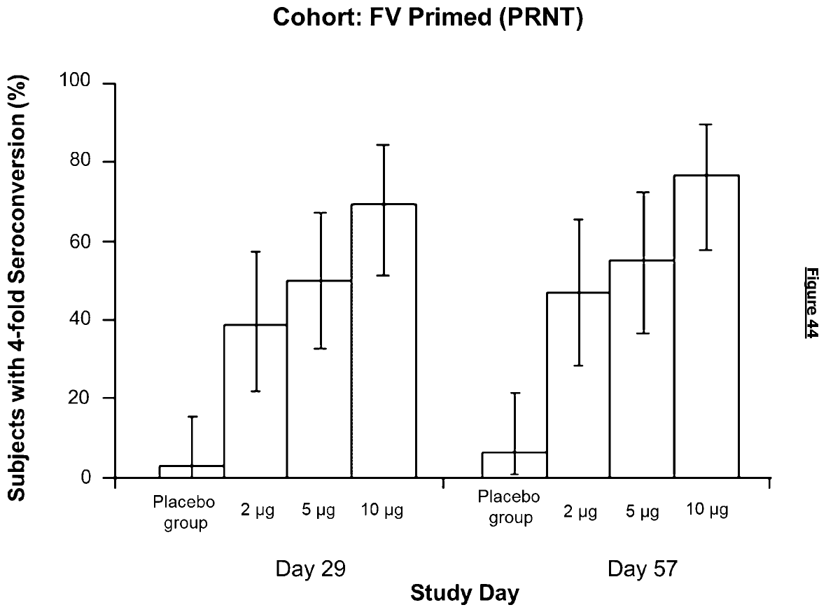

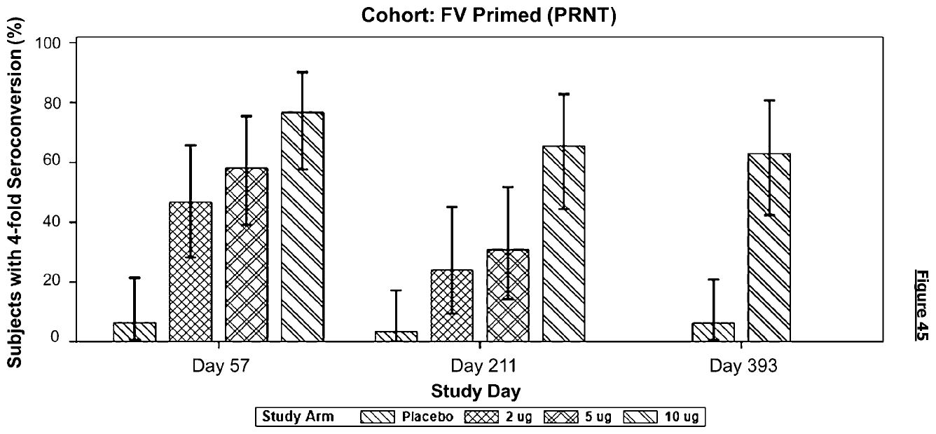

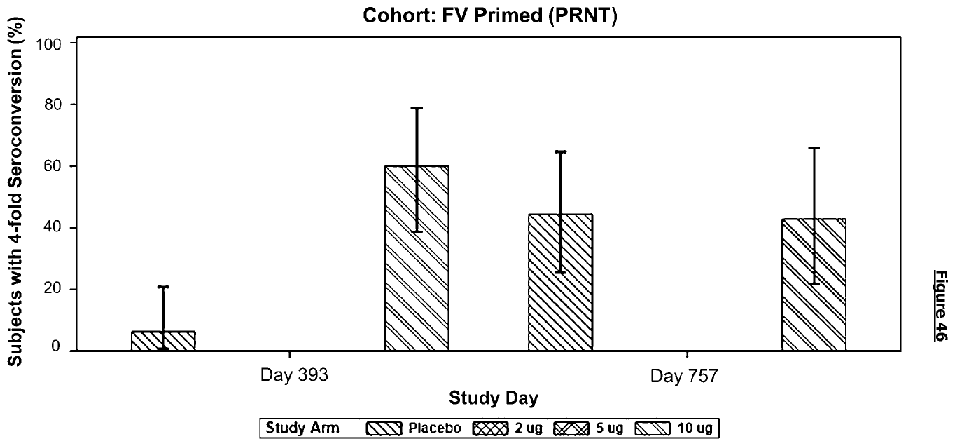

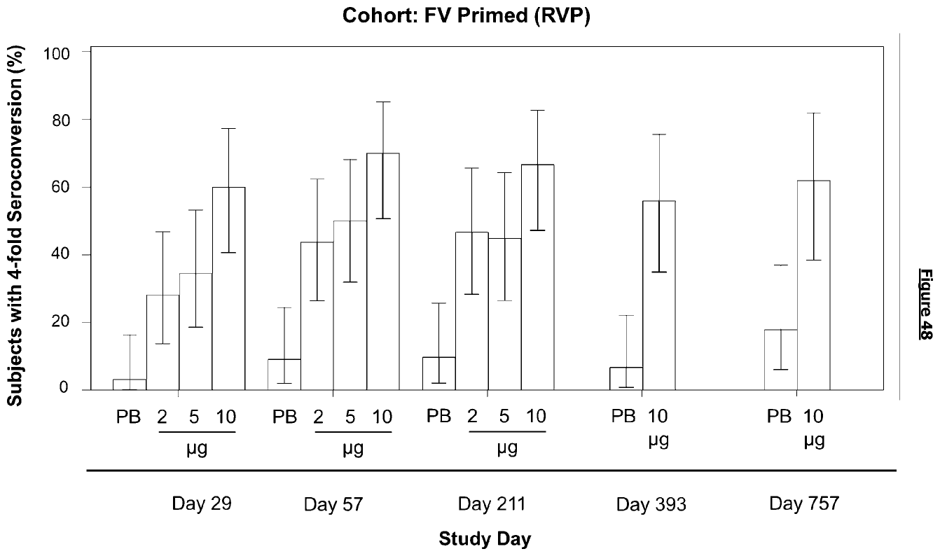

Tables 40 and 41 show the seroconversion rates as measured by PRNT 28 days post dose 1 and post dose 2. In addition, seroconversion rates as determined by PRNT and RVP are also shown graphically in FIGS. 41 to 48. The subject numbers at the specific time-points can be taken from Tables 34 to 37, respectively.

TABLE 40
Seroconversion rates as measured by PRNT in the flavivirus
naïve subject set (Per Protocol Set, PPS) with 95% confidence
interval (GMTs (95% CI)). The number of subjects (n) at the
specific time points can be taken from Table 34 above.

| Study | | | | |
|---|---|---|---|---|
| Day | Placebo | 2 μg PIZV | 5 μg PIZV | 10 μg PIZV |
| 29 | 0 | 72.00 | 82.14 | 96.42 |
| | | (50.61, 87.93) | (63.11, 93.94) | (81.65, 99.91) |
| 57 | 0 | 100.00 | 100.00 | 100.00 |
| | | (85.18, 100.00) | (87.66, 100.00) | (87.66, 100.00) |

TABLE 41
4-fold seroconversion rates as measured by PRNT in the flavivirus
primed subject set (Per Protocol Set, PPS) with 95% confidence
interval (GMTs (95% CI)). The number of subjects (n) at the
specific time points can be taken from Table 35 above.

| Study | | | | |
|---|---|---|---|---|
| Day | Placebo | 2 μg PIZV | 5 μg PIZV | 10 μg PIZV |
| 29 | 2.94 | 38.71 | 50.00 | 69.70 |
| | (0.07, 15.33) | (21.85, 57.81) | (32.43, 67.57) | (51.29, 84.41) |
| 57 | 6.45 | 46.67 | 54.84 | 76.67 |
| | (0.79, 21.42) | (28.34, 65.67) | (36.03, 72.68) | (57.72, 90.07) |

### Summary Immunogenicity in Flavivirus-Naïve Cohort

In the flavivirus-naïve cohort, vaccination elicited dose-dependent seroconversion shown by increases in neutralizing antibodies when assessed by either assay, whereas placebo recipients displayed no change. After the first dose of PIZV (day 29), PRNT seroconversion was observed in 18 (72%) of 25 participants in the 2 μg PIZV group, 23 (82%) of 28 in the 5 μg PIZV group, and 27 (96%) of 28 in the 10 μg PIZV group. The seroconversion rate was 100% in all PIZV groups after the second vaccination, whereas it remained at 0% in placebo recipients. At 6 months post-dose 2, 100% of initially flavivirus-naïve participants were still seropositive across the PIZV dosage groups, and this 100% rate persisted in the 10 μg group when assessed at 12 months post-dose 2. After 2 years post-dose 2, the seropositivity rate was still as high as 93.8%.

In the flavivirus-naive cohort, GMTs of neutralizing antibodies measured by PRNT were 41 (95% CI 19-91) in the 2 μg PIZV group, 94 (44-198) in the 5 μg PIZV group, and 291 (162-525) in the 10 μg PIZV group after the first dose (day 29), compared with the assigned value of 5 (half the lower limit of detection) for the placebo group. Although the 95% CIs around the PRNT GMTs for the 2 μg and 10 μg PIZV groups did not overlap, pairwise comparison of GMTs found no statistically significant differences after one vaccination. 4 weeks after the second vaccination (day 57), there were greater than ten-fold increases in antibody levels to 1130 (95% CI 749-1703) in the 2 μg PIZV group, 1992 (1401-2833) in the 5 μg PIZV group, and 3690 (2677-5086) in the 10 μg PIZV group. Pairwise comparisons showed that after two vaccinations, the GMT was significantly higher in the 10 μg PIZV group than the 2 μg PIZV group (GMT ratio 3:27 [95% CI 1:98-5:39]; p<0.0001) and the 5 μg PIZV group (GMT ratio 1:85 [1:15-2:98]; p=0.012). The 5 μg PIZV group GMT was also significantly higher than the 2 μg PIZV group (GMT ratio 1:76 [1:07-2:91]; p=0.027). Nevertheless, also with the 2 μg-dose, high neutralizing antibody titers were observed even after only one dose indicating that the PIZV is effective already at doses as low as 2 μg. Similar patterns of immune responses were observed using the Zika RVP. There was an apparent dose-dependent increase in RVP titers after the first PIZV vaccination, with ten-fold increases after the second dose, and no response in placebo recipients.

Flavivirus-naïve participants who received placebo remained seronegative through month 12 post-dose 2, but it was notable that 5 of 19 initially flavivirus-naïve participants assesses at month 24 post-dose 2 were seropositive, which may be an indication of natural infection due to circulating Zika virus, or potential cross-reactivity with antibodies against other flaviviruses.

### Summary Immunogenicity in Flavivirus-Primed Cohort

One dose of PIZV in the flavivirus-primed participants also elicited increases in Zika neutralizing antibody titers, in an apparent dose-dependent trend, with a four-fold increase in the 2 μg PIZV group and GMT of 514 (95% CI 260-1015), five-fold increase in the 5 μg PIZV group and GMT of 1003 (567-1774), and 21-fold increase in the 10 μg PIZV group achieving a GMT of 1573 (865-2860) 28 days post dose 1 as measured by PRNT. These levels were higher than those observed after one vaccination in the flavivirus-naive groups, but against the high baseline levels, the seroconversion rates were slightly lower. The GMTs after two doses of PIZV were 598 (95% CI 340-1049) in the 2 μg PIZV group, 1277 (806-2023) in the 5 μg PIZV group, and 2591 (1649-4069) in the 10 μg PIZV group as measured by PRNT. The Zika RVP assay confirmed these observations in the flavivirus-primed groups.

100% of the flavivirus-primed participants who received the 10 μg dose were still seropositive one year after the second vaccination and 76.2% were still seropositive two years after the second vaccination.

### CONCLUSION

The PIZV vaccine was well-tolerated and safe for all antigen doses evaluated in both, the flavivirus-naïve and -primed cohort. The intensity of solicited AEs was mild to moderate and no serious adverse events related to the investigational vaccine were observed. Local solicited AEs reported were also mild to moderate in intensity across the groups. The high neutralizing antibody titers even after the first dose indicate an early onset of protection, which is particularly beneficial in an outbreak situation or a traveler visiting an endemic area within a short period of time from the administration of the vaccine. Moreover, high antibody titers persisted even up to 2 years after the last vaccination. A low number of administrations, while maintaining nevertheless a high seroconversion rate, will offer multiple advantages regarding, for instance, costs and patient comfort.

### Example 7: Immunogenicity and Safety of a Purified Inactivated Zika Virus Vaccine (PIZV) Based on P6e Strain Evaluated in a Phase II Clinical Trial

The purified inactivated Zika virus vaccine (PIZV) is further evaluated in a Phase II clinical trial (ZIK-201) due to the excellent safety and immunogenicity results observed in Phase I (Example 6).

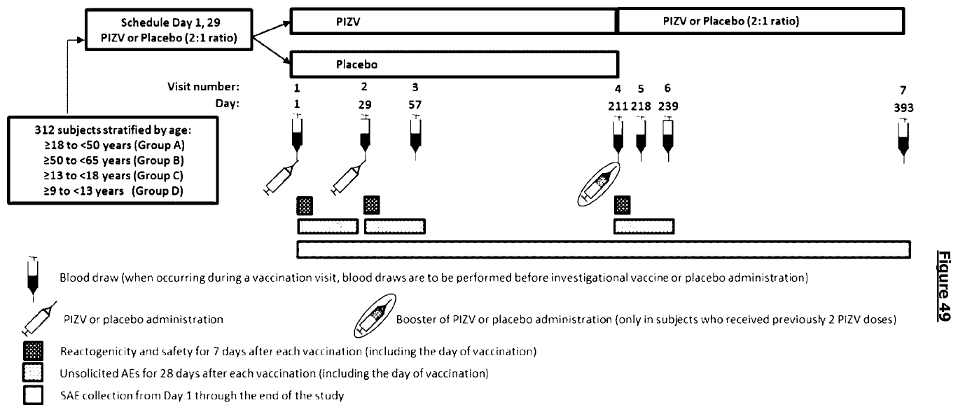

For the Phase II trial, the high antigen dose of Phase I was selected, i.e. 10 μg, for further investigation. A schematic representation of the Phase II trial design is given in FIG. 49.

The Phase II study is a randomized, observer-blind, placebo-controlled clinical trial to evaluate the safety and immunogenicity of PIZV administered on day 1 and 29, followed by a single booster dose administered 6 months post-dose 2 (study day 211). The administration occurs intramuscularly into the middle third of the deltoid muscle, preferably in the non-dominant arm at a volume of 0.5 mL. Placebo (sterile 0.9% sodium chloride solution) serves as the control.

### Study Population

The study is conducted in approximately 312 healthy male and female subjects aged 9 to 65 years in the US. Compared to the ZIK-101 study (Example 6), the age range is expanded to younger and older subjects to cover a broader target population range. The Phase II study is conducted in areas that are non-endemic for ZIKV and/or dengue virus. Thus, the major amount of the subjects (about 80% or more) are flavivirus naïve.

Key Inclusion Criteria: besides the age and health status the subjects must also be available for the duration of follow-up and all females of childbearing potential must have a negative pregnancy test prior to receiving any dose including the booster.

Key Exclusion Criteria: subjects with past or current Zika virus infection by self-report; subjects with current dengue virus, yellow fever virus, Japanese encephalitis virus, tick-borne encephalitis virus or West Nile virus infection by self-report; subjects who have traveled to flavivirus-endemic countries and US regions and territories (cf. website of the Centers for Disease Control and Prevention, CDC), within 4 weeks prior to anticipated enrollment or who have planned to travel to these countries/regions up to 28 days post dose 2 or booster dose; subjects with known hypersensitivity or allergy to any of the vaccine candidate components or the placebo; subjects that received a licensed vaccine before any dose: live attenuated vaccine within 28 days or non-live vaccine within 14 days; female subjects who are pregnant or breastfeeding, or are planning to become pregnant during the duration of the study; female subjects of childbearing potential who refuse to use an acceptable contraceptive method from trial entry through 2 months after the second dose of PIZV/placebo, and from 2 months before through 2 months after the booster dose; subjects with any history of progressive or severe neurological disorder, seizure disorder, or neuroinflammatory disease (e.g., Guillain-Barré syndrome); subjects with any illness or disease that, in the opinion of the investigator, may interfere with the subject's ability to participate in the trial.

Booster Eligibility Criteria: the subjects are re-randomized and treated in the Booster Period if they meet the following eligibility criteria: subjects continue to meet the initial trial inclusion and exclusion criteria; subjects who received 2 doses of PIZV (not placebo) during the 2-dose Vaccination Period; subjects whose personal safety data during the 2-dose Vaccination Period do not preclude them from receiving a booster dose in the opinion of the investigator.

In ZIK-201, four age groups will be evaluated (n=78 per group):
Group A: ≥18 to <50 years (PIZV=52; Placebo=26)
Group B: ≥50 to <65 years (PIZV=52; Placebo=26)
Group C: ≥13 to <18 years (PIZV=52; Placebo=26)
Group D: ≥9 to <13 years (PIZV=52; Placebo=26)

Subjects in each age group at enrollment are randomized in a 2:1 ratio (PIZV:Placebo). At 6 months post-dose 2, subjects who received PIZV at enrollment are re-randomized to receive a booster dose in a 2:1 ratio (PIZV:Placebo). All subjects (n=312) are followed for up to 7 months post dose 1 (2-dose Vaccination Period) and a subgroup (n=208) consisting of subjects that received the PIZV are followed up for 6 months more (Booster Period) for a total of 13 months.

### Primary Objectives:

The primary objectives are to describe the immune responses of the 2-dose PIZV vaccination schedule (Day 1, 29) 28 days post dose 2 as measured by neutralizing anti-Zika virus antibodies and to describe the safety of the 2-dose PIZV vaccination schedule (Day 1, 29) 28 days post dose 2.

### Secondary Objectives:

The secondary objectives are to describe the immune response after the 2-dose PIZV vaccination schedule (Day 1, 29) at 6 months post dose 2, as measured by neutralizing anti-Zika virus antibodies, to describe the safety up to 6 months post dose 2, to describe the immune response to a PIZV booster dose administered 6 months post dose 2 at 7 days, 28 days, and 6 months post booster dose as measured by neutralizing antibodies, and to describe safety of a PIZV booster, administered 6 months post dose 2 up to 6 months post booster. In addition, geometric mean fold rise (GMFR) as compared to before the booster vaccination is determined.

### Immune Responses:

Neutralizing antibody titers and therefrom, seropositivity rates (SPR), seroconversion rates (SCR) and geometric mean neutralizing antibody titers (GMTs) are determined (see above for definitions of SPR, SCR, and GMTs) 28 days and 6 months post dose 2, as well as 7 days, 28 days, and 6 months post booster. In addition, geometric mean fold rise (GMFR) as compared to before the booster vaccination is determined. For determination of neutralizing antibody titers, PRNT and RVP assay are applied (cf. Example 6 above).

### Safety

Collected are solicited local reactions (pain, erythema, swelling, and induration) for 7 days after each vaccination; solicited systemic adverse events (AEs) including fever, headache, fatigue, malaise, arthralgia, and myalgia for 7 days after each vaccination; unsolicited AEs for 28 days after each vaccination; and serious adverse events (SAEs) throughout the entire study period.

### Analysis Sets for the Assessment in the 2-Dose Vaccination Period:

Safety Set: This set consists of all randomized subjects who received at least one dose of PIZV or placebo. Data for subjects in the Safety Set are analyzed based on the first treatment received (PIZV or placebo).

Full Analysis Set (FAS): All randomized subjects who received at least one dose of PIZV or placebo and who provided valid flavivirus baseline results and at least one post-vaccination serology result. Data for subjects in the FAS are analyzed based on randomized treatment group.

Per-Protocol Set (PPS): All subjects in the FAS without any major protocol deviations. The major protocol deviation criteria are defined as part of the blinded data review prior to the unblinding of subject's PIZV/placebo assignment. The categories of major protocol deviations include (but are not limited to): not meeting selected entry criteria, receiving a wrong investigational vaccine/placebo, receiving prohibited therapies, and other major protocol deviations that may be identified during blinded data reviews.

### Analysis Sets for the Assessment in the Booster Period:

Booster Safety Set: All subjects who received two doses of the same treatment (PIZV or placebo) during the 2-dose Vaccination Period, were re-randomized and received a booster dose at 6 months post dose 2 (PIZV and placebo). Data for subjects in the Booster Safety Set are analyzed based on the booster treatment received (PIZV or placebo).

Booster FAS: All subjects who received two doses of the same treatment (PIZV or placebo) during the 2-dose Vaccination Period, were re-randomized and received a booster dose at 6 months post dose 2 (PIZV or placebo), and who had pre-booster and post-booster serology results. Data for subjects in the Booster FAS are analyzed based on second randomized treatment group.

Booster PPS: All subjects in the Booster FAS without any major protocol deviations. Subjects should have been in the PPS for the 2-dose Vaccination Period to qualify for the booster PPS.

All summaries and analyses of safety data are based on subjects in the Safety Set or Booster Safety Set. Analyses of immunogenicity, including immune response at 6 months post dose 2, as well as the booster response, are based on the PPS or Booster PPS. Subgroup analyses are performed for subgroups defined by flavivirus serostatus at baseline and age.

### FURTHER ITEMS OF THE DISCLOSURE

### First Item List of the Disclosure

1. A method for inducing an immune response against Zika virus and/or preventing Zika virus disease and/or preventing Zika virus infection in a human subject or a human subject population in need thereof, the method comprising administering to the human subject or the individuals of the human subject population a vaccine or immunogenic composition comprising an antigen from a Zika virus, wherein the antigen is an inactivated whole Zika virus and the human subject or the individuals of the human subject population are flavivirus primed.
2. The method according to item 1, wherein the human subject population comprises at least 20 individuals.
3. The method according to item 1 or 2, wherein the vaccine or immunogenic composition is administered as a first and a second administration, wherein the first and the second administration take place from about 1 to about 16 weeks apart or from about 1 to about 6 weeks apart or from about 1 to about 4 weeks apart.
4. The method according to item 3, wherein the vaccine or immunogenic composition is administered as a first and a second administration, wherein the first and the second administration take place from about 25 to 30 days apart, such as 28 days apart.
5. The method according to any one of items 1 to 4, wherein administering of the vaccine or immunogenic composition to the individuals of the human subject population induces a 4-fold seroconversion rate of at least 30%, or at least 40%, or at least 50%, or at least 60%, or at least 70% in said human subject population as determined by a plaque reduction neutralization test.
6. The method according to any one of items 1 to 5, wherein administering of the vaccine or immunogenic composition to the individuals of the human subject population induces a seropositivity rate of at least 70%, or at least 75%, or at least 80%, or at least 85%, or at least 90%, or at least 95%, or 100% in said human subject population as determined by a plaque reduction neutralization test.
7. The method according to any one of items 1 to 6, wherein administering of the vaccine or immunogenic composition to the individuals of the human subject population induces geometric mean neutralizing antibody titers of at least 100, or at least 250, or at least 500, or at least 800, or at least 1000, or at least 1500, or at least 2500 in said human subject population as determined by a plaque reduction neutralization test.
8. The method according to any one of the items above, wherein administering of the vaccine or immunogenic composition to the individuals of the human subject population induces 28 days after the first administration a 4-fold seroconversion rate of at least 30%, or at least 50%, or at least 65% in said human subject population as determined by a plaque reduction neutralization test.
9. The method according to any one of the items above, wherein administering of the vaccine or immunogenic composition to the individuals of the human subject population induces 28 days after the second administration a 4-fold seroconversion rate of at least 40%, or at least 50%, or at least 70% in said human subject population as determined by a plaque reduction neutralization test.
10. The method according to any one of the items above, wherein administering of the vaccine or immunogenic composition to the individuals of the human subject population induces 182 days after the second administration a 4-fold seroconversion rate of at least 20%, or at least 30%, or at least 60% in said human subject population as determined by a plaque reduction neutralization test.
11. The method according to any one of the items above, wherein administering of the vaccine or immunogenic composition to the individuals of the human subject population induces 364 days after the second administration a 4-fold seroconversion rate of at least 50%, or at least 55%, or at least 60% in said human subject population as determined by a plaque reduction neutralization test.
12. The method according to any one of the items above, wherein administering of the vaccine or immunogenic composition to the individuals of the human subject population induces 728 days after the second administration a 4-fold seroconversion rate of at least 40% in said human subject population as determined by a plaque reduction neutralization test.
13. The method according to any of the items above, wherein administering of the vaccine or immunogenic composition to the individuals of the human subject population induces 28 days after the first administration a seropositivity rate of at least 80%, or at least 85%, or at least 90%, or at least 95%, or 100% in said human subject population as determined by a plaque reduction neutralization test.
14. The method according to any of the items above, wherein administering of the vaccine or immunogenic composition to the individuals of the human subject population induces 28 days after the second administration a seropositivity rate of at least 80%, or at least 85%, or at least 90%, or at least 95%, or 100% in said human subject population as determined by a plaque reduction neutralization test.
15. The method according to any of the items above, wherein administering of the vaccine or immunogenic composition to the individuals of the human subject population induces 182 days after the second administration a seropositivity rate of at least 80%, or at least 85%, or at least 90%, or at least 95%, or 100% in said human subject population as determined by a plaque reduction neutralization test.
16. The method according to any of the items above, wherein administering of the vaccine or immunogenic composition to the individuals of the human subject population induces 364 days after the second administration a seropositivity rate of at least 80%, or at least 85%, or at least 90%, or at least 95%, or 100% in said human subject population as determined by a plaque reduction neutralization test.
17. The method according to any of the items above, wherein administering of the vaccine or immunogenic composition to the individuals of the human subject population induces 728 days after the second administration a seropositivity rate of at least 70% or at least 75% in said human subject population as determined by a plaque reduction neutralization test.
18. The method according to any of the items above, wherein administering of the vaccine or immunogenic composition to the individuals of the human subject population induces 28 days after the first administration geometric mean neutralizing antibody titers of at least 500, or at least 1000, or at least 1500 in said human subject population as determined by a plaque reduction neutralization test.
19. The method according to any of the items above, wherein administering of the vaccine or immunogenic composition to the individuals of the human subject population induces 28 days after the second administration geometric mean neutralizing antibody titers of at least 500, or at least 1000, or at least 2500 in said human subject population as determined by a plaque reduction neutralization test.
20. The method according to any of the items above, wherein administering of the vaccine or immunogenic composition to the individuals of the human subject population induces 182 days after the second administration geometric mean neutralizing antibody titers of at least 250, or at least 500, or at least 800 in said human subject population as determined by a plaque reduction neutralization test.
21. The method according to any of the items above, wherein administering of the vaccine or immunogenic composition to the individuals of the human subject population induces 364 days after the second administration geometric mean neutralizing antibody titers of at least 250 or at least 500 in said human subject population as determined by a plaque reduction neutralization test.
22. The method according to any of the items above, wherein administering of the vaccine or immunogenic composition to the individuals of the human subject population induces 728 days after the second administration geometric mean neutralizing antibody titers of at least 100 in said human subject population as determined by a plaque reduction neutralization test.
23. The method according to any of the items above, wherein administering of the vaccine or immunogenic composition to the individuals of the human subject population induces 28 days after the first administration geometric mean neutralizing antibody titers of at least 800, or at least 1500, or at least 2500 in said human subject population as determined by a reporter virus particle assay.
24. The method according to any of the items above, wherein administering of the vaccine or immunogenic composition to the individuals of the human subject population induces 28 days after the second administration geometric mean neutralizing antibody titers of at least 1500, or at least 3000, or at least 5000 in said human subject population as determined by a reporter virus particle assay.
25. The method according to any of the items above, wherein administering of the vaccine or immunogenic composition to the individuals of the human subject population induces 182 days after the second administration geometric mean neutralizing antibody titers of at least 1500, or at least 2000, or at least 2500, or at least 3000, or at least 3500 in said human subject population as determined by a reporter virus particle assay.
26. The method according to any of the items above, wherein administering of the vaccine or immunogenic composition to the individuals of the human subject population induces 364 days after the second administration geometric mean neutralizing antibody titers of at least 1000 or at least 1500 in said human subject population as determined by a reporter virus particle assay.
27. The method according to any of the items above, wherein administering of the vaccine or immunogenic composition to the individuals of the human subject population induces 728 days after the second administration geometric mean neutralizing antibody titers of at least 1000 in said human subject population as determined by a reporter virus particle assay.
28. The method according to any of the items above, wherein the inactivated whole Zika virus comprises an envelope protein having an amino acid sequence that has at least 70%, at least 75%, at least 80%, at least 85%, at least 90%, at least 95%, or 100% sequence identity with SEQ ID NO: 6.
29. The method according to item 28, wherein the inactivated whole Zika virus comprises an envelope protein represented by SEQ ID NO: 6.
30. The method according to any of the items above, wherein the inactivated whole Zika virus comprises an RNA genome sequence characterized by a DNA sequence having at least 70%, at least 75%, at least 80%, at least 85%, at least 90%, at least 95%, or 100% sequence identity with SEQ ID NO: 5.
31. The method according to item 30, wherein the inactivated whole Zika virus comprises an RNA genome sequence characterized by SEQ ID NO: 5.
32. The method according to any of items 1 to 29, wherein the inactivated whole Zika virus comprises an RNA genome sequence characterized by a DNA sequence having at least 70%, at least 75%, at least 80%, at least 85%, at least 90%, at least 95%, or 100% sequence identity with SEQ ID NO: 3.
33. The method according to item 32, wherein the inactivated whole Zika virus comprises an RNA genome sequence characterized by SEQ ID NO: 3.
34. The method according to any of the items above, wherein the inactivated whole Zika virus comprises a glycine at amino acid position 98 of SEQ ID NO: 8 or at a position equivalent to amino acid position 98 of SEQ ID NO: 8.
35. The method according to any of the items above, wherein the inactivated whole Zika virus was inactivated with formaldehyde.
36. The method according to item 35, wherein the numerical result of the multiplication of the formaldehyde concentration as measured in % (w/v) with the period of incubation with formaldehyde as measured in days is 0.025 to 0.5.
37. The method according to item 35 or 36, wherein the inactivated whole Zika virus was inactivated with 0.005 to 0.02% (w/v) formaldehyde.
38. The method according to any of items 35 to 37, wherein the inactivated whole Zika virus was inactivated with formaldehyde for six to fourteen days.
39. The method according to any of items 35 to 38, wherein the inactivated whole Zika virus was inactivated with formaldehyde at a temperature of 15° C. to 30° C.
40. The method according to any of the items 35 to 39, wherein the inactivated whole Zika virus was inactivated with 0.01% (w/v) formaldehyde.
41. The method according to any of the items 35 to 40, wherein the inactivated whole Zika virus was inactivated with 0.01% (w/v) formaldehyde at a temperature of 22° C.
42. The method according to any of the items 35 to 41, wherein the inactivated whole Zika virus was inactivated with 0.01% (w/v) formaldehyde at a temperature of 22° C. for 10 days.
43. The method according to any of the items above, wherein the vaccine or immunogenic composition comprises a residual formaldehyde content of less than 50 μg/mL.
44. The method according to item 43, wherein the residual formaldehyde content is determined by a method comprising the steps of:
   (a) providing a vaccine or immunogenic composition comprising a Zika virus which has been treated with formaldehyde;
   (b) mixing the vaccine or immunogenic composition of (a) with phosphoric acid and 2,4-dinitrophenylhydrazine (DNPH), thereby providing a mixture;
   (c) incubating the mixture of (b) under suitable conditions; and
   (d) analyzing the mixture for the presence of residual formaldehyde.
45. The method according to any of the items above, wherein the vaccine or immunogenic composition comprises less than 1.0 TCID₅₀ of residual replicating Zika virus.
46. The method according to item 45, wherein residual replicating Zika virus is determined by a method for determining the completeness of inactivation of a Zika virus preparation comprising the steps of:
   (i) inoculating cultured insect cells with a Zika virus preparation which was subjected to an inactivation step and incubating the insect cells for a first period of time, thereby producing an insect cell supernatant;
   (ii) inoculating cultured mammalian cells with the insect cell supernatant produced in (i) and incubating the mammalian cells for a second period of time; and
   (iii) determining whether the Zika virus preparation contains a residual replicating virus that produces a cytopathic effect on the mammalian cells.
47. The method according to any of the items above, wherein the vaccine or immunogenic composition comprises a dose of from about 1 μg to about 40 μg of the inactivated whole Zika virus.
48. The method according to any of the items above, wherein the vaccine or immunogenic composition comprises a dose of from about 1 μg to about 20 μg of the inactivated whole Zika virus.
49. The method according to any of the items above, wherein the vaccine or immunogenic composition comprises a dose of from about 1 μg to about 15 μg of the inactivated whole Zika virus.
50. The method according to any of the items above, wherein the vaccine or immunogenic composition comprises a dose of from about 5 μg to about 15 μg of the inactivated whole Zika virus.
51. The method according to any of the items above, wherein the vaccine or immunogenic composition comprises a dose of from about 6 μg to about 15 μg of the inactivated whole Zika virus.
52. The method according to any of the items above, wherein the vaccine or immunogenic composition comprises a dose of from about 7 μg to about 15 μg of the inactivated whole Zika virus.
53. The method according to any of the items above, wherein the vaccine or immunogenic composition comprises a dose of from about 7 μg to about 13 μg of the inactivated whole Zika virus.
54. The method according to any of the items above, wherein the vaccine or immunogenic composition comprises a dose of from about 7.5 μg to about 12.5 μg of the inactivated whole Zika virus.
55. The method according to any of the items above, wherein the vaccine or immunogenic composition comprises a dose of from about 8 μg to about 12 μg of the inactivated whole Zika virus.
56. The method according to any of the items above, wherein the vaccine or immunogenic composition comprises a dose of from about 8.5 μg to about 11.5 μg of the inactivated whole Zika virus.
57. The method according to any of the items above, wherein the vaccine or immunogenic composition comprises a dose of from about 9 μg to about 11 μg of the inactivated whole Zika virus.
58. The method according to any of the items above, wherein the vaccine or immunogenic composition comprises a dose of from about 2 μg to about 10 μg of the inactivated whole Zika virus.
59. The method according to any of the items above, wherein the vaccine or immunogenic composition comprises a dose of about 2 μg of the inactivated whole Zika virus.
60. The method according to any of the items above, wherein the vaccine or immunogenic composition comprises a dose of about 5 μg of the inactivated whole Zika virus.
61. The method according to any of the items above, wherein the vaccine or immunogenic composition comprises a dose of about 10 μg of the inactivated whole Zika virus.
62. The method of any one of items 47 to 61, wherein the main peak of the inactivated whole Zika virus when analyzed by size exclusion chromatography is more than 65% or more than 75% or more than 85% of the total area under the curve in the size exclusion chromatography.
63. The method according to any of the items above, wherein the vaccine or immunogenic composition comprises a dose of from about 6 μg to about 15 μg, such as about 10 μg, of inactivated whole Zika virus and wherein the main peak of the inactivated whole Zika virus when analyzed by size exclusion chromatography is more than 85% of the total area under the curve in the size exclusion chromatography.
64. The method according to any of the items above, wherein the vaccine or immunogenic composition comprises an adjuvant.
65. The method according to item 64, wherein the adjuvant is an aluminum salt.
66. The method according to item 65, wherein the vaccine or immunogenic composition comprises from about 100 μg to about 600 μg, from about 100 μg to about 300 μg, from about 150 μg to about 250 μg, from about 175 μg to about 225 μg, or about 200 μg of an aluminum salt adjuvant.
67. The method according to item 65 or 66, wherein the aluminum salt adjuvant is aluminum hydroxide or aluminum phosphate.
68. The method according to item 67, wherein the aluminum salt adjuvant is aluminum hydroxide.
69. The method according to any of items 64 to 68, wherein at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the antigen are adsorbed to the adjuvant.
70. The method according to any of the items above, wherein the vaccine or immunogenic composition comprises aluminum hydroxide as an adjuvant and wherein at least 95% of the antigen are adsorbed to the adjuvant.
71. The method according to any of the items above, wherein the vaccine or immunogenic composition comprises from about 150 μg to about 250 μg, such as 200 μg, of aluminum hydroxide as an adjuvant.
72. The method according to any of the items above, wherein the vaccine or immunogenic composition comprises from about 175 μg to about 225 μg, such as 200 μg, of aluminum hydroxide as an adjuvant.
73. The method according to any of the items above, wherein the vaccine or immunogenic composition comprises a dose of from about 6 to about 15 μg, such as 10 μg, of inactivated whole Zika virus and from about 150 μg to about 250 μg, such as 200 μg, of aluminum hydroxide as an adjuvant.
74. The method according to any of the items above, wherein the vaccine or immunogenic composition comprises a dose of from about 6 to about 15 μg, such as 10 μg, of inactivated whole Zika virus and from about 175 μg to about 225 μg, such as 200 μg, of aluminum hydroxide as an adjuvant.
75. The method according to item 73 or 74, wherein the main peak of the inactivated whole Zika virus when analyzed by size exclusion chromatography is more than 85% of the total area under the curve in the size exclusion chromatography.
76. The method according to any of the items above, wherein the vaccine or immunogenic composition is administered in a volume of about 0.5 mL per administration.
77. The method according to any one of the items above, wherein the vaccine or immunogenic composition is administered intramuscularly.
78. The method according to any of the items above, wherein the vaccine or immunogenic composition is administered as a first and a second administration and wherein the administration of the vaccine or immunogenic composition up to 7 days after the prime administration induces
   fever in less than 3%, or in 0%, and/or
   fatigue in less than 26%, or in less than 18%, or less than 10%, and/or
   arthralgia in less than 13%, or in less than 7%, and/or
   headache in less than 23%, or in less than 18%, or in less than 16%, and/or
   myalgia in less than 18%, or in less than 12%, and/or
   malaise in less than 23%, or in less than 13%
      of the individuals of the human subject population, optionally wherein the vaccine or immunogenic composition comprises a dose of from about 2 to about 15 μg of the inactivated whole Zika virus.
79. The method according to any of the items above, wherein the vaccine or immunogenic composition is administered as a first and a second administration and wherein the administration of the vaccine or immunogenic composition up to 7 days after the boost administration induces
   fever in less than in less than 7%, and/or
   headache in less than 16%, or in less than 12%, or in less than 7%, and/or
   arthralgia in less than 10%, or in less than 6%, and/or
   fatigue in less than 15%, or in less than 13%, or in less than 4%, and/or
   myalgia in less than 13%, or in less than 10%, and/or
   malaise in less than 13%, or in less than 4%
      of the individuals of the human subject population, optionally wherein the vaccine or immunogenic composition comprises a dose of from about 2 to about 15 μg of the inactivated whole Zika virus.
80. The method according to any of the items above, wherein the vaccine or immunogenic composition is administered as a first and a second administration and wherein the administration of the vaccine or immunogenic composition up to 7 days after the prime administration induces
   fever in 0%, and/or
   headache in less than 18%, and/or
   arthralgia in less than 6%, and/or
   fatigue in less than 18%, and/or
   myalgia in less than 12%, and/or
   malaise in less than 23%
      of the individuals of the human subject population, wherein the vaccine or immunogenic composition comprises a dose of from about 6 μg to about 15 μg, such as 10 μg, of the inactivated whole Zika virus.
81. The method according to any of the items above, wherein the vaccine or immunogenic composition is administered as a first and a second administration and wherein the administration of the vaccine or immunogenic composition up to 7 days after the boost administration induces
   fever in 0%, and/or
   headache in less than 12%, and/or
   arthralgia in less than 6%, and/or
   fatigue in less than 15%, and/or
   myalgia in less than 12%, and/or
   malaise in less than 9%,
      of the individuals of the human subject population, wherein the vaccine or immunogenic composition comprises a dose of from about 6 μg to about 15 μg, such as 10 μg, of the inactivated whole Zika virus.
82. A vaccine or immunogenic composition comprising an antigen from a Zika virus for use in a method of any one of items 1 to 81, wherein the antigen is an inactivated whole Zika virus.
83. Use of a vaccine or immunogenic composition comprising an antigen from a Zika virus in the manufacture of a medicament for a method of any one of items 1 to 81, wherein the antigen is an inactivated whole Zika virus.

### Second Item List of the Disclosure

1. A method for inducing an immune response against Zika virus and/or preventing Zika virus disease and/or preventing Zika virus infection in a human subject population in need thereof, the method comprising administering to the individuals of the human subject population a vaccine or immunogenic composition comprising an antigen from a Zika virus as a first and a second administration, wherein
   the antigen is an inactivated whole Zika virus,
   optionally the individuals of the human subject population are flavivirus naïve, and
   administering of the vaccine or immunogenic composition to the individuals of the human subject population induces 182 days after the second administration a seropositivity rate of at least 70%, at least 80%, at least 85%, at least 90%, at least 95%, at least 98%, or 100% in said human subject population as determined by a plaque reduction neutralization test.
2. A method for inducing an immune response against Zika virus and/or preventing Zika virus disease and/or preventing Zika virus infection in a human subject population in need thereof, optionally according to item 1, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 182 days after the second administration a seroconversion rate of at least 70%, at least 80%, at least 85%, at least 90%, at least 95%, at least 98%, or 100% in said human subject population as determined by a plaque reduction neutralization test.
3. A method for inducing an immune response against Zika virus and/or preventing Zika virus disease and/or preventing Zika virus infection in a human subject population in need thereof, optionally according to item 1 or 2, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 182 days after the second administration geometric mean neutralizing antibody titers of greater than 150, or greater than 350, or greater than 600 in said human subject population as determined by a plaque reduction neutralization test.
4. A method for inducing an immune response against Zika virus and/or preventing Zika virus disease and/or preventing Zika virus infection in a human subject population in need thereof, optionally according to any one of items 1 to 3, the method comprising administering to the individuals of the human subject population a vaccine or immunogenic composition comprising an antigen from a Zika virus as a first and a second
   administration, wherein
   the antigen is an inactivated whole Zika virus, and
   the individuals of the human subject population are flavivirus naïve, and
   administering of the vaccine or immunogenic composition to the individuals of the human subject population induces 182 days after the second administration a seroconversion rate of at least 70%, at least 80%, at least 85%, at least 90%, at least 95%, at least 98%, or 100% in said human subject population as determined by a reporter virus particle assay.
5. A method for inducing an immune response against Zika virus and/or preventing Zika virus disease and/or preventing Zika virus infection in a human subject population in need thereof, optionally according to any one of items 1 to 4, the method comprising administering to the individuals of the human subject population a vaccine or immunogenic composition comprising an antigen from a Zika virus as a first and a second administration, wherein
   the antigen is an inactivated whole Zika virus,
   optionally the individuals of the human subject population are flavivirus naïve, and
   administering of the vaccine or immunogenic composition to the individuals of the human subject population induces 182 days after the second administration a seropositivity rate of at least 70%, at least 80%, at least 85%, at least 90%, at least 95%, at least 98%, or 100% in said human subject population as determined by a reporter virus particle assay.
6. A method for inducing an immune response against Zika virus and/or preventing Zika virus disease and/or preventing Zika virus infection in a human subject population in need thereof, optionally according to any one of items 1 to 5, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 182 days after the second administration geometric mean neutralizing antibody titers of greater than 300, or greater than 600, or greater than 1200 in said human subject population as determined by a reporter virus particle assay.
7. A method for inducing an immune response against Zika virus and/or preventing Zika virus disease and/or preventing Zika virus infection in a human subject population in need thereof, optionally according to any one of items 1 to 6, the method comprising administering to the individuals of the human subject population a vaccine or immunogenic composition comprising an antigen from a Zika virus as a first and a second administration, wherein
   the antigen is an inactivated whole Zika virus, and
   the the individuals of the human subject population are flavivirus naïve, and
   administering of the vaccine or immunogenic composition to the individuals of the human subject population induces 364 days after the second administration a seroconversion rate of at least 70%, at least 80%, at least 85%, at least 90%, at least 95%, at least 98%, or 100% in said human subject population as determined by a plaque reduction neutralization test.
8. A method for inducing an immune response against Zika virus and/or preventing Zika virus disease and/or preventing Zika virus infection in a human subject population in need thereof, optionally according to any one of items 1 to 7, the method comprising administering to the individuals of the human subject population a vaccine or immunogenic composition comprising an antigen from a Zika virus as a first and a second administration, wherein
   the antigen is an inactivated whole Zika virus,
   optionally the individuals of the human subject population are flavivirus naïve, and
   administering of the vaccine or immunogenic composition to the individuals of the human subject population induces 364 days after the second administration a seropositivity rate of at least 70%, at least 80%, at least 85%, at least 90%, at least 95%, at least 98%, or 100% in said human subject population as determined by a plaque reduction neutralization test.
9. A method for inducing an immune response against Zika virus and/or preventing Zika virus disease and/or preventing Zika virus infection in a human subject population in need thereof, optionally according to any one of items 1 to 8, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 364 days after the second administration geometric mean neutralizing antibody titers of greater than 150, or greater than 350, or greater than 400 in said human subject population as determined by a plaque reduction neutralization test.
10. A method for inducing an immune response against Zika virus and/or preventing Zika virus disease and/or preventing Zika virus infection in a human subject population in need thereof, optionally according to any one of items 1 to 9, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 364 days after the second administration a seroconversion rate of at least 70%, at least 80%, at least 85%, at least 90%, at least 95%, at least 98%, or 100% in said human subject population as determined by a reporter virus particle assay.
11. A method for inducing an immune response against Zika virus and/or preventing Zika virus disease and/or preventing Zika virus infection in a human subject population in need thereof, optionally according to any one of items 1 to 10, the method comprising administering to the individuals of the human subject population a vaccine or immunogenic composition comprising an antigen from a Zika virus as a first and a second administration, wherein
   the antigen is an inactivated whole Zika virus,
   optionally the the individuals of the human subject population are flavivirus naïve, and
   administering of the vaccine or immunogenic composition to the individuals of the human subject population induces 364 days after the second administration a seropositivity rate of at least 70%, at least 80%, at least 85%, at least 90%, at least 95%, at least 98%, or 100% in said human subject population as determined by a reporter virus particle assay.
12. A method for inducing an immune response against Zika virus and/or preventing Zika virus disease and/or preventing Zika virus infection in a human subject population in need thereof, optionally according to any one of items 1 to 11, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 364 days after the second administration geometric mean neutralizing antibody titers of greater than 300, or greater than 600, or greater than 700 in said human subject population as determined by a reporter virus particle assay.
13. A method for inducing an immune response against Zika virus and/or preventing Zika virus disease and/or preventing Zika virus infection in a human subject population in need thereof, optionally according to any one of items 1 to 12, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 728 days after the second administration a seroconversion rate of at least 70%, at least 80%, at least 85%, at least 90%, or at least 95% in said human subject population as determined by a plaque reduction neutralization test.
14. A method for inducing an immune response against Zika virus and/or preventing Zika virus disease and/or preventing Zika virus infection in a human subject population in need thereof, optionally according to any one of items 1 to 13, the method comprising administering to the individuals of the human subject population a vaccine or immunogenic composition comprising an antigen from a Zika virus as a first and a second administration, wherein
   the antigen is an inactivated whole Zika virus,
   optionally the the individuals of the human subject population are flavivirus naïve, and
   administering of the vaccine or immunogenic composition to the individuals of the human subject population induces 728 days after the second administration a seropositivity rate of at least 70%, at least 80%, at least 85%, at least 90%, or at least 95% in said human subject population as determined by a plaque reduction neutralization test.
15. A method for inducing an immune response against Zika virus and/or preventing Zika virus disease and/or preventing Zika virus infection in a human subject population in need thereof, optionally according to any one of items 1 to 14, the method comprising administering to the individuals of the human subject population a vaccine or immunogenic composition comprising an antigen from a Zika virus as a first and a second administration, wherein
   the antigen is an inactivated whole Zika virus, and
   the the individuals of the human subject population are flavivirus naïve, and
   administering of the vaccine or immunogenic composition to the individuals of the human subject population induces 728 days after the second administration geometric mean neutralizing antibody titers of greater than 150, or greater than 350, or greater than 400 in said human subject population as determined by a plaque reduction neutralization test.
16. A method for inducing an immune response against Zika virus and/or preventing Zika virus disease and/or preventing Zika virus infection in a human subject population in need thereof, optionally according to any one of items 1 to 15, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 728 days after the second administration a seroconversion rate of at least 70%, at least 80%, at least 85%, at least 90%, or at least 95% in said human subject population as determined by a reporter virus particle assay.
17. A method for inducing an immune response against Zika virus and/or preventing Zika virus disease and/or preventing Zika virus infection in a human subject population in need thereof, optionally according to any one of items 1 to 16, the method comprising administering to the individuals of the human subject population a vaccine or immunogenic composition comprising an antigen from a Zika virus as a first and a second administration, wherein
   the antigen is an inactivated whole Zika virus,
   optionally the individuals of the human subject population are flavivirus naïve, and
   administering of the vaccine or immunogenic composition to the individuals of the human subject population induces 728 days after the second administration a seropositivity rate of at least 70%, at least 80%, at least 85%, at least 90%, or at least 95% in said human subject population as determined by a reporter virus particle assay.
18. A method for inducing an immune response against Zika virus and/or preventing Zika virus disease and/or preventing Zika virus infection in a human subject population in need thereof, optionally according to any one of items 1 to 17, the method comprising administering to the individuals of the human subject population a vaccine or immunogenic composition comprising an antigen from a Zika virus as a first and a second administration, wherein
   the antigen is an inactivated whole Zika virus, and
   the individuals of the human subject population are flavivirus naïve, and
   administering of the vaccine or immunogenic composition to the individuals of the human subject population induces 728 days after the second administration geometric mean neutralizing antibody titers of greater than 300, or greater than 500, or greater than 600 in said human subject population as determined by a reporter virus particle assay.
19. A method for inducing an immune response against Zika virus and/or preventing Zika virus disease and/or preventing Zika virus infection in a human subject population in need thereof, optionally according to any one of items 1 to 18, the method comprising administering to the individuals of the human subject population a vaccine or immunogenic composition comprising an antigen from a Zika virus as a first and a second administration, wherein
   the antigen is an inactivated whole Zika virus, and
   the ratio of the seropositivity rate induced in said human subject population at 182 days after the second administration and the seropositivity rate induced in said human subject population at 28 days after the second administration (SPR at 182 days post-dose 2/SPR at 28 days post-dose 2) is greater than 0.8 or greater than 0.9 or greater than 0.95 or 1 as determined by a plaque reduction neutralization test, optionally wherein the individuals of said human subject population are flavivirus naive.
20. A method for inducing an immune response against Zika virus and/or preventing Zika virus disease and/or preventing Zika virus infection in a human subject population in need thereof, optionally according to any one of items 1 to 19, the method comprising administering to the individuals of the human subject population a vaccine or immunogenic composition comprising an antigen from a Zika virus as a first and a second administration, wherein
   the antigen is an inactivated whole Zika virus, and
   the ratio of the seropositivity rate induced in said human subject population at 364 days after the second administration and the seropositivity rate induced in said human subject population at 28 days after the second administration (SPR at 364 days post-dose 2/SPR at 28 days post-dose 2) is greater than 0.8 or greater than 0.9 or greater than 0.95 or 1 as determined by a plaque reduction neutralization test, optionally wherein the individuals of said human subject population are flavivirus naive.
21. A method for inducing an immune response against Zika virus and/or preventing Zika virus disease and/or preventing Zika virus infection in a human subject population in need thereof, optionally according to any one of items 1 to 20, the method comprising administering to the individuals of the human subject population a vaccine or immunogenic composition comprising an antigen from a Zika virus as a first and a second administration, wherein
   the antigen is an inactivated whole Zika virus, and
   the ratio of the seropositivity rate induced in said human subject population at 728 days after the second administration and the seropositivity rate induced in said human subject population at 28 days after the second administration (SPR at 728 days post-dose 2/SPR at 28 days post-dose 2) is greater than 0.7, or greater than 0.8 or greater than 0.9 as determined by a plaque reduction neutralization test, optionally wherein the individuals of said human subject population are flavivirus naive.
22. The method according to any one of items 1 to 21, wherein the human subject population comprises at least 15 individuals.
23. The method according to any one of items 1 to 22, wherein the vaccine or immunogenic composition is administered as a first and a second administration, wherein the first and the second administration take place from about 1 to about 16 weeks apart or from about 1 to about 6 weeks apart or from about 1 to about 4 weeks apart.
24. The method according to item 23, wherein the vaccine or immunogenic composition is administered as a first and a second administration, wherein the first and the second administration take place from about 25 to 30 days apart, such as 28 days apart.
25. The method according to any of the items above, wherein the vaccine or immunogenic composition comprises a dose of from about 1 μg to about 40 μg of the inactivated whole Zika virus.
26. The method according to any of the items above, wherein the vaccine or immunogenic composition comprises a dose of from about 1 μg to about 20 μg of the inactivated whole Zika virus.
27. The method according to any of the items above, wherein the vaccine or immunogenic composition comprises a dose of from about 1 μg to about 15 μg of the inactivated whole Zika virus.
28. The method according to any of the items above, wherein the vaccine or immunogenic composition comprises a dose of from about 5 μg to about 15 μg of the inactivated whole Zika virus.
29. The method according to any of the items above, wherein the vaccine or immunogenic composition comprises a dose of from about 6 μg to about 15 μg of the inactivated whole Zika virus.
30. The method according to any of the items above, wherein the vaccine or immunogenic composition comprises a dose of from about 7 μg to about 15 μg of the inactivated whole Zika virus.
31. The method according to any of the items above, wherein the vaccine or immunogenic composition comprises a dose of from about 7 μg to about 13 μg of the inactivated whole Zika virus.
32. The method according to any of the items above, wherein the vaccine or immunogenic composition comprises a dose of from about 7.5 μg to about 12.5 μg of the inactivated whole Zika virus.
33. The method according to any of the items above, wherein the vaccine or immunogenic composition comprises a dose of from about 8 μg to about 12 μg of the inactivated whole Zika virus.
34. The method according to any of the items above, wherein the vaccine or immunogenic composition comprises a dose of from about 8.5 μg to about 11.5 μg of the inactivated whole Zika virus.
35. The method according to any of the items above, wherein the vaccine or immunogenic composition comprises a dose of from about 9 μg to about 11 μg of the inactivated whole Zika virus.
36. The method according to any of the items above, wherein the vaccine or immunogenic composition comprises a dose of from about 2 μg to about 10 μg of the inactivated whole Zika virus.
37. The method according to any of the items above, wherein the vaccine or immunogenic composition comprises a dose of about 2 μg of the inactivated whole Zika virus.
38. The method according to any of the items above, wherein the vaccine or immunogenic composition comprises a dose of about 5 μg of the inactivated whole Zika virus.
39. The method according to any of the items above, wherein the vaccine or immunogenic composition comprises a dose of about 10 μg of the inactivated whole Zika virus.
40. The method of any one of items 25 to 39, wherein the main peak of the inactivated whole Zika virus when analyzed by size exclusion chromatography is more than 85% of the total area under the curve in the size exclusion chromatography.
41. The method according to any of the items above, wherein the vaccine or immunogenic composition comprises a dose of from about 6 μg to about 15 μg, such as about 10 μg, of inactivated whole Zika virus and wherein the main peak of the inactivated whole Zika virus when analyzed by size exclusion chromatography is more than 85% of the total area under the curve in the size exclusion chromatography.
42. The method according to any of the items above, wherein the vaccine or immunogenic composition comprises an adjuvant.
43. The method according to item 42, wherein the adjuvant is an aluminum salt.
44. The method according to item 43, wherein the vaccine or immunogenic composition comprises from about 100 μg to about 600 μg, from about 100 μg to about 300 μg, from about 150 μg to about 250 μg, from about 175 μg to about 225 μg, or about 200 μg of an aluminum salt adjuvant.
45. The method according to item 43 or 44, wherein the aluminum salt adjuvant is aluminum hydroxide or aluminum phosphate.
46. The method according to item 45, wherein the aluminum salt adjuvant is aluminum hydroxide.
47. The method according to any of items 42 to 46, wherein at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the antigen are adsorbed to the adjuvant.
48. The method according to any of the items above, wherein the vaccine or immunogenic composition comprises aluminum hydroxide as an adjuvant and wherein at least 95% of the antigen are adsorbed to the adjuvant.
49. The method according to any of the items above, wherein the vaccine or immunogenic composition comprises from about 150 μg to about 250 μg, such as 200 μg, of aluminum hydroxide as an adjuvant.
50. The method according to any of the items above, wherein the vaccine or immunogenic composition comprises from about 175 μg to about 225 μg, such as 200 μg, of aluminum hydroxide as an adjuvant.
51. The method according to any of the items above, wherein the vaccine or immunogenic composition comprises a dose of from about 6 to about 15 μg, such as 10 μg, of inactivated whole Zika virus and from about 150 μg to about 250 μg, such as 200 μg, of aluminum hydroxide as an adjuvant.
52. The method according to any of the items above, wherein the vaccine or immunogenic composition comprises a dose of from about 6 to about 15 μg, such as 10 μg, of inactivated whole Zika virus and from about 175 μg to about 225 μg, such as 200 μg, of aluminum hydroxide as an adjuvant.
53. The method according to item 51 or 52, wherein the main peak of the inactivated whole Zika virus when analyzed by size exclusion chromatography is more than 65% or more than 75% or more than 85% of the total area under the curve in the size exclusion chromatography.
54. A method for inducing an immune response against Zika virus and/or preventing Zika virus disease and/or preventing Zika virus infection in a human subject population in need thereof, the method comprising administering to the individuals of the human subject population a vaccine or immunogenic composition comprising an antigen from a Zika virus as a first and a second administration, wherein
   the antigen is an inactivated whole Zika virus,
   the amount of the antigen in the vaccine or immunogenic composition is from about 6 μg to about 15 μg, such as about 10 μg,
   the vaccine or immunogenic composition further comprises from about 150 μg to 250 μg of an aluminum adjuvant, such as from about 175 μg to about 225 μg of an aluminum salt, optionally of aluminum hydroxide adjuvant,
   the first and the second administration take place from about 25 to 30 days, such as 28 days apart,
   optionally the individuals of the human subject population are flavivirus naïve,
   optionally the human subject population comprises at least 15 individuals, and
   administering of the vaccine or immunogenic composition to the individuals of the human subject population induces 182 days after the second administration a seropositivity rate of at least 84%, or at least 95%, or 100% in said human subject population as determined by a plaque reduction neutralization test.
55. A method for inducing an immune response against Zika virus and/or preventing Zika virus disease and/or preventing Zika virus infection in a human subject population in need thereof, the method comprising administering to the individuals of the human subject population a vaccine or immunogenic composition comprising an antigen from a Zika virus as a first and a second administration, wherein
   the antigen is an inactivated whole Zika virus,
   the amount of the antigen in the vaccine or immunogenic composition is from about 6 μg to about 15 μg, such as about 10 μg,
   the vaccine or immunogenic composition further comprises from about 150 μg to 250 μg of an aluminum adjuvant, such as from about 175 μg to about 225 μg of an aluminum salt, optionally of aluminum hydroxide adjuvant,
   the first and the second administration take place from about 25 to 30 days, such as 28 days apart,
   optionally the individuals of the human subject population are flavivirus naïve,
   optionally the human subject population comprises at least 15 individuals, and
   administering of the vaccine or immunogenic composition to the individuals of the human subject population induces 182 days after the second administration a seropositivity rate of 100% in said human subject population as determined by a plaque reduction neutralization test.
56. A method for inducing an immune response against Zika virus and/or preventing Zika virus disease and/or preventing Zika virus infection in a human subject population in need thereof, optionally according to item 54 or 55, the method comprising administering to the individuals of the human subject population a vaccine or immunogenic composition comprising an antigen from a Zika virus as a first and a second administration, wherein
   the antigen is an inactivated whole Zika virus,
   the amount of the antigen in the vaccine or immunogenic composition is from about 6 μg to about 15 μg, such as about 10 μg,
   the vaccine or immunogenic composition further comprises from about 150 μg to 250 μg of an aluminum adjuvant, such as from about 175 μg to about 225 μg of an aluminum salt, optionally of aluminum hydroxide adjuvant,
   the first and the second administration take place from about 25 to 30 days, such as 28 days apart,
   optionally the individuals of the human subject population are flavivirus naïve,
   optionally the human subject population comprises at least 15 individuals, and
   administering of the vaccine or immunogenic composition to the individuals of the human subject population induces 364 days after the second administration a seropositivity rate of at least 95% or 100% in said human subject population as determined by a plaque reduction neutralization test.
57. A method for inducing an immune response against Zika virus and/or preventing Zika virus disease and/or preventing Zika virus infection in a human subject population in need thereof, optionally according to any one of items 54 to 56, the method comprising administering to the individuals of the human subject population a vaccine or immunogenic composition comprising an antigen from a Zika virus as a first and a second administration, wherein
   the antigen is an inactivated whole Zika virus,
   the amount of the antigen in the vaccine or immunogenic composition is from about 6 μg to about 15 μg, such as about 10 μg,
   the vaccine or immunogenic composition further comprises from about 150 μg to 250 μg of an aluminum adjuvant, such as from about 175 μg to about 225 μg of an aluminum salt, optionally of aluminum hydroxide adjuvant,
   the first and the second administration take place from about 25 to 30 days, such as 28 days apart,
   optionally the individuals of the human subject population are flavivirus naïve,
   optionally the human subject population comprises at least 15 individuals, and
   administering of the vaccine or immunogenic composition to the individuals of the human subject population induces 728 days after the second administration a seropositivity rate of at least 75% or 90% in said human subject population as determined by a plaque reduction neutralization test.
58. A method for inducing an immune response against Zika virus and/or preventing Zika virus disease and/or preventing Zika virus infection in a human subject population in need thereof, the method comprising administering to the individuals of the human subject population a vaccine or immunogenic composition comprising an antigen from a Zika virus as a first and a second administration, wherein
   the antigen is an inactivated whole Zika virus,
   the amount of the antigen in the vaccine or immunogenic composition is from about 6 μg to about 15 μg, such as about 10 μg,
   the vaccine or immunogenic composition further comprises from about 150 μg to 250 μg of an aluminum adjuvant, such as from about 175 μg to about 225 μg of an aluminum salt, optionally of aluminum hydroxide adjuvant,
   the first and the second administration take place from about 25 to 30 days, such as 28 days apart,
   the individuals of the human subject population are flavivirus naïve, optionally wherein the human subject population comprises at least 15 individuals, and
   administering of the vaccine or immunogenic composition to the individuals of the human subject population induces 182 days after the second administration a seroconversion rate of at least 95% or 100% in said human subject population as determined by a plaque reduction neutralization test.
59. A method for inducing an immune response against Zika virus and/or preventing Zika virus disease and/or preventing Zika virus infection in a human subject population in need thereof, optionally according to item 58, the method comprising administering to the individuals of the human subject population a vaccine or immunogenic composition comprising an antigen from a Zika virus as a first and a second administration, wherein
   the antigen is an inactivated whole Zika virus,
   the amount of the antigen in the vaccine or immunogenic composition is from about 6 μg to about 15 μg, such as about 10 μg,
   the vaccine or immunogenic composition further comprises from about 150 μg to 250 μg of an aluminum adjuvant, such as from about 175 μg to about 225 μg of an aluminum salt, optionally of aluminum hydroxide adjuvant,
   the first and the second administration take place from about 25 to 30 days, such as 28 days apart,
   the individuals of the human subject population are flavivirus naïve, optionally wherein the human subject population comprises at least 15 individuals, and
   administering of the vaccine or immunogenic composition to the individuals of the human subject population induces 364 days after the second administration a seroconversion rate of at least 95% or 100% in said human subject population as determined by a plaque reduction neutralization test.
60. A method for inducing an immune response against Zika virus and/or preventing Zika virus disease and/or preventing Zika virus infection in a human subject population in need thereof, optionally according to item 58 or 59, the method comprising administering to the individuals of the human subject population a vaccine or immunogenic composition comprising an antigen from a Zika virus as a first and a second administration, wherein
   the antigen is an inactivated whole Zika virus,
   the amount of the antigen in the vaccine or immunogenic composition is from about 6 μg to about 15 μg, such as about 10 μg,
   the vaccine or immunogenic composition further comprises from about 150 μg to 250 μg of an aluminum adjuvant, such as from about 175 μg to about 225 μg of an aluminum salt, optionally of aluminum hydroxide adjuvant,
   the first and the second administration take place from about 25 to 30 days, such as 28 days apart,
   the individuals of the human subject population are flavivirus naïve, optionally wherein the human subject population comprises at least 15 individuals, and
   administering of the vaccine or immunogenic composition to the individuals of the human subject population induces 728 days after the second administration a seroconversion rate of at least 85% or 90% in said human subject population as determined by a plaque reduction neutralization test.
61. The method according to any of the items 54 to 60, wherein the main peak of the inactivated whole Zika virus when analyzed by size exclusion chromatography is more than 85% of the total area under the curve in the size exclusion chromatography.
62. The method according to any one of items 54 to 61, wherein at least about 95% of the antigen are adsorbed to the adjuvant.
63. The method according to any of the items above, wherein the inactivated whole Zika virus comprises an envelope protein having an amino acid sequence that has at least 70%, at least 75%, at least 80%, at least 85%, at least 90%, at least 95%, or 100% sequence identity with SEQ ID NO: 6.
64. The method according to item 63, wherein the inactivated whole Zika virus comprises an envelope protein represented by SEQ ID NO: 6.
65. The method according to any of the items above, wherein the inactivated whole Zika virus comprises an RNA genome sequence characterized by a DNA sequence having at least 70%, at least 75%, at least 80%, at least 85%, at least 90%, at least 95%, or 100% sequence identity with SEQ ID NO: 5.
66. The method according to item 65, wherein the inactivated whole Zika virus comprises an RNA genome sequence characterized by SEQ ID NO: 5.
67. The method according to any of items 1 to 64, wherein the inactivated whole Zika virus comprises an RNA genome sequence characterized by a DNA sequence having at least 70%, at least 75%, at least 80%, at least 85%, at least 90%, at least 95%, or 100% sequence identity with SEQ ID NO: 3.
68. The method according to item 67, wherein the inactivated whole Zika virus comprises an RNA genome sequence characterized by SEQ ID NO: 3.
69. The method according to any of the items above, wherein the inactivated whole Zika virus comprises a glycine at amino acid position 98 of SEQ ID NO: 8 or at a position equivalent to amino acid position 98 of SEQ ID NO: 8.
70. The method according to any of the items above, wherein the inactivated whole Zika virus was inactivated with formaldehyde.
71. The method according to item 70, wherein the numerical result of the multiplication of the formaldehyde concentration as measured in % (w/v) with the period of incubation with formaldehyde as measured in days is 0.025 to 0.5.
72. The method according to item 70 or 71, wherein the inactivated whole Zika virus was inactivated with 0.005 to 0.02% (w/v) formaldehyde.
73. The method according to any of items 70 to 72, wherein the inactivated whole Zika virus was inactivated with formaldehyde for six to fourteen days.
74. The method according to any of items 70 to 73, wherein the inactivated whole Zika virus was inactivated with formaldehyde at a temperature of 15° C. to 30° C.
75. The method according to any of the items 70 to 74, wherein the inactivated whole Zika virus was inactivated with 0.01% (w/v) formaldehyde.
76. The method according to any of the items 70 to 75, wherein the inactivated whole Zika virus was inactivated with 0.01% (w/v) formaldehyde at a temperature of 22° C.
77. The method according to any of the items 70 to 76, wherein the inactivated whole Zika virus was inactivated with 0.01% (w/v) formaldehyde at a temperature of 22° C. for 10 days.
78. The method according to any of the items above, wherein the vaccine or immunogenic composition comprises a residual formaldehyde content of less than 50 μg/mL.
79. The method according to item 78, wherein the residual formaldehyde content is determined by a method comprising the steps of:
   (a) providing a vaccine or immunogenic composition comprising a Zika virus which has been treated with formaldehyde;
   (b) mixing the vaccine or immunogenic composition of (a) with phosphoric acid and 2,4-dinitrophenylhydrazine (DNPH), thereby providing a mixture;
   (c) incubating the mixture of (b) under suitable conditions; and
   (d) analyzing the mixture for the presence of residual formaldehyde.
80. The method according to any of the items above, wherein the vaccine or immunogenic composition comprises less than 1.0 TCID₅₀ of residual replicating Zika virus.
81. The method according to item 80, wherein residual replicating Zika virus is determined by a method for determining the completeness of inactivation of a Zika virus preparation comprising the steps of:
   (i) inoculating cultured insect cells with a Zika virus preparation which was subjected to an inactivation step and incubating the insect cells for a first period of time, thereby producing an insect cell supernatant;
   (ii) inoculating cultured mammalian cells with the insect cell supernatant produced in (i) and incubating the mammalian cells for a second period of time; and
   (iii) determining whether the Zika virus preparation contains a residual replicating virus that produces a cytopathic effect on the mammalian cells.
82. A vaccine or immunogenic composition comprising an antigen from a Zika virus for use in a method of any one of items 1 to 81, wherein the antigen is an inactivated whole Zika virus.
83. Use of a vaccine or immunogenic composition comprising an antigen from a Zika virus in the manufacture of a medicament for a method of any one of items 1 to 81, wherein the antigen is an inactivated whole Zika virus.

### Third Item List of the Disclosure

1. A method for inducing an immune response against Zika virus in a human subject or a human subject population in need thereof, the method comprising administering to the human subject or the individuals of the human subject population a vaccine or immunogenic composition comprising an antigen from a Zika virus as a first and a second administration, wherein the antigen is an inactivated whole Zika virus and the method does not require a third (booster) administration, or wherein the method consists of a first and second administration.
2. A method of preventing Zika virus disease and/or preventing Zika virus infection in a human subject or a human subject population in need thereof, the method comprising administering to the human subject or the individuals of the human subject population a vaccine or immunogenic composition comprising an antigen from a Zika virus as a first and a second administration, wherein the antigen is an inactivated whole Zika virus and the method does not require a third (booster) administration, or wherein the method consists of a first and second administration.
3. A method of preventing Zika virus disease and/or preventing Zika virus infection in a human fetus or newborn in need thereof, the method comprising administering to a pregnant human subject or a human subject that intends to become pregnant or a woman of childbearing potential a vaccine or immunogenic composition comprising an antigen from a Zika virus as a first and a second administration, wherein the antigen is an inactivated whole Zika virus and the method does not require a third (booster) administration, or wherein the method consists of a first and second administration.
4. A method for inducing an immune response against Zika virus in a human subject or a human subject population in need thereof, the method comprising administering to the human subject or the individuals of the human subject population a vaccine or immunogenic composition comprising an antigen from a Zika virus as a first, second and third (booster) administration, wherein the antigen is an inactivated whole Zika virus.
5. A method of preventing Zika virus disease in a human subject or a human subject population in need thereof, the method comprising administering to the human subject or the individuals of the human subject population a vaccine or immunogenic composition comprising an antigen from a Zika virus as a first, second and third (booster) administration, wherein the antigen is an inactivated whole Zika virus.
6. A method of preventing Zika virus disease in a human fetus or newborn in need thereof, the method comprising administering to a pregnant human subject or a human subject that intends to become pregnant or a woman of childbearing potential a vaccine or immunogenic composition comprising an antigen from a Zika virus as a first, second and third (booster) administration, wherein the antigen is an inactivated whole Zika virus.
7. The method according to any one of items 4 to 6, wherein the third (booster) administration takes place from about 6 to about 24 months after the second administration.
8. The method according to item 7, wherein the third (booster) administration takes place from about 6 to about 12 months after the second administration.
9. The method according to item 7, wherein the third (booster) administration takes place from about 12 to about 24 months after the second administration.
10. The method according to item 7, wherein the third (booster) administration takes about place about 6 months after the second administration.
11. The method according to item 7, wherein the third (booster) administration takes about place about 12 months after the second administration.
12. The method according to item 7, wherein the third (booster) administration takes about place about 24 months after the second administration.
13. The method according to any one of items 4 to 6, wherein the third (booster) administration takes place from about 170 to about 200 days after the second administration.
14. The method according to item 13, wherein the third (booster) administration takes place from about 175 to about 190 days after the second administration.
15. The method according to item 13, wherein the third (booster) administration takes place from about 180 to about 185 days after the second administration.
16. The method according to item 13, wherein the third (booster) administration takes place about 182 days after the second administration.
17. The method according to any one of items 4 to 6, wherein the third (booster) administration takes place at least about 6 months after the second administration.
18. The method according to item 17, wherein the third (booster) administration takes place at least about 12 months after the second administration.
19. The method according to item 17, wherein the third (booster) administration takes place at least about 24 months after the second administration.
20. The method according to any one of items 4 to 6, wherein the third (booster) administration takes place at least about 170 days after the second administration.
21. The method according to item 20, wherein the third (booster) administration takes place at least about 175 days after the second administration.
22. The method according to item 20, wherein the third (booster) administration takes place at least about 180 days after the second administration.
23. The method according to item 20, wherein the third (booster) administration takes place at least about 182 days after the second administration.
24. The method according to any one of items 4 to 6, wherein the third (booster) administration takes place not earlier than about 6 months after the second administration.
25. The method according to any one of items 1 to 24, wherein the first and the second administration take place from about 1 to about 16 weeks apart or from about 1 to about 6 weeks apart or from about 1 to about 4 weeks apart.
26. The method according to any one of items 1 to 24, wherein the first and the second administration take place from about 25 to 30 days apart, such as 28 days apart.
27. The method according to any of the items above, wherein the inactivated whole Zika virus comprises an envelope protein having an amino acid sequence that has at least 70%, at least 75%, at least 80%, at least 85%, at least 90%, at least 95%, or 100% sequence identity with SEQ ID NO: 6.
28. The method according to item 27, wherein the inactivated whole Zika virus comprises an envelope protein represented by SEQ ID NO: 6.
29. The method according to any of the items above, wherein the inactivated whole Zika virus comprises an RNA genome sequence characterized by a DNA sequence having at least 70%, at least 75%, at least 80%, at least 85%, at least 90%, at least 95%, or 100% sequence identity with SEQ ID NO: 5.
30. The method according to item 29, wherein the inactivated whole Zika virus comprises an RNA genome sequence characterized by SEQ ID NO: 5.
31. The method according to any of items 1 to 28, wherein the inactivated whole Zika virus comprises an RNA genome sequence characterized by a DNA sequence having at least 70%, at least 75%, at least 80%, at least 85%, at least 90%, at least 95%, or 100% sequence identity with SEQ ID NO: 3.
32. The method according to item 31, wherein the inactivated whole Zika virus comprises an RNA genome sequence characterized by SEQ ID NO: 3.
33. The method according to any of the items above, wherein the inactivated whole Zika virus comprises a glycine at amino acid position 98 of SEQ ID NO: 8 or at a position equivalent to amino acid position 98 of SEQ ID NO: 8.
34. The method according to any of the items above, wherein the inactivated whole Zika virus was inactivated with formaldehyde.
35. The method according to item 34, wherein the numerical result of the multiplication of the formaldehyde concentration as measured in % (w/v) with the period of incubation with formaldehyde as measured in days is 0.025 to 0.5.
36. The method according to item 34 or 35, wherein the inactivated whole Zika virus was inactivated with 0.005 to 0.02% (w/v) formaldehyde.
37. The method according to any of items 34 to 36, wherein the inactivated whole Zika virus was inactivated with formaldehyde for six to fourteen days.
38. The method according to any of items 34 to 37, wherein the inactivated whole Zika virus was inactivated with formaldehyde at a temperature of 15° C. to 30° C.
39. The method according to any of the items 34 to 38, wherein the inactivated whole Zika virus was inactivated with 0.01% (w/v) formaldehyde.
40. The method according to any of the items 34 to 39, wherein the inactivated whole Zika virus was inactivated with 0.01% (w/v) formaldehyde at a temperature of 22° C.
41. The method according to any of the items 34 to 40, wherein the inactivated whole Zika virus was inactivated with 0.01% (w/v) formaldehyde at a temperature of 22° C. for 10 days.
42. The method according to any of the items above, wherein the vaccine or immunogenic composition comprises a residual formaldehyde content of less than 50 μg/mL.
43. The method according to item 42, wherein the residual formaldehyde content is determined by a method comprising the steps of:
   (a) providing a vaccine or immunogenic composition comprising a Zika virus which has been treated with formaldehyde;
   (b) mixing the vaccine or immunogenic composition of (a) with phosphoric acid and 2,4-dinitrophenylhydrazine (DNPH), thereby providing a mixture;
   (c) incubating the mixture of (b) under suitable conditions; and
   (d) analyzing the mixture for the presence of residual formaldehyde.
44. The method according to any of the items above, wherein the vaccine or immunogenic composition comprises less than 1.0 TCID₅₀ of residual replicating Zika virus.
45. The method according to item 44, wherein residual replicating Zika virus is determined by a method for determining the completeness of inactivation of a Zika virus preparation comprising the steps of:
   (i) inoculating cultured insect cells with a Zika virus preparation which was subjected to an inactivation step and incubating the insect cells for a first period of time, thereby producing an insect cell supernatant;
   (ii) inoculating cultured mammalian cells with the insect cell supernatant produced in (i) and incubating the mammalian cells for a second period of time; and
   (iii) determining whether the Zika virus preparation contains a residual replicating virus that produces a cytopathic effect on the mammalian cells.
46. The method according to any of the items above, wherein the vaccine or immunogenic composition comprises a dose of from about 1 μg to about 40 μg of the inactivated whole Zika virus.
47. The method according to any of the items above, wherein the vaccine or immunogenic composition comprises a dose of from about 1 μg to about 20 μg of the inactivated whole Zika virus.
48. The method according to any of the items above, wherein the vaccine or immunogenic composition comprises a dose of from about 1 μg to about 15 μg of the inactivated whole Zika virus.
49. The method according to any of the items above, wherein the vaccine or immunogenic composition comprises a dose of from about 5 μg to about 15 μg of the inactivated whole Zika virus.
50. The method according to any of the items above, wherein the vaccine or immunogenic composition comprises a dose of from about 6 μg to about 15 μg of the inactivated whole Zika virus.
51. The method according to any of the items above, wherein the vaccine or immunogenic composition comprises a dose of from about 7 μg to about 15 μg of the inactivated whole Zika virus.
52. The method according to any of the items above, wherein the vaccine or immunogenic composition comprises a dose of from about 7 μg to about 13 μg of the inactivated whole Zika virus.
53. The method according to any of the items above, wherein the vaccine or immunogenic composition comprises a dose of from about 7.5 μg to about 12.5 μg of the inactivated whole Zika virus.
54. The method according to any of the items above, wherein the vaccine or immunogenic composition comprises a dose of from about 8 μg to about 12 μg of the inactivated whole Zika virus.
55. The method according to any of the items above, wherein the vaccine or immunogenic composition comprises a dose of from about 8.5 μg to about 11.5 μg of the inactivated whole Zika virus.
56. The method according to any of the items above, wherein the vaccine or immunogenic composition comprises a dose of from about 9 μg to about 11 μg of the inactivated whole Zika virus.
57. The method according to any of the items above, wherein the vaccine or immunogenic composition comprises a dose of from about 2 μg to about 10 μg of the inactivated whole Zika virus.
58. The method according to any of the items above, wherein the vaccine or immunogenic composition comprises a dose of about 2 μg of the inactivated whole Zika virus.
59. The method according to any of the items above, wherein the vaccine or immunogenic composition comprises a dose of about 5 μg of the inactivated whole Zika virus.
60. The method according to any of the items above, wherein the vaccine or immunogenic composition comprises a dose of about 10 μg of the inactivated whole Zika virus.
61. The method of any one of items 46 to 60, wherein the main peak of the inactivated whole Zika virus when analyzed by size exclusion chromatography is more than 65% or more than 75% or more than 85% of the total area under the curve in the size exclusion chromatography.
62. The method according to any of the items above, wherein the vaccine or immunogenic composition comprises a dose of from about 6 μg to about 15 μg, such as about 10 μg, of inactivated whole Zika virus and wherein the main peak of the inactivated whole Zika virus when analyzed by size exclusion chromatography is more than 85% of the total area under the curve in the size exclusion chromatography.
63. The method according to any of the items above, wherein the vaccine or immunogenic composition comprises an adjuvant.
64. The method according to item 63, wherein the adjuvant is an aluminum salt.
65. The method according to item 64, wherein the vaccine or immunogenic composition comprises from about 100 μg to about 600 μg, from about 100 μg to about 300 μg, from about 150 μg to about 250 μg, from about 175 μg to about 225 μg, or about 200 μg of an aluminum salt adjuvant.
66. The method according to item 64 or 65, wherein the aluminum salt adjuvant is aluminum hydroxide or aluminum phosphate.
67. The method according to item 66, wherein the aluminum salt adjuvant is aluminum hydroxide.
68. The method according to any of items 63 to 67, wherein at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the antigen are adsorbed to the adjuvant.
69. The method according to any of the items above, wherein the vaccine or immunogenic composition comprises aluminum hydroxide as an adjuvant and wherein at least 95% of the antigen are adsorbed to the adjuvant.
70. The method according to any of the items above, wherein the vaccine or immunogenic composition comprises from about 150 μg to about 250 μg, such as 200 μg, of aluminum hydroxide as an adjuvant.
71. The method according to any of the items above, wherein the vaccine or immunogenic composition comprises from about 175 μg to about 225 μg, such as 200 μg, of aluminum hydroxide as an adjuvant.
72. The method according to any of the items above, wherein the vaccine or immunogenic composition comprises a dose of from about 6 to about 15 μg, such as 10 μg, of inactivated whole Zika virus and from about 150 μg to about 250 μg, such as 200 μg, of aluminum hydroxide as an adjuvant.
73. The method according to any of the items above, wherein the vaccine or immunogenic composition comprises a dose of from about 6 to about 15 μg, such as 10 μg, of inactivated whole Zika virus and from about 175 μg to about 225 μg, such as 200 μg, of aluminum hydroxide as an adjuvant.
74. The method according to item 72 or 73, wherein the main peak of the inactivated whole Zika virus when analyzed by size exclusion chromatography is more than 85% of the total area under the curve in the size exclusion chromatography.
75. The method according to any of the items above, wherein the vaccine or immunogenic composition is administered in a volume of about 0.5 mL per administration.
76. The method according to any one of the items above, wherein the vaccine or immunogenic composition is administered intramuscularly.
77. The method according to any one of the items above, wherein the (pregnant) human subject or the human subject that intends to become pregnant or the woman of childbearing potential, or the individuals of the human subject population are flavivirus naïve.
78. The method according to any one of the items above, wherein the (pregnant) human subject or the human subject that intends to become pregnant or the woman of childbearing potential, or the individuals of the human subject population are from about 9 to about 65 years of age.
79. The method according to any one of the items above, wherein administering of the vaccine or immunogenic composition induces 182 days after the second administration geometric mean neutralizing antibody titers of at least 500 or at least 600 in the human subject population as determined by a plaque reduction neutralization test.
80. The method according to any one of the items above, wherein administering of the vaccine or immunogenic composition induces 182 days after the second administration geometric mean neutralizing antibody titers of at least 600 or at least 1200 or at least 2400 or at least 3000 in the human subject population as determined by a reporter virus particle assay.
81. The method according to any one of the items above, wherein administering of the vaccine or immunogenic composition induces 182 days after the second administration a seropositivity rate of at least 80% or at least 90% or at least 95% or 100% in the human subject population as determined by a plaque reduction neutralization test.
82. The method according to any one of the items above, wherein administering of the vaccine or immunogenic composition induces 182 days after the second administration a seropositivity rate of at least 80% or at least 80%, or at least 85%, or at least 90% or at least 95% or 100% in the human subject population as determined by a reporter virus particle assay.
83. The method according to any one of the items above, wherein administering of the vaccine or immunogenic composition induces 182 days after the second administration a seroconversion rate of at least 80% or at least 90% or at least 95% or 100% in the human subject population as determined by a plaque reduction neutralization test, wherein the individuals of the human subject population are flavivirus naïve.
84. The method according to any one of the items above, wherein administering of the vaccine or immunogenic composition induces 182 days after the second administration a seroconversion rate of at least 80% or at least 90% or at least 95% or 100% in the human subject population as determined by a reporter virus particle assay, wherein the individuals of the human subject population are flavivirus naïve.
85. The method according to any one of the items above, wherein administering of the vaccine or immunogenic composition induces 182 days after the second administration a 4-fold seroconversion rate of at least 20% or at least 30% or at least 60% in the human subject population as determined by a plaque reduction neutralization test, wherein the individuals of the human subject population are flavivirus primed.
86. The method according to any one of the items above, wherein administering of the vaccine or immunogenic composition induces 182 days after the second administration a 4-fold seroconversion rate of at least 40% or at least 45% or at least 60% or at least 65% in the human subject population as determined by a reporter virus particle assay, wherein the individuals of the human subject population are flavivirus primed.
87. The method according to any of the items above, wherein the human subject population comprises at least 15 individuals.
88. Use of a vaccine or immunogenic composition comprising an antigen from a Zika virus in the manufacture of a medicament for a method according to any one of items 1 to 87, wherein the antigen is an inactivated whole Zika virus.
89. A vaccine or immunogenic composition comprising an antigen from a Zika virus for use in a method according to any one of items 1 to 87, wherein the antigen is an inactivated whole Zika virus.

1. A method of preventing Zika virus disease in a human subject or a human subject population in need thereof, the method comprising administering to the human subject or the individuals of the human subject population a vaccine or immunogenic composition comprising an antigen from a Zika virus as a first, second and third (booster) administration, wherein the antigen is an inactivated whole Zika virus.

2. The method according to claim 1, wherein the third (booster) administration takes place from about 6 to about 24 months after the second administration.

3. The method according to claim 1, wherein the third (booster) administration takes place from about 170 to about 200 days after the second administration.

4. The method according to claim 1, wherein the third (booster) administration takes place at least about 6 months or at least about 12 months or at least about 24 months after the second administration.

5. A method of preventing Zika virus disease in a human subject or a human subject population in need thereof, the method comprising administering to the human subject or the individuals of the human subject population a vaccine or immunogenic composition comprising an antigen from a Zika virus as a first and a second administration, wherein the antigen is an inactivated whole Zika virus and the method does not require a third (booster) administration, or wherein the method consists of a first and second administration.

6. A method for preventing Zika virus disease in a human subject or a human subject population in need thereof, the method comprising administering to the human subject or the individuals of the human subject population a vaccine or immunogenic composition comprising an antigen from a Zika virus, wherein the antigen is an inactivated whole Zika virus and the human subject or the individuals of the human subject population are flavivirus primed.

7. The method of claim 5, wherein
administering of the vaccine or immunogenic composition to the individuals of the human subject population induces 182 days after the second administration a seropositivity rate of at least 70%, at least 80%, at least 85%, at least 90%, at least 95%, at least 98%, or 100% in said human subject population as determined by a plaque reduction neutralization test.

8. The method according to claim 5, wherein the first and the second administration take place from about 1 to about 16 weeks apart or from about 1 to about 6 weeks apart or from about 1 to about 4 weeks apart.

9. The method according to claim 8, wherein the first and the second administration take place from about 25 to 30 days apart, such as 28 days apart.

10. The method according to claim 5, wherein the vaccine or immunogenic composition comprises a dose of from about 1 μg to about 40 μg or from about 6 μg to about 15 μg of the inactivated whole Zika virus.

11. (canceled)

12. The method according to claim 10, wherein the main peak of the inactivated whole Zika virus when analyzed by size exclusion chromatography is more than 65% or more than 75% or more than 85% of the total area under the curve in the size exclusion chromatography.

13. The method according to claim 5, wherein the vaccine or immunogenic composition comprises an aluminum salt adjuvant.

14. The method according to claim 13, wherein the vaccine or immunogenic composition comprises from about 100 μg to about 600 μg, from about 100 μg to about 300 μg, from about 150 μg to about 250 μg, from about 175 μg to about 225 μg, or about 200 μg of an aluminum salt adjuvant.

15. The method according to claim 13, wherein the aluminum salt adjuvant is aluminum hydroxide.

16. The method according to claim 13, wherein at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the antigen are adsorbed to the adjuvant.

17. The method according to claim 5, wherein the inactivated whole Zika virus comprises an envelope protein having an amino acid sequence that has at least 70%, at least 75%, at least 80%, at least 85%, at least 90%, at least 95%, or 100% sequence identity with SEQ ID NO: 6.

18. The method according to claim 5, wherein the inactivated whole Zika virus was inactivated with formaldehyde.

19. The method according to claim 5, wherein the vaccine or immunogenic composition comprises a residual formaldehyde content of less than 50 μg/mL.

20. The method according to claim 5, wherein the vaccine or immunogenic composition comprises less than 1.0 TCID₅₀ of residual replicating Zika virus.

21. The method according to claim 5, wherein instead of the inactivated whole Zika virus,
an antigen from the Zika virus selected from a subunit antigen or a live attenuated virus or a chimeric virus, or
a nucleic acid construct or a viral vector, which is able to express in a human cell a Zika virus antigen,
is administered to the human subject or the individuals of the human subject population.

22. (canceled)

23. (canceled)


SEQUENCE LISTING (11 sequences)

SEQ ID NO: 1 - DNA, 10675 bp, Zika virus strain PRVABC59
gttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaacaataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccaccaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttctgacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccgagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtatttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggatgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaacgggagttatgttagtgccatcacccaagggaggagggaggaagagactcctgttgagtgcttcgagccctcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggcatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggctctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgtcgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgcaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagagga

SEQ ID NO: 2 - protein, 3423 aa, Zika virus strain PRVABC59
MKNPKKKSGGFRIVNMLKRGVARVSPFGGLKRLPAGLLLGHGPIRMVLAILAFLRFTAIKPSLGLINRWGSVGKKEAMETIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYVATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 3 - DNA, 10807 bp, Artificial sequence
  Pre-MVS genomic sequence (P6e)
agttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaacaataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccaccaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttctgacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccgagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatggggagaggtccacagagattgcccgtgcctgtgaacgagctgccccacggctggaaggcttgggggaaatcgtatttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggatgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaacgggagttatgttagtgccatcacccaagggaggagggaggaagagactcctgttgagtgcttcgagccctcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggcatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggctctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgtcgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgcaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 4 - protein, 3423 aa, Artificial sequence
  Zika Pre-MVS polyprotein sequence (P6e)
MKNPKKKSGGFRIVNMLKRGVARVSPFGGLKRLPAGLLLGHGPIRMVLAILAFLRFTAIKPSLGLINRWGSVGKKEAMETIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMGRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYVATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 5 - DNA, 10763 bp, Artificial sequence
  Zika MVS genome sequence (P7e)
cagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaacaataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccaccaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttctgacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccgagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatggggagaggtccacagagattgcccgtgcctgtgaacgagctgccccacggctggaaggcttgggggaaatcgtatttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggatgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaacgggagttatgttagtgccatcacccaagggaggagggaggaagagactcctgttgagtgcttcgagccctcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggcatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggctctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgtcgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgcaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggc

SEQ ID NO: 6 - protein, 504 aa, Artificial sequence
  PRVABC59, Pre-MVS, and MVS envelope (E) protein sequence
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 7 - protein, 168 aa, Artificial sequence
  PRVABC59, Pre-MVS and MVS premembrane/membrane (prM/M) protein sequence
AEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYS

SEQ ID NO: 8 - protein, 351 aa, Artificial sequence
  Zika Pre-MVS and MVS NS1 protein sequence
DVGCSVDFSKKETRCGTGVFVYNDVEAWRDRYKYHPDSPRRLAAAVKQAWEDGICGISSVSRMENIMWRSVEGELNAILEENGVQLTVVVGSVKNPMGRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVT

SEQ ID NO: 9 - protein, 351 aa, Artificial sequence
  Zika virus strain PRVABC59 NS1 protein sequence
DVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVT

SEQ ID NO: 10 - DNA, 1053 bp, Artificial sequence
  Zika Pre-MVS and MVS NS1 gene sequence
gatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatggggagaggtccacagagattgcccgtgcctgtgaacgagctgccccacggctggaaggcttgggggaaatcgtatttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgact

SEQ ID NO: 11 - DNA, 1053 bp, Artificial sequence
  Zika virus strain PRVABC59 NS1 gene sequence
gatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtatttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgact